SEQUENCE LISTING (52 sequences)

SEQ ID NO: 1 - RNA, 75 nt, synthetic construct
  source 1..75: mol_type = other RNA
ggaaatcgcaaaatttgctcttcgcgttagatttcttttagttttctcgcaactagcaagctttttgttctcgcc

SEQ ID NO: 2 - RNA, 203 nt, synthetic construct
  source 1..203: mol_type = other RNA
ggaaatccccacaaccgcctcatatccaggctcaagaatagagctcagtgttttgttgtttaatcattccgacgtgttttgcgatattcgcgcaaagcagccagtcgcgcgcttgcttttaagtagagttgtttttccacccgtttgccaggcatctttaatttaacatatttttatttttcaggctaacctacgccgccacc

SEQ ID NO: 3 - RNA, 76 nt, synthetic construct
  source 1..76: mol_type = other RNA
ggaaataagagagaaaagaagagtaagaagaaatataagatctccctgagcttcagggagccccggcgccgccacc

SEQ ID NO: 4 - RNA, 89 nt, synthetic construct
  source 1..89: mol_type = other RNA
ggaaaccccccacccccgtaagagagaaaagaagagtaagaagaaatataagatctccctgagcttcagggagccccggcgccgccacc

SEQ ID NO: 5 - RNA, 59 nt, synthetic construct
  source 1..59: mol_type = other RNA
ggagaacttccgcttccgttggcgcaagcgctttcattttttctgctaccgtgactaag

SEQ ID NO: 6 - RNA, 46 nt, synthetic construct
  source 1..46: mol_type = other RNA
ggaaataagagagaaaagaagagtaagaagaaatataagagccacc

SEQ ID NO: 7 - RNA, 56 nt, synthetic construct
  source 1..56: mol_type = other RNA
ggaaataagagagaaaagaagagtaagaagaaatataagaccccggcgccgccacc

SEQ ID NO: 8 - RNA, 81 nt, synthetic construct
  source 1..81: mol_type = other RNA
ggaaatcgcaaaatttgctcttcgcgttagatttcttttagttttctcgcaactagcaagctttttgttctcgccgccgcc

SEQ ID NO: 9 - RNA, 81 nt, synthetic construct
  source 1..81: mol_type = other RNA
ggaaatcgcaaaattttcttttcgcgttagatttcttttagttttctttcaactagcaagctttttgttctcgccgccgcc

SEQ ID NO: 10 - RNA, 83 nt, synthetic construct
  source 1..83: mol_type = other RNA
  misc_difference 14..18: note = May be absent
  misc_difference 19: note = May be absent
  misc_difference 22: note = May be absent
  misc_difference 81..83: note = May be absent
  misc_difference 23..27: note = May be absent
ggaaatcgcaaaatttttgctctttttcgcgttagatttcttttagttttctykcaactagcaagctttttgttctcgccrcc

SEQ ID NO: 11 - RNA, 79 nt, synthetic construct
  source 1..79: mol_type = other RNA
ggaaaattttagcctggaacgttagataactgtcctgttgtctttatatacttggtccccaagtagtttgtcttccaaa

SEQ ID NO: 12 - RNA, 79 nt, synthetic construct
  source 1..79: mol_type = other RNA
ggaaactttatttagtgttactttattttctgtttatttgtgtttcttcagtgggtttgttctaatttccttggccgcc

SEQ ID NO: 13 - RNA, 79 nt, synthetic construct
  source 1..79: mol_type = other RNA
ggaaaatctgtattaggttggcgtgttctttggtcggttgttagtattgttgttgattcgtttgtggtcggttgccgcc

SEQ ID NO: 14 - RNA, 79 nt, synthetic construct
  source 1..79: mol_type = other RNA
ggaaaattattaacatcttggtattctcgataaccattcgttggattttattgtattcgtagtttgggttcctgccgcc

SEQ ID NO: 15 - RNA, 79 nt, synthetic construct
  source 1..79: mol_type = other RNA
ggaaattattattatttctagctacaatttatcattgtattattttagctattcatcattatttacttggtgatcaaca

SEQ ID NO: 16 - RNA, 79 nt, synthetic construct
  source 1..79: mol_type = other RNA
ggaaataggttgttaaccaagttcaagcctaataagcttggattctggtgacttgcttcaccgttggcgggcaccgatc

SEQ ID NO: 17 - RNA, 74 nt, synthetic construct
  source 1..74: mol_type = other RNA
ggaaatcgtagagagtcgtacttagtacatatcgactatcggtggacaccatcaagattataaaccaggccaga

SEQ ID NO: 18 - RNA, 79 nt, synthetic construct
  source 1..79: mol_type = other RNA
ggaaacccgcccaagcgaccccaacatatcagcagttgcccaatcccaactcccaacacaatccccaagcaacgccgcc

SEQ ID NO: 19 - RNA, 79 nt, synthetic construct
  source 1..79: mol_type = other RNA
ggaaagcgattgaaggcgtcttttcaactactcgattaaggttgggtatcgtcgtgggacttggaaatttgttgtttcc

SEQ ID NO: 20 - RNA, 79 nt, synthetic construct
  source 1..79: mol_type = other RNA
ggaaactaatcgaaataaaagagccccgtactcttttatttctattaggttaggagccttagcatttgtatcttaggta

SEQ ID NO: 21 - RNA, 79 nt, synthetic construct
  source 1..79: mol_type = other RNA
ggaaatgtgatttccagcaacttcttttgaatatattgaattcctaattcaaagcgaacaaatctacaagccatatacc

SEQ ID NO: 22 - RNA, 74 nt, synthetic construct
  source 1..74: mol_type = other RNA
ggaaatcgtagagagtcgtacttacgtggtcgccattgcatagcgcgcgaaagcaacaggaacaagaacgcgcc

SEQ ID NO: 23 - RNA, 74 nt, synthetic construct
  source 1..74: mol_type = other RNA
ggaaatcgtagagagtcgtacttagaataaacagagtcgggtcgacttgtctctgatactacgacgtcacaatc

SEQ ID NO: 24 - RNA, 79 nt, synthetic construct
  source 1..79: mol_type = other RNA
ggaaaatttgccttcggagttgcgtatcctgaactgcccagcctcctgatatacaactgttccgcttattcgggccgcc

SEQ ID NO: 25 - RNA, 79 nt, synthetic construct
  source 1..79: mol_type = other RNA
ggaaatctgagcaggaatcctttgtgcattgaagactttagattcctctctgcggtagacgtgcacttataagtatttg

SEQ ID NO: 26 - RNA, 79 nt, synthetic construct
  source 1..79: mol_type = other RNA
ggaaagcgattgaaggcgtcttttcaactactcgattaaggttgggtatcgtcgtgggacttggaaatttgttgccacc

SEQ ID NO: 27 - RNA, 104 nt, synthetic construct
  source 1..104: mol_type = other RNA
ggaaatttttttttgatattataagagtttttttttgatattaagaaaatttttttttgatattagaagagtaagaagaaatataagaccccggcgccgccacc

SEQ ID NO: 28 - RNA, 57 nt, synthetic construct
  source 1..57: mol_type = other RNA
ggaaataagagagaaaagaagagtaagaagaaatataagagccaaaaaaaaaaaacc

SEQ ID NO: 29 - RNA, 76 nt, synthetic construct
  source 1..76: mol_type = other RNA
ggaaatctccctgagcttcagggagtaagagagaaaagaagagtaagaagaaatataagaccccggcgccgccacc

SEQ ID NO: 30 - skipped

SEQ ID NO: 31 - RNA, 36 nt, synthetic construct
  source 1..36: mol_type = other RNA
ggactcactatttgttttcgcgcccagttgcaaaaa

SEQ ID NO: 32 - RNA, 119 nt, synthetic construct
  source 1..119: mol_type = other RNA
taaagctccccgggggcctcggtggcctagcttcttgccccttgggcctccccccagcccctcctccccttcctgcacccgtacccccgtggtctttgaataaagtctgagtgggcggc

SEQ ID NO: 33 - RNA, 119 nt, synthetic construct
  source 1..119: mol_type = other RNA
taagtctaagctggagcctcctgagagacctgtgtgaactattgagaagatcggaacagctccttactctgaggaagttggtacccccgtggtctttgaataaagtctgagtgggcggc

SEQ ID NO: 34 - RNA, 185 nt, synthetic construct
  source 1..185: mol_type = other RNA
taaagctccccggggcaaacaccattgtcacactccagcctcggtggcctagcttcttgccccttgggcccaaacaccattgtcacactccatccccccagcccctcctccccttcctgcacccgtaccccccaaacaccattgtcacactccagtggtctttgaataaagtctgagtgggcggc

SEQ ID NO: 35 - RNA, 170 nt, synthetic construct
  source 1..170: mol_type = other RNA
taaagctccccggggtccataaagtaggaaacactacagctggagcctcggtggcctagcttcttgccccttgggcccaaacaccattgtcacactccatccccccagcccctcctccccttcctgcacccgtacccccgtggtctttgaataaagtctgagtgggcggc

SEQ ID NO: 36 - RNA, 141 nt, synthetic construct
  source 1..141: mol_type = other RNA
taaagctccccgggggcctcggtggcctagcttcttgccccttgggcctccccccagcccctcctccccttcctgcacccgtaccccccaaacaccattgtcacactccagtggtctttgaataaagtctgagtgggcggc

SEQ ID NO: 37 - RNA, 185 nt, synthetic construct
  source 1..185: mol_type = other RNA
taagcccctccggggcaaacaccattgtcacactccagcctcggtggcctagcttcttgccccttgggcccaaacaccattgtcacactccatccccccagcccctcctccccttcctgcacccgtaccccccaaacaccattgtcacactccagtggtctttgaataaagtctgagtgggcggc

SEQ ID NO: 38 - RNA, 187 nt, synthetic construct
  source 1..187: mol_type = other RNA
taagcccctccggggtccataaagtaggaaacactacagcctcggtggcctagcttcttgccccttgggcctccataaagtaggaaacactacatccccccagcccctcctccccttcctgcacccgtaccccccgcattattactcacggtacgagtggtctttgaataaagtctgagtgggcggc

SEQ ID NO: 39 - RNA, 170 nt, synthetic construct
  source 1..170: mol_type = other RNA
taagcccctccggggtccataaagtaggaaacactacagctggagcctcggtggcctagcttcttgccccttgggcccaaacaccattgtcacactccatccccccagcccctcctccccttcctgcacccgtacccccgtggtctttgaataaagtctgagtgggcggc

SEQ ID NO: 40 - RNA, 141 nt, synthetic construct
  source 1..141: mol_type = other RNA
taagcccctccgggggcctcggtggcctagcttcttgccccttgggcctccccccagcccctcctccccttcctgcacccgtaccccccaaacaccattgtcacactccagtggtctttgaataaagtctgagtgggcggc

SEQ ID NO: 41 - RNA, 2835 nt, synthetic construct
  source 1..2835: mol_type = other RNA
ggaggaaataagagagaaaagaagagtaagaagaaatataagagccaccatgtgggccgcctgcaccaacttcagcagaaccagaaaggagatccttcttttcgccgagatcatcttgtgccttgtggcccctaaccaggagagcggcatgaagaccgccgacttcctcagagtgctaagcggccacttaatgcagaccagagaggccgagcctaagggcaccatcaccctggagctgatcgagcacaacgaggcctacacctggaccaaccctaccggcggcctggccgtgctggccaaggagcgtcctcctaacctcattgagttcctggccagctacctgctgaagaacaagggcgagcagtacttcaagagaaccatgcctagaatcagcaccctgaagaatctggaggacctggtgaccgagtaccctagaggcatcttcaccaaggaggacgccctgaagttcgtgcagctgaagcagaccggcaagatcaccgagagccctgagaagaccgtgctgacccaggaggccatcatcatcgtgaagggcgtgagcctgtcctcctatctcgaaggccaggcccctgccgtggaggtggctccagccggcgccttctacaaccctagcttcgaggaccaccagaccctgttgtcggacagactgagcaaccacatcagcagcctgttctgtgaggatcagatctacagaatcgaccactacctgggcagcaccgaacacaataaggagtgcctgatcaacatcttcaagtacaagttcagcctggtgatcagcggcctgaccgtgtggagcatcagagtgacgagcacggaggagtacctgcacctgaagcctgccagatacagaagaggcttcatcgagcagagaaacgtgagcggcggttacctggtgctgtgcaagatgaactacgccaccagaatcgtgacactggagtgctcctaccctgagactgaggaggagggcgaagcaattcctgtgagagacagcttctacagactggagaagagactgtggaagcagcagatgtacaccatcgccaagttctacttcttcacacttaagagagagagcaagagcgtgaggcgtctctcagaagaagaactgcctgccatcctgaagcttaagaagagtctgcagaagctggtgagcgactggaacagaggcgcccagctacgcaggcctagacactcacacctgaccagagccagaagatgccctggaggcctccctggccctgccatgaactatgacaagctgagcagaagcctgtgctactactacgagaagggcatcatgcagaaggtggccaccaaggccttcctggacggcttcaacgaagtggccagccttgaatggctgagatacttcgatgagaaggaacttaagtctggttatccagacatcggcttccctctgttcctgctgagtaagggtttcatcaagtccatcagaggcgtggacaaccctggcgtgcaccctgccaccttcgccgcctacactgcaccggcctactacccttacggccagttccagggcagcttcgtccaggccacccctcacaacagagtgagttacttgagagccttctggagatgcttcagaaccgtgggcaccgagaagtgcgaggtggtgatccagcacttcaacagaaagtacctgaagacccctcctggcatccctgcccctaccaagctggacgagctaaagaggcaggaggtgtcacagctccgcatgttacttaaggccaagatggacgccgagaaggccagcaatttgcagaagaataagacagccagcttcaccacaaaggagaaggagacgaagctgcctctgctgcagctcgaagatttggacagccgagtgctttattatttggccacaaagaacggcatcctgtacagagtgggcgacctgggcttacggagcggcagctacagcctgcctcgcattatccacatcgccagaagccagaacttcagaaagagcaagttcggcagcagacactccgcagaatcccagattttcaagcacctccttaagaaccttttcaagatcttctgcaagcagtctcagcacatgaccgaagtggtgagacggtggcctcaccacgagagatgcagcgacagcgacggcctgagccactacagcatctgggaggaagtagtcgcccgtagcttggcacgggtgttagtgttcgaggatgacgtgagaggagtcggcaagggcaagcgacgacctctgaacaacaacttcgagatcgccctttcactgtcaagaaacaaggtgttcgaccctgtgatcgtggaagctttcttagcctcactattcaacaggctgtacatctctcaggaatcagacaaggatcacctgagactggtggagagcgccaatgagaaggctaagcgggaggccctcctgcgcggtgtcgaaccaggtcctcaggagttcttcaagggctacttaaagggagagcacagcagactactctctctctggagagaggtcgtgacattctcccgccaggccagctctgactctgattctatactcttcctcaagagtggtatctccctgggctctcctttccactgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 42 - protein, 854 aa, synthetic construct
MWAACTNFSRTRKEILLFAEIILCLVAPNQESGMKTADFLRVLSGHLMQTREAEPKGTITLELIEHNEAYTWTNPTGGLAVLAKERPPNLIEFLASYLLKNKGEQYFKRTMPRISTLKNLEDLVTEYPRGIFTKEDALKFVQLKQTGKITESPEKTVLTQEAIIIVKGVSLSSYLEGQAPAVEVAPAGAFYNPSFEDHQTLLSDRLSNHISSLFCEDQIYRIDHYLGSTEHNKECLINIFKYKFSLVISGLTVWSIRVTSTEEYLHLKPARYRRGFIEQRNVSGGYLVLCKMNYATRIVTLECSYPETEEEGEAIPVRDSFYRLEKRLWKQQMYTIAKFYFFTLKRESKSVRRLSEEELPAILKLKKSLQKLVSDWNRGAQLRRPRHSHLTRARRCPGGLPGPAMNYDKLSRSLCYYYEKGIMQKVATKAFLDGFNEVASLEWLRYFDEKELKSGYPDIGFPLFLLSKGFIKSIRGVDNPGVHPATFAAYTAPAYYPYGQFQGSFVQATPHNRVSYLRAFWRCFRTVGTEKCEVVIQHFNRKYLKTPPGIPAPTKLDELKRQEVSQLRMLLKAKMDAEKASNLQKNKTASFTTKEKETKLPLLQLEDLDSRVLYYLATKNGILYRVGDLGLRSGSYSLPRIIHIARSQNFRKSKFGSRHSAESQIFKHLLKNLFKIFCKQSQHMTEVVRRWPHHERCSDSDGLSHYSIWEEVVARSLARVLVFEDDVRGVGKGKRRPLNNNFEIALSLSRNKVFDPVIVEAFLASLFNRLYISQESDKDHLRLVESANEKAKREALLRGVEPGPQEFFKGYLKGEHSRLLSLWREVVTFSRQASSDSDSILFLKSGISLGSPFH

SEQ ID NO: 43 - protein, 15 aa, synthetic construct
RASKGVSTSGYSYLH

SEQ ID NO: 44 - protein, 7 aa, synthetic construct
LASYLES

SEQ ID NO: 45 - protein, 9 aa, synthetic construct
QHSRDLPLT

SEQ ID NO: 46 - protein, 111 aa, synthetic construct [sequence omitted]

SEQ ID NO: 47 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 5 aa, synthetic construct
NYYMY

SEQ ID NO: 49 - protein, 17 aa, synthetic construct
GINPSNGGTNFNEKFKN

SEQ ID NO: 50 - protein, 11 aa, synthetic construct
RDYRFDMGFDY

SEQ ID NO: 51 - protein, 120 aa, synthetic construct [sequence omitted]

SEQ ID NO: 52 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2026/0124285 A1
(43) Pub. Date: May 7, 2026

(54) PERSONALIZED CANCER VACCINES

(71) Applicant: ModernaTX, Inc., Cambridge, MA (US)

(72) Inventors: Michelle Brown, Cambridge, MA (US); Igor Feldman, Cambridge, MA (US); Robert Meehan, Cambridge, MA (US); Celine Robert-Tissot, Cambridge, MA (US); Wei Zheng, Cambridge, MA (US)

(73) Assignee: ModernaTX, Inc., Cambridge, MA (US)

(21) Appl. No.: 19/265,457

(22) Filed: Jul. 10, 2025

Related U.S. Application Data
(63) Continuation of application No. PCT/US2024/011156, filed on Jan. 11, 2024.
(60) Provisional application No. 63/438,452, filed on Jan. 11, 2023.
(60) Provisional application No. 63/445,166, filed on Feb. 13, 2023.
(60) Provisional application No. 63/448,235, filed on Feb. 24, 2023.
(60) Provisional application No. 63/490,746, filed on Mar. 16, 2023.
(60) Provisional application No. 63/459,199, filed on Apr. 13, 2023.
(60) Provisional application No. 63/461,141, filed on Apr. 21, 2023.
(60) Provisional application No. 63/505,107, filed on May 31, 2023.
(60) Provisional application No. 63/509,406, filed on Jun. 21, 2023.
(60) Provisional application No. 63/589,621, filed on Oct. 11, 2023.

(51) Int. Cl. A61K 39/00; A61K 39/395; A61P 37/04; C12Q 1/6886; G01N 33/575

(52) U.S. Cl. CPC A61K 39/0011; A61K 39/3955; A61P 37/04; C12Q 1/6886; G01N 33/5758; A61K 2039/505; A61K 2039/53; A61K 2039/55555; A61K 2039/876; C12Q 2600/156; C12Q 2600/158; G01N 2333/70532

(57) ABSTRACT

Personalized mRNA cancer vaccines, as well as methods for their optimization and for their use in subjects are provided. In some embodiments, personalized mRNA cancer vaccines and their uses are provided for subjects having particular characteristics, e.g., the presence of certain biomarkers.

### RELATED APPLICATIONS

This application is a continuation of International Patent Application No. PCT/US2024/011156, filed Jan. 11, 2024, which claims priority under 35 U.S.C. § 119 (e) to U.S. Provisional Application Ser. No. 63/438,452, entitled “PERSONALIZED MRNA CANCER VACCINES”, filed Jan. 11, 2023; U.S. Provisional Application Ser. No. 63/445,166, entitled “PERSONALIZED MRNA CANCER VACCINES”, filed Feb. 13, 2023; U.S. Provisional Application Ser. No. 63/448,235, entitled “PERSONALIZED MRNA CANCER VACCINES”, filed Feb. 24, 2023; U.S. Provisional Application Ser. No. 63/490,746, entitled “PERSONALIZED MRNA CANCER VACCINES”, filed Mar. 16, 2023; U.S. Provisional Application Ser. No. 63/459,199, entitled “PERSONALIZED MRNA CANCER VACCINES”, filed Apr. 13, 2023; U.S. Provisional Application Ser. No. 63/461,141, entitled “PERSONALIZED MRNA CANCER VACCINES”, filed Apr. 21, 2023; U.S. Provisional Application Ser. No. 63/505,107, entitled “PERSONALIZED MRNA CANCER VACCINES”, filed May 31, 2023; U.S. Provisional Application Ser. No. 63/509,406, entitled “PERSONALIZED MRNA CANCER VACCINES”, filed Jun. 21, 2023; and U.S. Provisional Application Ser. No. 63/589,621, entitled “PERSONALIZED CANCER VACCINES”, filed Oct. 11, 2023; the entire contents of each of which are herein incorporated by reference.

### REFERENCE TO AN ELECTRONIC SEQUENCE LISTING

The present application contains a Sequence Listing submitted electronically in XML format, the contents of which are herein incorporated by reference in their entirety. Said XML file, created Jul. 30, 2025, is named “131986-7011_SL.xml” and is 55,392 bytes in size.

### BACKGROUND

Recent breakthroughs in cancer immunotherapy (e.g., checkpoint inhibitors and chimeric antigen receptor-T cell therapies) have demonstrated that powerful anti-tumor responses can be achieved by activating large numbers of T cells in a variety of cancer settings. Several checkpoint inhibitor biologic agents (e.g., anti-CTLA-4 [anti-cytotoxic T lymphocyte-associated antigen-4], anti-PD-1 [anti-programmed cell death protein 1], and anti-PD-L1 [anti-programmed death-ligand 1]) are currently approved for human use in several cancer types, including metastatic melanoma, non-small cell lung carcinoma and bladder carcinoma. These inhibitory receptors and their ligands play complementary roles in down-regulating adaptive immunity; PD-1/PD-L1 contributes to T cell exhaustion in peripheral tissues, and CTLA-4 inhibits earlier T cell activation events (Sharma and Allison 2015). Though it is clear that single agent checkpoint inhibitor therapy can provide significant benefit for some patients, many patients have incomplete or no response to therapy presenting a clear unmet need.

### SUMMARY

Provided herein are personalized cancer vaccines specific to mutations present in a subject's tumor. Also provided are methods of inducing an immune response to a tumor in a subject, e.g., by administering a personalized cancer vaccine to the subject. The present disclosure also provides methods for optimizing personalized cancer vaccines, e.g., to increase their efficacy in stimulating an immune response. The efficacy the vaccines and methods provided herein can, in some embodiments, be related to characteristics of subjects, e.g., certain biomarker(s) in the subjects. Such characteristics can, in some embodiments, be useful in identifying subjects for administration of personalized cancer vaccines and/or predicting subjects' responses to personalized cancer vaccines.

According to some aspects, methods of inducing an immune response against a tumor in a subject are provided herein, the method comprising:
(a) administering to a subject an effective amount of an immune checkpoint inhibitor;
(b) measuring one or more biomarkers or biomarker levels in a biological sample collected from the subject, wherein the measuring is conducted before or on the day of the administering of (c); and
(c) administering to the subject an effective amount of a personalized cancer vaccine, wherein the measurement of the one or more biomarkers or biomarker levels identifies the subject as likely to be responsive to the personalized cancer vaccine, and wherein the personalized cancer vaccine comprises:
(i) an mRNA comprising an open reading frame that encodes at least two cancer antigen epitopes expressed in the tumor in the subject; and
(ii) a lipid delivery vehicle, wherein the administration of the immune checkpoint inhibitor and the personalized cancer vaccine induces an immune response against the tumor in the subject.

In some embodiments, the measuring is conducted within 7 days prior to the administering of (c). In some embodiments, the measuring is conducted on the same day as the administering of (c).

In some embodiments, the measuring is conducted within 90 days prior to the time of the administering of (c).

In some embodiments, the measuring is conducted within 180 days prior to the time of the administering of (c).

In some embodiments, the measuring is conducted within 90 days from the time of the administering of (a). In some embodiments, the measuring is conducted at or approximately at day 90 following the administration of (a).

In some embodiments, the measuring is conducted within 180 days from the time of the administering of (a). In some embodiments, the measuring is conducted at or approximately at day 180 following the administration of (a).

In some embodiments, the method further comprises comparing the measurement of the one or more biomarkers or biomarker levels to predetermined reference values or ranges.

In some embodiments, the one or more biomarkers or biomarker levels comprise tumor mutational burden (TMB), T cell-inflamed gene expression profile (GEP) score, T cell cytotoxicity activity (CYT) score, PD-L1 expression, minimal residual disease (MRD) level, and/or γδ T cells or a sub-type of γδ T cells (e.g., regulatory γδ T cells).

In some embodiments, the one or more biomarkers comprise TMB, wherein the measurement of TMB in the biological sample collected from the subject is less than a predetermined reference value of TMB. In some embodiments, the predetermined reference value of TMB is 175 non-synonymous mutations with an allele frequency of at least 5% per exome.

In some embodiments, the one or more biomarkers comprise T cell-inflamed GEP score, wherein the measurement of T-cell inflamed GEP score in the biological sample collected from the subject is less than a predetermined reference value of T-cell inflamed GEP score. In some embodiments, the predetermined reference value of T cell-inflamed GEP score is 4.

In some embodiments, the one or more biomarkers comprise CYT score, wherein the measurement of CYT score in the biological sample collected from the subject is less than a predetermined reference value of CYT score. In some embodiments, the predetermined reference value of CYT score is 4.

In some embodiments, the one or more biomarkers comprise PD-L1 expression, wherein the measurement of PD-L1 expression in the biological sample collected from the subject is less than a predetermined reference value of PD-L1 expression. In some embodiments, the predetermined reference value of PD-L1 expression is 4, when normalized relative to one or more housekeeping genes.

In some embodiments, the one or more biomarkers comprise MRD level, wherein the measurement of MRD level in the biological sample collected from the subject is greater than a predetermined reference value of MRD level. In some embodiments, the predetermined reference value of MRD level is 500 copies per mL of a mutated gene present in the tumor but not in healthy cells of the subject, in a biological sample comprising circulating tumor DNA (ctDNA). In some embodiments, the predetermined reference value of MRD level is detectable ctDNA in a biological sample collected from the subject following primary treatment. In some embodiments, wherein the biological sample is a blood sample.

In some embodiments, the one or more biomarkers comprise γδ T cells or a sub-type of γδ T cells, wherein the measurement of γδ T cells or a sub-type of γδ T cells in the biological sample collected from the subject is less than a predetermined reference value of γδ T cells or a sub-type of γδ T cells, wherein the sub-type of Γδ T cells is regulatory γδ T cells. In some embodiments, the predetermined reference value of γδ T cells or the sub-type of Γδ T cells is 10% of T lymphocytes in peripheral blood mononuclear cells in a biological sample collected from the subject.

In some embodiments, the measurement of at least one of the one or more biomarkers or biomarker levels is higher than a predetermined reference value or range for the biomarker or biomarker level.

In some embodiments, the measurement of at least one of the one or more biomarkers or biomarker levels is lower than a predetermined reference value or range for the biomarker or biomarker level.

In some embodiments, metastasis of the tumor has not been detected in the subject prior to administration of the immune checkpoint inhibitor and/or the personalized cancer vaccine to the subject.

In some embodiments, the lipid delivery vehicle comprises a lipid nanoparticle, a liposome, or a lipoplex.

In some embodiments, the lipid delivery vehicle comprises a lipid nanoparticle comprising an ionizable cationic lipid, a neutral lipid, cholesterol, and a PEG-modified lipid. In some embodiments, the ionizable cationic lipid, the neutral lipid, the cholesterol, and the PEG-modified lipid are in a molar ratio of 20-60 mol % ionizable cationic lipid: 5-25 mol % neutral lipid: 25-55 mol % cholesterol: 0.5-15 mol % PEG-modified lipid. In some embodiments, the ionizable cationic lipid comprises

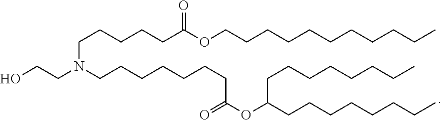 [SMILES: CCCCCCCCCCCOC(=O)CCCCCN(CCO)CCCCCCCC(=O)OC(CCCCCCCC)CCCCCCCC]

In some embodiments, the neutral lipid comprises 1,2-distearoyl-sn-glycero-3-phosphocholine (DSPC). In some embodiments, the PEG-modified lipid comprises 1,2-dimyristoyl-sn-glycerol methoxypolyethylene glycol (PEG-DMG).

In some embodiments, the immune checkpoint inhibitor is an antibody or fragment thereof. In some embodiments, the antibody or fragment thereof specifically binds to a molecule selected from the group consisting of PD-1, TIM-3, VISTA, A2AR, B7-H3, B7-H4, BTLA, CTLA-4, IDO, KIR and LAG3.

In some embodiments, the immune checkpoint inhibitor is an anti-PD-1 antibody or antigen-binding fragment thereof. In some embodiments, the anti-PD-1 antibody or antigen-binding fragment thereof comprises:
(i) light chain complementarity determining regions (CDRs) comprising a sequence of amino acids as set forth in SEQ ID NOs: 43, 44 and 45 and heavy chain CDRs comprising a sequence of amino acids as set forth in SEQ ID NOs: 48, 49 and 50;
(ii) a light chain variable region comprising SEQ ID NO:46 and a heavy chain variable region comprising SEQ ID NO:51; and/or
(iii) a light chain comprising SEQ ID NO: 47 and a heavy chain comprising SEQ ID NO:52. In some embodiments, the anti-PD-1 antibody or antigen-binding fragment thereof is pembrolizumab or a variant thereof.

In some embodiments, the immune checkpoint inhibitor and/or the personalized cancer vaccine is administered to the subject following surgical resection of a primary tumor from the subject.

In some embodiments, the immune response to the tumor comprises an increase in a population of T cells specific to at least one of the cancer antigen epitopes in a biological sample collected from the subject, relative to the population of T cells in a comparable biological sample collected from the subject prior to induction of the immune response to the tumor. In some embodiments, the population of T cells is detectable in a pre-treatment biological sample collected from the subject prior to administration of the personalized cancer vaccine and/or the immune checkpoint inhibitor to the subject.

In some embodiments, the biological sample comprises peripheral blood mononuclear cells.

In some embodiments, a first T cell response to one of the cancer antigen epitopes is detectable in the subject following administration of the personalized cancer vaccine to the subject. In some embodiments, additional T cell responses to an additional one or more of the cancer antigen epitopes are detectable in the subject following administration of the personalized cancer vaccine to the subject.

In some embodiments, the first T cell response is not detectable in the subject prior to administration of the personalized cancer vaccine to the subject. In some embodiments, the additional T cell responses are not detectable in the subject prior to administration of the personalized cancer vaccine to the subject.

In some embodiments, a preexisting T cell response to a first cancer antigen epitope of the cancer antigen epitopes is detectable in the subject prior to administration of the personalized cancer vaccine, and the magnitude of the preexisting T cell response is increased following administration of the personalized cancer vaccine to the subject relative to the magnitude prior to administration of the personalized cancer vaccine. In some embodiments, the magnitude of the preexisting T cell response corresponds to a ratio of T cells responsive to the first cancer antigen epitope to a total number of T cells in a biological sample, or the magnitude of the preexisting T cell response corresponds to an increased strength of response per cell to the first cancer antigen epitope.

In some embodiments, the first T cell response and/or the preexisting T cell response can be detected and/or quantified by collecting a biological sample comprising peripheral blood mononuclear cells (PBMCs) from the subject, stimulating the PBMCs with the cancer antigen epitopes, and subsequently measuring cytokine production by the PBMCs.

In some embodiments, the mRNA of the personalized cancer vaccine encodes 0, 1, 2, 3, 4, 5, 6, 7, 8, 9, 10, or more peptides corresponding to driver mutations, and/or the mRNA of the personalized cancer vaccine encodes 34 or about 34 cancer antigen epitopes expressed in the tumor in the subject.

In some embodiments, the tumor comprises resected stage III or stage IV melanoma. In some embodiments, the tumor comprises resected stage II melanoma. In some embodiments, the tumor comprises resected cutaneous melanoma. In some embodiments, the tumor has a BRAF mutation.

In some embodiments, the tumor comprises non-small cell lung cancer. In some embodiments, the non-small cell lung cancer comprises resected stage II non-small cell lung cancer, resected stage III non-small cell lung cancer, resected stage IIIA non-small cell lung cancer, or resected stage IIIB non-small cell lung cancer.

In some embodiments, the tumor comprises kidney cancer. In some embodiments, the tumor comprises renal cell carcinoma.

In some embodiments, the tumor comprises muscle invasive urothelial carcinoma (MIUC). In some embodiments, the tumor comprises muscle-invasive bladder cancer (MIBC). In some embodiments, the tumor comprises muscle-invasive urinary tract urothelial cancer (UTUC).

In some embodiments, the tumor comprises cutaneous squamous cell carcinoma (cSCC). In some embodiments, the tumor comprises resectable cSCC. In some embodiments, the tumor comprises locally advanced cSCC. In some embodiments, the tumor comprises stage II cSCC. In some embodiments, the tumor comprises stage III cSCC. In some embodiments, the tumor comprises stage IV cSCC.

According to some aspects, a method of inducing an immune response against a tumor in a subject disclosed herein comprises:
(a) administering to a subject an effective amount of an immune checkpoint inhibitor;
(b) administering to the subject an effective amount of a first personalized cancer vaccine, comprising: an mRNA comprising an open reading frame that encodes at least two cancer antigen epitopes expressed in the tumor in the subject; and a lipid delivery vehicle,
(c) measuring one or more biomarkers or biomarker levels in a biological sample collected from the subject, wherein the measuring is conducted after the administering of (b); and
(d) administering to the subject an effective amount of a second personalized cancer vaccine, comprising: an mRNA comprising an open reading frame that encodes at least two cancer antigen epitopes expressed in the tumor in the subject; and a lipid delivery vehicle, wherein the measurement of the one or more biomarkers or biomarker levels identifies the subject as likely to be responsive to the second personalized cancer vaccine,
wherein the administration of the immune checkpoint inhibitor and the first and second personalized cancer vaccine induces an immune response against the tumor in the subject. In some embodiments, the measuring is conducted within 7 days of the administering of (b). In some embodiments, the measuring is conducted on the same day as the administering of (b). In some embodiments, the measuring is conducted within 90 days from the time of the administering of (a). In some embodiments, the measuring is conducted within 90 days from the time of the administering of (d). In some embodiments, the measuring is conducted within 180 days from the time of the administering of (a). In some embodiments, the measuring is conducted within 180 days from the time of the administering of (d).

Each of the limitations of the disclosure can encompass various embodiments of the disclosure. It is, therefore, anticipated that each of the limitations of the disclosure involving any one element or combinations of elements can be included in each aspect of the disclosure. This disclosure is not limited in its application to the details of construction and the arrangement of components set forth in the following description or illustrated in the drawings. The disclosure is capable of other embodiments and of being practiced or of being carried out in various ways.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows recurrence-free survival (RFS) Kaplan-Meier curves for patients with high TMB values treated with pembrolizumab (“TMB high: pembrolizumab”; *), high TMB values treated with pembrolizumab and a personalized cancer vaccine (“TMB high: pembrolizumab+PCV”; +), low TMB values treated with pembrolizumab (“TMB low: pembrolizumab”; †), or low TMB values treated with pembrolizumab and personalized cancer vaccine (PCV) (“TMB low: pembrolizumab+PCV”; ‡).

FIGS. 2A-2C show recurrence-free survival (RFS) Kaplan-Meier curves for patients with high biomarker values treated with pembrolizumab (“High: pembrolizumab”; *), high biomarker values treated with pembrolizumab and a personalized cancer vaccine (“High: pembrolizumab+PCV”; +), low biomarker values treated with pembrolizumab (“Low: pembrolizumab”; †), or low biomarker values treated with pembrolizumab and PCV (“Low: pembrolizumab+PCV”; ‡). The biomarkers evaluated were T cell-inflamed gene expression score (“GEP”; FIG. 2A), cytotoxicity score (“CYT”; FIG. 2B), and PD-L1 expression (“CD274 (PD-L1)”; FIG. 2C).

FIG. 3 shows a neoantigen algorithm. Analysis of next-generation sequencing (NGS) results was used to identify neoantigens to be incorporated into personalized cancer vaccines. DNA-Seq, DNA sequencing; HLA, human leukocyte antigen; mRNA, messenger RNA; NGS, next-generation sequencing; RNA-Seq, RNA sequencing.

FIG. 4A shows Kaplan-Meier estimates for recurrence-free survival for the intention-to-treat population. The hazard ration and the 95% confidence interval for mRNA-1 plus pembrolizumab versus pembrolizumab was estimated using a Cox proportional hazards model with treatment group as a covariate, stratified by disease stage (stages IIIB or IIIC or IIID vs stage IV) used for randomization. The P-value is based on the one-sided log-rank test stratified by disease stage (stages IIIB or IIIC or IIID vs stage IV) used for randomization.

FIG. 4B shows Kaplan-Meier estimates for distant metastasis-free survival for the intention-to-treat population. The hazard ratio and the 95% confidence interval for mRNA-1 plus pembrolizumab versus pembrolizumab was estimated using a Cox proportional hazards model with treatment group as a covariate, stratified by disease stage (stages IIIB or IIIC or IIID vs stage IV) used for randomization. Distant metastasis-free survival was defined as the time from the date of the first dose of pembrolizumab to the date of the first occurrence of distant metastasis determined by the investigator or death (from any cause), whichever occurred first.

FIG. 5 shows Kaplan-Meier estimates for recurrence-free survival in the per-protocol population. The hazard ratio and 95% confidence interval for mRNA-1 plus pembrolizumab versus pembrolizumab is estimated using a Cox proportional hazards model with treatment group as a covariate, stratified by disease stage (stages IIIB or IIIC or IIID vs stage IV) used for randomization.

FIG. 6 shows a Forest plot of recurrence-free survival according to subgroup. Recurrence-free survival was defined for the purposes of this figure as the time from the date of first dose of pembrolizumab to the date of first recurrence (local, regional, or distant metastasis, including new primary melanoma) determined by the investigator or death (from any cause), whichever occurred first. The hazard ratio for mRNA-1 plus pembrolizumab versus pembrolizumab was estimated using an unstratified Cox proportional hazards regression model with treatment group as a covariate. Subgroups with a sample size <10 are not presented: stage at randomization IIID (n=4), ECOG PS missing (n=3). Indeterminate PD-L1: patients for whom there was no sample to send for PD-L1 evaluation or for whom sample quality or quantity was too low to perform the assay.

FIGS. 7A-7B show Kaplan-Meier estimates for recurrence-free survival in patients with high tumor mutational burden (FIG. 7A) and non-high tumor mutational burden (FIG. 7B). The hazard ratio and 95% confidence interval for mRNA-1 plus pembrolizumab versus pembrolizumab was estimated using an unstratified Cox proportional hazards model with treatment group as a covariate.

FIGS. 8A-8C show distribution of TMB (FIG. 8A), TIS (FIG. 8B), and PD-L1 expression (FIG. 8C) in baseline tumors of subjects in the pembrolizumab monotherapy (“Pembrolizumab”) or combination (“mRNA-1+pembrolizumab”) treatment arms. TMB, tumor mutational burden; TIS, tumor inflammation signature; PD-L1, programmed death ligand-1; CPS, combined positivity score.

FIG. 9 shows recurrence-free survival (RFS) Kaplan-Meier curves by treatment arm stratified by TMB status, for patients with non-high TMB values treated with pembrolizumab (“TMB-non-high: pembro”; †), non-high TMB values treated with pembrolizumab and PCV (“TMB-non-high: mRNA-1+pembro”; ‡), high TMB values treated with pembrolizumab (“TMB-high: pembro”; *), or high TMB values treated with pembrolizumab and a personalized cancer vaccine (“TMB-high: mRNA-1+pembro”; +).

FIG. 10 shows recurrence-free survival (RFS) Kaplan-Meier curves by treatment arm stratified by TIS status, for patients with low TIS values treated with pembrolizumab (“TIS-low: pembro”; †), low TIS values treated with pembrolizumab and PCV (“TIS-low: mRNA-1+pembro”; ‡), high TIS values treated with pembrolizumab (“TIS-high: pembro”; *), or high TIS values treated with pembrolizumab and a personalized cancer vaccine (“TIS-high: mRNA-1+pembro”; +).

FIG. 11 shows recurrence-free survival (RFS) Kaplan-Meier curves by treatment arm stratified by PD-L1 status, PD-L1-negative patients treated with pembrolizumab (“PD-L1-negative: pembro”; †), PD-L1-negative patients treated with pembrolizumab and PCV (“PD-L1-negative: mRNA-1+pembro”; ‡), PD-L1-positive patients treated with pembrolizumab (“PD-L1-positive: pembro”; *), or PD-L1-positive patients treated with pembrolizumab and a personalized cancer vaccine (“PD-L1-positive: mRNA-1+pembro”; +).

FIGS. 12A-12D show T-cell responses to individual mRNA-1 neoantigens in individual patients from the combination arm (FIG. 12A and FIG. 12B) and monotherapy arm (FIG. 12C and FIG. 12D). T-cell responses to individual mRNA-1 neoantigens (x-axis) were assessed directly ex vivo with an IFNγ ELISpot at baseline (PID1) and 8 days after the fourth combination treatment cycle (P6D8). SFU, spot forming unit.

FIG. 13 shows longitudinal pattern of distant metastasis-free survival (DMFS) during and after treatment with mRNA-1+pembrolizumab or pembrolizumab alone.

FIG. 14A shows recurrence-free survival (RFS) Kaplan-Meier curves by treatment arm stratified by circulating tumor DNA (ctDNA) status, for ctDNA-negative patients treated with pembrolizumab and mRNA-1 vaccine (“ctDNA-neg mRNA-1+pembrolizumab”), ctDNA-negative patients treated with pembrolizumab monotherapy (“ctDNA-neg pembrolizumab”), ctDNA-positive patients treated with pembrolizumab and mRNA-1 vaccine (“ctDNA-pos mRNA-1+pembrolizumab”), or ctDNA-positive patients treated with pembrolizumab monotherapy (“ctDNA-pos pembrolizumab”).

FIG. 14B shows distant metastasis-free survival (DMFS) Kaplan-Meier curves by treatment arm stratified by circulating tumor DNA (ctDNA) status, for ctDNA-negative patients treated with pembrolizumab and mRNA-1 vaccine (“ctDNA-neg mRNA-1+pembrolizumab”; ‡), ctDNA-negative patients treated with pembrolizumab monotherapy (“ctDNA-neg pembrolizumab”; †), ctDNA-positive patients treated with pembrolizumab and mRNA-1 vaccine (“ctDNA-pos mRNA-1+pembrolizumab”; +), or ctDNA-positive patients treated with pembrolizumab monotherapy (“ctDNA-pos pembrolizumab”; *).

FIG. 15A shows recurrence-free survival (RFS) Kaplan-Meier curves for ctDNA-negative patients by treatment group, and for ctDNA-positive patients by disease status. Curves are shown for ctDNA-negative patients treated with pembrolizumab monotherapy (“ctDNA-negative: pembrolizumab”; *), ctDNA-negative patients treated with mRNA-1 and pembrolizumab combination therapy (“ctDNA-negative: combination”; †), ctDNA-positive patients showing disease control following treatment (“ctDNA-positive (Disease Control)”; †), and ctDNA-positive patients without disease control following treatment (“ctDNA-positive (No Disease Control)”; +).

FIG. 15B shows distant metastasis-free survival (DMFS) Kaplan-Meier curves for ctDNA-negative patients by treatment group, and for ctDNA-positive patients by disease status. Curves are shown for ctDNA-negative patients treated with pembrolizumab monotherapy (“ctDNA-negative: pembrolizumab”; *), ctDNA-negative patients treated with mRNA-1 and pembrolizumab combination therapy (“ctDNA-negative: combination”; †), ctDNA-positive patients showing disease control following treatment (“ctDNA-positive (Disease Control)”; ‡), and ctDNA-positive patients without disease control following treatment (“ctDNA-positive (No Disease Control)”; +).

FIG. 16A shows recurrence-free survival (RFS) Kaplan-Meier curves for patients grouped by ctDNA longitudinal pattern. Curves are shown for patients who were ctDNA negative (“ctDNA negative”; *), patients who were ctDNA positive and were molecular responders (“ctDNA positive MR”; †), and patients who were ctDNA positive and were molecular non-responders (“ctDNA positive MNR”; ‡).

FIG. 16B shows distant metastasis-free survival (DMFS) Kaplan-Meier curves for patients grouped by ctDNA longitudinal pattern. Curves are shown for patients who were ctDNA negative (“ctDNA negative”; *), patients who were ctDNA positive and were molecular responders (“ctDNA positive MR”; †), and patients who were ctDNA positive and were molecular non-responders (“ctDNA positive MNR”; ‡).

FIG. 17A shows recurrence-free survival (RFS) Kaplan-Meier curves for patients with BRAF V600 [E/K] mutant tumors by treatment group. Curves are shown for patients treated with pembrolizumab monotherapy (“Pembrolizumab”; *) and patients treated with mRNA-1 and pembrolizumab combination therapy (“mRNA-1+Pembrolizumab”; +).

FIG. 17B shows RFS Kaplan-Meier curves for patients with BRAF wild-type tumors by treatment group. Curves are shown for patients treated with pembrolizumab monotherapy (“Pembrolizumab”; *) and patients treated with mRNA-1 and pembrolizumab combination therapy (“mRNA-1+Pembrolizumab”; +).

FIG. 17C shows RFS Kaplan-Meier curves for the subset of patients with BRAF V600 [E/K] mutant tumors who were also ctDNA-negative, by treatment group. Curves are shown for patients treated with pembrolizumab monotherapy (“Pembrolizumab”; *) and patients treated with mRNA-1 and pembrolizumab combination therapy (“mRNA-1+Pembrolizumab”; +).

FIG. 17D shows RFS Kaplan-Meier curves for the subset of patients with BRAF wild-type tumors who were also ctDNA-negative, by treatment group. Curves are shown for patients treated with pembrolizumab monotherapy (“Pembrolizumab”; *) and patients treated with mRNA-1 and pembrolizumab combination therapy (“mRNA-1+Pembrolizumab”; +).

FIG. 18A shows change in target lesion size and T cell responses to personalized cancer vaccine neoantigen peptide pools over time. Abbreviations: PD, progressive disease; SD, stable disease; PR, partial response; SFU, spot-forming unit; P #, pembrolizumab run-in timepoint #; C #, combination mRNA-1+pembrolizumab timepoint #; PMC #, pembrolizumab monotherapy post-vaccine timepoint; dFU, days of follow up (after end of treatment); LOD, limit of detection.

FIG. 18B shows results of phenotyping of neoantigen peptide pool-specific T cells at the C4 timepoint from FIG. 18A, after expansion and restimulation. Abbreviations: V, vehicle; P, neoantigen pool; P #, neoantigen pool #.

FIG. 19A-19C show schematics of mRNA-1 first-in-human phase 1 study design. FIG. 19A shows a schematic of dose escalation and dose expansion for mRNA-1 and lists criteria for Parts A-D of the study. FIG. 19B shows a schematic of a process for development of mRNA-1. Abbreviations for FIGS. 19A and 19B: CRC, colorectal cancer; DNA-Seq, DNA sequencing; HLA, human leukocyte antigen; HNSCC, head and neck squamous cell carcinoma; HPV, human papillomavirus virus; INT, individualized neoantigen therapy; MMR, major molecular response; MSI, microsatellite instability; NGS, next-generation sequencing; NSCLC, non-small cell lung cancer; RNA-Seq, RNA sequencing; TMB, tumor mutational burden; TML, tumor mutational load. FIG. 19C shows a detailed study design for mRNA-1 phase 1 study. Abbreviations: CID1, mRNA-1 cycle 1 day 1; C9D1, mRNA-1 cycle 9 day 1; FU, follow-up; NGS, next-generation sequencing; PID1, pembrolizumab cycle 1 day 1; P3D1, pembrolizumab cycle 3 day 1; P11D1, pembrolizumab cycle 11 day 1; Q3W, every 3 weeks; QC, quality control.

FIG. 20 shows duration of treatment and follow-up of patients treated with 1 mg mRNA-1 monotherapy (Part A) or 1 mg mRNA-1 in combination with pembrolizumab (Part D). Duration bars represent the treatment phase(s) and follow-up for each patient, which was the time from the first dose of study treatment to the last contact date or death, if any, whichever came first. NSCLC, non-small cell lung cancer.

FIGS. 21A-21K show T cell responses to immunogenic neoantigen pools in patients who received mRNA-1 monotherapy or mRNA-1 in combination with pembrolizumab. FIG. 21A Example ELISpot assay for response to neoantigen pools for patient 4 who received mRNA-1 monotherapy; FIG. 21B Quantification of ELISpot assay response to neoantigen pools for patient 4; FIG. 21C Data are plotted as sum of all responses to neoantigen pools for all patients in Part A at indicated timepoints during treatment; FIG. 21D Example ELISpot assay for response to neoantigen pools for patient 7 who received mRNA-1 in combination with pembrolizumab; FIG. 21E Sum of all ELISpot assay responses to neoantigen pools for all patients in Part D; FIGS. 21F, 21G, and 21H Durable T cell responses to neoantigen pools (using ELISpot assay) are plotted for patient 4, patient 1, and patient 7 respectively; FIG. 21I Example flow cytometry gating to quantify IFN-γ and TNF-α responses to neoantigen pools; FIG. 21J Representative quantification plot of IFN-γ and TNF-α responses to neoantigen pools at C4D8 in patient 7; FIG. 21K Summary of IFNγ and TNFα CD4 and CD8 T cell responses from all 10 evaluable patients in Parts A and D. Abbreviations: C4D8, Cycle 4 Day 8; IFNγ, interferon gamma; P, peptide; PHA, phytohemagglutinin; SFU, spot forming units; TNFα, tumor necrosis factor gamma; V, vehicle.

FIGS. 22A-22I show T cell responses to individual neoantigens in patients who received mRNA-1 monotherapy or mRNA-1 in combination with pembrolizumab. FIG. 22A Example ELIspot assay for response to neoantigen pools for patient 7 who received mRNA-1 in combination with pembrolizumab; FIG. 22B Example of immune responses to individual neoantigens for patient 7; FIG. 22C Total number of predicted class 1 and class 2 HLA alleles and the total number of immunogenic epitopes in all patients treated with combination therapy; FIG. 22D Pie charts indicating proportion of immunogenic neoantigens out of the total number of neoantigens included in mRNA-1 for all evaluable patients in Part A (left) and Part D (right); FIG. 22E Total number of epitope responses for all evaluable patients in Parts A (left, labeled “mRNA-1 monotherapy”) and D (right, labeled “mRNA-1+pembrolizumab”); FIG. 22F Summary of predicted HLA IFNγ CD4 and CD8 T cell responses to neoantigens from pre-treatment to C4D8 in patient 7; FIG. 22G Summary of predicted HLA class I and II presentation of neoantigens, and measured CD4 or CD8 T cell responses, in patient 8; FIG. 22H Summary of predicted HLA class I or II neoantigen presentations (left) and measured CD4 and CD8 T cell responses to neoantigens (right) in all evaluable patients in Parts A and D; FIG. 22I Proportion of CD4 and CD8 T cell responses to HLA class I (left), class I and II (middle) and class II (right) presented neoantigens. Abbreviations: C4D8, Cycle 4 Day 8; CEF, cytomegalovirus, Epstein-Barr virus, and influenza virus; HLA, human leukocyte antigen; IFNG, interferon gamma; ND, not determined; P, peptide; PHA, phytohemagglutinin; SFU, spot forming units.

FIGS. 23A-23C show frequency of pre-existing or de novo T cell responses in patients treated with mRNA-1 in combination with pembrolizumab and specificity of neoantigen reactivity in all patients who responded to treatment. FIG. 23A Total frequency of pre-existing (pre-treatment) and de novo (C4D8) responses to neoantigens for all 10 evaluable patients who received mRNA-1 in combination with pembrolizumab in Part D (n=immunogenic antigens across 7 patients analyzed); FIG. 23B Total frequencies of pre-existing (pre-treatment) and de novo (C4D8) HLA class 1 and class 2 alleles for all evaluable patients who received mRNA-1 in combination with pembrolizumab; FIG. 23C Specificity of immune reactivity to all neoantigens for select patients using wild type or mutant neoantigens pre-treatment and at C4D8. Abbreviations: C4D8, Cycle 4 Day 8; HLA, human leukocyte antigen; MT, mutant; WT, wildtype.

FIGS. 24A-24F show mRNA-1 in combination with pembrolizumab activates an adaptive immune response. FIG. 24A Breadth and magnitude of immune response across all patients in the immunogenicity evaluable population from Parts A and D; FIG. 24B Representative bulk PBMC phenotyping of CD8 and CD4 T cells directly ex vivo at pre-treatment and C4D8 from patient 7. FIGS. 24C and 24D Summary data comparing pre-treatment CD8 (FIG. 24C) and CD4 (FIG. 24D) T cell populations across high immune responders (n=2; patient 7 and 6) and low immune responders (n=2; patient 13 and 14) treated with mRNA-1 in combination with pembrolizumab. FIG. 24E For high and low immune responders, pre-treatment quantification of the percentage of CD4 and CD8 T cells expressing granzyme B (left) or PD-1 or TIM-3 (exhausted T cells; middle), and the Th1: Treg ratio (right); FIG. 24F For high and low immune responders, quantification of the change after treatment (values at C4D8 with those at pre-treatment subtracted) in the percentage of CD4 and CD8 T cells expressing granzyme B, the amount of granzyme B expressed from CD8 effector cells (median fluorescence intensity), and in the Th1: Treg ratio. Abbreviations: C4D8, Cycle 4 Day 8; FOXp3, forkhead box P3; GrzmB, granzyme B; PBMC, peripheral blood mononuclear cells; PD-1, program cell death protein 1; SFU, spot forming unit; T-bet, T-box expressed in T cells; Th1, T helper 1; TIM-3, T cell immunoglobulin and mucin domain-containing protein 3; TCM, central memory T cell; TEM, effector memory T cell; TEMRA, terminally differentiated effector memory T cells; Treg, regulatory T cell.

FIGS. 25A-25D show disease status of patients in the study compared with that of broader melanoma populations. FIG. 25A Tumor mutational burden; FIG. 25B tumor inflammation score; FIG. 25C CYT vs reference cohorts; FIG. 25D CD274 (PD-L1) vs reference cohorts. Boxplot designates the median and interquartile range. The thin lines outside the boxplot represent the 1.5*interquartile range. The shaded area represents the density to show the distribution shape of the data. Abbreviations: CYT, cytolytic activity score; INT, individualized neoantigen therapy; TCGA, The Cancer Genome Atlas; TIS, tumor inflammation score; TMB, tumor mutational burden.

FIG. 26 shows an example of characterization of INT neoantigen pool-specific T cells post expansion from a patient with melanoma. The bottom segment of each bar shows IFNγ+TNFα-values; the middle segment of each bar shows IFNγ-TNFα+ values; and the top segment of each bar shows IFNγ+TNFα+ values. Abbreviations: C4D8, Cycle 4 Day 8; INFγ, interferon gamma; P, peptide; TNFα, tumor necrosis factor alpha; V, vehicle.

FIGS. 27A-27F show patient responses to individual neoantigens at pre-treatment and C4D8 for patients who received mRNA-1 monotherapy (FIGS. 27A, 27B) or mRNA-1+pembrolizumab combination therapy (FIGS. 27C, 27D, 27E, 27F), measured using ELISpot assay. Abbreviations: C4D8, Cycle 4 Day 8; SFU, spot forming unit.

FIGS. 28A and 28B show predicted HLA alleles (FIG. 28A) and their associated immunogenic epitopes (FIG. 28B) in patients treated with mRNA-1 monotherapy. FIG. 28A shows total number of predicted HLA-A, HLA-B, and HLA-C alleles targeted by bioinformatics prediction for patients treated with mRNA-1 monotherapy. FIG. 28B shows total number of immunogenic HLA-A and HLA-B epitopes for patients treated with mRNA-1 monotherapy.

FIGS. 29A and 29B show predicted HLA alleles (FIG. 29A) and their associated immunogenic epitopes (FIG. 29B) in patients treated with mRNA-1+pembrolizumab combination therapy. FIG. 29A shows total number of predicted HLA-A, HLA-B, and HLA-C alleles targeted by bioinformatics prediction for patients treated with combination therapy. FIG. 29B shows total number of immunogenic HLA-A and HLA-B epitopes for patients treated with combination therapy.

FIG. 30 shows distribution of T cell subsets pre-treatment and after treatment (C4D8) for high (patient 7 and 6) and low (patient 13 and 14) immune responders. Abbreviations: C4D8, mRNA-1 cycle 4 day 8; DP, double positive; gdTCR, gamma delta T cell receptor; P1D1, pembrolizumab cycle 1 day 1; SP, single positive.

### DETAILED DESCRIPTION

In aspects, the present disclosure relates to methods for improving efficacy of cancer therapy using personalized cancer vaccines. The vaccines increase both the number and antitumor activity of a subject's T cells, such that the subject can mount an effective T cell response that recognizes tumor-specific mutations and/or neoantigens. The tumor mutations and their antigen presenting molecules (i.e., HLA) are unique to each subject, and a personalized antigen/HLA strategy, such as the personalized cancer vaccines of the invention, maximize the personalized immune response. The design of the vaccine which incorporates multiple, subject specific neoantigens may improve clinical benefit for subjects with a variety of cancer types. In some aspects the personalized cancer vaccines may help to prevent the patient's cancer from recurring by instructing their immune system to better identify cancerous tissue derived from the original cancer lesion.

In other aspects, the present disclosure relates to methods of optimizing personalized cancer vaccines, such as to increase their immunogenicity. Observations following administration of a personalized cancer vaccine can be used to generate optimized personalized cancer vaccines. For example, an optimized personalized cancer vaccine may result in immune responses to additional tumor antigens relative to an unoptimized personalized cancer vaccine, and/or may result in increased strength of immune responses to tumor antigens relative to an unoptimized vaccine.

The present disclosure also relates to characteristics for selecting subjects for treatment with personalized cancer vaccines, such as biomarkers that can inform the likelihood of a subject benefitting from being administered a personalized cancer vaccine.

In other aspects, the methods provided herein involve improving other anti-cancer therapies such as checkpoint inhibitor therapies. Immune checkpoint inhibitor efficacy may be driven by blocking the negative signals generated by engagement of these inhibitory receptors on T cells with their ligands on tumors and other immune cells, especially antigen presenting cells. The loss of inhibition following checkpoint blockade allows the subjects' T cells to recognize neoantigens as foreign. Combining the cancer vaccines of the invention with checkpoint inhibitor therapy leads to T cell-mediated destruction of the tumor cells by increasing both the number and antitumor activity of a subject's T cells that recognize tumor-specific mutations/neoantigens. In a newly diagnosed subject, the checkpoint therapy, such as pembrolizumab, may begin as soon as possible. At the same time the subject's tumor sample can be screened for neoantigens and a personalized cancer vaccine may be designed and synthesized. As soon as the vaccine is ready, the subject may be started on the combination treatment. The checkpoint inhibitor may be administered together with the vaccine (i.e., on the same day) or they may be administered separately on different schedules. Subsequently, the subject's tumor-specific immune response can be evaluated and an optimized personalized cancer vaccine can be prepared for subsequent administration to the subject.

The use of mRNA technology allows for induced production of a broad array of secreted, membrane-bound, and intracellular proteins in humans. Antigen-encoded mRNA is an attractive technology platform for neoantigen vaccination as an mRNA vaccine can deliver multiple neoantigens in a single molecule, a vaccine unique to each particular subject can be rapidly manufactured, and the neoantigens are endogenously translated and enter into the natural cellular antigen processing and presentation pathway. Moreover, this mRNA-based vaccine technology overcomes the challenges commonly associated with DNA-based vaccines, such as risk of genome integration or the high doses and devices needed for administration (e.g., electroporation).

Each mRNA cancer vaccine comprises an mRNA encoding multiple neoantigens designed specifically for each individual subject's tumor mutanome and HLA type. This allows for the inclusion of the maximum number of neoantigens while both maintaining a sufficient amount of flanking sequence to facilitate both HLA Class I and Class II presentation of the peptides and retaining an mRNA construct length that can be reliably and rapidly manufactured.

Thus, embodiments provide nucleic acid (e.g., RNA, such as mRNA) vaccines that include one or more nucleic acids having one or more open reading frames encoding peptide epitopes. As provided herein, nucleic acid cancer vaccines encoding peptide epitopes having different properties may be used to induce a balanced immune response, comprising cellular and/or humoral immunity. Methods of treating a patient having cancer with a cancer vaccine having a maximized anti-cancer efficacy for a given set of epitopes is also provided.

### Peptide Epitopes

The nucleic acid cancer vaccines of the disclosure may encode one or more peptide epitopes (which are portions of cancer antigens). Portions of cancer antigens are segments of cancer antigens that are less than the full-length cancer antigen. In some embodiments, the nucleic acid cancer vaccine is composed of open reading frames that may contain any number of peptide epitopes. In some embodiments, the nucleic acid cancer vaccine is composed of open reading frames encoding 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 45 or more, 50 or more, 55 or more, 60 or more, 65 or more, 70 or more, 75 or more, 80 or more, 85 or more, 90 or more, 95 or more, 100 or more, 105 or more, 110 or more, 115 or more, 120 or more, 125 or more, 130 or more, 135 or more, 140 or more, 145 or more, 150 or more, 155 or more, 160 or more, 165 or more, 170 or more, 175 or more, 180 or more, 185 or more, 190 or more, 195 or more, or 200 or more peptide epitopes. In other embodiments, the nucleic acid cancer vaccine is composed of open reading frames encoding 200 or less, 195 or less, 190 or less, 185 or less, 180 or less, 175 or less, 170 or less, 165 or less, 160 or less, 155 or less, 150 or less, 145 or less, 140 or less, 135 or less, 130 or less, 125 or less, 120 or less, 115 or less, 110 or less, 100 or less, 95 or less, 90 or less, 85 or less, 80 or less, 75 or less, 70 or less, 65 or less, 60 or less, 55 or less, 50 or less, 45 or less, 40 or less, 35 or less, 30 or less, 25 or less, 20 or less, 15 or less, or 10 or less, or 5 or less peptide epitopes. In other embodiments, the nucleic acid cancer vaccine is composed of open reading frames encoding up to 200, up to 195, up to 190, up to 185, up to 180, up to 175, up to 170, up to 165, up to 160, up to 155, up to 150, up to 145, up to 140, up to 135, up to 130, up to 125, up to 120, up to 115, up to 110, up to 100, up to 95, up to 90, up to 85, up to 80, up to 75, up to 70, up to 65, up to 60, up to 55, up to 50, up to 45, up to 40, up to 35, up to 30, up to 25, up to 20, up to 15, up to 10 peptide epitopes, up to 5 peptide epitopes, or up to 3 peptide epitopes.

In some embodiments, the nucleic acid vaccine comprises one open reading frame encoding up to 50 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) peptide epitopes. In some embodiments, the nucleic acid vaccine comprises one open reading frame encoding 20-40 (e.g., 25-40, 30-40, 30-35, 20-35, 20-30, 22-27, 26-31, 32-37, or 34-40) peptide epitopes. For example, in some embodiments, the nucleic acid vaccine comprises one open reading frame encoding 30, 31, 32, 33, 34, 35, 36, 37, 38, 39, or 40 peptide epitopes.

In some embodiments, the nucleic acid cancer vaccines and vaccination methods described herein include open reading frames that encode epitopes or antigens based on specific mutations (e.g., neoepitopes) and/or those expressed by cancer-germline genes (e.g., antigens common to tumors found in multiple patients). Some antigens that can be encoded by open reading frames of nucleic acid vaccines disclosed herein correspond to “driver mutations,” which initiate cancer formation or accelerate cancer progression. In some embodiments, the encoded open reading frames of the nucleic acid vaccines do not correspond to, and/or do not comprise portions corresponding to “driver mutations”, e.g., such that the vaccine does not contain any “driver mutations.”

An epitope, also known as an antigenic determinant, as used herein is a portion of an antigen that is recognized by the immune system in the appropriate context, specifically by antibodies, B cells, or T cells. Epitopes may include B cell epitopes (e.g., predicted B cell reactive epitopes) and T cell epitopes (e.g., predicted T cell reactive epitopes). B-cell epitopes (e.g., predicted B cell reactive epitopes) are peptide sequences which are required for recognition by specific antibody producing B-cells. B cell epitopes (e.g., predicted B cell reactive epitopes) refer to a specific region of the antigen that is recognized by an antibody. T-cell epitopes (e.g., predicted T cell reactive epitopes) are peptide sequences which, in association with proteins on APC, are required for recognition by specific T-cells. T cell epitopes (e.g., predicted T cell reactive epitopes) are processed intracellularly and presented on the surface of APCs, where they are bound to MHC molecules including MHC class II and MHC class I molecules. The portion of an antibody that binds to the epitope is called a paratope. An epitope may be a conformational epitope or a linear epitope, based on the structure and interaction with the paratope. A linear, or continuous, epitope is defined by the primary amino acid sequence of a particular region of a protein. The sequences that interact with the antibody are situated next to each other sequentially on the protein, and the epitope can usually be mimicked by a single peptide. Conformational epitopes are epitopes that are defined by the conformational structure of the native protein. These epitopes may be continuous or discontinuous (i.e., may be components of the epitope can be situated on disparate parts of the protein, which are brought close to each other in the folded native protein structure).

Each peptide epitope may be any length that is reasonable for an epitope. In some embodiments, the length of each peptide epitope is not necessarily equal. In some embodiments, each peptide epitope in a nucleic acid cancer vaccine is a different length. In certain embodiments, at least two (e.g., at least 3, at least 4, at least 5, at least 6, at least 7, at least 8, at least 9, at least 10, at least 11, at least 12, at least 13, at least 14, at least 15, and up to and including all) of the peptide epitopes in a nucleic acid cancer vaccine are different lengths.

In some embodiments, the length of at least one (such as one or more, two or more, or all) of the peptide epitopes is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, at least 55, at least 60, at least 65, at least 70, at least 75, at least 80, at least 85, at least 90, at least 95, or at least 100 amino acids. In other embodiments, the length of at least one of the peptide epitopes is 100 or less, 95 or less, 90 or less, 85 or less, 80 or less, 75 or less, 70 or less, 65 or less, 60 or less, 55 or less, 50 or less, 45 or less, 40 or less, 35 or less, 30 or less, 25 or less, 20 or less, 15 or less, 14 or less, 13 or less, 12 or less, 11 or less, 10 or less, 9 or less, 8 or less, 7 or less, 6 or less, 5 or less, 4 or less, 3 or less, or 2 or less amino acids. In other embodiments, the length of at least one of the peptide epitopes is up to 100, up to 95, up to 90, up to 85, up to 80, up to 75, up to 70, up to 65, up to 60, up to 55, up to 50, up to 45, up to 40, up to 35, up to 30, up to 25, up to 20, up to 15, or up to 10 amino acids.

In some embodiments, each of the peptide epitopes encoded by the nucleic acid cancer vaccine may have a different length. In certain embodiments, at least one of the peptide epitopes has a different length than another peptide epitope encoded by the nucleic acid cancer vaccine. Each peptide epitope may be any length that is reasonable for an epitope.

In some embodiments, different percentages of peptide epitope lengths are encoded by the nucleic acids.

All of the percentages described in the following listings may be approximate (i.e., within 5% of the stated amount). The use of the terms “approximate” and “about” is equivalent.

In some embodiments, the percentages of peptide epitope lengths encoded by the nucleic acids may be as follows: about 100%<15 amino acids, about 0%≥15 amino acids; about 95%<15 amino acids, about 5%≥15 amino acids; about 90%<15 amino acids, about 10%≥15 amino acids; about 85%<15 amino acids, about 15%≥15 amino acids; about 80%<15 amino acids, about 20%≥15 amino acids; about 75%<15 amino acids, about 25%≥15 amino acids; about 70%<15 amino acids, about 30%≥15 amino acids; about 65%<15 amino acids, about 35%≥15 amino acids; about 60%<15 amino acids, about 40%≥15 amino acids; about 55%<15 amino acids, about 45%≥15 amino acids; about 50%<15 amino acids, about 50%≥15 amino acids; about 45%<15 amino acids, about 55%≥15 amino acids; about 40%<15 amino acids, about 60%≥15 amino acids; about 35%<15 amino acids, about 65%≥15 amino acids; about 30%<15 amino acids, about 70%≥15 amino acids; about 25%<15 amino acids, about 75%≥15 amino acids; about 20%<15 amino acids, about 80%≥15 amino acids; about 15%<15 amino acids, about 85%≥15 amino acids; about 10%<15 amino acids, about 90%≥15 amino acids; about 5%<15 amino acids, about 95%≥15 amino acids; or about 0%<15 amino acids, about 100%≥15 amino acids.

In some embodiments, the peptide epitope lengths may be categorized in one of the following groups (for a total of 100%): 8-12 amino acids, 13-17 amino acids, 18-21 amino acids, 22-26 amino acids, or 27-31 amino acids. About 0%, 5%, 10%, 15%, 20%, 25%, 30%, 35%, 40%, 45%, 50%, 55%, 60%, 65%, 70%, 75%, 80%, 85%, 90%, 95%, or 100% of the peptide epitopes encoded by the open reading frames of the nucleic acids may be 8-12 amino acids in length. About 0%, 5%, 10%, 15%, 20%, 25%, 30%, 35%, 40%, 45%, 50%, 55%, 60%, 65%, 70%, 75%, 80%, 85%, 90%, 95%, or 100% of the peptide epitopes encoded by the open reading frames of the nucleic acids may be 13-17 amino acids in length. About 0%, 5%, 10%, 15%, 20%, 25%, 30%, 35%, 40%, 45%, 50%, 55%, 60%, 65%, 70%, 75%, 80%, 85%, 90%, 95%, or 100% of the peptide epitopes encoded by the open reading frames of the nucleic acids may be 18-21 amino acids in length. About 0%, 5%, 10%, 15%, 20%, 25%, 30%, 35%, 40%, 45%, 50%, 55%, 60%, 65%, 70%, 75%, 80%, 85%, 90%, 95%, or 100% of the peptide epitopes encoded by the open reading frames of the nucleic acids may be 22-26 amino acids in length. About 0%, 5%, 10%, 15%, 20%, 25%, 30%, 35%, 40%, 45%, 50%, 55%, 60%, 65%, 70%, 75%, 80%, 85%, 90%, 95%, or 100% of the peptide epitopes encoded by the open reading frames of the nucleic acids may be 27-31 amino acids in length. Several non-limiting examples of the percentages of peptide epitope lengths encoded by the open reading frames of the nucleic acids follow.

In some embodiments, the peptide epitopes comprise at least one MHC class I epitope and at least one MHC class II epitope. In some embodiments, at least 10% of the peptide epitopes are MHC class I epitopes. In some embodiments, at least 20% of the peptide epitopes are MHC class I epitopes. In some embodiments, at least 30% of the peptide epitopes are MHC class I epitopes. In some embodiments, at least 40% of the peptide epitopes are MHC class I epitopes. In some embodiments, at least 0%, 60%, 70%, 80%, 90%, or 100% of the peptide epitopes are MHC class I epitopes.

In some embodiments, none (0%) of the peptide epitopes are MHC class II epitopes. In some embodiments, at least 10% of the peptide epitopes are MHC class II epitopes. In some embodiments, at least 20% of the peptide epitopes are MHC class II epitopes. In some embodiments, at least 30% of the peptide epitopes are MHC class II epitopes. In some embodiments, at least 40% of the peptide epitopes are MHC class II epitopes. In some embodiments, at least 50%, 60%, 70%, 80%, 90% or 100% of the peptide epitopes are MHC class II epitopes.

In some embodiments, the ratio of MHC class I epitopes to MHC class II epitopes is a ratio selected from about 10%:about 90%; about 20%:about 80%; about 30%:about 70%; about 40%:about 60%; about 50%:about 50%; about 60%:about 40%; about 70%:about 30%; about 80%:about 20%; about 90%:about 10% MHC class I:MHC class II epitopes. In some embodiments, the ratio of MHC class I:MHC class II epitopes is 1:1. In some embodiments, the ratio of MHC class I:MHC class II epitopes is 2:1. In some embodiments, the ratio of MHC class I:MHC class II epitopes is 3:1. In one embodiment, the ratio of MHC class I:MHC class II epitopes is 4:1. In some embodiments, the ratio of MHC class I:MHC class II epitopes is 5:1. In some embodiments, the ratio of MHC class II epitopes to MHC class I epitopes is a ratio selected from about 10%:about 90%; about 20%:about 80%; about 30%:about 70%; about 40%:about 60%; about 50%:about 50%; about 60%:about 40%; about 70%:about 30%; about 80%:about 20%; about 90%:about 10% MHC class II:MHC class I epitopes. In some embodiments, the ratio of MHC class II:MHC class I epitopes is 1:1. In some embodiments, the ratio of MHC class II:MHC class I epitopes is 1:2. In one embodiment, the ratio of MHC class II:MHC class I epitopes is 1:3. In some embodiments, the ratio of MHC class II:MHC class I epitopes is 1:4. In some embodiments, the ratio of MHC class II:MHC class I epitopes is 1:5.

In some embodiments, at least one of the peptide epitopes of the cancer vaccine is a B cell epitope. In some embodiments, one or more predicted T cell reactive epitope of the cancer vaccine comprises between 8-11 amino acids. In some embodiments, one or more predicted B cell reactive epitope of the cancer vaccine comprises between 13-17 amino acids.

The cancer vaccine of the disclosure, in some aspects comprises an mRNA vaccine encoding multiple peptide epitope antigens arranged with an amino acid spacer (e.g., a single amino acid spacer, a double amino acid spacer, a triple amino acid spacer, etc.) between the peptide epitopes, a short linker between the peptide epitopes, or directly to one another without a spacer between the peptide epitopes, or any combination thereof (e.g., some peptide epitopes being directly adjacent to one another, some with a single amino acid spacer between the peptide epitopes, and/or some with a short linker between the peptide epitopes). The multiple epitope antigens may include a mixture of MHC class I epitopes and MHC class II epitopes.

The nucleic acid cancer vaccine, in some aspects, comprises a nucleic acid encoding one or more peptide epitopes that include a mutation causing a unique expressed peptide sequence. In some embodiments, a mutation causing a unique expressed peptide sequence may be, but is not limited to, an insertion, deletion, frameshift mutation, and/or splicing variant. In some embodiments, the nucleic acid cancer vaccine encodes multiple peptide epitope antigens including one or more single nucleotide polymorphism (SNP) mutations with flanking amino acids on each side of the SNP mutation. In some embodiments, the number of flanking amino acids on each side of the SNP mutation may be 5, 6, 7, 8, 9, 10, 11, 12, 13, 14, 15, 16, 17, 18, 19, 20, 22, 24, 26, 28, or 30. In some embodiments, the SNP mutation is centrally located and the number of flanking amino acids on each side of the SNP mutation is approximately the same. In some embodiments, the SNP mutation does not have an equivalent number of flanking amino acids on each side. In some embodiments, an epitope of the cancer vaccine comprises an SNP flanked by two Class I sequences, each sequence comprising seven amino acids. In some embodiments, an epitope of the cancer vaccine comprises a SNP flanked by two Class II sequences, each sequence comprising 10 amino acids. In some embodiments, an epitope may comprise a centrally located SNP and flanks which are both Class I sequences, both Class II sequences, or one Class I and one Class II sequence.

In some embodiments, the peptide epitopes are in the form of a concatemeric cancer antigen comprised of peptide epitopes. Any number of peptide epitopes may be used. In certain embodiments, the peptide epitopes are in the form of a concatemeric cancer antigen comprised of 5-200 peptide epitopes. In certain embodiments, the peptide epitopes are in the form of a concatemeric cancer antigen comprised of 5-130 peptide epitopes. In some embodiments, the concatemeric cancer antigen comprises one or more of: a) the peptide epitopes (e.g., the 5-200 or 5-130 peptide epitopes) are interspersed by cleavage sensitive sites; and/or b) each peptide epitope is linked directly to one another without a linker; and/or c) each peptide epitope is linked to one or another with a single amino acid linker; and/or d) each peptide epitope is linked to one or another with a short linker; and/or e) each peptide epitope comprises 8-31 amino acids and includes one or more SNP mutations (e.g., a centrally located SNP mutation); and/or f) each peptide epitope comprises 8-31 amino acids and includes a mutation causing a unique expressed peptide sequence; and/or g) the nucleic acids encoding the peptide epitopes are arranged such that the peptide epitopes are ordered to minimize pseudo-epitopes, and/or h) no class II MHC molecules peptide epitopes are present.

It will be appreciated that a concatemer of 2 or more peptides, e.g., 2 or more neoantigens, may create unintended new epitopes (pseudoepitopes) at peptide boundaries. To prevent or eliminate such pseudoepitopes, class I alleles may be scanned for hits across peptide boundaries in a concatemer. In some embodiments, the peptide order within the concatemer is shuffled to reduce or eliminate pseudoepitope formation. In some embodiments, a linker is used between peptides, e.g., a single amino acid linker such as glycine (Gly) or a double amino acid linker such as Gly-Gly, to reduce or eliminate pseudoepitope formation. In some embodiments, anchor amino acids can be replaced with other amino acids which will reduce or eliminate pseudoepitope formation. In some embodiments, peptides are trimmed at the peptide boundary within the concatemer to reduce or eliminate pseudoepitope formation.

In some embodiments, the multiple peptide epitope antigens are arranged and ordered to minimize pseudoepitopes. In some embodiments, glycine insertion can be used to disrupt pseudoepitopes. In other embodiments, the multiple peptide epitope antigens are a polypeptide that is free of pseudoepitopes. When the cancer antigen epitopes are arranged in a concatemeric structure in a head to tail formation, a junction is formed between each of the cancer antigen epitopes. That includes several, i.e., 1-10, amino acids from an epitope on a N-terminus of the peptide and several, i.e., 1-10, amino acids on a C-terminus of an adjacent directly linked epitope. It is important that the junction not be an immunogenic peptide that may produce an immune response. In some embodiments, the junction forms a peptide sequence that binds to an HLA protein of a subject for which the personalized cancer vaccine is designed with an IC50 greater than about 50 nM. In other embodiments, the junction peptide sequence binds to an HLA protein of a subject with an IC50 greater than about 10 nM, 150 nM, 200 nM, 250 nM, 300 nM, 350 nM, 400 nM, 450 nm, or 500 nM.

### Personalized Cancer Vaccines

In some aspects, the present disclosure provides a nucleic acid cancer vaccine comprising one or more nucleic acids, wherein each of the nucleic acids encodes at least one suitable cancer antigen such as a personalized antigen specific for a cancer subject. A personalized cancer antigen is a tumor-specific antigen, also referred to as a neoantigen, that is present in a tumor of an individual that is not expressed or expressed at low levels in normal non-cancerous tissue of the individual. The antigen may or may not be present in tumors of other individuals. Herein, a personalized cancer vaccine may also be referred to as a “nucleic acid (cancer) vaccine” and/or an “mRNA (cancer) vaccine”.

For instance, the nucleic acid cancer vaccine may include nucleic acids encoding one or more cancer antigens specific for each subject, referred to as neoepitopes. Antigens that are expressed in or by tumor cells are referred to as “tumor associated antigens.” A particular tumor associated antigen may or may not also be expressed in non-cancerous cells. Many tumor mutations are well known in the art. Tumor associated antigens that are not expressed or rarely expressed in non-cancerous cells, or whose expression in non-cancerous cells is sufficiently reduced in comparison to that in cancerous cells and that induce an immune response induced upon vaccination, are referred to as neoepitopes. Neoepitopes are completely foreign to the body and thus would not normally produce an immune response against healthy tissue or be masked by the protective components of the immune system. In some embodiments, personalized vaccines based on neoepitopes are desirable because such vaccines will maximize specificity against a patient's specific tumor. Mutation-derived neoepitopes can arise from point mutations, non-synonymous mutations leading to different amino acids in the protein; read-through mutations in which a stop codon is modified or deleted, leading to translation of a longer protein with a novel tumor-specific sequence at the C-terminus; splice site mutations that lead to the inclusion of an intron in the mature mRNA and thus a unique tumor-specific protein sequence; chromosomal rearrangements that give rise to a chimeric protein with tumor-specific sequences at the junction of 2 proteins (i.e., gene fusion); frameshift mutations or deletions that lead to a new open reading frame with a novel tumor-specific protein sequence; and/or translocations.

In some embodiments, the nucleic acid cancer vaccines and vaccination methods described herein may include peptide epitopes or antigens based on specific mutations (e.g., neoepitopes) and those expressed by cancer-germline genes (e.g., antigens common to tumors found in multiple patients, referred to herein as “traditional cancer antigens” or “shared cancer antigens”). In some embodiments, a traditional antigen is one that is known to be found in cancers or tumors generally or in a specific type of cancer or tumor. In some embodiments, a traditional cancer antigen is a non-mutated tumor antigen. In some embodiments, a traditional cancer antigen is a mutated tumor antigen.

In some embodiments, the nucleic acid cancer vaccines and methods described herein may include peptide epitopes based on cancer/testis (CT) antigens. Cancer/testis antigen expression is limited to male germ cells in healthy adults, but ectopic expression has been observed in tumor cells of multiple types of human cancer. Since male germ cells are devoid of HLA-class I molecules and cannot present antigens to T cells, cancer/testis antigens are generally considered neoantigens when expressed in cancer cells and have the capacity to elicit immune responses that are strictly cancer-specific. Cancer/testis antigens for use with the compositions and methods described herein may be any such cancer/testis antigen known in the field including, but not limited to, MAGEA1, MAGEA2, MAGEA3, MAGEA4, MAGEA5, MAGEA6, MAGEA8, MAGEA9, MAGEA10, MAGEA11, MAGEA12, BAGE, BAGE2, BAGE3, BAGE4, BAGE5, MAGEB1, MAGEB2, MAGEB5, MAGEB6, MAGEB3, MAGEB4, GAGE1, GAGE2A, GAGE3, GAGE4, GAGE5, GAGE6, GAGE7, GAGE8, SSX1, SSX2, SSX2b, SSX3, SSX4, CTAG1B, LAGE-1b, CTAG2, MAGEC1, MAGEC3, SYCP1, BRDT, MAGEC2, SPANXA1, SPANXB1, SPANXC, SPANXD, SPANXN1, SPANXN2, SPANXN3, SPANXN4, SPANXN5, XAGE1D, XAGE1C, XAGE1B, XAGE1, XAGE2, XAGE3, XAGE-3b, XAGE-4/RP11-167P23.2, XAGE5, DDX43, SAGE1, ADAM2, PAGE5, CT16.2, PAGE1, PAGE2, PAGE2B, PAGE3, PAGE4, LIPI, VENTXP1, IL13RA2, TSP50, CTAGE1, CTAGE-2, CTAGE5, SPA17, ACRBP, CSAG1, CSAG2, DSCR8, MMA1b, DDX53, CTCFL, LUZP4, CASC5, TFDP3, JARID1B, LDHC, MORC1, DKKL1, SPO11, CRISP2, FMR1NB, FTHL17, NXF2, TAF7L, TDRD1, TDRD6, TDRD4, TEX15, FATE1, TPTE, CT45A1, CT45A2, CT45A3, CT45A4, CT45A5, CT45A6, HORMAD1, HORMAD2, CT47A1, CT47A2, CT47A3, CT47A4, CT47A5, CT47A6, CT47A7, CT47A8, CT47A9, CT47A10, CT47A11, CT47B1, SLCO6A1, TAG, LEMD1, HSPB9, CCDC110, ZNF165, SPACA3, CXorf48, THEG, ACTL8, NLRP4, COX6B2, LOC348120, CCDC33, LOC196993, PASD1, LOC647107, TULP2, CT66/AA884595, PRSS54, RBM46, CT69/BC040308, CT70/BI818097, SPINLW1, TSSK6, ADAM29, CCDC36, LOC440934, SYCE1, CPXCR1, TSPY3, TSGA10, HIWI, MIWI, PIWI, PIWIL2, ARMC3, AKAP3, Cxorf61, PBK, C21orf99, OIP5, CEP290, CABYR, SPAG9, MPHOSPH1, ROPN1, PLAC1, CALR3, PRM1, PRM2, CAGE1, TTK, LY6K, IMP-3, AKAP4, DPPA2, KIAA0100, DCAF12, SEMG1, POTED, POTEE, POTEA, POTEG, POTEB, POTEC, POTEH, GOLGAGL2 FA, CDCA1, PEPP2, OTOA, CCDC62, GPATCH2, CEP55, FAM46D, TEX14, CTNNA2, FAM133A, LOC130576, ANKRD45, ELOVL4, IGSF11, TMEFF1, TMEFF2, ARX, SPEF2, GPAT2, TMEM108, NOL4, PTPN20A, SPAG4, MAEL, RQCD1, PRAME, TEX101, SPATA19, ODF1, ODF2, ODF3, ODF4, ATAD2, ZNF645, MCAK, SPAG1, SPAG6, SPAG8, SPAG17, FBXO39, RGS22, cyclin A1, C15orf60, CCDC83, TEKT5, NR6A1, TMPRSS12, TPPP2, PRSS55, DMRT1, EDAG, NDR, DNAJB8, CSAG3B, CTAG1A, GAGE12B, GAGE12C, GAGE12D, GAGE12E, GAGE12F, GAGE12G, GAGE12H, GAGE12I, GAGE12J, GAGE13, LOC728137, MAGEA2B, MAGEA9B/LOC728269, NXF2B, SPANXA2, SPANXB2, SPANXE, SSX4B, SSX5, SSX6, SSX7, SSX9, TSPY1D, TSPY1E, TSPY1F, TSPY1G, TSPY1H, TSPY1I, TSPY2, XAGE1E, XAGE2B/CTD-2267G17.3, and/or variants thereof.

In some embodiments, the nucleic acid cancer vaccines may further include one or more nucleic acids encoding for one or more non-mutated tumor antigens. In some embodiments, the nucleic acid cancer vaccines may further include one or more nucleic acids encoding for one or more mutated tumor antigens.

Many tumor antigens are known in the art. Cancer or tumor antigens (e.g., traditional cancer antigens) for use with the compositions and methods described herein may be any such cancer or tumor antigens known in the field. In some embodiments, the cancer or tumor antigen (e.g., the traditional cancer antigen) is one of the following antigens: CD2, CD19, CD20, CD22, CD27, CD33, CD37, CD38, CD40, CD44, CD47, CD52, CD56, CD70, CD79, CD137, 4-IBB, 5T4, AGS-5, AGS-16, Angiopoietin 2, B2M, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GlTR), gp100, gpA33, GPNMB, ICOS, IGF1R, Integrin av, Integrin αvβ, LAG-3, Lewis Y, Mesothelin, c-MET, MN Carbonic anhydrase IX, MUC1, MUC16, Nectin-4, NKGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, VEGFR-3, and/or variants thereof.

Epitopes can be identified using a free or commercial database (Lonza Epibase, antitope for example). Such tools are useful for predicting the most immunogenic epitopes within a target antigen protein. The selected peptides may then be synthesized and screened in human HLA panels, and the most immunogenic sequences are used to construct the nucleic acids encoding the peptide epitope(s). One strategy for mapping epitopes of Cytotoxic T-Cells based on generating equimolar mixtures of the four C-terminal peptides for each nominal 11-mer across a protein. This strategy would produce a library antigen containing all the possible active CTL epitopes.

The neoepitopes may be designed to optimally bind to MHC in order to promote a robust immune response. In some embodiments, each peptide epitope comprises an antigenic region and a MHC stabilizing region. An MHC stabilizing region is a sequence which stabilizes the peptide in the MHC.

All of the MHC stabilizing regions within the epitopes may be the same or they may be different. The MHC stabilizing regions may be at the N terminal portion of the peptide or the C terminal portion of the peptide. Alternatively the MHC stabilizing regions may be in the central region of the peptide.

The MHC stabilizing region may be 5-10, 5-15, 8-10, 1-5, 3-7, or 3-8 amino acids in length. In yet other embodiments, the antigenic region is 5-100 amino acids in length. The peptides interact with the molecules of MHC class I by competitive affinity binding within the endoplasmic reticulum, before they are presented on the cell surface. The affinity of an individual peptide is directly linked to its amino acid sequence and the presence of specific binding motifs in defined positions within the amino acid sequence. The peptide being presented in the MHC is held by the floor of the peptide-binding groove, in the central region of the α1/α2 heterodimer (a molecule composed of two nonidentical subunits). The sequence of residues of the peptide-binding groove's floor determines which particular peptide residues it binds.

Optimal binding regions may be identified by a computer assisted comparison of the affinity of a binding site (MHC pocket) for a particular amino acid at each amino acid in the binding site for each of the target epitopes to identify an ideal binder for all of the examined antigens. The MHC stabilization regions of the epitopes may be identified using amino acid prediction matrices of data points for a binding site. An amino acid prediction matrix is a table having a first and a second axis defining data points. Prediction matrices can be generated as shown in Singh, H. and Raghava, G.P.S. (2001), “ProPred: prediction of HLA-DR binding sites.” Bioinformatics, 17(12), 1236-37). In some embodiments, the prediction matrix is based on evolutionary conservation. In some embodiments, the prediction matrix uses physiochemical similarity to examine how similar a somatic amino acid is to the germline amino acid (e.g., Kim et al., J Immunol. 2017:3360-3368). The similarity of the somatic amino acid to the germline amino acid approximates how a mutation affects binding (e.g., T cell receptor recognition). In some embodiments, less similarity is indicative of improved binding (e.g., T cell receptor recognition).

In some embodiments, the MHC stabilizing region is designed based on the subject's particular MHC. In that way the MHC stabilizing region can be optimized for each patient.

The neoepitopes selected for inclusion in the cancer vaccine (e.g., nucleic acid cancer vaccine) will typically be high affinity binding peptides. In some aspects, the neoepitope binds an HLA protein with greater affinity than a wild-type peptide. The neoepitope has an IC50 of at least less than 5000 nM, at least less than 500 nM, at least less than 250 nM, at least less than 200 nM, at least less than 150 nM, at least less than 100 nM, at least less than 50 nM or less in some embodiments. Typically, peptides with predicted IC50<50 nM are generally considered medium to high affinity binding peptides and will be selected for testing their affinity empirically using biochemical assays of HLA-binding. Finally, it will be determined whether the human immune system can mount effective immune responses against these mutated tumor antigens and thus effectively kill tumor but not normal cells.

In some embodiments, the neoepitopes are 13 residues or less in length and may consist of between about 8 and about 11 residues, particularly 9 or 10 residues. In other embodiments, the neoepitopes may be designed to be longer. For instance, the neoepitopes may have extensions of 2-5 amino acids toward the N- and C-terminus of each corresponding gene product. The use of a longer peptide may allow endogenous processing by patient cells and may lead to more effective antigen presentation and induction of T cell responses.

Neoepitopes having the desired activity may be modified as necessary to provide certain desired attributes, e.g., improved pharmacological characteristics, while increasing or at least retaining substantially all of the biological activity of the unmodified peptide to bind the desired MHC molecule and activate the appropriate T cell or B cell. For instance, the neoepitopes may be subject to various changes, such as substitutions, either conservative or non-conservative, where such changes might provide for certain advantages in their use, such as improved MHC binding. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr. The effect of single amino acid substitutions may also be probed using D-amino acids. Such modifications may be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, Ill., Pierce), 2d Ed. (1984).

The neoepitopes can also be modified by extending or decreasing the compound's amino acid sequence, e.g., by the addition or deletion of amino acids. The peptides, polypeptides or analogs can also be modified by altering the order or composition of certain residues, it being readily appreciated that certain amino acid residues essential for biological activity, e.g., those at critical contact sites or conserved residues, may generally not be altered without an adverse effect on biological activity.

Typically, a series of peptides with single amino acid substitutions are employed to determine the effect of electrostatic charge, hydrophobicity, etc. on binding. For instance, a series of positively charged (e.g., Lys or Arg) or negatively charged (e.g., Glu) amino acid substitutions are made along the length of the peptide revealing different patterns of sensitivity towards various MHC molecules and T cell or B cell receptors. In addition, multiple substitutions using small, relatively neutral moieties such as Ala, Gly, Pro, or similar residues may be employed. The substitutions may be homo-oligomers or hetero-oligomers. The number and types of residues which are substituted or added depend on the spacing necessary between essential contact points and certain functional attributes which are sought (e.g., hydrophobicity versus hydrophilicity). Increased binding affinity for an MHC molecule or T cell receptor may also be achieved by such substitutions, compared to the affinity of the parent peptide. In any event, such substitutions should employ amino acid residues or other molecular fragments chosen to avoid, for example, steric and charge interference which might disrupt binding.

The neoepitopes may also comprise isosteres of two or more residues in the neoepitopes. An isostere as defined here is a sequence of two or more residues that can be substituted for a second sequence because the steric conformation of the first sequence fits a binding site specific for the second sequence. The term specifically includes peptide backbone modifications well known to those skilled in the art. Such modifications include modifications of the amide nitrogen, the alpha-carbon, amide carbonyl, complete replacement of the amide bond, extensions, deletions or backbone crosslinks. See, generally, Spatola, Chemistry and Biochemistry of Amino Acids, Peptides and Proteins, Vol. VII (Weinstein ed., 1983).

The consideration of immunogenicity is an important component in the selection of optimal neoepitopes for inclusion in a vaccine. As a set of non-limiting examples, immunogenicity may be assessed by analyzing the MHC binding capacity of a neoepitope, HLA promiscuity, mutation position, predicted T cell reactivity, actual T cell reactivity, structure leading to particular conformations and resultant solvent exposure, and representation of specific amino acids.

One important aspect of a neoepitope included in a vaccine is a lack of self-reactivity. The putative neoepitopes may be screened to confirm that the epitope is restricted to tumor tissue, for instance, arising as a result of genetic change within malignant cells. Ideally, the epitope should not be present in normal tissue of the patient and thus, self-similar epitopes are filtered out of the dataset. A personalized coding genome may be used as a reference for comparison of neoantigen candidates to determine lack of self-reactivity. In some embodiments, a personalized coding genome is generated from an individualized transcriptome and/or exome.

Checkpoint Inhibitors In other aspects the disclosure provides anti-cancer immunotherapies, such as immune checkpoint inhibitors, for use in combination with the cancer vaccines. Immune checkpoint modulators include both stimulatory checkpoint molecules and inhibitory checkpoint molecules (e.g., an anti-CTLA4 and/or an anti-PD1 antibody).

Stimulatory checkpoint inhibitors function by promoting the checkpoint process. Several stimulatory checkpoint molecules are members of the tumor necrosis factor (TNF) receptor superfamily (e.g., CD27, CD40, OX40, GITR, or CD137), while others belong to the B7-CD28 superfamily (e.g., CD28 or ICOS). OX40 (CD134), is involved in the expansion of effector and memory T cells. Anti-OX40 monoclonal antibodies have been shown to be effective in treating advanced cancer. MEDI0562 is a humanized OX40 agonist. GITR, Glucocorticoid-Induced TNFR family Related gene, is involved in T cell expansion. Several antibodies to GITR have been shown to promote anti-tumor responses. ICOS, Inducible T-cell costimulator, is important in T cell effector function. CD27 supports antigen-specific expansion of naïve T cells and is involved in the generation of T and B cell memory. Several agonistic anti-CD27 antibodies are in development. CD122 is the Interleukin-2 receptor beta sub-unit. NKTR-214 is a CD122-biased immune-stimulatory cytokine.

Inhibitory checkpoint molecules include, but are not limited to: PD-1, TIM-3, VISTA, A2AR, B7-H3, B7-H4, BTLA, CTLA-4, IDO, KIR and LAG3. CTLA-4, PD-1, and ligands thereof are members of the CD28-B7 family of co-signaling molecules that play important roles throughout all stages of T-cell function and other cell functions. CTLA-4, Cytotoxic T-Lymphocyte-Associated protein 4 (CD152), is involved in controlling T cell proliferation.

The PD-1 receptor is expressed on the surface of activated T cells (and B cells) and, under normal circumstances, binds to its ligands (PD-L1 and PD-L2) that are expressed on the surface of antigen-presenting cells, such as dendritic cells or macrophages. This interaction sends a signal into the T cell and inhibits it. Cancer cells take advantage of this system by driving high levels of expression of PD-L1 on their surface. This allows them to gain control of the PD-1 pathway and switch off T cells expressing PD-1 that may enter the tumor microenvironment, thus suppressing the anticancer immune response. Pembrolizumab (formerly MK-3475 and lambrolizumab, trade name KETRUDA) is a human antibody used in cancer immunotherapy and targets the PD-1 receptor.

The immune checkpoint inhibitor is a molecule such as a monoclonal antibody, a humanized antibody, a fully human antibody, a fusion protein or a combination thereof or a small molecule. For instance, the immune checkpoint inhibitor, in some embodiments, inhibits a checkpoint protein which may be CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. Ligands of checkpoint proteins include but are not limited to CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands.

In some embodiments, the anti-PD-1 antibody is BMS-936558 (nivolumab). In other embodiments, the anti-CTLA-4 antibody is ipilimumab (trade name Yervoy, formerly known as MDX-010 and MDX-101).

In some embodiments, the anti-PD-1 antibody, or antigen binding fragment thereof, comprises: (a) light chain CDRs comprising a sequence of amino acids as set forth in SEQ ID NOs: 43, 44 and 45 and heavy chain CDRs comprising a sequence of amino acids as set forth in SEQ ID NOs: 48, 49 and 50.

In some embodiments, the anti-PD-1 antibody or antigen binding fragment thereof is a human antibody. In other embodiments, the anti-PD-1 antibody or antigen binding fragment thereof is a humanized antibody. In other embodiments, the anti-PD-1 antibody or antigen binding fragment thereof is a chimeric antibody. In specific embodiments, the anti-PD-1 antibody or antigen binding fragment thereof is a monoclonal antibody.

In some embodiments, the anti-PD-1 antibody, or antigen binding fragment thereof, specifically binds to human PD-1 and comprises (a) a heavy chain variable region comprising an amino acid sequence as set forth in SEQ ID NO:51, or a variant thereof, and (b) a light chain variable region comprising an amino acid sequence selected from the group consisting of SEQ ID NO: 46.

A variant of a heavy chain variable region sequence or full-length heavy chain sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence or full-length light chain sequence is identical to the reference sequence except having up to five conservative amino acid substitutions in the framework region (i.e., outside of the CDRs), and preferably has less than four, three or two conservative amino acid substitution in the framework region.

In some embodiments, the anti-PD-1 antibody or antigen-binding fragment thereof is a monoclonal antibody which specifically binds to human PD-1 and comprises (a) a heavy chain comprising or consisting of a sequence of amino acids as set forth in SEQ ID NO:52, or a variant thereof; and (b) a light chain comprising or consisting of a sequence of amino acids as set forth in SEQ ID NO:47, or a variant thereof.

In some embodiments, the anti-PD-1 antibody or antigen-binding fragment thereof is a monoclonal antibody which specifically binds to human PD-1 and comprises (a) a heavy chain comprising or consisting of a sequence of amino acids as set forth in SEQ ID NO:52 and (b) a light chain comprising or consisting of a sequence of amino acids as set forth in SEQ ID NO:47.

In some embodiments, the anti-PD-1 antibody or antigen-binding fragment thereof is pembrolizumab or a variant thereof. In some embodiments, the anti-PD-1 antibody or antigen-binding fragment thereof is pembrolizumab.

TABLE A
Exemplary PD-1 Antibody Sequences

| | | |
|---|---|---|
| Antibody | | SEQ |
| Feature | Amino Acid Sequence | ID NO. |

Pembrolizumab Light Chain

| | | |
|---|---|---|
| CDR1 | RASKGVSTSGYSYLH | 43 |
| CDR2 | LASYLES | 44 |
| CDR3 | QHSRDLPLT | 45 |
| Variable | EIVLTQSPATLSLSPGERATLSCRASKGVSTSGYSYLHWYQQKP | 46 |
| Region | GQAPRLLIYLASYLESGVPARFSGSGSGTDFTLTISSLEPEDFAV | |
| | YYCQHSRDLPLTFGGGTKVEIK | |
| Light | EIVLTQSPATLSLSPGERATLSCRASKGVSTSGYSYLHWYQQKP | 47 |
| Chain | GQAPRLLIYLASYLESGVPARFSGSGSGTDFTLTISSLEPEDFAV | |
| | YYCQHSRDLPLTFGGGTKVEIKRTVAAPSVFIFPPSDEQLKSGTA | |
| | SVVCLLNNFYPREAKVQWKVDNALQSGNSQESVTEQDSKDST | |
| | YSLSSTLTLSKADYEKHKVYACEVTHQGLSSPVTKSFNRGEC | |

Pembrolizumab Heavy Chain

| | | |
|---|---|---|
| CDR1 | NYYMY | 48 |
| CDR2 | GINPSNGGTNFNEKFKN | 49 |
| CDR3 | RDYRFDMGFDY | 50 |
| Variable | QVQLVQSGVEVKKPGASVKVSCKASGYTFTNYYMYWVRQAP | 51 |
| Region | GQGLEWMGGINPSNGGTNFNEKFKNRVTLTTDSSTTTAYMELK | |
| | SLQFDDTAVYYCARRDYRFDMGFDYWGQGTTVTVSS | |
| Heavy | QVQLVQSGVEVKKPGASVKVSCKASGYTFTNYYMYWVRQAP | 52 |
| Chain | GQGLEWMGGINPSNGGTNFNEKFKNRVTLTTDSSTTTAYMELK | |
| | SLQFDDTAVYYCARRDYRFDMGFDYWGQGTTVTVSSASTKGP | |
| | SVFPLAPCSRSTSESTAALGCLVKDYFPEPVTVSWNSGALTSGV | |
| | HTFPAVLQSSGLYSLSSVVTVPSSSLGTKTYTCNVDHKPSNTKV | |
| | DKRVESKYGPPCPPCPAPEFLGGPSVFLFPPKPKDTLMISRTPEV | |
| | TCVVVDVSQEDPEVQFNWYVDGVEVHNAKTKPREEQFNSTYR | |
| | VVSVLTVLHQDWLNGKEYKCKVSNKGLPSSIEKTISKAKGQPR | |
| | EPQVYTLPPSQEEMTKNQVSLTCLVKGFYPSDIAVEWESNGQPE | |
| | NNYKTTPPVLDSDGSFFLYSRLTVDKSRWQEGNVFSCSVMHEA | |
| | LHNHYTQKSLSLSLGK | |

In some embodiments, the anti-PD-1 antibody or antigen-binding fragment thereof comprises a heavy chain constant region, e.g., a human constant region, such as g1, g2, g3, or g4 human heavy chain constant region or a variant thereof. In another embodiment, the anti-PD-1 antibody or antigen-binding fragment thereof comprises a light chain constant region, e.g., a human light chain constant region, such as lambda or kappa human light chain region or a variant thereof. By way of example, and not limitation, the human heavy chain constant region can be g4 and the human light chain constant region can be kappa. In an alternative embodiment, the Fc region of the antibody is g4 with a Ser228Pro mutation (Schuurman, J et. al., Mol. Immunol. 38:1-8, 2001). In some embodiments, different constant domains may be appended to humanized VL and VH regions derived from the CDRs provided herein. For example, if a particular intended use of an antibody (or fragment) of the present invention were to call for altered effector functions, a heavy chain constant domain other than human IgG1 may be used, or hybrid IgG1/IgG4 may be utilized. Although human IgG1 antibodies provide for long half-life and for effector functions, such as complement activation and antibody-dependent cellular cytotoxicity, such activities may not be desirable for all uses of the antibody. In such instances a human IgG4 constant domain, for example, may be used. The present invention includes the use of anti-PD-1 antibodies or antigen-binding fragments thereof which comprise an IgG4 constant domain. In one embodiment, the IgG4 constant domain can differ from the native human IgG4 constant domain (Swiss-Prot Accession No. P01861.1) at a position corresponding to position 228 in the EU system and position 241 in the KABAT system, where the native Ser108 is replaced with Pro, in order to prevent a potential inter-chain disulfide bond between Cys106 and Cys109 (corresponding to positions Cys 226 and Cys 229 in the EU system and positions Cys 239 and Cys 242 in the KABAT system) that could interfere with proper intra-chain disulfide bond formation. See Angal et al. (1993) Mol. Imunol. 30:105. In other instances, a modified IgG1 constant domain which has been modified to increase half-life or reduce effector function can be used.

In some embodiments, the anti-PD-1 antibody or antigen binding fragment thereof has a variable light domain and/or a variable heavy domain with at least 95%, 90%, 85%, 80%, 75% or 50% sequence identity to one of the variable light domains or variable heavy domains described above, and exhibits specific binding to PD-1. In another embodiment of the methods of treatment of the invention, the anti-PD-1 antibody or antigen binding fragment thereof comprises variable light and variable heavy domains having up to 1, 2, 3, 4, or 5 or more amino acid substitutions, and exhibits specific binding to PD-1.

KEYTRUDA™ (pembrolizumab) is approved for the treatment of patients across a number of indications. Pembrolizumab is approved for use in several cancer types, and is under investigation in several phases of clinical development for many more. Despite much progress in the field of immune-oncology therapeutics, not all subjects respond to pembrolizumab therapy, most responses are not complete, and it is only approved for use in limited tumor types. Combining pembrolizumab with mRNA cancer vaccine may allow more subjects to derive greater clinical benefit than with pembrolizumab monotherapy.

The dose of pembrolizumab, in some embodiments, is 200 mg administered every 3 weeks. The dose recently approved in the United States for treatment of cutaneous melanoma subjects is 2 mg/kg every 3 weeks. It has been concluded that a dose of 200 mg consistently across multiple tumor types is similar to 2 mg/kg. The dose of pembrolizumab, in some embodiments, is 400 mg administered every 6 weeks.

In some embodiments, an immune checkpoint inhibitor is administered to a patient on a regular basis (e.g., once a week, once every two weeks, once every three weeks, once every four weeks, once every five weeks, once every six weeks, once every seven weeks, once every eight weeks, once every nine weeks, etc.) for a specified total period of time, or until a particular endpoint is reached. The specified total period of time, in some embodiments, is the time corresponding to the administration of 2 doses, 3 doses, 4 doses, 5 doses, 6 doses, 7 doses, 8 doses, 9 doses, 10 doses, 11 doses, 12 doses, 13 doses, 14 doses, 15 doses, 16 doses, 17 doses, 18 doses, 19 doses, 20 doses, 21 doses, 22 doses, 23 doses, 24 doses or more. For example, an immune checkpoint inhibitor, in some embodiments, is administered to a patient once every six weeks. In some embodiments, the immune checkpoint inhibitor is administered until 18 doses have been administered, or until another endpoint is reached. In some embodiments, the other endpoint is disease recurrence, unacceptable toxicity, withdrawal of consent to be treated, or a total timeframe has been reached (e.g., until treatment has been ongoing for 6 months, 1 year, 2 years, 3 years, etc.).

In some embodiments, the cancer therapeutic agents, including the checkpoint inhibitors, are delivered in the form of mRNA encoding the cancer therapeutic agents. In other embodiments, the checkpoint inhibitors are delivered in the form of polypeptides.

### Methods for Preparation and Optimization

In other aspects, the disclosure provides a method for preparing a cancer vaccine, comprising a combination (e.g., some or all) of the following steps: a) identifying between 5-130 personalized cancer antigens for a patient; b) determining the anti-tumor efficacy of at least two peptide epitopes for each of the 5-130 personalized cancer antigens; and c) preparing a cancer vaccine in which the total anti-cancer efficacy of the cancer vaccine is maximized (e.g., the predicted total anti-cancer efficacy of the cancer vaccine is maximized) for a given total length of the cancer vaccine.

Methods for generating cancer vaccines according to the disclosure may involve identification of mutations using techniques such as deep nucleic acid or protein sequencing methods as described herein of tissue samples. In some embodiments, an initial identification of mutations in a subject's (e.g., a patient's) transcriptome is performed. The data from the subject's (e.g., the patient's) transcriptome is compared with sequence information from the subject's (e.g., the patient's) exome in order to identify patient specific and tumor specific mutations that are expressed. The comparison produces a dataset of putative neoepitopes, referred to as a mutanome. The mutanome may include approximately 100-10,000 candidate mutations per patient. In some embodiments, an mRNA neoantigen vaccine is designed and manufactured. The patient is then treated with the vaccine. In certain embodiments, such a neoantigen-containing vaccine may be a polycistronic vaccine including multiple neoepitopes or one or more single RNA vaccines or a combination thereof.

In some embodiments, the entire method from the initiation of the mutation identification process to the start of patient treatment is achieved in less than 2 months. In other embodiments, the whole process is achieved in 7 weeks or less, 6 weeks or less, 5 weeks or less, 4 weeks or less, 3 weeks or less, 2 weeks or less or less than 1 week. In some embodiments, the whole method is performed in less than 30 days.

In a personalized cancer vaccine, the subject specific cancer antigens may be identified in a sample of a patient. The term “biological sample” refers to a sample that contains biological materials such as a DNA, a RNA and/or a protein. In some embodiments, the biological sample may suitably comprise a bodily fluid from a subject. The bodily fluids can be fluids isolated from anywhere in the body of the subject, preferably a peripheral location, including but not limited to, for example, blood, plasma, serum, urine, sputum, spinal fluid, cerebrospinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid and combinations thereof. In some embodiments, the sample may be a tissue sample or a tumor sample. For instance, a sample of one or more tumor cells may be examined for the presence of subject specific cancer antigens.

Once an mRNA vaccine is synthesized, it is administered to the patient. In some embodiments, the vaccine is administered on a schedule for up to two months, up to three months, up to four month, up to five months, up to six months, up to seven months, up to eight months, up to nine months, up to ten months, up to eleven months, up to 1 year, up to 1 and ½ years, up to two years, up to three years, or up to four years. The schedule may be the same or varied. In some embodiments, the schedule is weekly for the first 3 weeks and then monthly thereafter.

At any point in the treatment the patient may be examined to determine whether the mutations in the vaccine are still appropriate. Based on that analysis, the vaccine may be adjusted or reconfigured to include one or more different mutations or to remove one or more mutations.

It has been recognized and appreciated that, by analyzing certain properties of cancer associated mutations, optimal neoepitopes may be assessed and/or selected for inclusion in a cancer vaccine. A property of a neoepitope or set of neoepitopes may include, for instance, an assessment of gene or transcript-level expression in patient RNA-seq or other nucleic acid analysis, tissue-specific expression in available databases, known oncogenes/tumor suppressors, variant call confidence score, RNA-seq allele-specific expression, conservative vs. non-conservative AA substitution, position of point mutation (Centering Score for increased TCR engagement), position of point mutation (Anchoring Score for differential HLA binding), Selfness: <100% core epitope homology with patient WES data, HLA-A and -B IC50 for 8 mers-11 mers, HLA-DRB1 IC₅₀ for 15 mers-20 mers, promiscuity Score (i.e., number of patient HLAs predicted to bind), HLA-C IC₅₀ for 8 mers-11 mers, HLA-DRB3-5 IC₅₀ for 15 mers-20 mers, HLA-DQB1/A1 IC₅₀ for 15 mers-20 mers, HLA-DPB1/A1 IC₅₀ for 15 mers-20 mers, Class I vs Class II proportion, Diversity of patient HLA-A, -B and DRB1 allotypes covered, proportion of point mutation vs complex epitopes (e.g., frameshifts), and/or pseudo-epitope HLA binding scores.

In some embodiments, the properties of cancer associated mutations used to identify optimal neoepitopes are properties related to the type of mutation, abundance of mutation in patient sample, immunogenicity, lack of self-reactivity, and nature of peptide composition.

The type of mutation should be determined and considered as a factor in determining whether a putative epitope should be included in a vaccine. The type of mutation may vary. In some instances it may be desirable to include multiple different types of mutations in a single vaccine. In other instances a single type of mutation may be more desirable. A value for each particular mutation can be weighted and calculated. In some embodiments, a particular mutation is a single nucleotide polymorphism (SNP). In some embodiments, a particular mutation is a complex variant, for example, a peptide sequence resulting from intron retention, complex splicing events, or insertion/deletion mutations changing the reading frame of a sequence.

The abundance of the mutation in a patient sample may also be scored and factored into the decision of whether a putative epitope should be included in a vaccine. Highly abundant mutations may promote a more robust immune response.

In some embodiments, methods for generating cancer vaccines comprise steps or methods described in International Patent Application Pub. No. WO2020/006242 (published Jan. 2, 2020, entitled “PERSONALIZED CANCER VACCINE EPITOPE SELECTION”), the contents of which are herein incorporated by reference in their entirety for this purpose.

In other aspects, the disclosure provides a method for optimizing a cancer vaccine, comprising preparing a personalized vaccine (e.g., using a method provided herein), administering the personalized cancer vaccine to the subject for whom it was prepared, evaluating immune responses in the subject to the peptides encoded by the personalized vaccine, and preparing an optimized personalized cancer vaccine. In some embodiments, preparing an optimized personalized cancer vaccine comprises analyzing the immune responses evaluated in the subject to the peptides encoded by the first personalized vaccine. Such analysis can inform revisions to be incorporated into an optimized personalized cancer vaccine, such as removal of certain peptides from the vaccine, addition of new peptides to the vaccine, and duplication of certain peptides in the vaccine.

In some embodiments, a method to optimize a personalized cancer vaccine comprises a step of determining the immunogenicity of peptides encoded by a personalized cancer vaccine. Immunogenicity of a peptide can be determined in vitro and/or ex vivo, for example by stimulating immune cells (e.g., peripheral blood mononuclear cells (PBMCs), such as PBMCs from a sample collected from the subject to be administered the vaccine, or who has previously been administered the vaccine) with the peptide and subsequently measuring immune activation signals (e.g., cytokine production) from the immune cells. Immunogenicity of a peptide can also be determined by a method described in U.S. Patent Application Pub. No. US2022/0236253A1, the contents of which are herein incorporated by reference in their entirety for this purpose.

In some embodiments, a method to optimize a personalized cancer vaccine comprises a step of selecting a subset of peptides encoded by a personalized cancer vaccine for inclusion in an optimized personalized cancer vaccine, e.g., based on their determined immunogenicity. The selection may, for example, result in exclusion of certain peptides from the optimized personalized cancer vaccine, e.g., if they are poorly immunogenic in subject following administration of the unoptimized vaccine. The selection may also, for example, result in identification of certain neoantigen(s) (e.g., corresponding to certain peptide(s) of the unoptimized vaccine) that are represented multiple times (e.g., 2, 3, 4, 5, 6, 7, 9, or more times) in the optimized cancer vaccine. In such embodiments, the multiple representations of the neoantigen(s) may involve expression of multiple copies of the same peptide by the nucleic acid (e.g., mRNA), or may involve expression of multiple distinct peptides that each correspond to the same neoantigen(s). For example, if neoantigen A is selected for multiple representations in the optimized vaccine, peptide A1 corresponding to neoantigen A may be encoded multiple times in the open reading frame of the nucleic acid (e.g., mRNA), or peptides A1, A2, A3, etc., each corresponding to neoantigen A but with distinct amino acid sequences may each be encoded in the open reading frame.

In some embodiments, a method to optimize a personalized cancer vaccine comprises selection of additional neoantigens from the subject but not represented in an unoptimized vaccine. This may include any neoantigens identified in the subject but that were excluded from the unoptimized vaccine. The selection of additional neoantigens can be made according to the methods provided herein. For example, one or more neoantigens having a lower predicted efficacy than those included in the unoptimized vaccine may be selected to be included in the optimized vaccine. Peptide(s) corresponding to the additional neoantigen(s), in some embodiments, are encoded by the optimized personalized cancer vaccine (e.g., an mRNA of the optimized personalized cancer vaccine).

In some embodiments, an optimized personalized cancer vaccine encodes for more peptides corresponding to driver mutations (e.g., 1 more, 2 more, 3 more, 4 more, 5 more, 6 more, 7 more, 8 more, 9 more, 10 more, or more) relative to a corresponding unoptimized personalized cancer vaccine. In some embodiments, an optimized personalized cancer vaccine encodes for fewer peptides corresponding to driver mutations (e.g., 1 fewer, 2 fewer, 3 fewer, 4 fewer, 5 fewer, 6 fewer, 7 fewer, 8 fewer, 9 fewer, 10 fewer, or more) relative to a corresponding unoptimized personalized cancer vaccine. In some embodiments, 1 or more (e.g., 1, 2, 3, 4, 5, 6, 7, 8, 9, 10, or more) peptides corresponding to driver mutations are added to an optimized personalized cancer vaccine relative to a corresponding unoptimized personalized cancer vaccine. In some embodiments, 1 or more (e.g., 1, 2, 3, 4, 5, 6, 7, 8, 9, 10, or more) peptides corresponding to driver mutations are removed from an optimized personalized cancer vaccine relative to a corresponding unoptimized personalized cancer vaccine.

In some embodiments, methods for optimizing cancer vaccines comprise steps or methods described in International Patent Application Pub. No. WO2020/006242 (published Jan. 2, 2020, entitled “PERSONALIZED CANCER VACCINE EPITOPE SELECTION”), the contents of which are herein incorporated by reference in their entirety for this purpose.

In some embodiments, the personalized mRNA cancer vaccines described herein may be used for treatment of cancer. As one non-limiting example, the disclosure provides methods for treating a patient having cancer, comprising: a) analyzing a sample derived from the patient is in order to identify one or more personalized cancer antigens; b) determining the anti-tumor efficacy of at least two peptide epitopes for each of the identified personalized cancer antigens; c) preparing a cancer vaccine in which the total anti-cancer efficacy of the cancer vaccine is maximized (e.g., the predicted total anti-cancer efficacy of the cancer vaccine is maximized) for a given total length of the cancer vaccine; and d) administering the cancer vaccine to the patient, and optionally further preparing an optimized personalized cancer vaccine and administering the optimized vaccine to the patient.

Cancer vaccines (e.g., nucleic acid cancer vaccines) may be administered prophylactically or therapeutically as part of an active immunization scheme to healthy individuals or early in cancer or late stage and/or metastatic cancer. Cancer vaccines, in some embodiments, may be administered in an adjuvant setting, i.e., after a primary treatment (e.g., surgical resection) has been administered to the patient. Adjuvant treatment can prevent or delay recurrence or progression of the cancer in the patient. In some embodiments, the effective amount of the cancer vaccine (e.g., nucleic acid cancer vaccines) provided to a cell, a tissue or a subject may be enough for immune activation, and in particular antigen specific immune activation.

In some embodiments, the cancer vaccine (e.g., nucleic acid cancer vaccine) may be administered with an anti-cancer therapeutic agent. The cancer vaccine (e.g., nucleic acid cancer vaccine) and anti-cancer therapeutic can be combined to enhance immune therapeutic responses even further. The cancer vaccine (e.g., nucleic acid cancer vaccines) and other therapeutic agent may be administered simultaneously or sequentially. When the other therapeutic agents are administered simultaneously they can be administered in the same or separate formulations, but are administered at the same time. The other therapeutic agents are administered sequentially with one another and with the cancer vaccine (e.g., nucleic acid cancer vaccine), when the administration of the other therapeutic agents and the cancer vaccine (e.g., nucleic acid cancer vaccine) is temporally separated. The separation in time between administrations of these compounds may be a matter of minutes or it may be longer, e.g., hours, days, weeks, months. Other therapeutic agents include but are not limited to anti-cancer therapeutic, adjuvants, cytokines, antibodies, antigens, etc. Examples of anti-cancer therapeutics include, but are not limited to, DNA-alkylating agents (e.g., cyclophosphamide, ifosfamide), antimetabolites (e.g., methotrexate, a folate antagonist, and 5-fluorouracil, a pyrimidine antagonist), microtubule disrupters (e.g., vincristine, vinblastine, paclitaxel), DNA intercalators (e.g., doxorubicin, daunomycin, cisplatin), hormone therapy (e.g., tamoxifen, flutamide), and gene-targeted therapies, such as protein-tyrosine kinase inhibitors (e.g. imatinib; the EGFR kinase inhibitor, erlotinib). In some embodiments, the anti-cancer therapeutic is pembrolizumab.

In some embodiments, the progression of the cancer can be monitored to identify changes in the expressed antigens. Thus, in some embodiments, the method also involves at least one month after the administration of a cancer mRNA vaccine, identifying at least 2 cancer antigens from a sample of the subject to produce a second set of cancer antigens, and administering to the subject a mRNA vaccine having an open reading frame encoding the second set of cancer antigens to the subject. The mRNA vaccine having an open reading frame encoding second set of antigens, in some embodiments, is administered to the subject 2 months, 3 months, 4 months, 5 months, 6 months, 8 months, 10 months, or 1 year after the mRNA vaccine having an open reading frame encoding the first set of cancer antigens. In other embodiments, the mRNA vaccine having an open reading frame encoding second set of antigens is administered to the subject 1½, 2, 2½, 3, 3½, 4, 4½, or 5 years after the mRNA vaccine having an open reading frame encoding the first set of cancer antigens.

### Hotspot/Driver Mutations as Neoantigens

In population analyses of cancer, certain mutations occur in a higher percentage of patients than would be expected by chance. These “recurrent” or “hotspot” mutations have often been shown to have a “driver” role in the tumor, producing some change in the cancer cell function that is important to tumor initiation, maintenance, or metastasis, and is therefore selected for in the evolution of the tumor. These mutations are often also termed “driver” mutations. In addition to their importance in tumor biology and therapy, recurrent mutations provide the opportunity for precision medicine, in which the patient population is stratified into groups more likely to respond to a particular therapy, including but not limited to targeting the mutated protein itself.

Therefore, in some embodiments, the cancer vaccine further comprises one or more cancer hotspot neoepitopes in addition the personalized cancer epitopes. In some embodiments, one or more cancer hotspot neoepitopes are cancer hotspot antigens. In some embodiments, cancer hotspot mutations that occur over a threshold prevalence in an indication of interest are included in the vaccine. The threshold prevalence, in some embodiments, is greater than 2%, 3%, 4%, 5%, 6%, 7%, 8%, 9%, or 10%.

In some embodiments, a nucleic acid (e.g., mRNA) cancer vaccine provided herein encodes 0, 1, 2, 3, 4, 5, 6, 7, 8, 9, 10, 11, 12, 13, 14, 15, 16, 17, 18, 19, 20, or more peptides corresponding to driver mutations. In some embodiments, the nucleic acid (e.g., mRNA) cancer vaccine encodes at least 5 (e.g., 5, 6, 7, 8, 9, 10, 11, 12, 13, 14, 15, 16, 17, 18, 19, 20, or more) peptides corresponding to driver mutations. In some embodiments, the nucleic acid (e.g., mRNA) cancer vaccine encodes fewer than 15 (e.g., 14, 13, 12, 11, 10, 9, 8, 7, 6, 5, 4, 3, 2, 1, or 0) peptides corresponding to driver mutations.

Indications of interest include, but are not limited to bladder cancer, bladder urothelial carcinoma (BLCA), colon adenocarcinoma (COAD), esophageal carcinoma (ESCA), hepatocellular carcinoma (HCC), head and neck squamous cell carcinoma (HNSC), lung adenocarcinoma (LUAD), muscle-invasive bladder cancer (MIBC), muscle invasive urothelial carcinoma (MIUC), non-small cell lung cancer (NSCLC), pancreatic adenocarcinoma (PAAD), prostate adenocarcinoma (PRAD), rectum adenocarcinoma (READ), renal cell carcinoma (RCC), small cell lung cancer (SCLC), skin cutaneous melanoma (SKCM), serous ovarian cancer (SOC), stomach adenocarcinoma (STAD), squamous cell carcinoma (SCC), uterine endometrial cancer (UEC), and muscle-invasive urinary tract urothelial cancer (UTUC). In some embodiments, the indication is melanoma (e.g., stage II, stage III, or stage IV melanoma) following complete resection. In some embodiments, the indication is NSCLC (e.g., stage II, stage III (e.g., stage IIIA, stage IIIB)) following complete resection. In some embodiments, the indication is MIUC. In some embodiments, the indication is MIBC. In some embodiments, the indication is UTUC. In some embodiments, the indication is RCC. In some embodiments, the indication is SCC. In some embodiments, the indication is cutaneous SCC (cSCC).

Exemplary mutations are provided in Table B below.

TABLE B
Exemplary mutations

| Gene | Mutated position |
|---|---|
| KRAS | G12, G13 |
| NRAS | Q61 |
| BRAF | V600 |
| PIK3CA | R88, E545, H1047 |
| TP53 | R175, R282 |
| EGFR | L858 |
| FGFR3 | S249 |
| ERBB2 | S310 |
| PTEN | R130 |
| BCOR | N1459 |

Much effort and research on recurrent mutations has focused on non-synonymous (or “missense”) single nucleotide variants (SNVs), but population analyses have revealed that a variety of more complex (non-SNV) variant classifications, such as synonymous (or “silent”), splice site, multi-nucleotide variants, insertions, and deletions, can also occur at high frequencies.

The p53 gene (official symbol TP53) is mutated more frequently than any other gene in human cancers. Large cohort studies have shown that, for most p53 mutations, the genomic position is unique to one or only a few patients and the mutation cannot be used as recurrent neoantigens for therapeutic vaccines designed for a specific population of patients. Surprisingly, a small subset of p53 loci do, however, exhibit a “hotspot” pattern, in which several positions in the gene are mutated with relatively high frequency. Strikingly, a large portion of these recurrently mutated regions occur near exon-intron boundaries, disrupting the canonical nucleotide sequence motifs recognized by the mRNA splicing machinery. Mutation of a splicing motif can alter the final mRNA sequence even if no change to the local amino acid sequence is predicted (i.e., for synonymous or intronic mutations). Therefore, these mutations are often annotated as “noncoding” by common annotation tools and neglected for further analysis, even though they may alter mRNA splicing in unpredictable ways and exert severe functional impact on the translated protein. If an alternatively spliced isoform produces an in-frame sequence change (i.e., no PTC is produced), it can escape depletion by NMD and be readily expressed, processed, and presented on the cell surface by the HLA system. Further, mutation-derived alternative splicing is usually “cryptic”, i.e., not expressed in normal tissues, and therefore may be recognized by T-cells as non-self neoantigens.

Mutations are typically obtained from a patient's DNA sequencing data to derive neo-epitopes for prior art peptide vaccines. mRNA expression, however, is a more direct measurement of the global space of possible neo-epitopes. For example, some tumor-specific neo-epitopes may arise from splicing changes, insertions/deletions (InDels) resulting in frameshifts, alternative promoters, or epigenetic modifications that are not easily identified using only the exome sequencing data. In some aspects, the neoantigens from InDels are enriched for predicted high-affinity binders versus nsSNVs. Such neoantigens may be immunogenic. For example, frameshift InDels have been found to be significantly associated with checkpoint inhibitor responses across three melanoma cohorts.

Some aspects comprise methods for identifying patient specific complex mutations and formulating these mutations into effective personalized cancer vaccines (e.g., nucleic acid cancer vaccines). The methods can involve the use of short read RNA-Seq. A major challenge inherent to using short reads for RNA-seq is the fact that multiple mRNA transcript isoforms can be obtained from the same genomic locus, due to alternative splicing and other mechanisms. Due to the sequencing reads being much shorter than the full-length mRNA transcript, it becomes difficult to map a set of reads back to the correct corresponding isoform within a known gene annotation model. As a result, complex variants that diverge from the known gene annotations (as are common in cancer) can be difficult to discover by standard approaches. However, short peptides may be identified rather than the exact exon composition of the full-length transcript. The methods for identifying short peptides that will be representative of these complex mutations involve a short k-mer counting approach to neo-epitope prediction of complex variants.

### Biomarkers

Characterization of patient features, e.g., by sequence analysis of patient data can facilitate identification of patients most likely to benefit from administration of a personalized cancer vaccine. Next-generation sequencing analysis of patient data (e.g., pre-treatment biopsies) facilitates understanding and characterization of the mutation landscape, expression level of key genes, and tumor microenvironment of patients, e.g., prior to treatment. Analyzing this data prior to vaccination can be used to select patients for vaccination and to inform details of their treatment. By correlating to neoantigen-specific T cell responses post-vaccination, these data can also provide important biomarkers to assist in patient/therapeutic selection for personalized neoantigen cancer vaccines and/or for development of optimized personalized cancer vaccines.

Biomarkers include microsatellite instability (MSI) value, tumor mutational burden (TMB), T cell-inflamed gene expression profile (GEP) score, interferon-gamma (IFN-γ) signature score, immune gene signature score, T cell cytotoxicity activity (CYT) score, PD-L1 expression, minimal residual disease (MRD) level, level of γδ T cells or level of a sub-type of γδ T cell (e.g., regulatory γδ T cells), TCR clonotyping value (e.g., DE50 or Gini coefficient), and Th1 cell population level.

The TMB value represents the frequency of mutations (e.g., number of non-synonymous mutations per exome) in a given tumor. TMB is generally assessed in a tumor sample from biopsy or surgical resection. Unless indicated otherwise, TMB is expressed as a number of mutations per exome having an allele frequency of at least 5% in the tumor sample. However, TMB can also be expressed as the number of mutations per megabase in the tumor sample (e.g., as determined by an FDA-approved test). One example of an FDA-approved test that can be used to determine TMB is the FoundationOne® CDx test (Foundation Medicine, Cambridge, MA), details regarding which are provided in “Summary of Safety and Effectiveness Data” for Premarket Approval Application Number P170019 (FDA, Document P170019B, Dec. 14, 2017), and in “FoundationOneR CDx Technical Information” (Foundation Medicine, Document RAL-0003-18, Oct. 11, 2022), the contents of each of which are herein incorporated by reference in their entireties for this purpose. In some embodiments, the mutations each have an allele frequency of at least 5% (or another set allele frequency, such as 1%, 2%, 3%, 4%, 6%, 7%, 8%, 9%, 10%, or more) in the tumor sample. TMB can also be expressed as the total number of mutations having an allele frequency of at least 5% (or another set allele frequency, such as 1%, 2%, 3%, 4%, 6%, 7%, 8%, 9%, 10%, or more) within whole exome sequencing data measured in the tumor sample. In some embodiments, a subject may be selected for treatment (e.g., with a personalized cancer vaccine and/or an immune checkpoint inhibitor) if their TMB value is greater than a set value. In some embodiments, a subject may be selected for treatment (e.g., with a personalized cancer vaccine and/or an immune checkpoint inhibitor) if their TMB value is less than a set value. In some embodiments, the set value is 7, 10, 15, 20, 25, 30, 40, 50, 60, 70, 80, 90, 100, 110, 120, 130, 140, 150, 160, 170, 175, 180, 190, 200, 210, 220, 230, 240, 250, 260, 270, 280, 290, 300, 400, 500, 600, 700, 800, 900, or 1000 mutations (e.g., non-synonymous mutations) per exome having at least a set allele frequency (e.g., having an allele frequency of at least 1%, at least 2%, at least 3%, at least 4%, at least 5%, at least 6%, at least 7%, at least 8%, at least 9%, at least 10%, or more, preferably at least 5%). In some embodiments, the set value for TMB is 175 mutations per exome having at least the set allele frequency.

The T cell-inflamed GEP score incorporates expression levels of 18 genes: CXCR6, TIGIT, CD27, CD274 (PD-L1), PDCD1LG2 (PD-L2), LAG3, NKG7, PSMB10, CMKLR1, CD8A, IDO1, CCL5, CXCL9, HLA-DQA1, CD276 (B7-H3), HLA-DRB1, STAT1, HLA-E. See Cristescu et al., “Pan-tumor genomic biomarkers for PD-1 checkpoint blockade-based immunotherapy” Science 2018; 362 (6411): eaar3593; and Ayers et al., “IFN-γ-related mRNA profile predicts clinical response to PD-1 blockade” J Clin Invest. 2017; 127(8):2930-2940; the entire contents of each of which are herein incorporated by reference for this purpose. The T cell-inflamed GEP score is calculated by averaging the expression of the 18 genes in a biological sample, e.g., a tumor sample from a subject who may benefit from treatment with a personalized cancer vaccine and/or immune checkpoint inhibitor. In some embodiments, the expression of each of the 18 genes incorporated in the T cell-inflamed GEP score is weighted (e.g., a weighted mean of the normalized gene expression of each of the 18 genes is used to calculate the score, wherein each gene is attributed an individual weight). In a preferred embodiment, the expression of each of the 18 genes incorporated in the T cell-inflamed GEP score is weighted equally (e.g., an arithmetic mean of the normalized gene expression of each of the 18 genes is used to calculate the score). In some embodiments, a subject may be selected for treatment (e.g., with a personalized cancer vaccine and/or an immune checkpoint inhibitor) if their T cell-inflamed GEP score is greater than a set value. In some embodiments, a subject may be selected for treatment (e.g., with a personalized cancer vaccine and/or an immune checkpoint inhibitor) if their T cell-inflamed GEP score is less than a set value. In some embodiments, the set value is based on an average or median T cell-inflamed GEP score measured in a population of subjects (e.g., a population of subjects diagnosed as having a particular type of cancer, or a population of subjects having received primary treatment such as surgical resection of a particular type of cancer). For example, in some embodiments, the set value is 20%, 25%, 30%, 35%, 40%, 45%, 50%, 55%, 60%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, or 200% of the average or median T cell-inflamed GEP score measured in a population of subjects. For example, in some embodiments, a subject may be selected for treatment if their T cell-inflamed GEP score is greater than 100% of the average or median T cell-inflamed GEP score measured in a population of subjects. In some embodiments, a subject may be selected for treatment if their T cell-inflamed GEP score is less than 100% of the average or median T cell-inflamed GEP score measured in a population of subjects. In some embodiments, the set value is a numerical value. For example, in some embodiments, the set value is 1, 1.5, 2, 2.5, 3, 3.5, 4, 4.1, 4.2, 4.3, 4.4, 4.5, 4.6, 4.7, 4.8, 4.9, 5, 5.1, 5.2, 5.3, 5.4, 5.5, 6, or higher. For example, in some embodiments, a subject may be selected for treatment if their T cell-inflamed GEP score is greater than 4.5 or about 4.5, or less than 4.5 or about 4.5.

The interferon-gamma (IFN-γ) signature score incorporates expression levels of 6 genes: IDO1, CXCL10, CXCL9, HLA-DRA, STAT1, and IFNG. See Ayers et al., “IFN-γ-related mRNA profile predicts clinical response to PD-1 blockade” J Clin Invest. 2017; 127(8):2930-2940; the entire contents of which are herein incorporated by reference for this purpose. The IFN-γ signature score is calculated by averaging the expression of the 6 genes in a biological sample, e.g., a tumor sample from a subject who may benefit from treatment with a personalized cancer vaccine and/or immune checkpoint inhibitor. In some embodiments, the expression of each of the 6 genes incorporated in the IFN-γ signature score is weighted (e.g., a weighted mean of the normalized gene expression of each of the 6 genes is used to calculate the score, wherein each gene is attributed an individual weight). In a preferred embodiment, the expression of each of the 6 genes incorporated in the IFN-γ signature score is weighted equally (e.g., an arithmetic mean of the normalized gene expression of each of the 18 genes is used to calculate the score). In some embodiments, a subject may be selected for treatment (e.g., with a personalized cancer vaccine and/or an immune checkpoint inhibitor) if their IFN-γ signature score is greater than a set value. In some embodiments, a subject may be selected for treatment (e.g., with a personalized cancer vaccine and/or an immune checkpoint inhibitor) if their IFN-γ signature score is less than a set value. In some embodiments, the set value is based on an average or median IFN-γ signature score measured in a population of subjects (e.g., a population of subjects diagnosed as having a particular type of cancer, or a population of subjects having received primary treatment such as surgical resection of a particular type of cancer). For example, in some embodiments, the set value is 20%, 25%, 30%, 35%, 40%, 45%, 50%, 55%, 60%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, or 200% of the average or median IFN-γ signature score measured in a population of subjects. For example, in some embodiments, a subject may be selected for treatment if their IFN-γ signature score is greater than 100% of the average or median IFN-γ signature score measured in a population of subjects. In some embodiments, a subject may be selected for treatment if their IFN-γ signature score is less than 100% of the average or median IFN-γ signature score measured in a population of subjects. In some embodiments, the set value is a numerical value. For example, in some embodiments, the set value is 1, 1.5, 2, 2.5, 3, 3.5, 4, 4.1, 4.2, 4.3, 4.4, 4.5, 4.6, 4.7, 4.8, 4.9, 5, 5.1, 5.2, 5.3, 5.4, 5.5, 6, or higher. For example, in some embodiments, a subject may be selected for treatment if their IFN-γ signature score is greater than 4.5 or about 4.5, or less than 4.5 or about 4.5.

The immune gene signature score incorporates expression levels of 18 genes: CD3D, IDO1, CIITA, CD3E, CCL5, GZMK, CD2, HLA-DRA, CXCL13, IL2RG, NKG7, HLA-E, CXCR6, LAG3, TAGAP, CXCL10, STAT1, and GZMB. See Ayers et al., “IFN-γ-related mRNA profile predicts clinical response to PD-1 blockade” J Clin Invest. 2017; 127(8):2930-2940; the entire contents of which are herein incorporated by reference for this purpose. The immune gene signature score is calculated by averaging the expression of the 6 genes in a biological sample, e.g., a tumor sample from a subject who may benefit from treatment with a personalized cancer vaccine and/or immune checkpoint inhibitor. In some embodiments, the expression of each of the 6 genes incorporated in the immune gene signature score is weighted (e.g., a weighted mean of the normalized gene expression of each of the 6 genes is used to calculate the score, wherein each gene is attributed an individual weight). In a preferred embodiment, the expression of each of the 6 genes incorporated in the immune gene signature score is weighted equally (e.g., an arithmetic mean of the normalized gene expression of each of the 18 genes is used to calculate the score). In some embodiments, a subject may be selected for treatment (e.g., with a personalized cancer vaccine and/or an immune checkpoint inhibitor) if their immune gene signature score is greater than a set value. In some embodiments, a subject may be selected for treatment (e.g., with a personalized cancer vaccine and/or an immune checkpoint inhibitor) if their immune gene signature score is less than a set value. In some embodiments, the set value is based on an average or median immune gene signature score measured in a population of subjects (e.g., a population of subjects diagnosed as having a particular type of cancer, or a population of subjects having received primary treatment such as surgical resection of a particular type of cancer). For example, in some embodiments, the set value is 20%, 25%, 30%, 35%, 40%, 45%, 50%, 55%, 60%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, or 200% of the average or median immune gene signature score measured in a population of subjects. For example, in some embodiments, a subject may be selected for treatment if their immune gene signature score is greater than 100% of the average or median immune gene signature score measured in a population of subjects. In some embodiments, a subject may be selected for treatment if their immune gene signature score is less than 100% of the average or median immune gene signature score measured in a population of subjects. In some embodiments, the set value is a numerical value. For example, in some embodiments, the set value is 1, 1.5, 2, 2.5, 3, 3.5, 4, 4.1, 4.2, 4.3, 4.4, 4.5, 4.6, 4.7, 4.8, 4.9, 5, 5.1, 5.2, 5.3, 5.4, 5.5, 6, or higher. For example, in some embodiments, a subject may be selected for treatment if their immune gene signature score is greater than 4.5 or about 4.5, or less than 4.5 or about 4.5.

The CYT score incorporates expression of granzyme B (GZMB) and perforin-1 (PRF1). See Ayers, et al., “IFN-γ-related mRNA profile predicts clinical response to PD-1 blockade” J Clin Invest. 2017; 127(8):2930-2940. The CYT score is calculated by averaging the expression of GZMB and PRF1 in a biological sample, e.g., a tumor sample from a subject who may benefit from treatment with a personalized cancer vaccine and/or immune checkpoint inhibitor. In some embodiments, a subject may be selected for treatment (e.g., with a personalized cancer vaccine and/or an immune checkpoint inhibitor) if their CYT score is greater than a set value. In some embodiments, a subject may be selected for treatment (e.g., with a personalized cancer vaccine and/or an immune checkpoint inhibitor) if their CYT score is less than a set value. In some embodiments, the set value is based on an average or median CYT score measured in a population of subjects (e.g., a population of subjects diagnosed as having a particular type of cancer, or a population of subjects having received primary treatment such as surgical resection of a particular type of cancer). For example, in some embodiments, the set value is 20%, 25%, 30%, 35%, 40%, 45%, 50%, 55%, 60%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, or 200% of the average or median CYT score measured in a population of subjects. For example, in some embodiments, a subject may be selected for treatment if their CYT score is greater than 100% of the average or median CYT score measured in a population of subjects, or in some embodiments, a subject may be selected for treatment if their CYT score is less than 100% of the average or median CYT score measured in a population of subjects. In some embodiments, the set value is a numerical value. For example, in some embodiments, the set value for CYT score is 1, 1.5, 2, 2.5, 3, 3.5, 4, 4.1, 4.2, 4.3, 4.4, 4.5, 4.6, 4.7, 4.8, 4.9, 5, 5.1, 5.2, 5.3, 5.4, 5.5, 6, or higher.

PD-L1 expression for use as a biomarker may be calculated as a normalized gene expression value in a biological sample, e.g., a tumor sample from a subject who may benefit from treatment with a personalized cancer vaccine and/or immune checkpoint inhibitor. PD-L1 expression can be measured, for example, by gene expression analysis methods known in the art, including qRT-PCR, microarray, Northern blotting, immunohistochemical staining and optionally subsequent quantification of the staining (e.g., with an anti-PD-L1 antibody used for staining of a histological sample, such as of a resected tumor or tumor biopsy), or RNA sequencing (RNA-seq). Unless indicated otherwise, PD-L1 expression is measured by RNA sequencing. In some embodiments, a subject may be selected for treatment (e.g., with a personalized cancer vaccine and/or an immune checkpoint inhibitor) if their PD-L1 expression is greater than a set value. In some embodiments, a subject may be selected for treatment (e.g., with a personalized cancer vaccine and/or an immune checkpoint inhibitor) if their PD-L1 expression is less than a set value. In some embodiments, the set value is based on an average or median PD-L1 expression measured in a population of subjects (e.g., a population of subjects diagnosed as having a particular type of cancer, or a population of subjects having received primary treatment such as surgical resection of a particular type of cancer). For example, in some embodiments, the set value is 20%, 25%, 30%, 35%, 40%, 45%, 50%, 55%, 60%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, or 200% of the average or median PD-L1 expression measured in a population of subjects. For example, in some embodiments, a subject may be selected for treatment if their PD-L1 expression is greater than 100% of the average or median PD-L expression measured in a population of subjects, or in some embodiments, a subject may be selected for treatment if their PD-L1 expression is less than 100% of the average or median PD-L1 expression measured in a population of subjects. In some embodiments, the set value is a numerical value. For example, in some embodiments, the set value for normalized PD-L1 expression is 1, 1.5, 2, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 6, or higher, when normalized relative to one or more housekeeping genes (e.g., STK11IP, ZBTB34, TBC1D10B, OAZ1, POLR2A, G6PD, ABCF1, C14orf102, UBB, TBP, SDHA).

MRD level reflects the number of cancer cells remaining in a patient's body after a cancer treatment (e.g., a surgical resection of a tumor). The presence of these cells may predispose a patient to disease recurrence. MRD can be measured in a number of ways, including flow cytometry (e.g., to detect cancer cells in a biological sample or count the number of cancer cells in a biological sample), polymerase chain reaction (PCR; e.g., to quantify the relative amount of a given nucleotide sequence or sequences in a biological sample), and next-generation sequencing (e.g., to quantify the amount a given nucleotide sequence or sequences in a biological sample). Unless indicated otherwise, MRD is measured by next generation sequencing, preferably to detect and/or quantify circulating tumor DNA (ctDNA). In some embodiments, ctDNA is measured by RaDaR™ next generation sequencing assay (Inivata® Limited, Research Triangle Park, NC, USA). In some embodiments, a subject may be selected for treatment (e.g., with a personalized cancer vaccine and/or an immune checkpoint inhibitor) if their MRD level is greater than a set value. In some embodiments, a subject may be selected for treatment (e.g., with a personalized cancer vaccine and/or an immune checkpoint inhibitor) if their MRD level is less than a set value. In some embodiments, the set value is based on an average or median MRD level measured in a population of subjects (e.g., a population of subjects diagnosed as having a particular type of cancer, or a population of subjects having received primary treatment such as surgical resection of a particular type of cancer). For example, in some embodiments, the set value is 20%, 25%, 30%, 35%, 40%, 45%, 50%, 55%, 60%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, or 200% of the average or median MRD level measured in a population of subjects. For example, in some embodiments, a subject may be selected for treatment if their MRD level is greater than 100% of the average or median MRD level measured in a population of subjects, or in some embodiments, a subject may be selected for treatment if their MRD level is less than 100% of the average or median MRD level measured in a population of subjects. In some embodiments, the set value is a numerical value. For example, in some embodiments, the set value for MRD level is 10,000 copies per mL, 9,000 copies per mL, 8,000 copies per mL, 7,000 copies per mL, 6,000 copies per mL, 5,000 copies per mL, 4,000 copies per mL, 3,000 copies per mL, 2,000 copies per mL, 1,000 copies per mL, 900 copies per mL, 800 copies per mL, 700 copies per mL, 600 copies per mL, 500 copies per mL, 400 copies per mL, 300 copies per mL, 200 copies per mL, 100 copies per mL, 90 copies per mL, 80 copies per mL, 75 copies per mL, 70 copies per mL, 65 copies per mL, 60 copies per mL, 55 copies per mL, 50 copies per mL, 45 copies per mL, 40 copies per mL, 35 copies per mL, 30 copies per mL, 25 copies per mL, 20 copies per mL, 15 copies per mL, 10 copies per mL, or 5 copies per mL of a mutated gene or mutated genes in a biological sample (e.g. a biological sample comprising circulating tumor DNA, such as a blood sample). In some embodiments, the set value for MRD level is based on variant allele frequency (VAF) in a biological sample collected from the subject, and is 5×10⁻⁶ VAF, 1×10⁻⁶ VAF, 9×10⁻⁵ VAF, 8×10⁻⁵ VAF, 7×10⁻⁵ VAF, 6×10⁻⁵ VAF, 5×10⁻⁵ VAF, 4×10⁻⁵ VAF, 3×10⁻⁵ VAF, 2×10⁻⁵ VAF, 1×10⁻⁵ VAF, 9×10⁻⁴ VAF, 8×10⁻⁴ VAF, 7×10⁻⁴ VAF, 6×10⁻⁴ VAF, 5×10⁻⁴ VAF, 4×10⁻⁴ VAF, 3×10⁻⁴ VAF, 2×10⁻⁴ VAF, 1×10⁻⁴ VAF, 9×10⁻³ VAF, 8×10⁻³ VAF, 7×10⁻³ VAF, 6×10⁻³ VAF, 5×10⁻³ VAF, 4×10⁻³ VAF, 3×10⁻³ VAF, 2×10⁻³ VAF, 1×10⁻³ VAF, 9×10⁻² VAF, 8×10⁻² VAF, 7×10⁻² VAF, 6×10⁻² VAF, 5×10⁻² VAF, 4×10⁻² VAF, 3×10⁻² VAF, 2×10⁻² VAF, 1×10⁻² VAF, 9×10⁻¹ VAF, 8×10⁻¹ VAF, 7×10⁻¹ VAF, 6×10⁻¹ VAF, 5×10⁻¹ VAF, 4×10⁻¹ VAF, 3×10⁻¹ VAF, 2×10⁻¹ VAF, or 1×10⁻¹ VAF in the biological sample. In some embodiments, the set value for MRD level is based on the abundance of tumor-associated sequence(s) detected in a biological sample collected from the subject, and is 1 part per million (PPM), 5 PPM, 10 PPM, 15 PPM, 20 PPM, 25 PPM, 30 PPM, 35 PPM, 40 PPM, 45 PPM, 50 PPM, 55 PPM, 60 PPM, 65 PPM, 70 PPM, 75 PPM, 80 PPM, 85 PPM, 90 PPM, 95 PPM, 100 PPM, 110 PPM, 120 PPM, 130 PPM, 140 PPM, 150 PPM, 160 PPM, 170 PPM, 180 PPM, 190 PPM, 200 PPM, 300 PPM, 400 PPM, 500 PPM, 600 PPM, 700 PPM, 800 PPM, 900 PPM, 1000 PPM, or more in the biological sample.

In some embodiments, a subject may be selected for treatment (e.g., with a personalized cancer vaccine and/or an immune checkpoint inhibitor) if they have detectable MRD (e.g., if ctDNA is detectable in a biological sample such as a blood sample collected from the subject) following primary treatment (e.g., curative-intent surgery).

In some embodiments, ctDNA is used to predict probability of recurrence of a cancer in a subject. In some embodiments, ctDNA is used to select subjects for administration of a personalized cancer vaccine. In some embodiments, the results of ctDNA analysis (e.g., quantification and/or characterization, such as of sequences present or absent in the ctDNA) are used to predict probability of recurrence and/or to select subjects for administration of a personalized cancer vaccine. In some embodiments, results of ctDNA analysis are used to identify subjects as being likely to have a therapeutic response to administration of a personalized cancer vaccine.

In some embodiments, analysis of ctDNA (e.g., of sequences present or absent in the ctDNA, such as sequences comprising mutations relative to a reference genome or to non-tumor DNA of the subject from whom the ctDNA sample was collected) is used to select sequence variants for measurement in the subject. In some embodiments, the selected sequence variants are measured in longitudinal samples (e.g., blood samples collected at various timepoints following administration of an immune checkpoint inhibitor and/or a personalized cancer vaccine). In some embodiments, measurements of the selected sequence variants in longitudinal samples are used to monitor responses to a personalized cancer vaccine in a subject, such as to identify subjects for whom to develop an optimized personalized cancer vaccine, e.g., by a method provided herein.

Level of γδ T cells reflects the percentage of γδ T cells (or a sub-type/subset thereof) relative to other white blood cells (e.g., T lymphocytes) in a subject or in a biological sample. γδ T cell level can be measured in a number of ways, including flow cytometry (e.g., to quantify the percentage of γδ T cells relative to other cells in a biological sample), scRNA-seq, or SITE-seq. Unless indicated otherwise, γδ T cell level is measured using flow cytometry. In some embodiments, a subject may be selected for treatment (e.g., with a personalized cancer vaccine and/or an immune checkpoint inhibitor) if their γδ T cell or a sub-type/subset of γδ T cell (e.g., regulatory γδ T cell) level is greater than a set value. In some embodiments, a subject may be selected for treatment (e.g., with a personalized cancer vaccine and/or an immune checkpoint inhibitor) if their γδ T cell or a sub-type/subset of γδ T cell (e.g., regulatory γδ T cell) level is less than a set value. In some embodiments, the set value is based on an average or median γδ T cell level measured in a population of subjects (e.g., a population of subjects diagnosed as having a particular type of cancer, or a population of subjects having received primary treatment such as surgical resection of a particular type of cancer). For example, in some embodiments, the set value is 20%, 25%, 30%, 35%, 40%, 45%, 50%, 55%, 60%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, or 200% of the average or median γδ T cell level measured in a population of subjects. For example, in some embodiments, a subject may be selected for treatment if their γδ T cell level is greater than 100% of the average or median γδ T cell level measured in a population of subjects. In some embodiments, the set value is a numerical value. For example, in some embodiments, the set value for γδ T cell or sub-type/subset of γδ T cell level is 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, or 20% of T lymphocytes (e.g., CD3 lymphocytes) in a biological sample (e.g., in a blood sample, such as in peripheral blood mononuclear cells in a blood sample).

A T cell receptor (TCR) clonotyping value reflects TCR diversity and abundance in a subject or in a biological sample. TCR clonotyping can be conducted in a number of ways known in the art, including by isolating a sample comprising T cells, sequencing the TCR genes of the T cells, and analyzing diversity of the TCR genes and relative abundance of different TCR genes.

In some embodiments, a TCR clonotyping value is a DE50 (diversity evenness score) value, which indicates the degree of clonality in a given data set (e.g., sequencing data from a biological sample). DE50 represents the ratio between the number of sequences accounting for 50% of the total repertoire abundance (cumulative frequency of each of these sequences) and the repertoire richness. Put another way, DE50 is the ratio of how many clonotypes amongst the most frequent in a data set are necessary to account for 50% of the total read counts, relative to the total number of read counts present. DE50 is described in Chiffelle et al. “T-cell repertoire analysis and metrics of diversity and clonality” Curr Opin Biotech. 2020, 65:284-295 (DOI: 10.1016/j.copbio.2020.07.010), and Hosoi, et al. “Increased diversity with reduced ‘diversity evenness’ of tumor infiltrating T-cells for the successful cancer immunotherapy” Sci Rep. 2018, 8:1058 (DOI: 10.1038/s41598-018-19548-y), the entire contents of each of which are herein incorporated by reference for this purpose. A low DE50 value indicates a high clonality level, whereas a high DE50 value indicates that the different clonotypes are evenly represented in the sample in terms of their frequency. In some embodiments, a subject may be selected for treatment (e.g., with a personalized cancer vaccine and/or an immune checkpoint inhibitor) if their TCR clonotyping value of DE50 is greater than a set value. In some embodiments, a subject may be selected for treatment (e.g., with a personalized cancer vaccine and/or an immune checkpoint inhibitor) if their TCR clonotyping value of DE50 is less than a set value.

In some embodiments, a TCR clonotyping value is a Gini coefficient value, which measures the inequality among values of a frequency distribution. The Gini coefficient is calculated according to the following formula:

$$G i n {i}_{c} = \frac{{\sum }_{i = 1}^{N} {\sum }_{j = 1}^{N} \vert {p}_{i} - {p}_{j} \vert}{2 {N}^{2} \overline{p}} ,$$

wherein pᵢ and pⱼ represent the frequency of the respective i^{th} and j^{th} sequences in the repertoire, and p̅ represents the average of the clone frequencies. The Gini coefficient ranges from 0, representing maximal diversity of the repertoire (i.e., equal abundance of each sequence) to 1, representing extreme inequality (i.e., high clonality towards one sequence). The Gini coefficient is described in Chiffelle et al. “T-cell repertoire analysis and metrics of diversity and clonality” Curr Opin Biotech. 2020, 65:284-295 (DOI: 10.1016/j.copbio.2020.07.010), the entire contents of which are herein incorporated by reference for this purpose. In some embodiments, a subject may be selected for treatment (e.g., with a personalized cancer vaccine and/or an immune checkpoint inhibitor) if their TCR clonotyping value of Gini coefficient is greater than a set value. In some embodiments, a subject may be selected for treatment (e.g., with a personalized cancer vaccine and/or an immune checkpoint inhibitor) if their TCR clonotyping value of Gini coefficient is less than a set value.

These TCR clonotyping values and techniques, as well as others, are described in Arankumar et al., “T-Cell Receptor Repertoire Analysis with Computational Tools—An Immunologist's Perspective” Cells 2021, 10, 3582 (doi: 10.3390/cells10123582), the entire contents of which are herein incorporated by reference for this purpose.

In some embodiments, a subject may be selected for treatment (e.g., with a personalized cancer vaccine and/or an immune checkpoint inhibitor) if their TCR clonotyping value (e.g., DE50 or Gini coefficient) is greater than a set value. In some embodiments, a subject may be selected for treatment (e.g., with a personalized cancer vaccine and/or an immune checkpoint inhibitor) if their TCR clonotyping value (e.g., DE50 or Gini coefficient) is less than a set value. In some embodiments, the set value is based on an average or median TCR clonotyping value measured in a population of subjects (e.g., a population of subjects diagnosed as having a particular type of cancer, or a population of subjects having received primary treatment such as surgical resection of a particular type of cancer). For example, in some embodiments, the set value is 20%, 25%, 30%, 35%, 40%, 45%, 50%, 55%, 60%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, or 200% of the average or median TCR clonotyping value measured in a population of subjects. For example, in some embodiments, a subject may be selected for treatment if their TCR clonotyping value is greater than 100% of the average or median TCR clonotyping value measured in a population of subjects. As another example, in some embodiments, a subject may be selected for treatment if their TCR clonotyping value is less than 100% of the average or median TCR clonotyping value measured in a population of subjects. In some embodiments, the set value is a numerical value. For example, in some embodiments, the set value for DE50 is 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, or 49.5%. As another example, in some embodiments, the set value for Gini coefficient is 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, or 0.95.

Th1 cell population level reflects the percentage of Th1 cells (or a sub-type/subset thereof) relative to other white blood cells (e.g., total T lymphocytes, CD4 T lymphocytes, or total PBMCs) in a subject or in a biological sample. Th1 cell population level can be measured in a number of ways, including flow cytometry (e.g., to quantify the percentage of Th1 cells relative to other cells in a biological sample), scRNA-seq, or SITE-seq. Unless indicated otherwise, Th1 cell population level is measured using flow cytometry. In some embodiments, a subject may be selected for treatment (e.g., with a personalized cancer vaccine and/or an immune checkpoint inhibitor) if their Th1 cell population level is greater than a set value. In some embodiments, a subject may be selected for treatment (e.g., with a personalized cancer vaccine and/or an immune checkpoint inhibitor) if their Th1 cell population level is less than a set value. In some embodiments, the set value is based on an average or median Th1 cell population level measured in a population of subjects (e.g., a population of subjects diagnosed as having a particular type of cancer, or a population of subjects having received primary treatment such as surgical resection of a particular type of cancer). For example, in some embodiments, the set value is 20%, 25%, 30%, 35%, 40%, 45%, 50%, 55%, 60%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, or 200% of the average or median Th1 cell population level measured in a population of subjects. For example, in some embodiments, a subject may be selected for treatment if their Th1 cell population level is greater than 100% of the average or median Th1 cell population level measured in a population of subjects, or a subject may be selected for treatment if their Th1 cell population level is less than 100% of the average or median Th1 cell population level measured in a population of subjects. In some embodiments, the set value is a numerical value. For example, in some embodiments, the set value for Th1 cell population level is 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, or 45% of T lymphocytes (e.g., CD4 T lymphocytes) in a biological sample (e.g., in a blood sample, such as in peripheral blood mononuclear cells in a blood sample).

Methods of measuring gene expression, e.g., to measure a biomarker, are known by those of skill in the art, and include NanoString nCounter® gene expression analysis as well as protein expression measurement techniques such as flow cytometry.

In some embodiments, the subject may be selected for treatment based on a threshold value of a biomarker provided herein.

### Nucleic Acids/Polynucleotides

Cancer vaccines (e.g., nucleic acid cancer vaccines), as provided herein, comprise at least one (one or more) nucleic acid having an open reading frame encoding at least one peptide epitope. The term “nucleic acid,” in its broadest sense, includes any compound and/or substance that comprises a polymer of nucleotides. These polymers are also referred to as polynucleotides.

Nucleic acids may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof.

As a non-limiting example, when a DNA nucleic acid cancer vaccine is delivered to a cell, the DNA is transcribed into RNA, and the RNA will be processed into a polypeptide by the intracellular machinery which can then process the polypeptide into immunosensitive fragments capable of stimulating an immune response against a tumor or population of cancerous cells. As a non-limiting example, when an RNA (e.g., mRNA) nucleic acid cancer vaccine is delivered to a cell, the RNA (e.g., mRNA) will be processed into a polypeptide by the intracellular machinery which can then process the polypeptide into immunosensitive fragments capable of stimulating an immune response against a tumor or population of cancerous cells.

In some embodiments, nucleic acids function as messenger RNA (mRNA). “Messenger RNA” (mRNA) refers to any nucleic acid that encodes a (at least one) polypeptide (a naturally-occurring, non-naturally-occurring, or modified polymer of amino acids) and can be translated to produce the encoded polypeptide in vitro, in vivo, in situ or ex vivo.

The basic components of an mRNA molecule typically include at least one coding region, a 5′ untranslated region (UTR), a 3′ UTR, a 5′ cap and a poly-A tail. Nucleic acids may function as mRNA but can be distinguished from wild-type mRNA in their functional and/or structural design features which serve to overcome existing problems of effective polypeptide expression using nucleic-acid based therapeutics.

Polynucleotides, in some embodiments, are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art-non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity with a naturally-occurring or wild-type sequence (e.g., a naturally-occurring or wild-type mRNA sequence encoding a polypeptide or protein of interest (e.g., an antigenic protein or polypeptide). In some embodiments, a codon optimized sequence shares less than 90% sequence identity with a naturally-occurring or wild-type sequence (e.g., a naturally-occurring or wild-type mRNA sequence encoding a polypeptide or protein of interest (e.g., an antigenic protein or polypeptide). In some embodiments, a codon optimized sequence shares less than 85% sequence identity with a naturally-occurring or wild-type sequence (e.g., a naturally-occurring or wild-type mRNA sequence encoding a polypeptide or protein of interest (e.g., an antigenic protein or polypeptide). In some embodiments, a codon optimized sequence shares less than 80% sequence identity with a naturally-occurring or wild-type sequence (e.g., a naturally-occurring or wild-type mRNA sequence encoding a polypeptide or protein of interest (e.g., an antigenic protein or polypeptide). In some embodiments, a codon optimized sequence shares less than 75% sequence identity with a naturally-occurring or wild-type sequence (e.g., a naturally-occurring or wild-type mRNA sequence encoding a polypeptide or protein of interest (e.g., an antigenic protein or polypeptide).

In some embodiments, a codon optimized sequence shares between 65% and 85% (e.g., between about 67% and about 85% or between about 67% and about 80%) sequence identity with a naturally-occurring or wild-type sequence (e.g., a naturally-occurring or wild-type mRNA sequence encoding a polypeptide or protein of interest (e.g., an antigenic protein or polypeptide). In some embodiments, a codon optimized sequence shares between 65% and 75% or about 80% sequence identity with a naturally-occurring or wild-type sequence (e.g., a naturally-occurring or wild-type mRNA sequence encoding a polypeptide or protein of interest (e.g., an antigenic protein or polypeptide).

In some embodiments, a codon optimized RNA may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemical Modifications

### Modified Nucleotide Sequences Encoding Epitope Antigen Polypeptides

In some embodiments, the nucleic acid cancer vaccine of the invention comprises one or more chemically modified nucleobases. Some aspects include modified polynucleotides comprising a polynucleotide described herein (e.g., a nucleic acid comprising a nucleotide sequence encoding one or more cancer peptide epitopes). The modified nucleic acids can be chemically modified and/or structurally modified. When the nucleic acids are chemically and/or structurally modified the polynucleotides can be referred to as “modified nucleic acids.”

The present disclosure provides for modified nucleosides and nucleotides of a nucleic acid (e.g., RNA polynucleotides, such as mRNA polynucleotides) encoding one or more cancer peptide epitopes. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside including a phosphate group. Modified nucleotides can by synthesized by any useful method, such as, for example, chemically, enzymatically, or recombinantly, to include one or more modified or non-natural nucleosides. Nucleic acids can comprise a region or regions of linked nucleosides. Such regions can have variable backbone linkages. The linkages can be standard phosphodiester linkages, in which case the polynucleotides would comprise regions of nucleotides.

The modified nucleic acids disclosed herein can comprise various distinct modifications. In some embodiments, the modified polynucleotides contain one, two, or more (optionally different) nucleoside or nucleotide modifications. In some embodiments, a modified polynucleotide introduced to a cell can exhibit one or more desirable properties such as, e.g., improved protein expression, reduced immunogenicity, or reduced degradation in the cell, as compared to an unmodified polynucleotide.

In some embodiments, a nucleic acid disclosed herein (e.g., a nucleic acid encoding one or more peptide epitopes) is structurally modified. As used herein, a “structural” modification is one in which two or more linked nucleosides are inserted, deleted, duplicated, inverted, or randomized in a polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” can be chemically modified to “AT-5meC-G.” The same polynucleotide can be structurally modified from “ATCG” to “ATCCCG.” Here, the dinucleotide “CC” has been inserted, resulting in a structural modification to the nucleic acid.

In some embodiments, the nucleic acids of the instant disclosure are chemically modified. As used herein in reference to a nucleic acid, the terms “chemical modification” or, as appropriate, “chemically modified” refer to modification with respect to adenosine (A), guanosine (G), uridine (U), or cytidine (C) ribo- or deoxyribonucleosides in one or more of their position, pattern, percentage, or population. Generally, herein, these terms are not intended to refer to the ribonucleotide modifications in naturally occurring 5′-terminal mRNA cap moieties.

In some embodiments, the nucleic acids of the instant disclosure can have a uniform chemical modification of all or any of the same nucleoside type or a population of modifications produced by mere downward titration of the same starting modification in all or any of the same nucleoside type, or a measured percent of a chemical modification of all any of the same nucleoside type but with random incorporation, such as where all uridines are replaced by a uridine analog, e.g., pseudouridine or 5-methoxyuridine. In some embodiments, the polynucleotides can have a uniform chemical modification of two, three, or four of the same nucleoside type throughout the entire polynucleotide (such as all uridines and all cytosines, etc. are modified in the same way).

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine, or uracil. Any combination of base/sugar or linker can be incorporated into polynucleotides.

The skilled artisan will appreciate that, except where otherwise noted, nucleic acid sequences set forth in the instant application will recite “T”s in a representative DNA sequence but where the sequence represents RNA, the “T”s would be substituted for “U”s.

Cancer vaccines comprise, in some embodiments, at least one nucleic acid (e.g., RNA) having an open reading frame encoding at least one (e.g., 5-200 or 5-130) peptide epitope(s), wherein the nucleic acid comprises nucleotides and/or nucleosides that can be standard (unmodified) or modified as is known in the art. In some embodiments, nucleotides and nucleosides comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in International Patent Application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB2017/051367 all of which are incorporated by reference herein for this purpose.

Hence, nucleic acids of the disclosure (e.g., DNA nucleic acids and RNA nucleic acids, such as mRNA nucleic acids) can comprise standard nucleotides and nucleosides, naturally-occurring nucleotides and nucleosides, non-naturally-occurring nucleotides and nucleosides, or any combination thereof.

Nucleic acids of the disclosure (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., DNA nucleic acids or RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids.

In some embodiments, modified nucleobases in nucleic acids (e.g., RNA nucleic acids, such as mRNA nucleic acids) comprise 1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a RNA nucleic acid of the disclosure comprises 1-methyl-pseudouridine (m1ψ) substitutions at one or more or all uridine positions of the nucleic acid.

In some embodiments, a RNA nucleic acid of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises pseudouridine (ψ) substitutions at one or more or all uridine positions of the nucleic acid.

In some embodiments, a RNA nucleic acid of the disclosure comprises pseudouridine (ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the poly A tail). In some embodiments, all nucleotides X in a nucleic acid (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U, or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90%, or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

In some embodiments, the nucleic acid can include any useful linker between the nucleosides. Such linkers, including backbone modifications, that are useful in the composition include, but are not limited to the following: 3′-alkylene phosphonates, 3′-amino phosphoramidate, alkene containing backbones, aminoalkylphosphoramidates, aminoalkylphosphotriesters, boranophosphates, —CH₂—O—N(CH₃)—CH₂—, —CH₂—N(CH₃)—N(CH₃)—CH₂—, —CH₂—NH—CH₂—, chiral phosphonates, chiral phosphorothioates, formacetyl and thioformacetyl backbones, methylene (methylimino), methylene formacetyl and thioformacetyl backbones, methyleneimino and methylenehydrazino backbones, morpholino linkages, —N(CH₃)—CH₂—CH₂—, oligonucleosides with heteroatom internucleoside linkage, phosphinates, phosphoramidates, phosphorodithioates, phosphorothioate internucleoside linkages, phosphorothioates, phosphotriesters, PNA, siloxane backbones, sulfamate backbones, sulfide sulfoxide and sulfone backbones, sulfonate and sulfonamide backbones, thionoalkylphosphonates, thionoalkylphosphotriesters, and thionophosphoramidates.

The modified nucleosides and nucleotides (e.g., building block molecules), which can be incorporated into a nucleic acid (e.g., RNA or mRNA, as described herein), can be modified on the sugar of the ribonucleic acid. For example, the 2′ hydroxyl group (OH) can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C1-6 alkylene or C1-6 heteroalkylene bridge to the 4′-carbon of the same ribose sugar, where exemplary bridges included methylene, propylene, ether, or amino bridges; aminoalkyl; aminoalkoxy; amino; and amino acid.

Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar. Such sugar modifications are described in, for example, International Patent Application Publication Nos. WO2013052523 and WO2014093924, the contents of each of which are incorporated herein by reference in their entireties for this purpose.

The nucleic acids of the disclosure (e.g., a nucleic acid encoding one or more peptide epitopes or a functional fragment or variant thereof) can include a combination of modifications to the sugar, the nucleobase, and/or the internucleoside linkage. These combinations can include any one or more modifications described herein.

The nucleic acid cancer vaccines disclosed herein are compositions, including pharmaceutical compositions. The disclosure also encompasses methods for the selection, design, preparation, manufacture, formulation, and/or use of nucleic acid cancer vaccines as provided herein. Also provided are systems (e.g., computerized systems), processes, devices and kits for the selection, design, and/or utilization of the nucleic acid cancer vaccines described herein.

In Vitro Transcription of RNA (e.g., mRNA)

Cancer vaccines may comprise at least one nucleic acid (e.g., an RNA polynucleotide, such as an mRNA (messenger RNA) or an mmRNA (modified mRNA)). mRNA, for example, is transcribed in vitro from template DNA, referred to as an “in vitro transcription template.” In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the mRNA encoded by the template.

In some embodiments, a nucleic acid includes 15 to 3,000 nucleotides. For example, a polynucleotide may include 15 to 50, 15 to 100, 15 to 200, 15 to 300, 15 to 400, 15 to 500, 15 to 600, 15 to 700, 15 to 800, 15 to 900, 15 to 1000, 15 to 1200, 15 to 1400, 15 to 1500, 15 to 1800, 15 to 2000, 15 to 2500, 15 to 3000, 50 to 100, 50 to 200, 50 to 300, 50 to 400, 50 to 500, 50 to 600, 50 to 700, 50 to 800, 50 to 900, 50 to 1000, 50 to 1200, 50 to 1400, 50 to 1500, 50 to 1800, 50 to 2000, 50 to 2500, 50 to 3000, 100 to 200, 100 to 300, 100 to 400, 100 to 500, 100 to 600, 100 to 700, 100 to 800, 100 to 900, 100 to 1000, 100 to 1200, 100 to 1400, 100 to 1500, 100 to 1800, 100 to 2000, 100 to 2500, 100 to 3000, 200 to 300, 200 to 400, 200 to 500, 200 to 600, 200 to 700, 200, to 800, 200 to 900, 200 to 1000, 200 to 1500, 200 to 3000, 500 to 1000, 500 to 1500, 500 to 2000, 500 to 2500, 500 to 3000, 1000 to 1500, 1000 to 2000, 1000 to 2500, 1000 to 3000, 1500 to 3000, 2500 to 3000, or 2000 to 3000 nucleotides).

In other aspects, the disclosure relates to a method for preparing a nucleic acid cancer vaccine (e.g., an mRNA cancer vaccine) by IVT methods. In vitro transcription (IVT) methods permit template-directed synthesis of RNA molecules of almost any sequence. The size of the RNA molecules that can be synthesized using IVT methods range from short oligonucleotides to long nucleic acid polymers of several thousand bases. IVT methods permit synthesis of large quantities of RNA transcript (e.g., from microgram to milligram quantities). See Beckert et al., Synthesis of RNA by in vitro transcription, Methods Mol Biol. 703:29-41(2011); Rio et al. RNA: A Laboratory Manual. Cold Spring Harbor: Cold Spring Harbor Laboratory Press, 2011, 205-220; Cooper, Geoffery M. The Cell: A Molecular Approach. 4th ed. Washington D.C.: ASM Press, 2007. 262-299, each of which is herein incorporated by reference for this purpose. Generally, IVT utilizes a DNA template featuring a promoter sequence upstream of a sequence of interest. The promoter sequence is most commonly of bacteriophage origin (e.g., the T7, T3 or SP6 promoter sequence) but many other promotor sequences can be tolerated including those designed de novo. Transcription of the DNA template is typically best achieved by using the RNA polymerase corresponding to the specific bacteriophage promoter sequence. Exemplary RNA polymerases include, but are not limited to T7 RNA polymerase, T3 RNA polymerase, or SP6 RNA polymerase, among others. IVT is generally initiated at a dsDNA but can proceed on a single strand.

It will be appreciated that nucleic acid cancer vaccines (e.g., mRNA cancer vaccines), e.g., mRNAs encoding peptide epitope(s), such as cancer antigen peptide epitope(s), may be made using any appropriate synthesis method. For example, in some embodiments, mRNA vaccines are made using IVT from a single bottom strand DNA as a template and complementary oligonucleotide that serves as promotor. The single bottom strand DNA may act as a DNA template for in vitro transcription of RNA, and may be obtained from, for example, a plasmid, a PCR product, or chemical synthesis. In some embodiments, the single bottom strand DNA is linearized from a circular template. The single bottom strand DNA template generally includes a promoter sequence, e.g., a bacteriophage promoter sequence, to facilitate IVT. Methods of making RNA using a single bottom strand DNA and a top strand promoter complementary oligonucleotide are known in the art. An exemplary method includes, but is not limited to, annealing the DNA bottom strand template with the top strand promoter complementary oligonucleotide (e.g., T7 promoter complementary oligonucleotide, T3 promoter complementary oligonucleotide, or SP6 promoter complementary oligonucleotide), followed by IVT using an RNA polymerase corresponding to the promoter sequence, e.g., aT7 RNA polymerase, a T3 RNA polymerase, or an SP6 RNA polymerase.

IVT methods can also be performed using a double-stranded DNA template. For example, in some embodiments, the double-stranded DNA template is made by extending a complementary oligonucleotide to generate a complementary DNA strand using strand extension techniques available in the art. In some embodiments, a single bottom strand DNA template containing a promoter sequence and sequence encoding one or more peptide epitopes of interest is annealed to a top strand promoter complementary oligonucleotide and subjected to a PCR-like process to extend the top strand to generate a double-stranded DNA template. Alternatively or additionally, a top strand DNA containing a sequence complementary to the bottom strand promoter sequence and complementary to the sequence encoding one or more peptide epitopes of interest is annealed to a bottom strand promoter oligonucleotide and subjected to a PCR-like process to extend the bottom strand to generate a double-stranded DNA template. In some embodiments, the number of PCR-like cycles ranges from 1 to 20 cycles, e.g., 3 to 10 cycles. In some embodiments, a double-stranded DNA template is synthesized wholly or in part by chemical synthesis methods. The double-stranded DNA template can be subjected to in vitro transcription as described herein.

In another aspect, nucleic acid cancer vaccines comprising, e.g., mRNAs encoding peptide epitope(s), such as cancer antigen peptide epitope(s), may be made using two DNA strands that are complementary across an overlapping portion of their sequence, leaving single-stranded overhangs (i.e., sticky ends) when the complementary portions are annealed. These single-stranded overhangs can be made double-stranded by extending using the other strand as a template, thereby generating double-stranded DNA. In some cases, this primer extension method can permit larger ORFs to be incorporated into the template DNA sequence, e.g., as compared to sizes incorporated into the template DNA sequences obtained by top strand DNA synthesis methods. In the primer extension method, a portion of the 3′-end of a first strand (in the 5′-3′ direction) is complementary to a portion the 3′-end of a second strand (in the 3′-5′ direction). In some such embodiments, the single first strand DNA may include a sequence of a promoter (e.g., T7, T3, or SP6), optionally a 5′-UTR, and some or all of an ORF (e.g., a portion of the 5′-end of the ORF). In some embodiments, the single second strand DNA may include complementary sequences for some or all of an ORF (e.g., a portion complementary to the 3′-end of the ORF), and optionally a 3′-UTR, a stop sequence, and/or a poly(A) tail. Methods of making RNA using two synthetic DNA strands may include annealing the two strands with overlapping complementary portions, followed by primer extension using one or more PCR-like cycles to extend the strands to generate a double-stranded DNA template. In some embodiments, the number of PCR-like cycles ranges from 1 to 20 cycles, e.g., 3 to 10 cycles. Such double-stranded DNA can be subjected to in vitro transcription as described herein.

In another aspect, nucleic acid vaccines comprising, e.g., mRNAs encoding peptide epitope(s), such as cancer antigen peptide epitope(s), may be made using synthetic double-stranded linear DNA molecules, such as gBlocks® (Integrated DNA Technologies, Coralville, Iowa), as the double-stranded DNA template. An advantage to such synthetic double-stranded linear DNA molecules is that they provide a longer template from which to generate mRNAs. For example, gBlocks® can range in size from 45-1000 (e.g., 125-750 nucleotides). In some embodiments, a synthetic double-stranded linear DNA template includes a full length 5′-UTR, a full length 3′-UTR, or both. A full length 5′-UTR may be up to 100 nucleotides in length, e.g., about 40-60 nucleotides. A full length 3′-UTR may be up to 300 nucleotides in length, e.g., about 100-150 nucleotides.

To facilitate generation of longer constructs, two or more double-stranded linear DNA molecules and/or gene fragments that are designed with overlapping sequences on the 3′ strands may be assembled together using methods known in art. For example, the Gibson Assembly™ Method (Synthetic Genomics, Inc., La Jolla, CA) may be performed with the use of a mesophilic exonuclease that cleaves bases from the 5′-end of the double-stranded DNA fragments, followed by annealing of the newly formed complementary single-stranded 3′-ends, polymerase-dependent extension to fill in any single-stranded gaps, and finally, covalent joining of the DNA segments by a DNA ligase.

In another aspect, nucleic acid cancer vaccines of the present disclosure comprising, e.g., mRNAs encoding peptide epitope(s), such as cancer antigen peptide epitope(s), may be made using chemical synthesis of the RNA. Methods, for instance, involve annealing a first polynucleotide comprising an open reading frame encoding the polypeptide and a second polynucleotide comprising a 5′-UTR to a complementary polynucleotide conjugated to a solid support. The 3′-terminus of the second polynucleotide is then ligated to the 5′-terminus of the first polynucleotide under suitable conditions. Suitable conditions include the use of a DNA Ligase. The ligation reaction produces a first ligation product. The 5′ terminus of a third polynucleotide comprising a 3′-UTR is then ligated to the 3′-terminus of the first ligation product under suitable conditions. Suitable conditions for the second ligation reaction include an RNA Ligase. A second ligation product is produced in the second ligation reaction. The second ligation product is released from the solid support to produce an mRNA encoding a polypeptide of interest. In some embodiments, the mRNA is between 30 and 1000 nucleotides.

An mRNA encoding one or more peptide epitopes may also be prepared by binding a first nucleic acid comprising an open reading frame encoding the nucleic acid to a second nucleic acid comprising 3′-UTR to a complementary nucleic acid conjugated to a solid support. The 5′-terminus of the second nucleic acid is ligated to the 3′-terminus of the first nucleic acid under suitable conditions (including, e.g., a DNA Ligase). The method produces a first ligation product. A third nucleic acid comprising a 5′-UTR is ligated to the first ligation product under suitable conditions (including, e.g., an RNA Ligase, such as T4 RNA) to produce a second ligation product. The second ligation product is released from the solid support to produce an mRNA encoding one or more peptide epitopes.

In some embodiments, the first nucleic acid features a 5′-triphosphate and a 3′-OH. In other embodiments, the second nucleic acid comprises a 3′-OH. In yet other embodiments, the third nucleic acid comprises a 5′-triphosphate and a 3′-OH. The second nucleic acid may also include a 5′-cap structure. The method may also involve the further step of ligating a fourth nucleic acid comprising a poly-A region at the 3′-terminus of the third nucleic acid. The fourth nucleic acid may comprise a 5′-triphosphate.

The method may or may not comprise reverse phase purification. The method may also include a washing step wherein the solid support is washed to remove unreacted nucleic acids. The solid support may be, for instance, a capture resin. In some embodiments, the method involves dT purification.

In accordance with the present disclosure, template DNA encoding the nucleic acid (e.g., mRNA) cancer vaccines includes an open reading frame (ORF) encoding one or more peptide epitopes. In some embodiments, the template DNA includes an ORF of up to 1000 nucleotides, e.g., about 10-350, 30-300 nucleotides or about 50-250 nucleotides. In some embodiments, the template DNA includes an ORF of about 150 nucleotides. In some embodiments, the template DNA includes an ORF of about 200 nucleotides.

In some embodiments, IVT transcripts are purified from the components of the IVT reaction mixture after the reaction takes place. For example, the crude IVT mix may be treated with RNase-free DNase to digest the original template. The nucleic acid (e.g., mRNA) can be purified using methods known in the art, including but not limited to, precipitation using an organic solvent or column based purification method. Commercial kits are available to purify RNA, e.g., MEGACLEAR™ Kit (Ambion, Austin, TX). The nucleic acid (e.g., mRNA) can be quantified using methods known in the art, including but not limited to, commercially available instruments, e.g., NanoDrop. Purified nucleic acids (e.g., mRNAs) can be analyzed, for example, by agarose gel electrophoresis to confirm the nucleic acid is the proper size and/or to confirm that no degradation of the nucleic acid has occurred.

### Untranslated Regions (UTRs)

Untranslated regions (UTRs) are sections of a nucleic acid before a start codon (5′ UTR) and after a stop codon (3′ UTR) that are not translated. In some embodiments, a nucleic acid (e.g., a ribonucleic acid (RNA), e.g., a messenger RNA (mRNA)) of the disclosure comprising an open reading frame (ORF) encoding one or more peptide epitopes further comprises one or more UTR (e.g., a 5′ UTR or functional fragment thereof, a 3′ UTR or functional fragment thereof, or a combination thereof).

A UTR can be homologous or heterologous to the coding region in a nucleic acid. In some embodiments, the UTR is homologous to the ORF encoding the one or more peptide epitopes. In some embodiments, the UTR is heterologous to the ORF encoding the one or more peptide epitopes. In some embodiments, the nucleic acid comprises two or more 5′ UTRs or functional fragments thereof, each of which have the same or different nucleotide sequences. In some embodiments, the nucleic acid comprises two or more 3′ UTRs or functional fragments thereof, each of which have the same or different nucleotide sequences.

In some embodiments, the 5′ UTR or functional fragment thereof, 3′ UTR or functional fragment thereof, or any combination thereof is sequence optimized.

In some embodiments, the 5′ UTR or functional fragment thereof, 3′ UTR or functional fragment thereof, or any combination thereof comprises at least one chemically modified nucleobase, e.g., 5-methoxyuracil.

UTRs can have features that provide a regulatory role, e.g., increased or decreased stability, localization, and/or translation efficiency. A nucleic acid comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′ UTRs bear features that play roles in translation initiation. They harbor signatures like Kozak sequences that are commonly known to be involved in the process by which the ribosome initiates translation of many genes. 5′ UTRs also have been known to form secondary structures that are involved in elongation factor binding.

By engineering the features typically found in abundantly expressed genes of specific target organs, one can enhance the stability and protein production of a nucleic acid. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of nucleic acids in hepatic cell lines or liver. Likewise, use of 5′ UTRs from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin), and for lung epithelial cells (e.g., SP-A/B/C/D).

In some embodiments, UTRs are selected from a family of transcripts whose proteins share a common function, structure, feature, or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new nucleic acid.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

International Patent Application No. PCT/US2014/021522 (Publ. No. WO2014/164253) provides a listing of exemplary UTRs that may be utilized in the nucleic acids as flanking regions to an ORF. This publication is incorporated by reference herein for this purpose.

Additional exemplary UTRs that may be utilized in the nucleic acids include, but are not limited to, one or more 5′ UTRs and/or 3′ UTRs derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV; e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′ UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

In some embodiments, the 5′ UTR is selected from the group consisting of a β-globin 5′ UTR; a 5′ UTR containing a strong Kozak translational initiation signal; a cytochrome b-245 α polypeptide (CYBA) 5′ UTR; a hydroxysteroid (17-β) dehydrogenase (HSD17B4) 5′ UTR; a Tobacco etch virus (TEV) 5′ UTR; a Venezuelen equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′ UTR; a DEN 3′ UTR; a PAV barley yellow dwarf virus (BYDV-PAV) 3′ UTR; an elongation factor 1 al (EEF1A1) 3′ UTR; a manganese superoxide dismutase (MnSOD) 3′ UTR; a β subunit of mitochondrial H (+)-ATP synthase (β-mRNA) 3′ UTR; a GLUT1 3′ UTR; a MEF2A 3′ UTR; a β-F1-ATPase 3′ UTR; functional fragments thereof and combinations thereof.

In some embodiments, the 5′ UTR comprises a sequence provided in Table C below or a sequence with at least 80%, 85%, 90%, 95%, 96%, 97%, 98%, 99% or 100% identity to a 5′ UTR sequence provided in the following Table, or a variant or a fragment thereof.

TABLE C
5′ UTR sequences

| SEQ ID | |
|---|---|
| NO: | Sequence |
| 1 | GGAAAUCGCAAAAUUUGCUCUUCGCGUUAGAUUUCUUUUAGUUUUCU |
| | CGCAACUAGCAAGCUUUUUGUUCUCGCC |
| 2 | GGAAAUCCCCACAACCGCCUCAUAUCCAGGCUCAAGAAUAGAGCUCA |
| | GUGUUUUGUUGUUUAAUCAUUCCGACGUGUUUUGCGAUAUUCGCGCA |
| | AAGCAGCCAGUCGCGCGCUUGCUUUUAAGUAGAGUUGUUUUUCCACC |
| | CGUUUGCCAGGCAUCUUUAAUUUAACAUAUUUUUAUUUUUCAGGCUA |
| | ACCUACGCCGCCACC |
| 3 | GGAAAUAAGAGAGAAAAGAAGAGUAAGAAGAAAUAUAAGAUCUCCC |
| | UGAGCUUCAGGGAGCCCCGGCGCCGCCACC |
| 4 | GGAAACCCCCCACCCCCGUAAGAGAGAAAAGAAGAGUAAGAAGAAAU |
| | AUAAGAUCUCCCUGAGCUUCAGGGAGCCCCGGCGCCGCCACC |
| 5 | GGAGAACUUCCGCUUCCGUUGGCGCAAGCGCUUUCAUUUUUUCUGCU |
| | ACCGUGACUAAG |
| 6 | GGAAAUAAGAGAGAAAAGAAGAGUAAGAAGAAAUAUAAGAGCCACC |
| 7 | GGAAAUAAGAGAGAAAAGAAGAGUAAGAAGAAAUAUAAGACCCCGG |
| | CGCCGCCACC |
| 8 | GGAAAUCGCAAAAUUUGCUCUUCGCGUUAGAUUUCUUUUAGUUUUCU |
| | CGCAACUAGCAAGCUUUUUGUUCUCGCCGCCGCC |
| 9 | GGAAAUCGCAAAAUUUUCUUUUCGCGUUAGAUUUCUUUUAGUUUUCU |
| | UUCAACUAGCAAGCUUUUUGUUCUCGCCGCCGCC |
| 10 | G G A A A U C G C A A A A (N₂)x (N₃)x C U (N₄)x |
| | (N₅)x C G C G U U A G A U U U C U U U U A G U |
| | U U U C U N₆ N₇ C A A C U A G C A A G C U U U U |
| | U G U U C U C G C C (N₈ C C)x |
| | (N₂)x is a uracil and x is an integer from 0 to 5, |
| | e.g., wherein x = 3 or 4; |
| | (N₃)x is a guanine and x is an integer from 0 to 1; |
| | (N₄)x is a cytosine and x is an integer from 0 to 1; |
| | (N₅)x is a uracil and x is an integer from 0 to 5, |
| | e.g., wherein x = 2 or 3; |
| | N₆ is a uracil or cytosine; |
| | N₇ is a uracil or guanine; |
| | N₈ is adenine or guanine and x is an integer from 0 to 1. |
| 11 | GGAAAAUUUUAGCCUGGAACGUUAGAUAACUGUCCUGUUGUCUUUAU |
| | AUACUUGGUCCCCAAGUAGUUUGUCUUCCAAA |
| 12 | GGAAACUUUAUUUAGUGUUACUUUAUUUUCUGUUUAUUUGUGUUUC |
| | UUCAGUGGGUUUGUUCUAAUUUCCUUGGCCGCC |
| 13 | GGAAAAUCUGUAUUAGGUUGGCGUGUUCUUUGGUCGGUUGUUAGUA |
| | UUGUUGUUGAUUCGUUUGUGGUCGGUUGCCGCC |
| 14 | GGAAAAUUAUUAACAUCUUGGUAUUCUCGAUAACCAUUCGUUGGAUU |
| | UUAUUGUAUUCGUAGUUUGGGUUCCUGCCGCC |
| 15 | GGAAAUUAUUAUUAUUUCUAGCUACAAUUUAUCAUUGUAUUAUUUU |
| | AGCUAUUCAUCAUUAUUUACUUGGUGAUCAACA |
| 16 | GGAAAUAGGUUGUUAACCAAGUUCAAGCCUAAUAAGCUUGGAUUCUG |
| | GUGACUUGCUUCACCGUUGGCGGGCACCGAUC |
| 17 | GGAAAUCGUAGAGAGUCGUACUUAGUACAUAUCGACUAUCGGUGGAC |
| | ACCAUCAAGAUUAUAAACCAGGCCAGA |
| 18 | GGAAACCCGCCCAAGCGACCCCAACAUAUCAGCAGUUGCCCAAUCCCA |
| | ACUCCCAACACAAUCCCCAAGCAACGCCGCC |
| 19 | GGAAAGCGAUUGAAGGCGUCUUUUCAACUACUCGAUUAAGGUUGGGU |
| | AUCGUCGUGGGACUUGGAAAUUUGUUGUUUCC |
| 20 | GGAAACUAAUCGAAAUAAAAGAGCCCCGUACUCUUUUAUUUCUAUUA |
| | GGUUAGGAGCCUUAGCAUUUGUAUCUUAGGUA |
| 21 | GGAAAUGUGAUUUCCAGCAACUUCUUUUGAAUAUAUUGAAUUCCUAA |
| | UUCAAAGCGAACAAAUCUACAAGCCAUAUACC |
| 22 | GGAAAUCGUAGAGAGUCGUACUUACGUGGUCGCCAUUGCAUAGCGCG |
| | CGAAAGCAACAGGAACAAGAACGCGCC |
| 23 | GGAAAUCGUAGAGAGUCGUACUUAGAAUAAACAGAGUCGGGUCGACU |
| | UGUCUCUGAUACUACGACGUCACAAUC |
| 24 | GGAAAAUUUGCCUUCGGAGUUGCGUAUCCUGAACUGCCCAGCCUCCU |
| | GAUAUACAACUGUUCCGCUUAUUCGGGCCGCC |
| 25 | GGAAAUCUGAGCAGGAAUCCUUUGUGCAUUGAAGACUUUAGAUUCCU |
| | CUCUGCGGUAGACGUGCACUUAUAAGUAUUUG |
| 26 | GGAAAGCGAUUGAAGGCGUCUUUUCAACUACUCGAUUAAGGUUGGGU |
| | AUCGUCGUGGGACUUGGAAAUUUGUUGCCACC |
| 27 | GGAAAUUUUUUUUUGAUAUUAUAAGAGUUUUUUUUUGAUAUUAAGA |
| | AAAUUUUUUUUUGAUAUUAGAAGAGUAAGAAGAAAUAUAAGACCCC |
| | GGCGCCGCCACC |
| 28 | GGAAAUAAGAGAGAAAAGAAGAGUAAGAAGAAAUAUAAGAGCCAAA |
| | AAAAAAAAACC |
| 29 | GGAAAUCUCCCUGAGCUUCAGGGAGUAAGAGAGAAAAGAAGAGUAAG |
| | AAGAAAUAUAAGACCCCGGCGCCGCCACC |
| 30 | GCCRCC, wherein R = A or G |
| 31 | GGACUCACUAUUUGUUUUCGCGCCCAGUUGCAAAAA |

In some embodiments, the 3′ UTR comprises a sequence provided in the following Table or a sequence with at least 80%, 85%, 90%, 95%, 96%, 97%, 98%, 99% or 100% identity to a 3′ UTR sequence provided in Table D below, or a variant or a fragment thereof.

TABLE D
UTR sequences (stop cassette is italicized;
miR binding sites are boldened)

| SEQ | |
|---|---|
| ID NO: | Sequence |
| 32 | UAAAGCUCCCCGGGGGCCUCGGUGGCCUAGCUUCUUGCCCCUUGGG |
| | CCUCCCCCCAGCCCCUCCUCCCCUUCCUGCACCCGUACCCCCGUGGUCU |
| | UUGAAUAAAGUCUGAGUGGGCGGC |
| 33 | UAAGUCUAAGCUGGAGCCUCCUGAGAGACCUGUGUGAACUAUUGAG |
| | AAGAUCGGAACAGCUCCUUACUCUGAGGAAGUUGGUACCCCCGUGGUC |
| | UUUGAAUAAAGUCUGAGUGGGCGGC |
| 34 | UAAAGCUCCCCGGGGCAAACACCAUUGUCACACUCCAGCCUCGGU |
| | GGCCUAGCUUCUUGCCCCUUGGGCCCAAACACCAUUGUCACACUCCA |
| | UCCCCCCAGCCCCUCCUCCCCUUCCUGCACCCGUACCCCCCAAACACCA |
| | UUGUCACACUCCAGUGGUCUUUGAAUAAAGUCUGAGUGGGCGGC |
| | (miR122 binding sites boldened) |
| 35 | UAAAGCUCCCCGGGGUCCAUAAAGUAGGAAACACUACAGCUGGAG |
| | CCUCGGUGGCCUAGCUUCUUGCCCCUUGGGCCCAAACACCAUUGUCA |
| | CACUCCAUCCCCCCAGCCCCUCCUCCCCUUCCUGCACCCGUACCCCCGU |
| | GGUCUUUGAAUAAAGUCUGAGUGGGCGGC |
| | (miR-142-3p and miR 122 binding sites boldened) |
| 36 | UAAAGCUCCCCGGGGGCCUCGGUGGCCUAGCUUCUUGCCCCUUGGG |
| | CCUCCCCCCAGCCCCUCCUCCCCUUCCUGCACCCGUACCCCCCAAACAC |
| | CAUUGUCACACUCCAGUGGUCUUUGAAUAAAGUCUGAGUGGGGGC |
| | (miR122 binding site boldened) |
| 37 | UAAGCCCCUCCGGGGCAAACACCAUUGUCACACUCCAGCCUCGGU |
| | GGCCUAGCUUCUUGCCCCUUGGGCCCAAACACCAUUGUCACACUCCA |
| | UCCCCCCAGCCCCUCCUCCCCUUCCUGCACCCGUACCCCCCAAACACCA |
| | UUGUCACACUCCAGUGGUCUUUGAAUAAAGUCUGAGUGGGCGGC |
| | (miR 122 binding sites boldened) |
| 38 | UAAGCCCCUCCGGGGUCCAUAAAGUAGGAAACACUACAGCCUCGG |
| | UGGCCUAGCUUCUUGCCCCUUGGGCCUCCAUAAAGUAGGAAACACUA |
| | CAUCCCCCCAGCCCCUCCUCCCCUUCCUGCACCCGUACCCCCCGCAUUA |
| | UUACUCACGGUACGAGUGGUCUUUGAAUAAAGUCUGAGUGGGCGGC |
| | (miR-142-3p and miR-126-3p binding sites boldened) |
| 39 | UAAGCCCCUCCGGGGUCCAUAAAGUAGGAAACACUACAGCUGGAG |
| | CCUCGGUGGCCUAGCUUCUUGCCCCUUGGGCCCAAACACCAUUGUCA |
| | CACUCCAUCCCCCCAGCCCCUCCUCCCCUUCCUGCACCCGUACCCCCGU |
| | GGUCUUUGAAUAAAGUCUGAGUGGGCGGC |
| | (miR-142-3p and miR 122 binding sites boldened) |
| 40 | UAAGCCCCUCCGGGGGCCUCGGUGGCCUAGCUUCUUGCCCCUUGGG |
| | CCUCCCCCCAGCCCCUCCUCCCCUUCCUGCACCCGUACCCCCCAAACAC |
| | CAUUGUCACACUCCAGUGGUCUUUGAAUAAAGUCUGAGUGGGCGGC |
| | (miR122 binding site boldened) |

In some embodiments, the polynucleotide comprises a stop element and 3′-UTR, wherein the sequence is (stop element is italicized): UAAAGCUCCCCGGGGGCCUCGGUGGCCUAGCUUCUUGCCCCUUGGGCCUCCCCCCAGC CCCUCCUCCCCUUCCUGCACCCGUACCCCCGUGGUCUUUGAAUAAAGUCUGAGUGGG CGGC (SEQ ID NO:32) or a variant or fragment thereof (e.g., a fragment that lacks the first one, two, three, four, five, six, or more nucleotides of nucleotides of SEQ ID NO:32.

Wild-type UTRs derived from any gene or mRNA can be incorporated into the nucleic acids of the disclosure. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, and sequences available at addgene.org/Derrick_Rossi/, the contents of each are incorporated herein by reference in their entirety. UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the nucleic acid may comprise multiple UTRs, e.g., a double, a triple or a quadruple 5′ UTR or 3′ UTR. For example, a double UTR comprises two copies of the same UTR either in series or substantially in series. For example, a double beta-globin 3′ UTR can be used (see, for example, US Patent Application Publication No. US2010/0129877, the contents of which are incorporated herein by reference for this purpose).

The nucleic acids of the disclosure can comprise combinations of features. For example, the ORF can be flanked by a 5′ UTR that comprises a strong Kozak translational initiation signal and/or a 3′ UTR comprising an oligo (dT) sequence for templated addition of a poly-A tail. A 5′ UTR can comprise a first nucleic acid fragment and a second nucleic acid fragment from the same and/or different UTRs (see, e.g., US Patent Application Publication No. US2010/0293625, herein incorporated by reference in its entirety for this purpose).

Other non-UTR sequences can be used as regions or subregions within the nucleic acids of the disclosure. For example, introns or portions of intron sequences can be incorporated into the nucleic acids of the disclosure. Incorporation of intronic sequences can increase protein production as well as nucleic acid expression levels. In some embodiments, the nucleic acid of the disclosure comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the nucleic acid comprises an IRES instead of a 5′ UTR sequence. In some embodiments, the nucleic acid comprises an ORF and a viral capsid sequence. In some embodiments, the nucleic acid comprises a synthetic 5′ UTR in combination with a non-synthetic 3′ UTR.

In some embodiments, the UTR can also include at least one translation enhancer nucleic acid, translation enhancer element, or translational enhancer elements (collectively, “TEE,” which refers to nucleic acid sequences that increase the amount of polypeptide or protein produced from a polynucleotide. As a non-limiting example, the TEE can include those described in US Patent Application Publication No. US2009/0226470, incorporated herein by reference in its entirety for this purpose, and others known in the art. As a non-limiting example, the TEE can be located between the transcription promoter and the start codon. In some embodiments, the 5′ UTR comprises a TEE. In one aspect, a TEE is a conserved element in a UTR that can promote translational activity of a nucleic acid such as, but not limited to, cap-dependent or cap-independent translation. In one non-limiting example, the TEE comprises the TEE sequence in the 5′-leader of the Gtx homeodomain protein. See Chappell et al., PNAS 2004 101:9590-9594, incorporated herein by reference in its entirety for this purpose.

The terms “translational enhancer polynucleotide” or “translation enhancer polynucleotide sequence” refer to a nucleic acid that includes one or more of the TEE provided herein and/or known in the art (see, e.g., U.S. Pat. Nos. 6,310,197, 6,849,405, 7,456,273, and 7,183,395; US Patent Application Publication Nos. US2009/0226470, US2007/0048776, US2011/0124100, US2009/0093049, and US2013/0177581; International Patent Application Publication Nos. WO2009/075886, WO2007/025008, WO2012/009644, WO2001/055371, and WO1999/024595; and European Patent Application Publication Nos. EP2610341A1, and EP2610340A1; the contents of each of which are incorporated herein by reference in their entirety for this purpose), or their variants, homologs, or functional derivatives. In some embodiments, the nucleic acid of the disclosure comprises one or multiple copies of a TEE. The TEE in a translational enhancer nucleic acid can be organized in one or more sequence segments. A sequence segment can harbor one or more of the TEEs provided herein, with each TEE being present in one or more copies. When multiple sequence segments are present in a translational enhancer nucleic acid, they can be homogenous or heterogeneous. Thus, the multiple sequence segments in a translational enhancer nucleic acid can harbor identical or different types of the TEE provided herein, identical or different number of copies of each of the TEE, and/or identical or different organization of the TEE within each sequence segment. In some embodiments, the nucleic acid of the disclosure comprises a translational enhancer nucleic acid sequence.

In some embodiments, a 5′ UTR and/or 3′ UTR comprising at least one TEE described herein can be incorporated in a monocistronic sequence such as, but not limited to, a vector system or a nucleic acid vector. In some embodiments, a 5′ UTR and/or 3′ UTR of a polynucleotide of the disclosure comprises a TEE or portion thereof described herein. In some embodiments, the TEEs in the 3′ UTR can be the same and/or different from the TEE located in the 5′ UTR.

In some embodiments, a 5′ UTR and/or 3′ UTR of a nucleic acid of the disclosure can include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55, or more than 60 TEE sequences. In some embodiments, the 5′ UTR of a nucleic acid of the disclosure can include 1-60, 1-55, 1-50, 1-45, 1-40, 1-35, 1-30, 1-25, 1-20, 1-15, 1-10, 9, 8, 7, 6, 5, 4, 3, 2, or 1 TEE sequences. The TEE sequences in the 5′ UTR of the nucleic acid of the disclosure can be the same or different TEE sequences. A combination of different TEE sequences in the 5′ UTR of the nucleic acid of the disclosure can include combinations in which more than one copy of any of the different TEE sequences are incorporated.

In some embodiments, the 5′ UTR and/or 3′ UTR comprises a spacer to separate two TEE sequences. As a non-limiting example, the spacer can be a 15 nucleotide spacer and/or other spacers known in the art (e.g., in multiples of three nucleotides). As another non-limiting example, the 5′ UTR and/or 3′ UTR comprises a TEE sequence-spacer module repeated at least once, at least twice, at least 3 times, at least 4 times, at least 5 times, at least 6 times, at least 7 times, at least 8 times, at least 9 times, at least 10 times, or more than 10 times in the 5′ UTR and/or 3′ UTR, respectively. In some embodiments, the 5′ UTR and/or 3′ UTR comprises a TEE sequence-spacer module repeated 1, 2, 3, 4, 5, 6, 7, 8, 9, or 10 times.

### 3′ UTR and the AU Rich Elements

In certain embodiments, a nucleic acid (e.g., a nucleic acid encoding a peptide epitope of the disclosure) further comprises a 3′ UTR.

A 3′-UTR is the section of mRNA that immediately follows the translation termination codon and often contains regulatory regions that post-transcriptionally influence gene expression. Regulatory regions within the 3′-UTR can influence polyadenylation, translation efficiency, localization, and stability of the mRNA. In some embodiments, the 3′-UTR useful for the disclosure comprises a binding site for regulatory proteins or microRNAs. In some embodiments, the 3′-UTR has a silencer region, which binds to repressor proteins and inhibits the expression of the mRNA. In other embodiments, the 3′-UTR comprises an AU-rich element (AREs). Proteins bind AREs to affect the stability or decay rate of transcripts in a localized manner or affect translation initiation. In other embodiments, the 3′-UTR comprises the sequence AAUAAA that directs addition of several hundred adenine residues called the poly(A) tail to the end of the mRNA transcript.

Natural or wild type 3′ UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes (Chen et al., 1995): Class I AREs contain several dispersed copies of an AUUUA motif within U-rich regions. C-Myc and MyoD contain class I AREs. Class II AREs possess two or more overlapping UUAUUUA (U/A) (U/A) nonamers. Molecules containing this type of AREs include GM-CSF and TNF-a. Class III ARES do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of nucleic acids of the disclosure. When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

Regions having a 5′ Cap

The nucleic acid cancer vaccine described herein may be an mRNA cancer vaccine comprising one or more mRNA having open reading frames that encode peptide epitopes. Each of these mRNA may have a 5′ Cap.

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule (cap). This 5′-guanylate cap can then be methylated to generate an N7-methyl-guanylate residue (cap-0). The ribose sugars of the terminal and/or anteterminal transcribed nucleotides of the 5′ end of the mRNA can optionally also be 2′-O-methylated (e.g., with a 2′-hydroxy group on the first ribose sugar (cap-1); or with a 2′-hydroxy group on the first two ribose sugars (cap-2)). 5′-decapping through hydrolysis and cleavage of the guanylate cap structure can target a nucleic acid molecule, such as an mRNA molecule, for degradation.

In some embodiments, nucleic acids (e.g., a nucleic acid encoding a peptide epitope) incorporate a cap moiety.

In some embodiments, nucleic acids (e.g., a nucleic acid encoding a peptide epitope) comprise a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, MA) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide that functions as an mRNA molecule. Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides of the disclosure.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′mppp-G; which can equivalently be designated 3′ O-Me-m7G (5′)ppp(5′) G). The 3′-O atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

In some embodiments, the cap is a dinucleotide cap analog. As a non-limiting example, the dinucleotide cap analog can be modified at different phosphate positions with a boranophosphate group or a phophoroselenoate group such as the dinucleotide cap analogs described in U.S. Pat. No. 8,519,110, the contents of which are herein incorporated by reference in its entirety for this purpose.

In some embodiments, the cap is a cap analog is a N7-(4-chlorophenoxyethyl) substituted dicucleotide form of a cap analog known in the art and/or described herein. Non-limiting examples of a N7-(4-chlorophenoxyethyl) substituted dicucleotide form of a cap analog include a N7-(4-chlorophenoxyethyl)-G (5′)ppp(5′) G and a N7-(4-chlorophenoxyethyl)-m3′-OG(5′)ppp(5′) G cap analog (see, e.g., the various cap analogs and the methods of synthesizing cap analogs described in Kore et al. Bioorganic & Medicinal Chemistry 2013 21:4570-4574; the contents of which are herein incorporated by reference in its entirety for this purpose). In some embodiments, a cap analog is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, can lead to reduced translational competency and reduced cellular stability.

Nucleic acids of the disclosure (e.g., a nucleic acids encoding peptide antigens) can also be capped post-manufacture (e.g., through IVT or chemical synthesis), using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., or which outperforms the corresponding endogenous, wild-type, natural or physiological feature in one or more respects. Non-limiting examples of more authentic 5′cap structures are those that, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the cap-1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap-0), 7mG(5′)ppp(5′)NlmpNp (cap-1), and 7mG(5′)-ppp(5′)NlmpN2mp (cap-2).

As a non-limiting example, capping chimeric nucleic acids post-manufacture can be more efficient as nearly 100% of the chimeric nucleic acids can be capped. This is in contrast to ˜80% when a cap analog is linked to a chimeric nucleic acids in the course of an in vitro transcription reaction.

According to the present disclosure, 5′ terminal caps can include endogenous caps or cap analogs. According to the present disclosure, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

### Poly-A Tails

In some embodiments, the nucleic acids (e.g., a nucleic acid encoding peptide epitopes) further comprise a poly-A tail. In further embodiments, terminal groups on the poly-A tail can be incorporated for stabilization. In other embodiments, a poly-A tail comprises des-3′ hydroxyl tails.

During RNA processing, a long chain of adenine nucleotides (poly-A tail) can be added to a nucleic acid such as an mRNA molecule in order to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240, or 250 residues long. In some embodiments, the poly A tail comprises about 100 nucleotides.

PolyA tails can also be added after the construct is exported from the nucleus.

According to the present disclosure, terminal groups on the poly A tail can be incorporated for stabilization. Polynucleotides can include des-3′ hydroxyl tails. They can also include structural moieties or 2′-O-methyl modifications as taught by Junjie Li, et al. (Current Biology, Vol. 15, 1501-1507 Aug. 23, 2005, the contents of which are incorporated herein by reference in its entirety for this purpose).

The nucleic acids can be designed to encode transcripts with alternative polyA tail structures including histone mRNA. According to Norbury, “[t]erminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs” (Norbury, “Cytoplasmic RNA: a case of the tail wagging the dog,” Nature Reviews Molecular Cell Biology; AOP, published online 29 Aug. 2013; doi: 10.1038/nrm3645) the contents of which are incorporated herein by reference in its entirety for this purpose.

Unique poly-A tail lengths provide certain advantages to the nucleic acids. Generally, the length of a poly-A tail, when present, is greater than 30 nucleotides in length. In some embodiments, the poly-A tail is greater than 35 nucleotides in length (e.g., at least or greater than about 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, or 3,000 nucleotides).

In some embodiments, the nucleic acid or region thereof includes from about 15 to about 3,000 nucleotides (e.g., from 15 to 50, 15 to 100, 15 to 200, 15 to 300, 15 to 400, 15 to 500, 15 to 600, 15 to 700, 15 to 800, 15 to 900, 15 to 1000, 15 to 1200, 15 to 1400, 15 to 1500, 15 to 1800, 15 to 2000, 15 to 2500, 15 to 3000, 50 to 100, 50 to 200, 50 to 300, 50 to 400, 50 to 500, 50 to 600, 50 to 700, 50 to 800, 50 to 900, 50 to 1000, 50 to 1200, 50 to 1400, 50 to 1500, 50 to 1800, 50 to 2000, 50 to 2500, 50 to 3000, 100 to 200, 100 to 300, 100 to 400, 100 to 500, 100 to 600, 100 to 700, 100 to 800, 100 to 900, 100 to 1000, 100 to 1200, 100 to 1400, 100 to 1500, 100 to 1800, 100 to 2000, 100 to 2500, 100 to 3000, 200 to 300, 200 to 400, 200 to 500, 200 to 600, 200 to 700, 200, to 800, 200 to 900, 200 to 1000, 200 to 1500, 200 to 3000, 500 to 1000, 500 to 1500, 500 to 2000, 500 to 2500, 500 to 3000, 1000 to 1500, 1000 to 2000, 1000 to 2500, 1000 to 3000, 1500 to 3000, 2500 to 3000, or 2000 to 3000 nucleotides).

In some embodiments, the poly-A tail is designed relative to the length of the overall nucleic acid or the length of a particular region of the nucleic acid. This design can be based on the length of a coding region, the length of a particular feature or region or based on the length of the ultimate product expressed from the nucleic acids.

In this context, the poly-A tail can be 10, 20, 30, 40, 50, 60, 70, 80, 90, or 100% greater in length than the nucleic acid or feature thereof. The poly-A tail can also be designed as a fraction of the nucleic acid to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of nucleic acids for Poly-A binding protein can enhance expression.

Additionally, multiple distinct nucleic acids can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr, and/or day 7 post-transfection.

In some embodiments, the nucleic acids are designed to include a polyA-G Quartet region. The G-quartet is a cyclic hydrogen bonded array of four guanine nucleotides that can be formed by G-rich sequences in both DNA and RNA. In these embodiments, the G-quartet is incorporated at the end of the poly-A tail. The resultant nucleic acid is assayed for stability, protein production, and other parameters including half-life at various time points. It has been discovered that the poly A-G quartet results in protein production from an mRNA equivalent to at least 75% of that seen using a poly-A tail of 120 nucleotides alone.

### Start Codon Region

The disclosure also includes a nucleic acid that comprises both a start codon region and the nucleic acid described herein (e.g., a nucleic acid comprising a nucleotide sequence encoding peptide epitopes). In some embodiments, the nucleic acids can have regions that are analogous to or function like a start codon region.

In some embodiments, the translation of a nucleic acid can initiate on a codon that is not the start codon AUG. Translation of the nucleic acid can initiate on an alternative start codon such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PLOS ONE, 2010 5:11; the contents of each of which are herein incorporated by reference in its entirety for this purpose).

As a non-limiting example, the translation of a nucleic acid begins on the alternative start codon ACG. As another non-limiting example, nucleic acid translation begins on the alternative start codon CTG or CUG. As yet another non-limiting example, the translation of a nucleic acid begins on the alternative start codon GTG or GUG.

Nucleotides flanking a codon that initiates translation such as, but not limited to, a start codon or an alternative start codon, are known to affect the translation efficiency, the length and/or the structure of the nucleic acid. (See, e.g., Matsuda and Mauro PLOS ONE, 2010 5:11; the contents of which are herein incorporated by reference in its entirety for this purpose). Masking any of the nucleotides flanking a codon that initiates translation can be used to alter the position of translation initiation, translation efficiency, length, and/or structure of a polynucleotide.

In some embodiments, a masking agent can be used near the start codon or alternative start codon in order to mask or hide the codon to reduce the probability of translation initiation at the masked start codon or alternative start codon. Non-limiting examples of masking agents include antisense locked nucleic acids (LNA) nucleic acids and exon-junction complexes (EJCs) (See, e.g., Matsuda and Mauro describing masking agents LNA polynucleotides and EJCs (PLOS ONE, 2010 5:11); the contents of which are herein incorporated by reference in its entirety for this purpose).

In some embodiments, a masking agent can be used to mask a start codon of a nucleic acid in order to increase the likelihood that translation will initiate on an alternative start codon. In some embodiments, a masking agent can be used to mask a first start codon or alternative start codon in order to increase the chance that translation will initiate on a start codon or alternative start codon downstream to the masked start codon or alternative start codon.

In some embodiments, the start codon of a nucleic acid can be removed from the nucleic acid sequence in order to have the translation of the nucleic acid begin on a codon that is not the start codon. Translation of the nucleic acid can begin on the codon following the removed start codon or on a downstream start codon or an alternative start codon. In a non-limiting example, the start codon ATG or AUG is removed as the first 3 nucleotides of the nucleic acid sequence in order to have translation initiate on a downstream start codon or alternative start codon. The nucleic acid sequence where the start codon was removed can further comprise at least one masking agent for the downstream start codon and/or alternative start codons in order to control or attempt to control the initiation of translation, the length of the nucleic acid and/or the structure of the nucleic acid.

### Stop Codon Region

The disclosure also includes a nucleic acid that comprises both a stop codon region and the nucleic acid described herein (e.g., a nucleic acid encoding peptide epitopes). In some embodiments, the nucleic acids can include at least two stop codons before the 3′ untranslated region (UTR). The stop codon can be selected from TGA, TAA and TAG in the case of DNA, or from UGA, UAA and UAG in the case of RNA. In some embodiments, the nucleic acids include the stop codon TGA in the case of DNA, or the stop codon UGA in the case of RNA, and one additional stop codon. In some embodiments, the addition stop codon can be TAA or UAA. In some embodiments, the nucleic acids include three consecutive stop codons, four stop codons, or more.

### Insertions and Substitutions

The disclosure also includes a nucleic acid that further comprises insertions and/or substitutions.

In some embodiments, the 5′ UTR of the nucleic acid can be replaced by the insertion of at least one region and/or string of nucleosides of the same base. The region and/or string of nucleotides can include, but is not limited to, at least 3, at least 4, at least 5, at least 6, at least 7, or at least 8 nucleotides and the nucleotides can be natural and/or unnatural. As a non-limiting example, the group of nucleotides can include 5-8 adenine, cytosine, thymine, a string of any of the other nucleotides disclosed herein and/or combinations thereof.

In some embodiments, the 5′ UTR of the nucleic acid can be replaced by the insertion of at least two regions and/or strings of nucleotides of two different bases such as, but not limited to, adenine, cytosine, thymine, any of the other nucleotides disclosed herein, and/or combinations thereof. For example, the 5′ UTR can be replaced by inserting 5-8 adenine bases followed by the insertion of 5-8 cytosine bases. In another example, the 5′ UTR can be replaced by inserting 5-8 cytosine bases followed by the insertion of 5-8 adenine bases.

In some embodiments, the nucleic acid can include at least one substitution and/or insertion downstream of the transcription start site that can be recognized by an RNA polymerase. As a non-limiting example, at least one substitution and/or insertion can occur downstream of the transcription start site by substituting at least one nucleic acid in the region just downstream of the transcription start site (such as, but not limited to, +1 to +6). Changes to region of nucleotides just downstream of the transcription start site can affect initiation rates, increase apparent nucleotide triphosphate (NTP) reaction constant values, and increase the dissociation of short transcripts from the transcription complex curing initial transcription (Brieba et al, Biochemistry (2002) 41:5144-5149; herein incorporated by reference in its entirety for this purpose). The modification, substitution, and/or insertion of at least one nucleoside can cause a silent mutation of the sequence or can cause a mutation in the amino acid sequence.

In some embodiments, the nucleic acid can include the substitution of at least 1, at least 2, at least 3, at least 4, at least 5, at least 6, at least 7, at least 8, at least 9, at least 10, at least 11, at least 12, or at least 13 guanine bases downstream of the transcription start site.

In some embodiments, the nucleic acid can include the substitution of at least 1, at least 2, at least 3, at least 4, at least 5, or at least 6 guanine bases in the region just downstream of the transcription start site. As a non-limiting example, if the nucleotides in the region are GGGAGA, the guanine bases can be substituted by at least 1, at least 2, at least 3, or at least 4 adenine nucleotides. In another non-limiting example, if the nucleotides in the region are GGGAGA the guanine bases can be substituted by at least 1, at least 2, at least 3, or at least 4 cytosine bases. In another non-limiting example, if the nucleotides in the region are GGGAGA the guanine bases can be substituted by at least 1, at least 2, at least 3, or at least 4 thymine, and/or any of the nucleotides described herein.

In some embodiments, the nucleic acid can include at least one substitution and/or insertion upstream of the start codon. For the purpose of clarity, one of skill in the art would appreciate that the start codon is the first codon of the protein coding region whereas the transcription start site is the site where transcription begins. The nucleic acid can include, but is not limited to, at least 1, at least 2, at least 3, at least 4, at least 5, at least 6, at least 7, or at least 8 substitutions and/or insertions of nucleotide bases. The nucleotide bases can be inserted or substituted at 1, at least 1, at least 2, at least 3, at least 4, or at least 5 locations upstream of the start codon. The nucleotides inserted and/or substituted can be the same base (e.g., all A, or all C, or all T, or all G), two different bases (e.g., A and C, A and T, or C and T), three different bases (e.g., A, C and T, or A, C and T) or at least four different bases.

As a non-limiting example, the guanine base upstream of the coding region in the nucleic acid can be substituted with adenine, cytosine, thymine, or any of the nucleotides described herein. In another non-limiting example, the substitution of guanine bases in the nucleic acid can be designed so as to leave one guanine base in the region downstream of the transcription start site and before the start codon (see Esvelt et al. Nature (2011) 472(7344): 499-503; the contents of which is herein incorporated by reference in its entirety for this purpose). As a non-limiting example, at least 5 nucleotides can be inserted at 1 location downstream of the transcription start site but upstream of the start codon and the at least 5 nucleotides can be the same base type.

According to the present disclosure, two regions or parts of a chimeric nucleic acid may be joined or ligated, for example, using triphosphate chemistry. In some embodiments, a first region or part of 100 nucleotides or less is chemically synthesized with a 5′-monophosphate and terminal 3′-desOH or blocked OH. If the region is longer than 80 nucleotides, it may be synthesized as two or more strands that will subsequently be chemically linked by ligation. If the first region or part is synthesized as a non-positionally modified region or part using IVT, conversion to the 5′-monophosphate with subsequent capping of the 3′-terminus may follow. Monophosphate protecting groups may be selected from any of those known in the art. A second region or part of the chimeric nucleic acid may be synthesized using either chemical synthesis or IVT methods, e.g., as described herein. IVT methods may include use of an RNA polymerase that can utilize a primer with a modified cap. Alternatively, a cap may be chemically synthesized and coupled to the IVT region or part.

It is noted that for ligation methods, ligation with DNA T4 ligase followed by DNAse treatment (to eliminate the DNA splint required for DNA T4 Ligase activity) should readily prevent the undesirable formation of concatenation products.

The entire chimeric polynucleotide need not be manufactured with a phosphate-sugar backbone. If one of the regions or parts encodes a polypeptide, then it is preferable that such region or part comprise a phosphate-sugar backbone.

Ligation may be performed using any appropriate technique, such as enzymatic ligation, click chemistry, orthoclick chemistry, solulink, or other bioconjugate chemistries known to those in the art. In some embodiments, the ligation is directed by a complementary oligonucleotide splint. In some embodiments, the ligation is performed without a complementary oligonucleotide splint.

### Methods of Treatment

Provided herein are compositions (e.g., pharmaceutical compositions), methods, kits, and reagents for prevention and/or treatment of cancer in humans (e.g., subjects or patients) and other mammals. Nucleic acid cancer vaccines may be used as therapeutic or prophylactic agents in medicine to prevent and/or treat cancer. In exemplary aspects, the cancer vaccines are used to provide prophylactic protection from cancer. Prophylactic protection from cancer can be achieved following administration of a cancer vaccine. Vaccines can be administered once, twice, three times, four times, or more but it may be sufficient to administer the vaccine once (optionally followed by a single booster). It may also be desirable to administer the vaccine to an individual having cancer to achieve a therapeutic response. Dosing may need to be adjusted accordingly.

Once a cancer vaccine (e.g., a nucleic acid cancer vaccine) is synthesized, it is administered to the patient. In some embodiments, the vaccine is administered on a schedule for up to two months, up to three months, up to four months, up to five months, up to six months, up to seven months, up to eight months, up to nine months, up to ten months, up to eleven months, up to 1 year, up to 1 and ½ years, up to two years, up to three years, or up to four years. The schedule may be the same or varied. In some embodiments, the schedule is weekly for the first 3 weeks and then monthly thereafter. The schedule may be determined or varied by one of skill in the art (e.g., a medical doctor) depending on the individual patient or subject's criteria (e.g., weight, age, type of cancer, etc.).

In some embodiments, a cancer vaccine (e.g., nucleic acid cancer vaccine) is administered to a patient on a regular basis (e.g., once a week, once every two weeks, once every three weeks, once every four weeks, etc.) for a specified total period of time, or until a particular endpoint is reached. The specified total period of time, in some embodiments, is the time corresponding to the administration of 2 doses, 3 doses, 4 doses, 5 doses, 6 doses, 7 doses, 8 doses, 9 doses, 10 doses, 11 doses, 12 doses, 13 doses, 14 doses, 15 doses, 16 doses, or more. For example, a cancer vaccine, in some embodiments, is administered to a patient once every three weeks until 9 doses have been administered, or until another endpoint is reached. In some embodiments, the other endpoint is disease recurrence, unacceptable toxicity, or withdrawal of consent to be treated.

The vaccine may be administered by any route. In some embodiments, the vaccine is administered by an intradermal, intramuscular, intravascular, intratumoral, and/or subcutaneous route.

In some embodiments, the nucleic acid cancer vaccine may also be administered with an additional anti-cancer therapeutic agent. The nucleic acid cancer vaccine and other therapeutic agent may be administered simultaneously or sequentially. When the other therapeutic agents are administered simultaneously they can be administered in the same or separate formulations, but are administered at the same time or substantially the same time. The other therapeutic agents are administered sequentially with one another and with the nucleic acid cancer vaccine, when the administration of the other therapeutic agents and the nucleic acid cancer vaccine is temporally separated. The separation in time between administrations of these compounds may be a matter of minutes or it may be longer, e.g., hours, days, weeks, months. Other therapeutic agents include but are not limited to anti-cancer therapeutic, adjuvants, cytokines, antibodies, antigens, etc.

In some embodiments, a cancer vaccine (e.g., a personalized cancer vaccine) may be administered in combination with an additional anti-cancer therapeutic agent (e.g., an immune checkpoint modulator, such as an immune checkpoint inhibitor). In some embodiments, the cancer vaccine is administered prior to initiation of the additional anti-cancer therapeutic agent treatment. In some embodiments, the cancer vaccine is administered 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, 3 weeks 4 weeks, 5 weeks, 6 weeks, 7 weeks, 8 weeks, 9 weeks, 10 weeks, 11 weeks, 12 weeks, or more, prior to initiation of the additional anti-cancer therapeutic agent treatment. In some embodiments, the cancer vaccine is administered after initiation of the additional anti-cancer therapeutic agent treatment. In some embodiments, the cancer vaccine is administered 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, 3 weeks 4 weeks, 5 weeks, 6 weeks, 7 weeks, 8 weeks, 9 weeks, 10 weeks, 11 weeks, 12 weeks, or more, after initiation of the additional anti-cancer therapeutic agent treatment. In some embodiments, the cancer vaccine is administered within 8 weeks, 7 weeks, 6 weeks, 5 weeks, 4 weeks, 3 weeks, 2 weeks, or 1 week from the initiation of the additional anti-cancer therapeutic agent treatment. For example, in some embodiments, the cancer vaccine is administered within about 6 weeks (e.g., within 4 weeks, within 5 weeks, within 6 weeks, within 7 weeks, or within 8 weeks) following the first administration of the additional anti-cancer therapeutic agent.

At any point in the treatment the patient may be examined to determine whether the mutations in the vaccine are still appropriate. Based on that analysis the vaccine may be adjusted or reconfigured to include one or more different neoantigens or to remove one or more neoantigens. This may be done according to a method of optimizing a personalized cancer vaccine, as provided herein.

In exemplary embodiments, a cancer vaccine (e.g., a personalized cancer vaccine) containing RNA polynucleotides as described herein can be administered to a subject (e.g., a mammalian subject, such as a human subject), and the RNA polynucleotides are translated in vivo to produce an antigenic polypeptide.

The cancer vaccines may be induced for translation of a polypeptide (e.g., antigen or immunogen) in a cell, tissue or organism. In exemplary embodiments, such translation occurs in vivo, although there can be envisioned embodiments where such translation occurs ex vivo, in culture or in vitro. In exemplary embodiments, the cell, tissue or organism is contacted with an effective amount of a composition containing a cancer vaccine that contains a polynucleotide that has at least one a translatable region encoding an antigenic polypeptide.

An “effective amount” of a cancer vaccine (e.g., a personalized cancer vaccine) may be provided based, at least in part, on the target tissue, target cell type, means of administration, physical characteristics of the polynucleotide (e.g., size, and extent of modified nucleosides) and other components of the cancer vaccine, and other determinants. In general, an effective amount of the cancer vaccine composition provides an induced or boosted immune response as a function of antigen production in the cell, preferably more efficient than a composition containing a corresponding unmodified polynucleotide encoding the same antigen or a peptide antigen. Increased antigen production may be demonstrated by increased cell transfection (the percentage of cells transfected with the cancer vaccine), increased protein translation from the polynucleotide, decreased nucleic acid degradation (as demonstrated, for example, by increased duration of protein translation from a modified polynucleotide), or altered antigen specific immune response of the host cell.

Cancer vaccines (e.g., personalized cancer vaccines) may be administered prophylactically or therapeutically as part of an active immunization scheme to healthy individuals or early in cancer or during active cancer after onset of symptoms. In some embodiments, the amount of vaccines provided to a cell, a tissue or a subject may be an amount effective for immune prophylaxis.

Cancer vaccines (e.g., personalized cancer vaccines) may be administered in an adjuvant setting, i.e., after the patient has received a primary treatment for their cancer. For example, a cancer vaccine (e.g., a personalized cancer vaccine) may be administered to a subject in an adjuvant setting after a tumor has been surgically resected from the subject, and/or after the subject has received treatment with an anti-cancer agent (e.g., an anti-cancer drug).

Cancer vaccines (e.g., personalized cancer vaccines) may be administered to a subject identified as having or being likely to have high responsiveness to another cancer therapy, e.g., an immune checkpoint modulator therapy. In some embodiments, a cancer vaccine (e.g., a personalized cancer vaccine) may be administered to a subject having characteristics associated with high responsiveness to immune checkpoint modulator therapy, e.g., immune checkpoint inhibitor therapy, such as anti-PD-1 therapy. For example, in some embodiments, a cancer vaccine (e.g., a personalized cancer vaccine) may be administered to a subject having biomarker(s) associated with high responsiveness to immune checkpoint modulator therapy. In some embodiments, a cancer vaccine (e.g., a personalized cancer vaccine) may be administered to a subject having previously received an immune checkpoint modulator therapy to which they demonstrated a high responsiveness, e.g., as determined through a clinical metric such as a laboratory or radiological test. In some embodiments, administration of a cancer vaccine (e.g., a personalized cancer vaccine) to such a subject results in greater responsiveness to the immune checkpoint modulator therapy, relative to the responsiveness if the therapy was given without the cancer vaccine.

In some embodiments, a cancer vaccine (e.g., a personalized cancer vaccine) may be administered to a subject having a particular biomarker, biomarker level, set of biomarkers, or set of biomarker levels, e.g., in a tumor sample collected from the subject. For example, a cancer vaccine (e.g., a personalized cancer vaccine) may be administered to a subject having high or low tumor immunogenicity, e.g., as measured by tumor mutational burden (TMB), T cell-inflamed gene expression profile (GEP) score, T cell cytotoxicity (CYT) score, PD-L1 expression, etc. In some embodiments, a cancer vaccine may be administered to a subject having high tumor immunogenicity, e.g., as measured by TMB, T cell-inflamed GEP score, CYT score, PD-L1 expression, etc.

In some embodiments, a cancer vaccine (e.g., a personalized cancer vaccine) may be administered to a subject having high tumor mutational burden (TMB), e.g., having TMB of greater than 75, greater than 100, greater than 125, greater than 150, greater than 175, greater than 200, greater than 225, greater than 250, greater than 275, greater than 300, greater than 400, greater than 500, greater than 600, greater than 700, greater than 800, greater than 900, greater than 1000, or more mutations (e.g., non-synonymous mutations) per exome (e.g., in a whole exome sequencing data set). In some embodiments, TMB is determined by an FDA-approved test, such as FoundationOne® CDx test (Foundation Medicine, Cambridge, MA). In some embodiments, the mutations (e.g., non-synonymous mutations) accounted for in the TMB value each have a specific allele frequency (e.g., an allele frequency of at least 1%, at least 2%, at least 3%, at least 4%, at least 5%, at least 6%, at least 7%, at least 8%, at least 9%, at least 10%, or more, preferably of at least 5%), e.g., as measured in a whole exome sequencing data set.

In some embodiments, a cancer vaccine (e.g., a personalized cancer vaccine) may be administered to a subject having a high T cell-inflamed GEP score, e.g., having a T cell-inflamed GEP score of greater than 1, 1.5, 2, 2.5, 3, 3.5, 4, 4.1, 4.2, 4.3, 4.4, 4.5, 4.6, 4.7, 4.8, 4.9, 5, 5.1, 5.2, 5.3, 5.4, 5.5, 6, or higher.

In some embodiments, a cancer vaccine (e.g., a personalized cancer vaccine) may be administered to a subject having a high CYT score, e.g., having a CYT score of greater than 1, 1.5, 2, 2.5, 3, 3.5, 4, 4.1, 4.2, 4.3, 4.4, 4.5, 4.6, 4.7, 4.8, 4.9, 5, 5.1, 5.2, 5.3, 5.4, 5.5, 6, or higher.

In some embodiments, a cancer vaccine may be administered to a subject having a high PD-L1 expression level, e.g., a PD-L1 expression level of greater than 1, 1.5, 2, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 6, or higher, when normalized relative to one or more housekeeping genes (e.g., STK11IP, ZBTB34, TBC1D10B, OAZ1, POLR2A, G6PD, ABCF1, C14orf102, UBB, TBP, SDHA).

Cancer vaccines (e.g., personalized cancer vaccines) may be administered to a subject identified as having or being likely to have a low responsiveness to another cancer therapy, e.g., an immune checkpoint modulator therapy. In some embodiments, a cancer vaccine (e.g., a personalized cancer vaccine) may be administered to a subject having characteristics associated with low responsiveness to immune checkpoint modulator therapy, e.g., immune checkpoint inhibitor therapy, such as anti-PD-1 therapy. For example, in some embodiments, a cancer vaccine (e.g., a personalized cancer vaccine) may be administered to a subject having biomarker(s) and/or biomarker level(s) associated with a low responsiveness to immune checkpoint modulator therapy. In some embodiments, a cancer vaccine (e.g., a personalized cancer vaccine) may be administered to a subject having previously received an immune checkpoint modulator therapy to which they demonstrated a low responsiveness, e.g., as determined through a clinical metric such as a laboratory or radiological test. In some embodiments, administration of a cancer vaccine (e.g., a personalized cancer vaccine) to such a subject results in greater responsiveness to the immune checkpoint modulator therapy, relative to the responsiveness if the therapy was given without the cancer vaccine.

In some embodiments, a cancer vaccine (e.g., a personalized cancer vaccine) may be administered to a subject having a low tumor immunogenicity, e.g., as measured by tumor mutational burden (TMB), T cell-inflamed gene expression profile (GEP) score, T cell cytotoxicity (CYT) score, PD-L1 expression, etc.

In some embodiments, a cancer vaccine may be administered to a subject having low tumor mutational burden (TMB), e.g., having TMB of fewer than 300, fewer than 275, fewer than 250, fewer than 225, fewer than 200, fewer than 175, fewer than 150, fewer than 125, fewer than 100, fewer than 75, fewer than 50, fewer than 25, or fewer mutations (e.g., non-synonymous mutations) per exome (e.g., in a whole exome sequencing data set). In some embodiments, TMB is determined by an FDA-approved test, such as FoundationOne® CDx test (Foundation Medicine, Cambridge, MA). In some embodiments, the mutations (e.g., non-synonymous mutations) accounted for in the TMB value each have a specific allele frequency (e.g., an allele frequency of at least 1%, at least 2%, at least 3%, at least 4%, at least 5%, at least 6%, at least 7%, at least 8%, at least 9%, at least 10%, or more, preferably of at least 5%), e.g., as measured in a whole exome sequencing data set.

In some embodiments, a cancer vaccine (e.g., a personalized cancer vaccine) may be administered to a subject having a low T cell-inflamed GEP score, e.g., having a T cell-inflamed GEP score of less than 6, 5.5, 5.4, 5.3, 5.2, 5.1, 5.0, 4.9, 4.8, 4.7, 4.6, 4.5, 4.4, 4.3, 4.2, 4.1, 4, 3.5, 3, 2.5, 2, 1.5, 1, or lower.

In some embodiments, a cancer vaccine (e.g., a personalized cancer vaccine) may be administered to a subject having a low CYT score, e.g., having a CYT score of less than 6, 5.5, 5.4, 5.3, 5.2, 5.1, 5.0, 4.9, 4.8, 4.7, 4.6, 4.5, 4.4, 4.3, 4.2, 4.1, 4, 3.5, 3, 2.5, 2, 1.5, 1, or lower.

In some embodiments, a cancer vaccine (e.g., a personalized cancer vaccine) may be administered to a subject having a low PD-L1 expression level, e.g., a PD-L1 expression level of less than 6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.0, 1.5, 1.0, or lower, when normalized relative to one or more housekeeping genes (e.g., STK11IP, ZBTB34, TBC1D10B, OAZ1, POLR2A, G6PD, ABCF1, C14orf102, UBB, TBP, SDHA).

Cancer vaccines (e.g., personalized cancer vaccines) may be administered to a subject having low or undetectable levels of metastatic tumor cells. For example, in some embodiments, a cancer vaccine may be administered to a subject having received results of a medical diagnostic test (e.g., a radiological study/studies and/or a laboratory test(s)) indicating that no metastatic foci and/or cells were detected in the subject. In some embodiments, a cancer vaccine (e.g., a personalized cancer vaccine) may be administered to a subject having fewer than 10 (e.g., fewer than 9, fewer than 8, fewer than 7, fewer than 6, fewer than 5, fewer than 4, fewer than 3, fewer than 2, 1, or no) detectable metastatic foci.

Cancer vaccines (e.g., personalized cancer vaccines) may be administered to a subject having detectable or high levels of metastatic tumor cells. For example, in some embodiments, a cancer vaccine may be administered to a subject having received results of a medical diagnostic test (e.g., a radiological study/studies and/or a laboratory test(s)) indicating that metastatic foci and/or cells were detected in the subject. In some embodiments, a cancer vaccine may be administered to a subject having greater than 1 (e.g., greater than 2, greater than 3, greater than 4, greater than 5, greater than 6, greater than 7, greater than 8, greater than 9, greater than 10, or more) detectable metastatic foci.

Cancer vaccines (e.g., personalized cancer vaccines) may be administered to a subject having a particular score according to a patient evaluation metric. For example, in some embodiments, a cancer vaccine (e.g., a personalized cancer vaccine) is administered to a subject having a given Eastern Cooperative Oncology Group (ECOG) performance status score. In some embodiments, a cancer vaccine (e.g., a personalized cancer vaccine) is administered to a subject having an ECOG performance status score of 0, 1, 2, 3, or 4 (e.g., 0, 1, or 2). In some embodiments, a cancer vaccine (e.g., a personalized cancer vaccine) is administered to a subject having an ECOG performance status score in the range of 0-1. ECOG performance status score is determined according to the scale:

| | |
|---|---|
| 0 | Fully active, able to carry on all pre-disease performance without restriction |
| 1 | Restricted in physically strenuous activity but ambulatory and able to carry out work of a |
| | light or sedentary nature, e.g., light house work, office work |
| 2 | Ambulatory and capable of all selfcare but unable to carry out any work activities; up and |
| | about more than 50% of waking hours |
| 3 | Capable of only limited selfcare; confined to bed or chair more than 50% of waking hours |
| 4 | Completely disabled; cannot carry on any selfcare; totally confined to bed or chair |
| 5 | Dead |

The ECOG performance status score is described in Oken, et al. “Toxicity and response criteria of the Eastern Cooperative Oncology Group” Am J Clin Oncol. 5 (6): 649-655 (1982), the entire contents of which are incorporated by reference herein for this purpose.

Cancer vaccines (e.g., personalized cancer vaccines) may be administered with other prophylactic or therapeutic compounds in addition to checkpoint inhibitors. As a non-limiting example, a prophylactic or therapeutic compound may be an immune potentiator or a booster. As used herein, when referring to a composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In exemplary embodiments, the time of administration between the initial administration of the prophylactic composition and the booster may be, but is not limited to, 1 week, 2 weeks, 3 weeks, 1 month, 2 months, 3 months, 6 months or 1 year.

The cancer vaccines may be utilized in various settings depending on the severity of the cancer or the degree or level of unmet medical need. As a non-limiting example, the cancer vaccines may be utilized to treat any stage of cancer.

In some embodiments the cancer vaccines and/or checkpoint inhibitors may be used to treat PD-L1 positive tumors. In other embodiments the cancer vaccines and/or checkpoint inhibitors may be used to treat PD-L1 negative tumors. While emerging data support the use of PD-1 inhibitors such as pembrolizumab in tumors where PD-L1 expression can be demonstrated, the use of the combinations of the invention in treating PD-1 “negative” tumors is envisioned. Mechanistically, there may be an adaptive component to PD-L1 expression by tumors, i.e., tumors may initially appear PD-L1 negative but upregulate PD-L1 expression in response to IFN-γ secretion by infiltrating tumor lymphocytes. This has translated clinically in some cases, such that the response rates of PD-L1 negative tumors to the combination of PD-1 and CTLA-4 blockade is higher than the response rate to single agent PD-1 inhibitors in both cutaneous melanoma and lung cancer. Aspects of the invention relate to the use of a personalized cancer vaccine to induce PD-L1 expression in PD-L1 low tumors, in combination with a PD-1 inhibitor.

In some embodiments, the cancer vaccines and/or checkpoint inhibitors may be used to treat tumors having a high tumor mutation burden (TMB). Thus, in some embodiments, a pool of subjects may be tested for TMB and the subjects having a TMB value over a threshold level may be treated with a cancer vaccine and/or checkpoint inhibitor (e.g., a combination therapy) disclosed herein. In some embodiments, the cancer vaccines and/or checkpoint inhibitors may be used to treat tumors having a low tumor mutation burden (TMB). Thus, in some embodiments, a pool of subjects may be tested for TMB and the subjects having a TMB value below a threshold level may be treated with a cancer vaccine and/or checkpoint inhibitor (e.g., a combination therapy) disclosed herein.

A non-limiting list of cancers that the cancer vaccines may treat is presented below. Peptide epitopes or antigens may be derived from any antigen of these cancers or tumors. Such epitopes may be referred to as cancer or tumor antigens. Cancer cells may differentially express cell surface molecules during different phases of tumor progression. For example, a cancer cell may express a cell surface antigen in a benign state, yet down-regulate that particular cell surface antigen upon metastasis. As such, it is envisioned that the tumor or cancer antigen may encompass antigens produced during any stage of cancer progression. The methods of the disclosure may be adjusted to accommodate for these changes. For instance, several different cancer vaccines may be generated for a particular patient. For instance, a first vaccine may be used at the start of the treatment. At a later time point, a new cancer vaccine may be generated and administered to the patient to account for different antigens being expressed.

Cancers or tumors include but are not limited to neoplasms, malignant tumors, metastases, or any disease or disorder characterized by uncontrolled cell growth such that it would be considered cancerous. The cancer may be a primary or metastatic cancer. Specific cancers that can be treated according to the present disclosure include, but are not limited to, those listed below (for a review of such disorders, see Fishman et al., 1985, Medicine, 2d Ed., J.B. Lippincott Co., Philadelphia). Cancers for use with the instantly described methods and compositions may include, but are not limited to, biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; kidney cancer; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma, teratomas; tumor mutational burden high tumors; choriocarcinomas; stromal tumors and germ cell tumors; thyroid cancer including thyroid adenocarcinoma and medullar carcinoma; and renal cancer including adenocarcinoma and Wilms' tumor.

In some embodiments, the cancer is any one of melanoma, bladder carcinoma, cSCC, HPV negative HNSCC, MIBC, MIUC, NSCLC, RCC, SCLC, UTUC, MSI-High tumors, or TMB (tumor mutational burden) High cancers.

In some embodiments, the cancer is selected from the group consisting of non-small cell lung cancer (NSCLC), small cell lung cancer, melanoma, bladder urothelial carcinoma, HPV-negative head and neck squamous cell carcinoma (HNSCC), and a solid malignancy that is microsatellite instability high (MSI H)/mismatch repair (MMR) deficient. In some embodiments, the NSCLC lacks an EGFR sensitizing mutation and/or an ALK translocation. In some embodiments, the solid malignancy that is microsatellite instability high (MSI H)/mismatch repair (MMR) deficient is selected from the group consisting of colorectal cancer, stomach adenocarcinoma, esophageal adenocarcinoma, and endometrial cancer.

In some embodiments, the cancer is melanoma. In some embodiments, the cancer is resected stage II melanoma. In some embodiments, the cancer is resected high-risk stage III melanoma. In some embodiments, the cancer is resected high-risk stage IV melanoma. In some embodiments, the cancer is resected cutaneous melanoma.

In some embodiments, the cancer is NSCLC. In some embodiments, the cancer is resected stage II NSCLC. In some embodiments, the cancer is resected stage III NSCLC. In some embodiments, the cancer is resected stage IIIA NSCLC. In some embodiments, the cancer is resected stage IIIB NSCLC.

In some embodiments, the cancer is kidney cancer. In some embodiments, the cancer is RCC.

In some embodiments, the cancer is MIUC. In some embodiments, the cancer is MIBC. In some embodiments, the cancer is UTUC.

In some embodiments, the cancer is cSCC. In some embodiments, the cancer is resectable cSCC. In some embodiments, the cancer is locally advanced cSCC. In some embodiments, the cancer is stage II cSCC. In some embodiments, the cancer is stage III cSCC. In some embodiments, the cancer is stage IV cSCC. In some embodiments, the cancer is resectable locally advanced stage II cSCC. In some embodiments, the cancer is resectable locally advanced stage III cSCC. In some embodiments, the cancer is resectable locally advanced stage IV cSCC.

In some embodiments, the tumor has a mutation in the BRAF gene. In some embodiments, the mutation is at V600. In some embodiments, the tumor has a V600K mutation. In some embodiments, the tumor has a V600E mutation.

In some embodiments, a patient has received at least one dose of adjuvant treatment with standard of care platinum doublet chemotherapy.

Pembrolizumab monotherapy (10 mg/kg dosed every 2 weeks) has been used in subjects with advanced solid tumors that express PD-L1 which have not responded to current therapy or for which current therapy is not appropriate. For instance, in subjects with small cell lung cancer (SCLC) it was concluded that pembrolizumab is generally well tolerated and has promising antitumor activity in subjects with PD-L1+SCLC who have progressed on prior platinum-based therapy. In another study of patients with advanced urothelial cancer who were given 10 mg/kg pembrolizumab every 2 weeks, it was concluded that pembrolizumab demonstrates durable antitumor activity in subjects with advanced urothelial cancer. The combination therapy is also useful for treating Microsatellite Instability High Cancers, such as colorectal cancer, endometrial tumors, adenocarcinoma of the stomach or gastro-esophageal junction or gastric cancer.

Provided herein are pharmaceutical compositions including cancer vaccines and RNA vaccine compositions and/or complexes optionally in combination with one or more pharmaceutically acceptable excipients. Cancer vaccines (e.g., personalized cancer vaccines) may be formulated or administered alone or in conjunction with one or more other components as described herein.

In some embodiments, the cancer vaccines described herein may be combined with any other therapy useful for treating the patient. For instance, a patient may be treated with the cancer vaccine and an anti-cancer agent. Thus, in some embodiments, the methods of the disclosure can be used in conjunction with one or more cancer therapeutics, for example, in conjunction with an anti-cancer agent, a traditional cancer vaccine, chemotherapy, radiotherapy, etc. (e.g., simultaneously, or as part of an overall treatment procedure). Parameters of cancer treatment that may vary include, but are not limited to, dosages, timing of administration or duration or therapy; and the cancer treatment can vary in dosage, timing, or duration. Another treatment for cancer is surgery, which can be utilized either alone or in combination with any of the previous treatment methods. Any agent or therapy (e.g., traditional cancer vaccines, chemotherapies, radiation therapies, surgery, hormonal therapies, and/or biological therapies/immunotherapies) which is known to be useful, or which has been used or is currently being used for the prevention or treatment of cancer can be used in combination with a composition of the disclosure in accordance with the disclosure described herein. One of ordinary skill in the medical arts can determine an appropriate treatment for a subject.

Examples of such agents (i.e., anti-cancer agents) include, but are not limited to, DNA-interactive agents including, but not limited to, the alkylating agents (e.g., nitrogen mustards, e.g., Chlorambucil, Cyclophosphamide, Isofamide, Mechlorethamine, Melphalan, Uracil mustard; Aziridine such as Thiotepa; methanesulphonate esters such as Busulfan; nitroso ureas, such as Carmustine, Lomustine, Streptozocin; platinum complexes, such as Cisplatin, Carboplatin; bioreductive alkylator, such as Mitomycin, and Procarbazine, Dacarbazine and Altretamine); the DNA strand-breakage agents, e.g., Bleomycin; the intercalating topoisomerase II inhibitors, e.g., Intercalators, such as Amsacrine, Dactinomycin, Daunorubicin, Doxorubicin, Idarubicin, Mitoxantrone, and nonintercalators, such as Etoposide and Teniposide; the nonintercalating topoisomerase II inhibitors, e.g., Etoposide and Teniposde; and the DNA minor groove binder, e.g., Plicamydin; the antimetabolites including, but not limited to, folate antagonists such as Methotrexate and trimetrexate; pyrimidine antagonists, such as Fluorouracil, Fluorodeoxyuridine, CB3717, Azacitidine and Floxuridine; purine antagonists such as Mercaptopurine, 6-Thioguanine, Pentostatin; sugar modified analogs such as Cytarabine and Fludarabine; and ribonucleotide reductase inhibitors such as hydroxyurea; tubulin Interactive agents including, but not limited to, colchicine, Vincristine and Vinblastine, both alkaloids and Paclitaxel and cytoxan; hormonal agents including, but not limited to, estrogens, conjugated estrogens and Ethinyl Estradiol and Diethylstilbesterol, Chlortrianisen and Idenestrol; progestins such as Hydroxyprogesterone caproate, Medroxyprogesterone, and Megestrol; and androgens such as testosterone, testosterone propionate; fluoxymesterone, methyltestosterone; adrenal corticosteroid, e.g., Prednisone, Dexamethasone, Methylprednisolone, and Prednisolone; leutinizing hormone releasing hormone agents or gonadotropin-releasing hormone antagonists, e.g., leuprolide acetate and goserelin acetate; antihormonal antigens including, but not limited to, antiestrogenic agents such as Tamoxifen, antiandrogen agents such as Flutamide; and antiadrenal agents such as Mitotane and Aminoglutethimide; cytokines including, but not limited to, IL-1α, IL-1β, IL-2, IL-3, IL-4, IL-5, IL-6, IL-7, IL-8, IL-9, IL-10, IL-11, IL-12, IL-13, IL-18, TGF-β, GM-CSF, M-CSF, G-CSF, TNF-α, TNF-β, LAF, TCGF, BCGF, TRF, BAF, BDG, MP, LIF, OSM, TMF, PDGF, IFN-α, IFN-β, IFN-γ, and Uteroglobins (U.S. Pat. No. 5,696,092); anti-angiogenics including, but not limited to, agents that inhibit VEGF (e.g., other neutralizing antibodies), soluble receptor constructs, tyrosine kinase inhibitors, antisense strategies, RNA aptamers and ribozymes against VEGF or VEGF receptors, immunotoxins and coaguligands, tumor vaccines, and antibodies.

Specific examples of anti-cancer agents which can be used in accordance with the methods of the disclosure include, but not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs which may be used with the instant compositions and methods include, but are not limited to: 20-epi-1,25 dihydroxyvitamin D3; 5-ethynyluracil; angiogenesis inhibitors; anti-dorsalizing morphogenetic protein-1; ara-CDP-DL-PTBA; BCR/ABL antagonists; CaRest M3; CARN 700; casein kinase inhibitors (ICOS); clotrimazole; collismycin A; collismycin B; combretastatin A4; crambescidin 816; cryptophycin 8; curacin A; dehydrodidemnin B; didemnin B; dihydro-5-azacytidine; dihydrotaxol, duocarmycin SA; kahalalide F; lamellarin-N triacetate; leuprolide+estrogen+progesterone; lissoclinamide 7; monophosphoryl lipid A+myobacterium cell wall sk; N-acetyldinaline; N-substituted benzamides; 06-benzylguanine; placetin A; placetin B; platinum complex; platinum compounds; platinum-triamine complex; rhenium Re 186 etidronate; RII retinamide; rubiginone B 1; SarCNU; sarcophytol A; sargramostim; senescence derived inhibitor 1; spicamycin D; tallimustine; 5-fluorouracil; thrombopoietin; thymotrinan; thyroid stimulating hormone; variolin B; thalidomide; velaresol; veramine; verdins; verteporfin; vinorelbine; vinxaltine; vitaxin; zanoterone; zeniplatin; and zilascorb.

The disclosure also encompasses administration of a composition comprising a cancer vaccine in combination with radiation therapy comprising the use of x-rays, gamma rays and other sources of radiation to destroy the cancer cells. In certain embodiments, the radiation treatment is administered as external beam radiation or teletherapy wherein the radiation is directed from a remote source. In other embodiments, the radiation treatment is administered as internal therapy or brachytherapy wherein a radioactive source is placed inside the body close to cancer cells or a tumor mass.

In specific embodiments, an appropriate anti-cancer regimen is selected depending on the type of cancer (e.g., by a physician). For instance, a patient with ovarian cancer may be administered a prophylactically or therapeutically effective amount of a composition comprising a cancer vaccine in combination with a prophylactically or therapeutically effective amount of one or more other agents useful for ovarian cancer therapy, including but not limited to, intraperitoneal radiation therapy, such as P32 therapy, total abdominal and pelvic radiation therapy, cisplatin, the combination of paclitaxel (Taxol®) or docetaxel (Taxotere®) and cisplatin or carboplatin, the combination of cyclophosphamide and cisplatin, the combination of cyclophosphamide and carboplatin, the combination of 5-FU and leucovorin, etoposide, liposomal doxorubicin, gemcitabine or topotecan. Cancer therapies and their dosages, routes of administration and recommended usage are known in the art and have been described in such literature as the Physician's Desk Reference (56th ed., 2002).

In some embodiments, the cancer therapeutic agent is a targeted therapy. The targeted therapy may be a BRAF inhibitor such as vemurafenib (PLX4032) or dabrafenib. The BRAF inhibitor may be PLX 4032, PLX 4720, PLX 4734, GDC-0879, PLX 4032, PLX-4720, PLX 4734 and Sorafenib Tosylate. BRAF is a human gene that makes a protein called B-Raf, also referred to as proto-oncogene B-Raf and v-Raf murine sarcoma viral oncogene homolog B1. The B-Raf protein is involved in sending signals inside cells, which are involved in directing cell growth. Vemurafenib, a BRAF inhibitor, was approved by FDA for treatment of late-stage melanoma.

In other embodiments, the cancer therapeutic agent is a cytokine. In yet other embodiments, the cancer therapeutic agent is a vaccine comprising a population based tumor specific antigen. In yet other embodiments, the cancer therapeutic agent is vaccine containing one or more traditional antigens expressed by cancer-germline genes (antigens common to tumors found in multiple patients, also referred to as “shared cancer antigens”). In some embodiments, a traditional antigen is one that is known to be found in cancers or tumors generally or in a specific type of cancer or tumor. In some embodiments, a traditional cancer antigen is a non-mutated tumor antigen. In some embodiments, a traditional cancer antigen is a mutated tumor antigen.

The p53 gene (official symbol TP53) is mutated more frequently than any other gene in human cancers. Large cohort studies have shown that, for most p53 mutations, the genomic position is unique to one or only a few patients and the mutation cannot be used as recurrent neoantigens for therapeutic vaccines designed for a specific population of patients. A small subset of p53 loci do, however, exhibit a “hotspot” or “driver” pattern (described elsewhere herein), in which several positions in the gene are mutated with relatively high frequency. Strikingly, a large portion of these recurrently mutated regions occur near exon-intron boundaries, disrupting the canonical nucleotide sequence motifs recognized by the mRNA splicing machinery.

Mutation of a splicing motif can alter the final mRNA sequence even if no change to the local amino acid sequence is predicted (i.e., for synonymous or intronic mutations). Therefore, these mutations are often annotated as “noncoding” by common annotation tools and neglected for further analysis, even though they may alter mRNA splicing in unpredictable ways and exert severe functional impact on the translated protein. If an alternatively spliced isoform produces an in-frame sequence change (i.e., no pretermination codon (PTC) is produced), it can escape depletion by nonsense-mediated mRNA decay (NMD) and be readily expressed, processed, and presented on the cell surface by the HLA system. Further, mutation-derived alternative splicing is usually “cryptic”, i.e., not expressed in normal tissues, and therefore may be recognized by T-cells as non-self neoantigens.

In some instances, the cancer therapeutic agent is a vaccine which includes one or more neoantigens which are recurrent polymorphisms (“hotspot mutations”). For example, among other things, the present disclosure provides neoantigen peptide sequences resulting from certain recurrent somatic cancer mutations in p53. Hotspot mutations are described in further detail above.

In some embodiments, methods provided herein result in immune responses to one or more peptide antigens encoded by the mRNA cancer vaccine. In some embodiments, methods provided herein result in immune responses to epitopes other than those encoded by the mRNA cancer vaccine, e.g., through epitope spreading.

In some embodiments, methods provided herein (e.g., comprising administration of a cancer vaccine and/or an immune checkpoint inhibitor) result in changes in recurrence-free survival (RFS), distant metastasis-free survival (DMFS), overall survival, and/or quality of life in an individual, or on average in a population of individuals. For example, in some embodiments, administration of a cancer vaccine and/or an immune checkpoint inhibitor to a population of individuals results in improvements in RFS, DMFS, overall survival, and/or quality of life in the population relative to a population not receiving the treatment (e.g., not receiving the cancer vaccine, or only receiving the immune checkpoint inhibitor without the cancer vaccine).

### Immune Responses

Provided herein are methods relating to inducing an immune response to a tumor in a subject, e.g., by administering a cancer vaccine (e.g., a nucleic acid cancer vaccine such as an mRNA cancer vaccine) and/or an immune checkpoint modulator to the subject. An induced immune response to a tumor in a subject can comprise a variety of components, such as cellular responses (e.g., T cell responses) and antibody responses.

In some embodiments, an induced immune response to a tumor comprises a cellular response to one or more antigens (e.g., neoantigens) expressed in the tumor. In some embodiments, a cellular response comprises a T cell response, e.g., a CD4 T cell response and/or a CD8 T cell response. In some embodiments, a T cell response comprises generation of one or more de novo T cell responses to a tumor antigen. For example, in some embodiments, a T cell response to a tumor antigen results in the presence of a T cell with specificity for the tumor antigen, wherein the T cell with specificity for the tumor antigen was not previously present or was not previously detectable (e.g., in a subject or in a biological sample collected from a subject). Such a T cell response to a tumor antigen can result from the immune system's response to a neoantigen, or to a peptide corresponding to the neoantigen (e.g., a peptide encoded by a nucleic acid vaccine provided herein). In some embodiments, a T cell response to a tumor antigen is not detectable in a subject prior to administration to the subject of a cancer vaccine, but is detectable in the subject after administration of the vaccine.

A T cell response to a specific antigen can be detected, for example, by collecting a sample comprising immune cells (e.g., peripheral blood mononuclear cells (PBMCs), such as PBMCs from a blood sample), stimulating the immune cells with the specific antigen, and subsequently measuring immune activation signals (e.g., cytokine production) from the immune cells. T cells with specificity for the specific antigen produce activation signals (e.g., cytokines) in response to the stimulation, and can thereby be detected. A T cell response to a specific antigen can also be detected by a method described in U.S. Patent Application Pub. No. US2022/0236253A1, the contents of which are herein incorporated by reference in their entirety for this purpose.

In some embodiments, a T cell response comprises an increase in an existing T cell responses to a tumor antigen in the subject. This increase can be the result of an increase in the individual strength of the reaction of the antigen-specific T cells to the antigen, an increase in the size of the population of T cells specific for the antigen, and/or a decrease in immunosuppressive signals (e.g., a decrease in the size of a population of cells which suppress T cell activity against the antigen, such as regulatory T cells (Tregs)).

An increase in the individual strength of the reaction of antigen-specific T cells to the antigen can be measured, e.g., as described above, by first selecting for antigen-specific T cells and normalizing the measured immune activation signals (e.g., cytokines) to the total number of antigen-specific T cells.

An increase in the size of a population of antigen-specific T cells can be detected by comparing the measured immune activation signals (e.g., cytokines) from a defined number of T cells (e.g., from PBMCs) in a sample collected prior to the immune response induction (e.g., prior to the administration of a cancer vaccine) with that in a sample collected after the immune response induction. Sizes of populations of cells (e.g., antigen-specific T cells and cells which suppress T cell activity) can also be measured, for example, by flow cytometric analysis using markers for the particular population(s) of interest. Such flow cytometric analysis can, for example, allow one to determine the ratio of a specific population of T cells (e.g., antigen-specific T cells) to a broader population of cells (e.g., to all T cells) in a biological sample.

In embodiments in which an immune response (e.g., an immune response to a tumor) is or is not detected to a specific antigen (e.g., a cancer neoantigen), such detection or lack thereof can inform optimization of the vaccine (e.g., personalized cancer vaccine). For example, if an immune response to a specific antigen is not detected following administration of a personalized cancer vaccine encoding a peptide corresponding to that antigen, that peptide may be removed from an optimized personalized cancer vaccine. Similarly, if a new immune response (or an increase in a preexisting immune response) to a specific antigen is detected following administration of a personalized cancer vaccine encoding a peptide corresponding to that antigen, more than one copy of that peptide may be encoded by an optimized personalized cancer vaccine, and/or additional similar peptides corresponding to that antigen may be added to the optimized personalized cancer vaccine.

### Lipid Compositions

In some embodiments, the nucleic acids are formulated in a lipid delivery vehicle, such as a lipid nanoparticle, a liposome, and/or a lipoplex. In some embodiments, nucleic acids are formulated as lipid nanoparticle (LNP) compositions. Lipid nanoparticles typically comprise amino lipid, non-cationic lipid, structural lipid, and PEG lipid components along with the nucleic acid cargo of interest. The lipid nanoparticles can be generated using components, compositions, and methods as are generally known in the art, see for example, International Patent Application Nos. PCT/US2016/052352; PCT/US2016/068300; PCT/US2017/037551; PCT/US2015/027400; PCT/US2016/047406; PCT/US2016000129; PCT/US2016/014280; PCT/US2017/038426; PCT/US2014/027077; PCT/US2014/055394; PCT/US2016/52117; PCT/US2012/069610; PCT/US2017/027492; PCT/US2016/059575; PCT/US2016/069491; PCT/US2016/069493; and PCT/US2014/66242, all of which are incorporated by reference herein in their entirety.

In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises a molar ratio of 20-60% ionizable amino lipid, 5-25% non-cationic lipid, 25-55% structural lipid, and 0.5-15% PEG-modified lipid.

In some embodiments, the lipid nanoparticle comprises a molar ratio of 20-60% ionizable amino lipid, 5-30% non-cationic lipid, 10-55% structural lipid, and 0.5-15% PEG-modified lipid.

In some embodiments, the lipid nanoparticle comprises 40-50 mol % ionizable lipid, optionally 45-50 mol %, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %.

In some embodiments, the lipid nanoparticle comprises 20-60 mol % ionizable amino lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, the lipid nanoparticle comprises 20 mol %, 30 mol %, 40 mol %, 50 mol %, or 60 mol % ionizable amino lipid. In some embodiments, the lipid nanoparticle comprises 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, or 55 mol % ionizable amino lipid.

In some embodiments, the lipid nanoparticle comprises 45-55 mole percent (mol %) ionizable amino lipid. For example, lipid nanoparticle may comprise 45, 46, 47, 48, 49, 50, 51, 52, 53, 54, or 55 mol % ionizable amino lipid.

### Ionizable Amino Lipids

### Formula (AI)

In some embodiments, the ionizable amino lipid is a compound of Formula (AI):

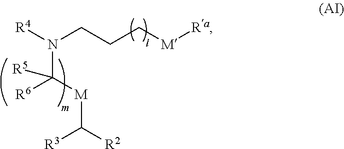

or its N-oxide, or a salt or isomer thereof,
wherein R′^{a} is R′^{branched}, wherein
R′^{branched} is:

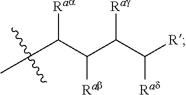

wherein

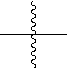

denotes a point of attachment;
wherein R_{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

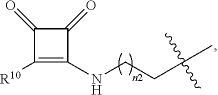

wherein

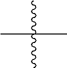

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of the compounds of Formula (AI), R′^{a} is R′_{branched}. R′_{branched} is

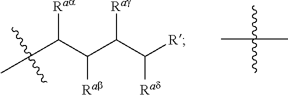

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of the compounds of Formula (AI), R′^{a} is R′^{branched}. R′^{branched} is

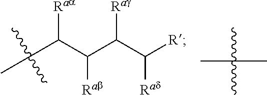

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of the compounds of Formula (AI), R′^{a} is R′^{branched}. R′^{branched} is

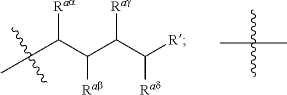

denotes a point of attachment; R^{aα} is C₂₋₁₂ alkyl; R^{aβ}, R^{aγ}, and R^{aδ} are each H; R² and R³ are each C₁₋₁₄ alkyl; R⁴ is

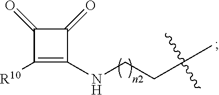

R¹⁰ NH(C₁₋₆ alkyl); n2 is 2; R⁵ is H; each R⁶ is H; M and M′ are each —C(O)O—; R′ is a C₁₋₁₂ alkyl; 1 is 5; and m is 7.

In some embodiments of the compounds of Formula (AI), R′^{a} is R′^{branched}, R′^{branched} is

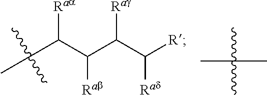

denotes a point of attachment; R^{aα}, R^{aβ}, and R^{aδ} are each H; R_{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the compound of Formula (AI) is selected from:

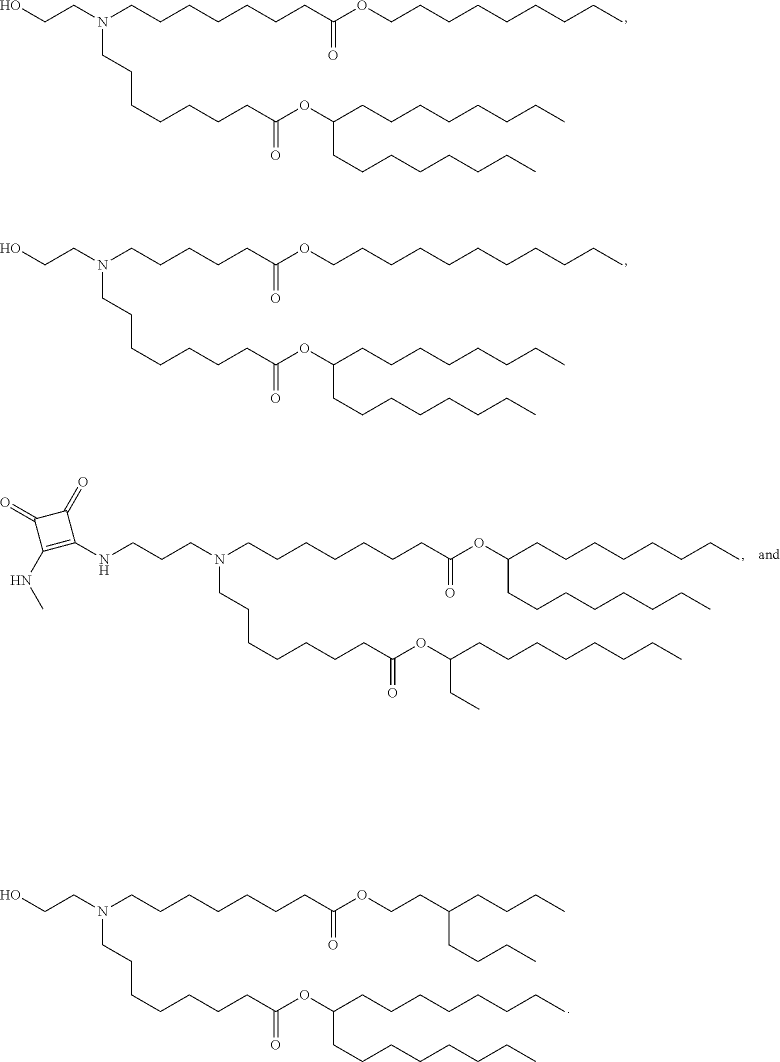 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the ionizable amino lipid of Formula (AI) is a compound of Formula (AIa):

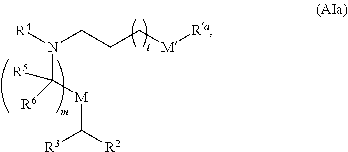

or its N-oxide, or a salt or isomer thereof,
wherein R′^{a} is R′^{branched}, wherein
R′^{branched} is:

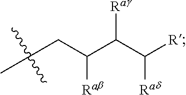

wherein

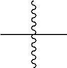

denotes a point of attachment;
wherein R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

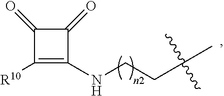

wherein

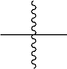

denotes a point of attachment; wherein
   R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, the ionizable amino lipid of Formula (AI) is a compound of Formula (AIb):

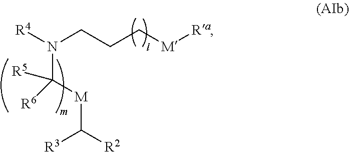

or its N-oxide, or a salt or isomer thereof,
wherein R′^{a} is R′^{branched}, wherein
R′^{branched} is:

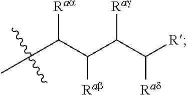

wherein

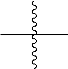

denotes a point of attachment;
wherein R^{aα}, R^{aβ}, R^{aδ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is —(CH₂)ₙOH, wherein n is selected from the group consisting of 1, 2, 3, 4, and 5;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of Formula (AI) or (AIb), R′^{a} is R′^{branched}; R′^{branched} is

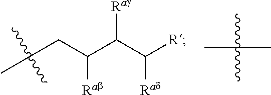

denotes a point of attachment; R^{aβ}, R^{aδ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of Formula (AI) or (AIb), R′^{a} is R′^{branched}. R′^{branched} is

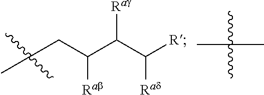

denotes a point of attachment; R^{aβ}, R^{aδ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 3; and m is 7.

In some embodiments of Formula (AI) or (AIb), R′^{a} is R′^{branched}. R′^{branched} is

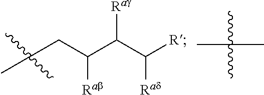

denotes a point of attachment; R^{aβ} and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the ionizable amino lipid of Formula (AI) is a compound of Formula (AIc):

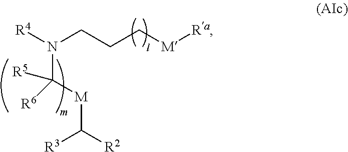

or its N-oxide, or a salt or isomer thereof,
wherein R′^{a} is R′^{branched}, wherein
R′^{branched} is:

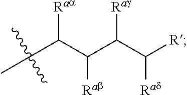

wherein

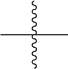

denotes a point of attachment;
wherein R^{aα}, R^{aβ}, R^{aδ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is

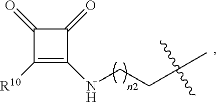

wherein

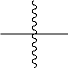

denotes a point of attachment; wherein R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, R′^{a} is R′^{branched}. R′^{branched} is

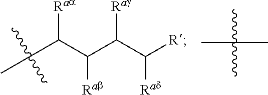

denotes a point of attachment; R^{aβ}, R^{aδ}, and R^{aδ} are each H; R^{aα} is C₂₋₁₂ alkyl; R² and R³ are each C₁₋₁₄ alkyl; R⁴ is

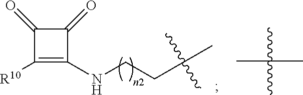

denotes a point of attachment; R¹⁰ is NH(C₁₋₆ alkyl); n2 is 2; each R⁵ is H; each R⁶ is H; M and M′ are each —C(O)O—; R′ is a C₁₋₁₂ alkyl; 1 is 5; and m is 7.

In some embodiments, the compound of Formula (AIc) is:

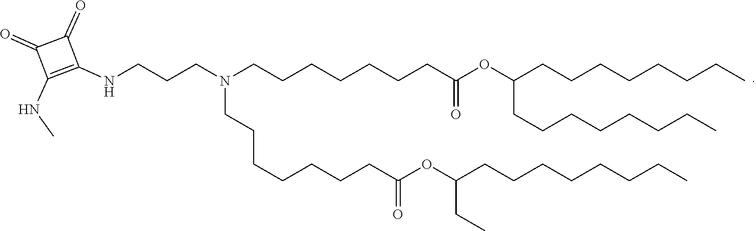 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

### Formula (AII)

In some embodiments, the ionizable amino lipid is a compound of Formula (AII):

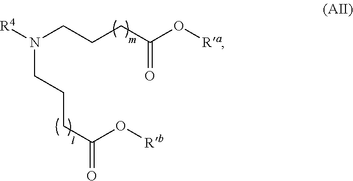

or its N-oxide, or a salt or isomer thereof,
wherein R′^{a} is R′^{branched} or R′^{cyclic}, wherein
R′^{branched} is:

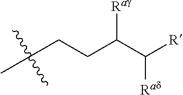

and R′cyclic is:

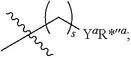

and
R′^{b} is:

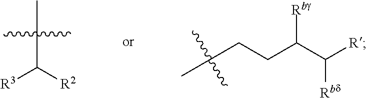

wherein

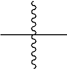

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

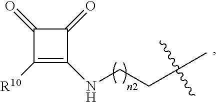

wherein

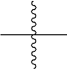

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₅ alkenyl; and
s is 2 or 3;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-a):

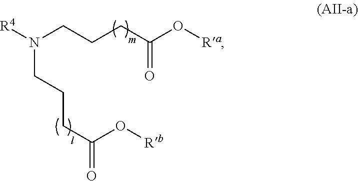

or its N-oxide, or a salt or isomer thereof,
wherein R′^{a} is R′^{branched} or R′^{cyclic}, wherein
R′^{branched} is:

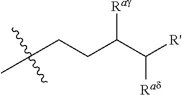

and R′^{b} is:

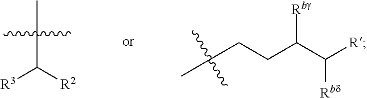

wherein

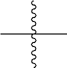

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

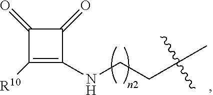

wherein

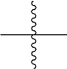

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-b):

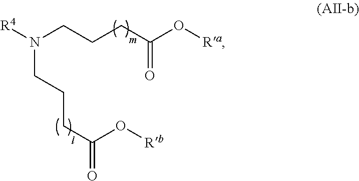

or its N-oxide, or a salt or isomer thereof,
wherein R′^{a} is R′^{branched} or R′^{cyclic}, wherein
R′^{branched} is:

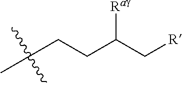

and R′^{b} is:

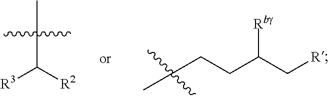

wherein

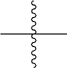

denotes a point of attachment;
R^{aγ} and R^{bγ} are each independently selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

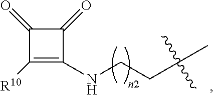

wherein

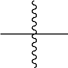

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-c):

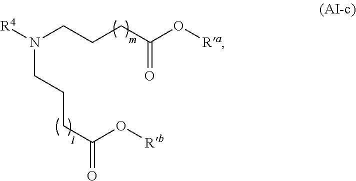

or its N-oxide, or a salt or isomer thereof,
wherein R′ₐ is R′^{branched} or R′^{cyclic}, wherein
R′^{branched} is:

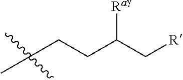

and R′^{b} is:

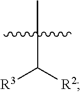

wherein

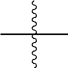

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

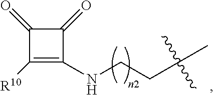

wherein

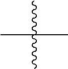

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-d):

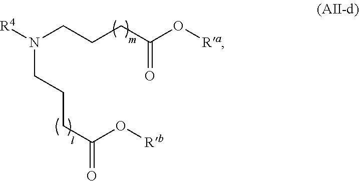

or its N-oxide, or a salt or isomer thereof,
wherein R′^{a} is R′^{branched} or R′^{cyclic}, wherein
R′^{branched} is:

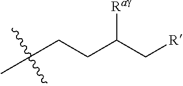

and R′^{b} is:

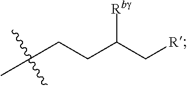

wherein

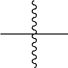

denotes a point of attachment;
wherein R^{aγ} and R^{bγ} are each independently selected from the group consisting of C₁₋₁₂ alkyl and C₂₋₁₂ alkenyl;
R⁴ is selected from the group consisting of —(CH₂)ₙOH wherein n is selected from the group consisting of 1, 2, 3, 4, and 5, and

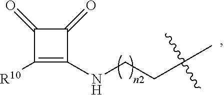

wherein

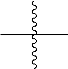

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-e):

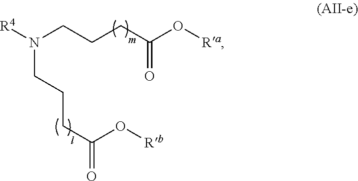

or its N-oxide, or a salt or isomer thereof,
wherein R′^{a} is R′^{branched} or R′^{cyclic}, wherein
R′^{branched} is:

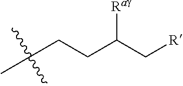

and R′^{b} is:

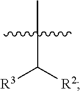

wherein

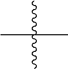

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments of the compound of Formula (AII), (AII-a), (AII-b), (AII-c), (AII-d), or (AII-e), m and l are each independently selected from 4, 5, and 6. In some embodiments of the compound of Formula (AII), (AII-a), (AII-b), (AII-c), (AII-d), or (AII-e), m and 1 are each 5.

In some embodiments of the compound of Formula (AII), (AII-a), (AII-b), (AII-c), (AII-d), or (AII-e), each R′ independently is a C₁₋₁₂ alkyl. In some embodiments of the compound of Formula (AII), (AII-a), (AII-b), (AII-c), (AII-d), or (AII-e), each R′ independently is a C₂₋₅ alkyl.

In some embodiments of the compound of Formula (AII), (AII-a), (AII-b), (AII-c), (AII-d), or (AII-e), R′^{b} is:

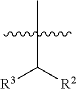

and R² and R³ are each independently a C₁₋₁₄ alkyl. In some embodiments of the compound of Formula (AII), (AII-a), (AII-b), (AII-c), (AII-d), or (AII-e), R′^{b} is:

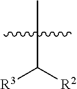

and R² and R³ are each independently a C₆₋₁₀ alkyl. In some embodiments of the compound of Formula (AII), (AII-a), (AII-b), (AII-c), (AII-d), or (AII-e), R′^{b} is:

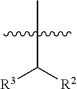

and R² and R³ are each a C₈ alkyl.

In some embodiments of the compound of Formula (AII), (AII-a), (AII-b), (AII-c), (AII-d), or (AII-e), R′^{branched} is:

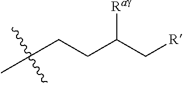

### and R′^{b} is:

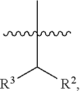

R^{aγ} is a C₁₋₁₂ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

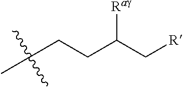

### and R′^{b} is:

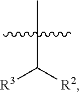

R^{aγ} is a C₂₋₆ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

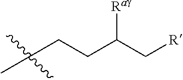

### and R′^{b} is:

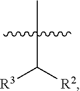

R^{aγ} is a C₂₋₆ alkyl, and R² and R³ are each a C₈ alkyl.

In some embodiments of the compound of Formula (AII), (AII-a), (AII-b), (AII-c), (AII-d), or (AII-e), R′^{branched} is:

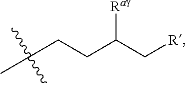

### R′^{b} is:

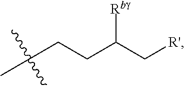

and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

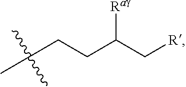

### R′^{b} is:

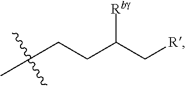

and R^{aγ} and R^{bγ} are each a C₂₋₆ alkyl.

In some embodiments of the compound of Formula (AII), (AII-a), (AII-b), (AII-c), (AII-d), or (AII-e), m and l are each independently selected from 4, 5, and 6 and each R′ independently is a C₁₋₁₂ alkyl. In some embodiments of the compound of Formula (AII), (AII-a), (AII-b), (AII-c), (AII-d), or (AII-e), m and l are each 5 and each R′ independently is a C₂₋₅ alkyl.

In some embodiments of the compound of (AII), (AII-a), (AII-b), (AII-c), (AII-d), or (AII-e), R′^{branched} is:

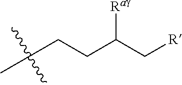

### R′^{b} is:

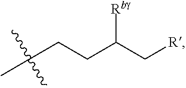

m and l are each independently selected from 4, 5, and 6, each R′ independently is a C₁₋₁₂ alkyl, and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

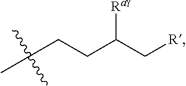

### R′^{b} is:

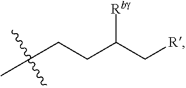

m and l are each 5, each R′ independently is a C₂₋₅ alkyl, and R^{aγ} and R^{bγ} are each a C₂₋₆ alkyl.

In some embodiments of the compound of Formula (AII), (AII-a), (AII-b), (AII-c), (AII-d), or (AII-e), R′^{branched} is:

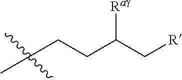

### and R′^{b} is:

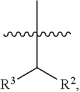

m and l are each independently selected from 4, 5, and 6, R′ is a C₁₋₁₂ alkyl, R^{aγ} is a C₁₋₁₂ alkyl and R² and R³ are each independently a C₆₋₁₀ alkyl.

In some embodiments of the compound of Formula (AII), (AII-a), (AII-b), (AII-c), (AII-d), or (AII-e), R′^{branched} is:

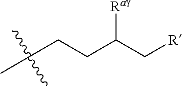

### and R′^{b} is:

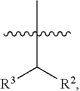

m and l are each 5, R′ is a C₂₋₅ alkyl, R^{aγ} is a C₂₋₆ alkyl, and R² and R³ are each a C₈ alkyl.

In some embodiments of the compound of (AII), (AII-a), (AII-b), (AII-c), (AII-d), or (AII-e), R⁴ is

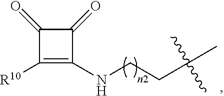

wherein R¹⁰ is NH(C₁₋₆ alkyl) and n2 is 2. In some embodiments of the compound of Formula (AII), (AII-a), (AII-b), (AII-c), (AII-d), or (AII-e), R⁴ is

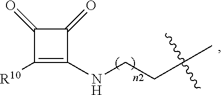

wherein R¹⁰ is NH(CH₃) and n2 is 2.

In some embodiments of the compound of Formula (AII), (AII-a), (AII-b), (AII-c), (AII-d), or (AII-e), R′^{branched} is:

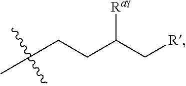

### R′^{b} is:

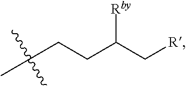

m and l are each independently selected from 4, 5, and 6, each R′ independently is a C₁₋₁₂ alkyl, R^{aγ} and R^{bγ} are each a C₁₋₁₂ alkyl, and R⁴ is

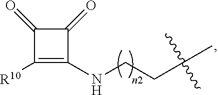

wherein R¹⁰ is NH(C₁₋₆ alkyl), and n2 is 2. In some embodiments of the compound of Formula (AII), (AII-a), (AII-b), (AII-c), (AII-d), or (AII-e), R′^{branched} is:

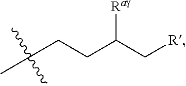

### R′^{b} is:

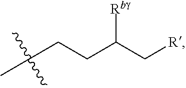

m and l are each 5, each R′ independently is a C₂₋₅ alkyl, R^{aγ} and R^{bγ} are each a C₂₋₆ alkyl, and R⁴ is

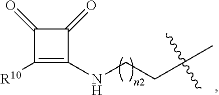

wherein R¹⁰ is NH(CH₃) and n2 is 2.

In some embodiments of the compound of Formula (AII), (AII-a), (AII-b), (AII-c), (AII-d), or (AII-e), R′^{branched} is:

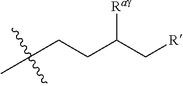

### and R′^{b} is:

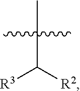

m and l are each independently selected from 4, 5, and 6, R′ is a C₁₋₁₂ alkyl, R² and R³ are each independently a C₆₋₁₀ alkyl, R^{aγ} is a C₁₋₁₂ alkyl, and R⁴ is

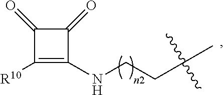

wherein R¹⁰ is NH(C₁₋₆ alkyl) and n2 is 2. In some embodiments of the compound of Formula (AII), (AII-a), (AII-b), (AII-c), (AII-d), or (AII-e), R′^{branched} is:

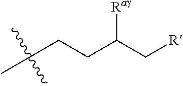

### and R′^{b} is:

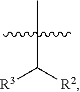

m and l are each 5, R′ is a C₂₋₅ alkyl, R^{aγ} is a C₂₋₆ alkyl, R² and R³ are each a C₈ alkyl, and R⁴ is

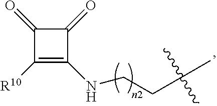

wherein R¹⁰ is NH(CH₃) and n2 is 2.

In some embodiments of the compound of Formula (AII), (AII-a), (AII-b), (AII-c), (AII-d), or (AII-e), R⁴ is —(CH₂)ₙOH and n is 2, 3, or 4. In some embodiments of the compound of Formula (AII), (AII-a), (AII-b), (AII-c), (AII-d), or (AII-e), R⁴ is —(CH₂)ₙOH and n is 2.

In some embodiments of the compound of Formula (AII), (AII-a), (AII-b), (AII-c), (AII-d), or (AII-e), R′^{branched} is:

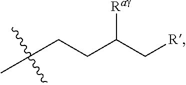

### R′^{b} is:

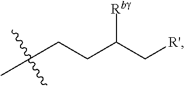

m and l are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4. In some embodiments of the compound of Formula (AII), (AII-a), (AII-b), (AII-c), (AII-d), or (AII-e), R′^{branched} is:

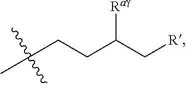

### R′^{b} is:

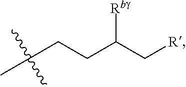

m and l are each 5, each R′ independently is a C₂₋₅ alkyl, R^{aγ} and R^{bγ} are each a C₂₋₆ alkyl, R⁴ is —(CH₂)ₙOH, and n is 2.

In some embodiments, the ionizable amino lipid of Formula (AII) is a compound of Formula (AII-f):

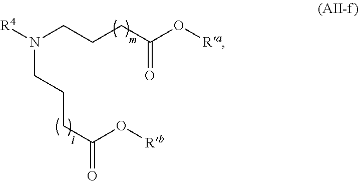

or its N-oxide, or a salt or isomer thereof,
wherein R′^{a} is R′^{branched} or R′^{cyclic}, wherein
R′^{branched} is:

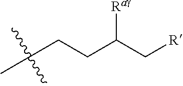

and R′^{b} is:

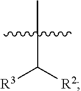

wherein

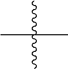

denotes a point of attachment;
R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
l is selected from 4, 5, and 6.

In some embodiments of the compound of Formula (AII-f), m and l are each 5, and n is 2, 3, or 4.

In some embodiments of the compound of Formula (AII-f) R′ is a C₂₋₅ alkyl, R^{aγ} is a C₂₋₆ alkyl, and R² and R³ are each a C₆₋₁₀ alkyl.

In some embodiments of the compound of Formula (AII-f), m and l are each 5, n is 2, 3, or 4, R′ is a C₂₋₅ alkyl, R^{aγ} is a C₂₋₆ alkyl, and R² and R³ are each a C₆₋₁₀ alkyl.

In some embodiments, the ionizable amino lipid of Formula (AII) is a compound of Formula (AII-g):

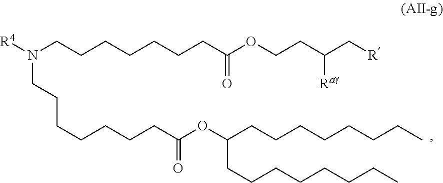

or its N-oxide, or a salt or isomer thereof; wherein
R^{aγ} is a C₂₋₆ alkyl;
R′ is a C₂₋₅ alkyl; and
R⁴ is selected from the group consisting of —(CH₂)ₙOH wherein n is selected from the group consisting of 3, 4, and 5, and

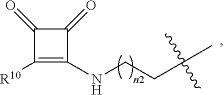

wherein

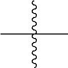

denotes a point of attachment, R¹⁰ is NH(C₁₋₆ alkyl), and n2 is selected from the group consisting of 1, 2, and 3.

In some embodiments, the ionizable amino lipid of Formula (AII) is a compound of Formula (AII-h):

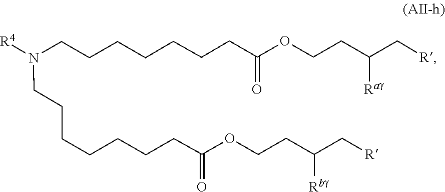

or its N-oxide, or a salt or isomer thereof; wherein
R^{aγ} and R^{bγ} are each independently a C₂₋₆ alkyl;
each R′ independently is a C₂₋₅ alkyl; and
R⁴ is selected from the group consisting of —(CH₂)ₙOH wherein n is selected from the group consisting of 3, 4, and 5, and

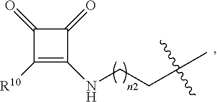

wherein

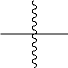

denotes a point of attachment, R¹⁰ is NH(C₁₋₆ alkyl), and n2 is selected from the group consisting of 1, 2, and 3.

In some embodiments of the compound of Formula (AII-g) or (AII-h), R⁴ is

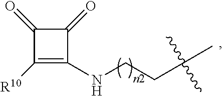

wherein
R¹⁰ is NH(CH₃) and n2 is 2.

In some embodiments of the compound of Formula (AII-g) or (AII-h), R⁴ is —(CH₂)₂OH.

### Formula (AIII)

In some embodiments, the ionizable amino lipids may be one or more of compounds of Formula (AIII):

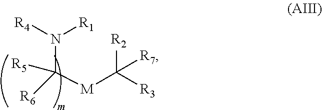

or their N-oxides, or salts or isomers thereof, wherein:
R₁ is selected from the group consisting of C₅₋₃₀ alkyl, C₅₋₂₀ alkenyl, —R*YR″, —YR″, and —R″M′R′;
R₂ and R₅ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of hydrogen, a C₃₋₆ carbocycle, —(CH₂)ₙQ, —(CH₂)ₙCHQR,
—CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —N(R)S(O)₂R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected
   from —C(O)O—, —OC(O)—, —OC(O)-M″-C(O)O—, —C(O)N(R′)—, —N(R′) C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13; and wherein when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (AIII) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′) C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR,
—N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′) C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′) C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′) C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′) C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In certain embodiments, a subset of compounds of Formula (AIII) includes those of Formula (AIII-A):

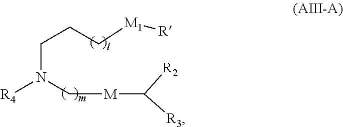

or its N-oxide, or a salt or isomer thereof, wherein 1 is selected from 1, 2, 3, 4, and 5; m is selected from 5, 6, 7, 8, and 9; M₁ is a bond or M′; R₄ is hydrogen, unsubstituted C₁₋₃ alkyl, or —(CH₂)ₙQ, in which Q is
—OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (AIII) includes those of Formula (AIII-B):

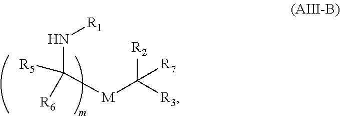

or its N-oxide, or a salt or isomer thereof in which all variables are as defined herein. For example, m is selected from 5, 6, 7, 8, and 9; R₄ is hydrogen, unsubstituted C₁₋₃ alkyl, or —(CH₂)ₙQ, in which Q is H, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (AIII) includes those of Formula (AIII-C):

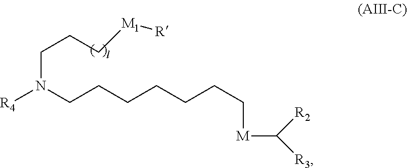

or its N-oxide, or a salt or isomer thereof, wherein 1 is selected from 1, 2, 3, 4, and 5; M₁ is a bond or M′; R₄ is hydrogen, unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, the compounds of Formula (AIII) are of Formula (AIII-D),

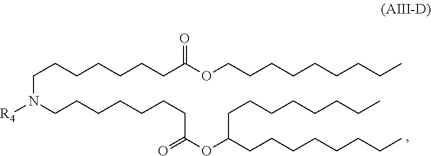

or their N-oxides, or salts or isomers thereof, wherein R₄ is as described herein.

In another embodiment, the compounds of Formula (AIII) are of Formula (AIII-E),

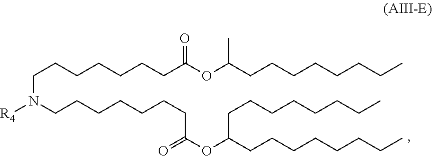

or their N-oxides, or salts or isomers thereof, wherein R₄ is as described herein.

In some embodiments, the compounds of Formula (AIII) are of Formula (AIII-F) or (AIII-G)

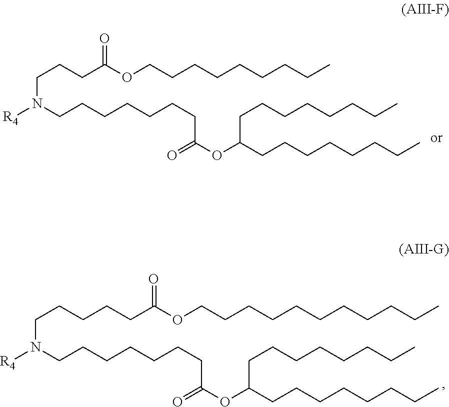

or their N-oxides, or salts or isomers thereof, wherein R₄ is as described herein.

In some embodiments, the compounds of Formula (AIII) are of Formula (AIII-H):

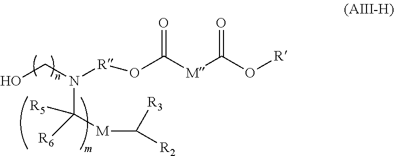

or their N-oxides, or salts or isomers thereof,

wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In some embodiments, the compounds of Formula (AIII) are of Formula (AIII-I):

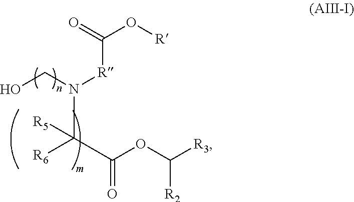

or their N-oxides, or salts or isomers thereof, wherein n is 2, 3, or 4; and m, R′, R″, and R₂ through R₆ are as described herein. For example, each of R₂ and R₃ may be independently selected from the group consisting of C₅₋₁₄ alkyl and C₅-14 alkenyl.

In some embodiments, an ionizable amino lipid of the disclosure comprises a compound having structure:

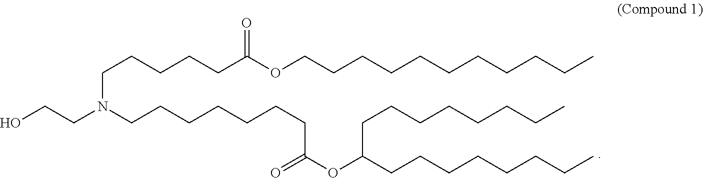 [SMILES: CCCCCCCCCCCOC(=O)CCCCCN(CCO)CCCCCCCC(=O)OC(CCCCCCCC)CCCCCCCC]

In some embodiments, an ionizable amino lipid of the disclosure comprises a compound having structure:

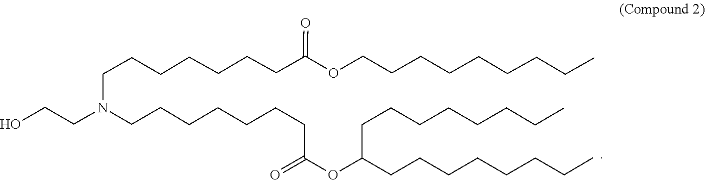 [SMILES: CCCCCCCCCOC(=O)CCCCCCCN(CCO)CCCCCCCC(=O)OC(CCCCCCCC)CCCCCCCC]

In some embodiments, the compounds of Formula (AIII) are of Formula (AIII-J),

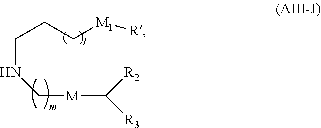

or their N-oxides, or salts or isomers thereof, wherein 1 is selected from 1, 2, 3, 4, and 5; m is selected from 5, 6, 7, 8, and 9; M₁ is a bond or M′; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the ionizable amino lipids are of Formula (AIII), or salts or isomers thereof, wherein:
R₁ is —R″M′R′;
R₂ and R₃ are each independently selected from C₁₋₁₄ alkyl and C₂₋₁₄ alkenyl;
R₄ is —(CH₂)ₙQ, wherein Q is OH and n is selected from 3, 4, and 5;
M and M′ are each independently —OC(O)—;
R₅, R₆, and R₇ are each H;
R′ is a linear C₁₋₁₂ alkyl, or C₁₋₁₂ alkyl substituted with C₆₋₉ alkyl;
R″ is C₃₋₁₄ alkyl;
m is selected from 5, 6, 7, 8, 9, 10, 11, 12, and 13.

In some embodiments, the ionizable amino lipids are of Formula (AIII), or salts or isomers thereof, wherein:
R₁ is R″M′R′;
R₂ and R₅ are each independently C₁₋₁₄ alkyl;
R₄ is —(CH₂)ₙQ, wherein Q is OH and n is 4;
M and M′ are each independently —OC(O)—;
R₅, R₆, and R₇ are each H;
R′ is C₁₋₁₂ alkyl substituted with C₆₋₉ alkyl;
R″ is C₃₋₁₄ alkyl; and
m is 6.

In some embodiments, an ionizable amino lipid of the disclosure comprises a compound having structure:

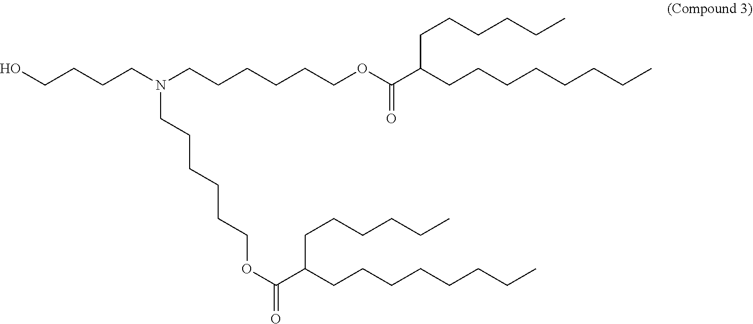 [SMILES: CCCCCCCCC(CCCCCC)C(=O)OCCCCCCN(CCCCO)CCCCCCOC(=O)C(CCCCCC)CCCCCCCC]

In some embodiments, the ionizable amino lipids are of Formula (AIII), or salts or isomers thereof, wherein:
R₁ is C₅₋₂₀ alkenyl;
R₂ and R₃ are each independently selected from C₁₋₁₄ alkyl and C₂₋₁₄ alkenyl;
R₄ is —(CH₂)ₙQ, wherein Q is OH and n is selected from 3, 4, and 5;
M and M′ are each independently C(O)O—;
R₅, R₆, and R₇ are each H;
R′ is a linear C₁₋₁₂ alkyl, or C₁₋₁₂ alkyl substituted with C₆₋₉ alkyl;
R″ is C₃₋₁₄ alkyl;
m is selected from 5, 6, 7, 8, 9, 10, 11, 12, and 13.

In some embodiments, the ionizable amino lipids are of Formula (AIII), or salts or isomers thereof, wherein:
R₁ is C₅₋₂₀ alkenyl;
R₂ and R₃ are each independently C₁₋₁₄ alkyl;
R₄ is —(CH₂)ₙQ, wherein Q is OH and n is 3;
M is —C(O)O—;
R₅, R₆, and R₇ are each H; and
m is 6.

In some embodiments, an ionizable amino lipid of the disclosure comprises a compound having structure:

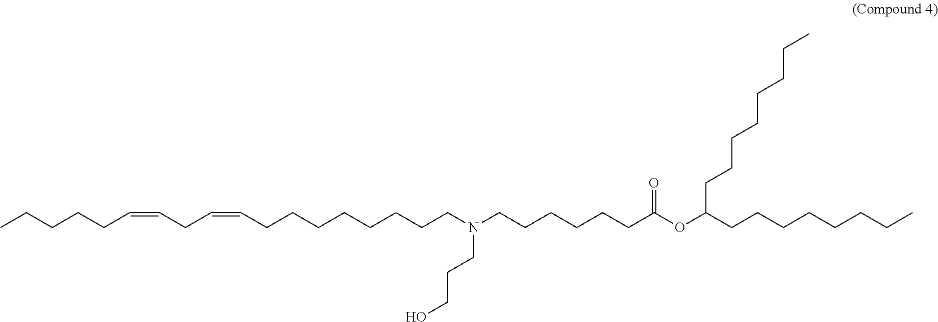 [SMILES: CCCCC/C=C\C/C=C\CCCCCCCCN(CCCO)CCCCCCC(=O)OC(CCCCCCCC)CCCCCCCC]

In some embodiments, the ionizable amino lipids are one or more of the compounds described in U.S. Patent Application Nos. 62/220,091, 62/252,316, 62/253,433, 62/266,460, 62/333,557, 62/382,740, 62/393,940, 62/471,937, 62/471,949, 62/475,140, and 62/475,166, and International Patent Application No. PCT/US2016/052352.

The central amine moiety of a lipid according to Formula (AIII), (AIII-A), (AIII-B), (AIII-C), (AIII-D), (AIII-E), (AIII-F), (AIII-G), (AIII-H), (AIII-I), or (AIII-J) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

### Formula (AIV)

In some embodiments, the ionizable amino lipids may be one or more of compounds of formula (AIV),

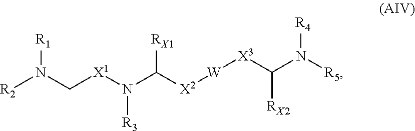

or salts or isomers thereof, wherein
W is

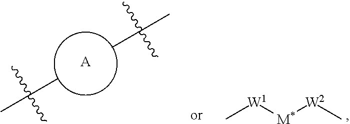

ring A is

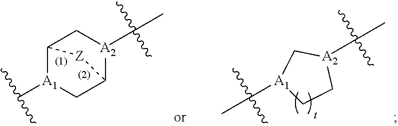

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
R_{X1} and R_{X2} are each independently H or C₁₋₃ alkyl;
each M is independently selected from the group consisting of —C(O)O—, —OC(O)—, —OC(O)O—, —C(O)N(R′)—, —N(R′) C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group; M* is C₁-C₆ alkyl,
W¹ and W² are each independently selected from the group consisting of —O— and —N(R₆)—;
each R₆ is independently selected from the group consisting of H and C₁₋₅ alkyl;
X¹, X², and X³ are independently selected from the group consisting of a bond, —CH₂—, (CH₂)₂—, —CHR—, —CHY—, —C(O)—, —C(O)O—, —OC(O)—, —(CH₂)ₙ—C(O)—, —C(O)—(CH₂)ₙ—, (CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6;
wherein when ring A is

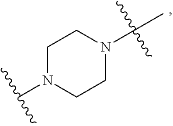 [SMILES: *N1CCN(*)CC1]

then
i) at least one of X¹, X², and X³ is not —CH₂—; and/or
ii) at least one of R₁, R₂, R₃, R₄, and R₅ is-R″MR′.

In some embodiments, the compound is of any of formulae (AIVa)-(AIVh):

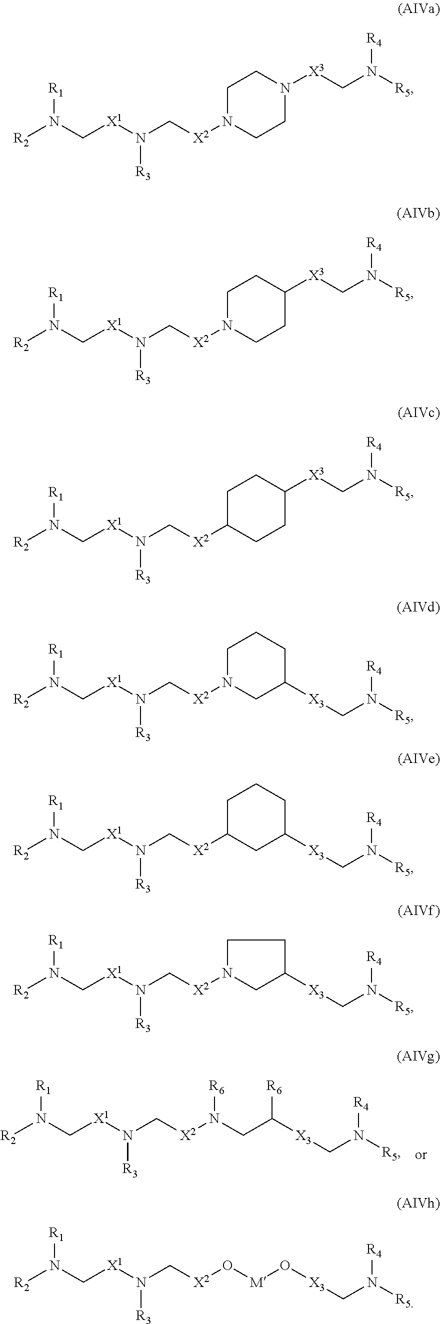

In some embodiments, the ionizable amino lipid is

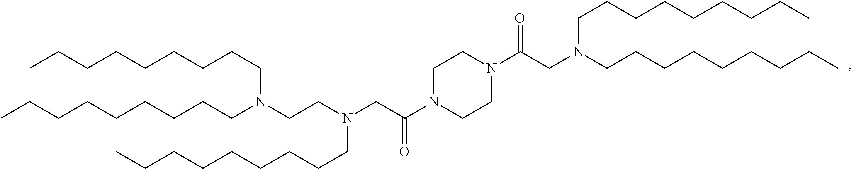 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (AIV), (AIVa), (AIVb), (AIVc), (AIVd), (AIVe), (AIVf), (AIVg), or (AIVh) may be protonated at a physiological pH. Thus, a lipid may have a positive or partial positive charge at physiological pH.

### Formula (AV)

In some embodiments, the lipid nanoparticle comprises a lipid having the structure:

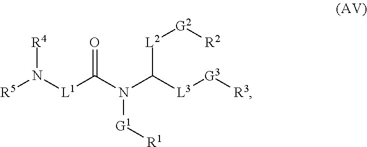

or a pharmaceutically acceptable salt, tautomer, or stereoisomer thereof, wherein:
R¹ is optionally substituted C₁-C₂₄ alkyl or optionally substituted C₂-C₂₄ alkenyl;
R² and R³ are each independently optionally substituted C₁-C₃₆ alkyl;
R⁴ and R⁵ are each independently optionally substituted C₁-C₆ alkyl, or R⁴ and R⁵ join, along with the N to which they are attached, to form a heterocyclyl or heteroaryl;
L¹, L², and L³ are each independently optionally substituted C₁-C₁₈ alkylene;
G¹ is a direct bond, —(CH₂)ₙO(C═O)—, —(CH₂)ₙ(C═O)O—, or —(C═O)—;
G² and G³ are each independently —(C═O)O— or —O(C═O)—; and n is an integer greater than 0.

### Formula (AVI)

In some embodiments, the lipid nanoparticle comprises a lipid having the structure:

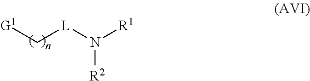

or a pharmaceutically acceptable salt, tautomer, or stereoisomer thereof, wherein:
G^{l} is —N(R³) R⁴ or —OR⁵;
R¹ is optionally substituted branched, saturated or unsaturated C₁₂-C₃₆ alkyl;
R² is optionally substituted branched or unbranched, saturated or unsaturated C₁₂-C₃₆ alkyl when L is —C(═O)—; or R² is optionally substituted branched or unbranched, saturated or unsaturated C₄-C₃₆ alkyl when L is C₆-C₁₂ alkylene, C₆-C₁₂ alkenylene, or C₂-C₆ alkynylene;
R³ and R⁴ are each independently H, optionally substituted branched or unbranched, saturated or unsaturated C₁-C₆ alkyl; or R³ and R⁴ are each independently optionally substituted branched or unbranched, saturated or unsaturated C₁-C₆ alkyl when L is C₆-C₁₂ alkylene, C₆-C₁₂ alkenylene, or C₂-C₆ alkynylene; or R³ and R⁴, together with the nitrogen to which they are attached, join to form a heterocyclyl;
R⁵ is H or optionally substituted C₁-C₆ alkyl;
L is —C(═O)—, C₆-C₁₂ alkylene, C₆-C₁₂ alkenylene, or C₂-C₆ alkynylene; and n is an integer from 1 to 12.

### Formula (AVII)

In some embodiments, the lipid nanoparticle comprises a lipid having the structure:

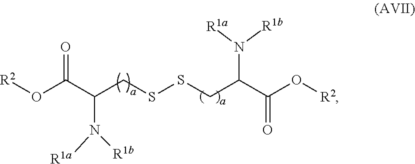

or a pharmaceutically acceptable salt thereof, wherein:
each R^{1a} is independently hydrogen, R^{1c}, or R^{1d},
each R^{1b} is independently R^{1c} or R^{1d},
each R^{1c} is independently —[CH₂]₂C(O) X¹R³;
each R^{1d} is independently —C(O)R⁴;
each R² is independently —[C(R^{2a})₂]_{c}R^{2b};
each R^{2a} is independently hydrogen or C₁-C₆ alkyl;
R^{2b} is —N(L₁-B)₂; —(OCH₂CH₂)₆OH; or —(OCH₂CH₂)₆OCH₃;
each R³ and R⁴ is independently C₆-C₃₀ aliphatic;
each I_{.3} is independently C₁-C₁₀ alkylene;
each B is independently hydrogen or an ionizable nitrogen-containing group;
each X¹ is independently a covalent bond or O;
each a is independently an integer of 1-10;
each b is independently an integer of 1-10; and
each c is independently an integer of 1-10.

### Formula (AVIII)

In some embodiments, the lipid nanoparticle comprises a lipid having the structure:

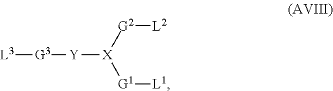

or a pharmaceutically acceptable salt, prodrug or stereoisomer thereof, wherein:
X is N, and Y is absent; or X is CR, and Y is NR;
L¹ is —O(C═O)R¹, —(C═O)OR¹, —C(═O)R¹, —OR¹, —S(O) «R¹, —S—SR¹, —C(═O) SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, or —NR^{a}C(═O)OR¹;
L² is —O(C═O)R², —(C═O)OR², —C(═O)R², —OR², —S(O)ₓR², —S—SR², —C(═O) SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
L³ is —O(C═O)R³ or —(C═O)OR³;
G¹ and G² are each independently C₂-C₁₂ alkylene or C₂-C₁₂ alkenylene;
G³ is C₁-C₂₄ alkylene, C₂-C₂₄ alkenylene, C₁-C₂₄ heteroalkylene or C₂-C₂₄ heteroalkenylene when X is CR, and Y is NR; and G³ is C₁-C₂₄ heteroalkylene or C₂-C₂₄ heteroalkenylene when X is N, and Y is absent;
R^{a}, R^{b}, R^{d} and R^{e} are each independently H or C₁-C₁₂ alkyl or C₁-C₁₂ alkenyl;
R^{e} and R^{f} are each independently C₁-C₁₂ alkyl or C₂-C₁₂ alkenyl;
each R is independently H or C₁-C₁₂ alkyl;
R¹, R² and R³ are each independently C₁-C₂₄ alkyl or C₂-C₂₄ alkenyl; and x is 0, 1 or 2, and
wherein each alkyl, alkenyl, alkylene, alkenylene, heteroalkylene and heteroalkenylene is independently substituted or unsubstituted unless otherwise specified.

### Formula (AIX)

In some embodiments, the lipid nanoparticle comprises a lipid having the structure:

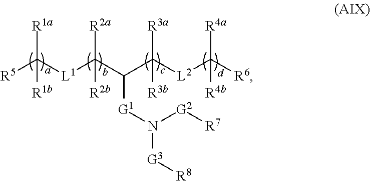

or a pharmaceutically acceptable salt, tautomer, prodrug or stereoisomer thereof, wherein:
L¹ and L² are each independently —O(C═O)—, —(C═O)O—, —C(═O)—, —O—, —S(O)x-, —S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O— or a direct bond;
G¹ is C₁-C₂ alkylene, —(C═O)—, —O(C═O)—, —SC(═O)—, —NR^{a}C(═O)— or a direct bond;
G² is —C(O)—, —(CO)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is H or C₁-C₂₀ alkyl;
R⁸ is OH, —N(R⁹)(C═O)R¹⁰, —(C═O)NR⁹R¹⁰, —NR⁹R¹⁰, —(C═O)OR″¹ or —O(C═O)R″, provided that G³ is C₄-C₆ alkylene when R⁸ is —NR⁹R¹⁰,
R⁹ and R¹⁰ are each independently H or C₁-C₁₂ alkyl;
R″ is aralkyl;
a, b, c and d are each independently an integer from 1 to 24; and x is 0, 1 or 2,
wherein each alkyl, alkylene and aralkyl is optionally substituted.

### Formula (AX)

In some embodiments, the lipid nanoparticle comprises a lipid having the structure:

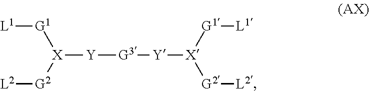

or a pharmaceutically acceptable salt, prodrug or stereoisomer thereof, wherein:
X and X′ are each independently N or CR;
Y and Y′ are each independently absent, —O(C═O)—, —(C═O)O— or NR, provided that:
a) Y is absent when X is N;
b) Y′ is absent when X′ is N;
c) Y is —O(C═O)—, —(C═O)O— or NR when X is CR; and
d) Y′ is —O(C═O)—, —(C═O)O— or NR when X′ is CR,
L¹ and L^{1′} are each independently —O(C═O)R′, —(C═O)OR′, —C(═O)R′, —OR¹, —S(O)₂R′, —S—SR¹, —C(═O) SR′, —SC(═O)R′, —NR^{a}C(═O)R′, —C(═O)NR^{b}R^{c}, —NR^{a}C(═O)NR^{b}R^{c}, —OC(═O)NR^{b}R^{c} or —NR^{a}C(═O)OR′;
L² and L²′ are each independently —O(C═O)R², —(C═O)OR², —C(═O)R², —OR², —S(O)_{z}R², —S—SR², —C(═O) SR², —SC(═O)R², —NR^{d}C(═O)R², —C(═O)NR^{e}R^{f}, —NR^{d}C(═O)NRᵣR^{f}, —OC(═O)NRₑR^{f}; —NR^{d}C(═O)OR² or a direct bond to R²;
G¹. G¹, G² and G² are each independently C₂-C₁₂ alkylene or C₂-C₁₂ alkenylene;
G is C₂-C₂₄ heteroalkylene or C₂-C₂₄ heteroalkenylene;
R^{a}, R^{b}, R^{d} and R^{e} are, at each occurrence, independently H, C₁-C₁₂ alkyl or C₂-C₁₂ alkenyl;
R^{e} and R^{f} are, at each occurrence, independently C₁-C₁₂ alkyl or C₂-C₁₂ alkenyl;
R is, at each occurrence, independently H or C₁-C₁₂ alkyl;
R¹ and R² are, at each occurrence, independently branched C₆-C₂₄ alkyl or branched C₆-C₂₄ alkenyl;
z is 0, 1 or 2, and wherein each alkyl, alkenyl, alkylene, alkenylene, heteroalkylene and heteroalkenylene is independently substituted or unsubstituted unless otherwise specified.

### Formula (AXI)

In some embodiments, the lipid nanoparticle comprises a lipid having the structure:

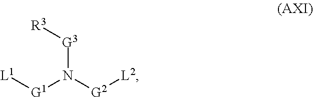

or a pharmaceutically acceptable salt, prodrug or stereoisomer thereof, wherein:
L¹ is —O(C═O)R¹, —(C═O)OR¹, —C(═O)R¹, —OR¹, —S(O)ₓR¹, —S—SR¹, —C(═O) SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{e} or —NR^{a}C(═O)OR¹;
L² is —O(C═O)R², —(C═O)OR², —C(═O)R², —OR², —S(O) ÆR², —S—SR², —C(═O) SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G¹ and G² are each independently C₂-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₁-C₁₂ alkenyl;
R^{e} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R³ is —N(R⁴) R⁵;
R⁴ is C₁-C₁₂ alkyl;
R⁵ is substituted C₁-C₁₂ alkyl; and
x is 0, 1 or 2, and
wherein each alkyl, alkenyl, alkylene, alkenylene, cycloalkylene, cycloalkenylene, aryl and aralkyl is independently substituted or unsubstituted unless otherwise specified.

In some embodiments, the lipid nanoparticle comprises a lipid having the structure:

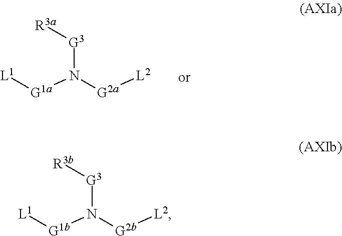

or a pharmaceutically acceptable salt, prodrug or stereoisomer thereof, wherein:
L¹ is —O(C═O)R¹, —(C═O)OR¹, —C(═O)R¹, —OR¹, —S(O) \R¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O) \R², —S—SR², —C(═O) SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G^{1a} and G^{2b} are each independently C₂-C₁₂ alkylene or C₂-C₁₂ alkenylene;
G^{1b} and G^{2b} are each independently C₁-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₂-C₁₂ alkenyl;
R^{e} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R^{3a} is —C(═O)N(R^{4a}) R^{5a} or —C(═O)OR⁶;
R^{3b} is —NR^{4b}C(═O)R^{5b};
R^{4a} is C₁-C₁₂ alkyl;
R^{4b} is H, C₁-C₁₂ alkyl or C₂-C₁₂ alkenyl;
R^{5a} is H, C₁-C₈ alkyl or C₂-C₈ alkenyl;
R^{5b} is C₂-C₁₂ alkyl or C₂-C₁₂ alkenyl when R^{4b} is H; or R^{5b} is C₁-C₁₂ alkyl or C₂-C₁₂ alkenyl
when R^{4b} is C₁-C₁₂ alkyl or C₂-C₁₂ alkenyl;
R⁶ is H, aryl or aralkyl; and
x is 0, 1 or 2, and
wherein each alkyl, alkenyl, alkylene, alkenylene, cycloalkylene, cycloalkenylene, aryl and aralkyl is independently substituted or unsubstituted.

### Formula (AXII)

In some embodiments, the lipid nanoparticle comprises a lipid having the structure:

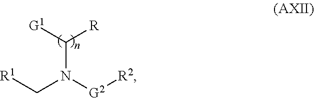

or a pharmaceutically acceptable salt, prodrug or stereoisomer thereof, wherein:
G¹ is —OH, —R³R⁴, —(C═O)R⁵ or —R³ (C═O)R⁵;
G² is —CH₂— or —(C═O)—;
R is, at each occurrence, independently H or OH;
R¹ and R² are each independently optionally substituted branched, saturated or unsaturated C₁₂-C₃₆ alkyl;
R³ and R⁴ are each independently H or optionally substituted straight or branched, saturated or unsaturated C₁-C₆ alkyl;
R⁵ is optionally substituted straight or branched, saturated or unsaturated C₁-C₆ alkyl; and n is an integer from 2 to 6.

### Formula (AXIII)

In some embodiments, the lipid nanoparticle comprises a lipid having the structure:

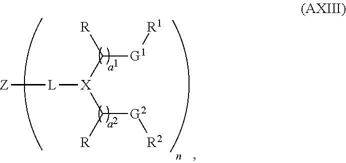

or a pharmaceutically acceptable salt, prodrug or stereoisomer thereof, wherein:
one of G¹ or G² is, at each occurrence, —O(C═O)—, —(C═O)O—, —C(═O)—, —O—, —S(O), —S—S—, —C(═O)S—, SC(═O)—, —N(R^{a}) C(═O)—, —C(═O)N(R^{a})—, —N(R^{a}) C(═O)N(R^{a})—, —OC(═O)N(R^{a})— or —N(R^{a}) C(═O)O—, and the other of G¹ or G² is, at each occurrence, —O(C═O)—, —(C═O)O—, —C(═O)—, —O—, —S(O), —S—S—, —C(═O)S—, —SC(═O)—, —N(R^{a})C(═O)—, —C(═O)N(R^{a})—, —N(R^{a})C(═O)N(R^{a})—, —OC(═O)N(R^{a})— or —N(R^{a}) C(═O)O— or a direct bond;
L is, at each occurrence, ˜O(C═O)—, wherein ˜ represents a covalent bond to X; X is CR^{a},
Z is alkyl, cycloalkyl or a monovalent moiety comprising at least one polar functional group when n is 1; or Z is alkylene, cycloalkylene or a polyvalent moiety comprising at least one polar functional group when n is greater than 1;
R^{a} is, at each occurrence, independently H, C₁-C₁₂ alkyl, C₁-C₁₂ hydroxylalkyl, C₁-C₁₂ aminoalkyl, C₁-C₁₂ alkylaminylalkyl, C₁-C₁₂ alkoxyalkyl, C₁-C₁₂ alkoxycarbonyl, C₁-C₁₂ alkylcarbonyloxy, C₁-C₁₂ alkylcarbonyloxyalkyl or C₁-C₁₂ alkylcarbonyl;
R is, at each occurrence, independently either: (a) H or C₁-C₁₂ alkyl; or (b) R together with the carbon atom to which it is bound is taken together with an adjacent R and the carbon atom to which it is bound to form a carbon-carbon double bond;
R¹ and R² have, at each occurrence, the following structure, respectively:

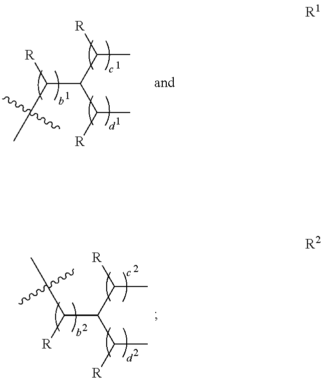

a¹ and a² are, at each occurrence, independently an integer from 3 to 12; b¹ and b² are, at each occurrence, independently 0 or 1;
c¹ and c² are, at each occurrence, independently an integer from 5 to 10; d¹ and d² are, at each occurrence, independently an integer from 5 to 10; y is, at each occurrence, independently an integer from 0 to 2; and n is an integer from 1 to 6,
wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

### Formula (AXIV)

In some embodiments, the lipid nanoparticle comprises a lipid having the structure:

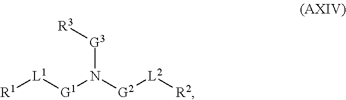

or a pharmaceutically acceptable salt, prodrug or stereoisomer thereof, wherein:
one of L¹ or L² is —O(C═O)—, —(C═O)O—, —C(═O)—, —O—, —S(O)—, —S—S—, —C(═O)S—, —SC(═O)—, —R^{a}C(═O)—, —C(═O)R^{a}—, R^{a}C(═O)R^{a}—, —OC(═O)R^{a}— or —R^{a}C(═O)O—, and the other of L¹ or L² is —O(C═O)—, —(C═O)O—, —C(═O)—, —O—, —S(O)ₓ—, —S—S—, —C(═O)S—, SC(═O)—, —R^{a}C(═O)—, —C(═O)R^{a}—, R^{a}C(═O)R^{a}—, —OC(═O)R^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
Rₐ is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —R^{a}C(═O)R⁴;
R⁴ is C₁-C₁₂ alkyl;
R⁵ is H or C₁-C₆ alkyl; and
x is 0, 1 or 2.

### Formula (AXV)

In some embodiments, the lipid nanoparticle comprises a lipid having the structure:

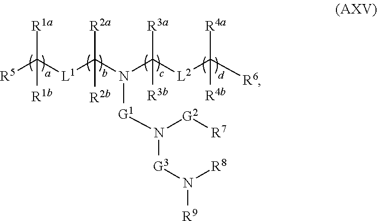

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —R^{a}C(═O)—, —C(═O)R^{a}—, —R^{a}C(═O)R^{a}—, —OC(═O)R^{a}—, —R^{a}C(═O)O— or a direct bond;
G¹ is C₁-C₂ alkylene, —(C═O)—, —O(C═O)—, —SC(═O)—, —R^{a}C(═O)— or a direct bond;
G² is —C(═O)—, —(C═O)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and x is 0, 1 or 2.

### Formula (AXVI)

In some embodiments, the lipid nanoparticle comprises a lipid having the structure:

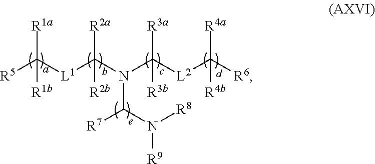

or a pharmaceutically acceptable salt, tautomer, prodrug or stereoisomer thereof, wherein:
L¹ and L² are each independently —O(C═O)—, —(C═O)O— or a carbon-carbon double bond;
R₁ₐ and R_{1b} are, at each occurrence, independently either (a) H or C₁-C₁₂ alkyl, or (b) R^{1a} is H or C₁-C₁₂ alkyl, and Rib together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond; R⁵ and R⁶ are each independently methyl or cycloalkyl;
R⁷ is, at each occurrence, independently H or C₁-C₁₂ alkyl; R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24; b and c are each independently an integer from 1 to 24; and e is 1 or 2,
provided that:
at least one of R₁ₐ, R^{2a}, R^{3a} or R^{4a} is C₁-C₁₂ alkyl, or at least one of L¹ or L² is —O(C═O)— or —(C═O)O—; and
R^{1a} and R^{1b} are not isopropyl when a is 6 or n-butyl when a is 8.

### Formula (AXVII)

In some embodiments, the lipid nanoparticle comprises a lipid having the structure:

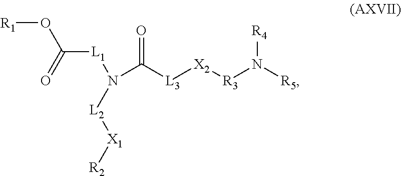

or a pharmaceutically acceptable salt thereof, wherein
R₁ and R₂ are the same or different, each a linear or branched alkyl with 1-9 carbons, or as alkenyl or alkynyl with 2 to 11 carbon atoms,
L₁ and L₂ are the same or different, each a linear alkyl having 5 to 18 carbon atoms, or form a heterocycle with N,
X₁ is a bond, or is —CG-G-whereby L2-CO—O—R₂ is formed,
X² is S or O,
L³ is a bond or a lower alkyl, or form a heterocycle with N,
R³ is a lower alkyl, and
R⁴ and R⁵ are the same or different, each a lower alkyl.
   Compounds (A₁)-(A₁₁)

In some embodiments, the lipid nanoparticle comprises an ionizable lipid having the structure:

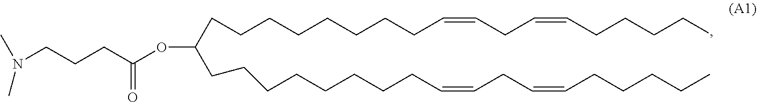

or a pharmaceutically acceptable salt thereof.

In some embodiments, the lipid nanoparticle comprises a lipid having the structure:

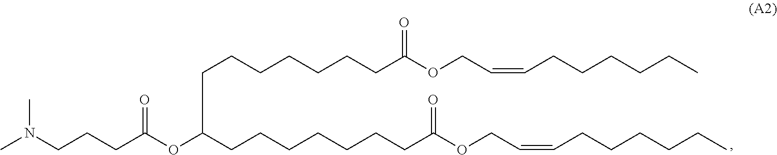 [SMILES: CCCCCC/C=C\COC(=O)CCCCCCCC(CCCCCCCC(=O)OC/C=C\CCCCCC)OC(=O)CCCN(C)C]

or a pharmaceutically acceptable salt thereof.

In some embodiments, the lipid nanoparticle comprises a lipid having the structure:

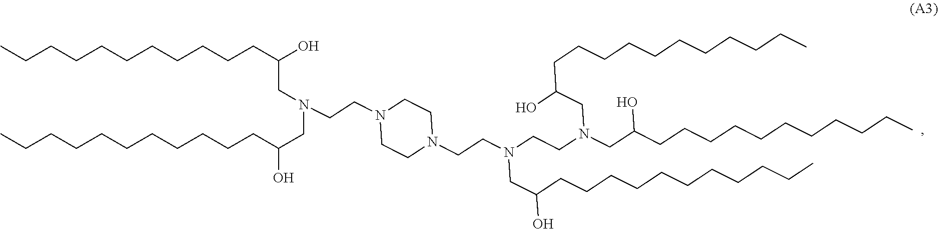 [SMILES: CCCCCCCCCCCC(O)CN(CCN1CCN(CCN(CC(O)CCCCCCCCCCC)CC(O)CCCCCCCCCCC)CC1)CCN(CC(O)CCCCCCCCCCC)CC(O)CCCCCCCCCCC]

or a pharmaceutically acceptable salt thereof.

In some embodiments, the lipid nanoparticle comprises a lipid having the structure:

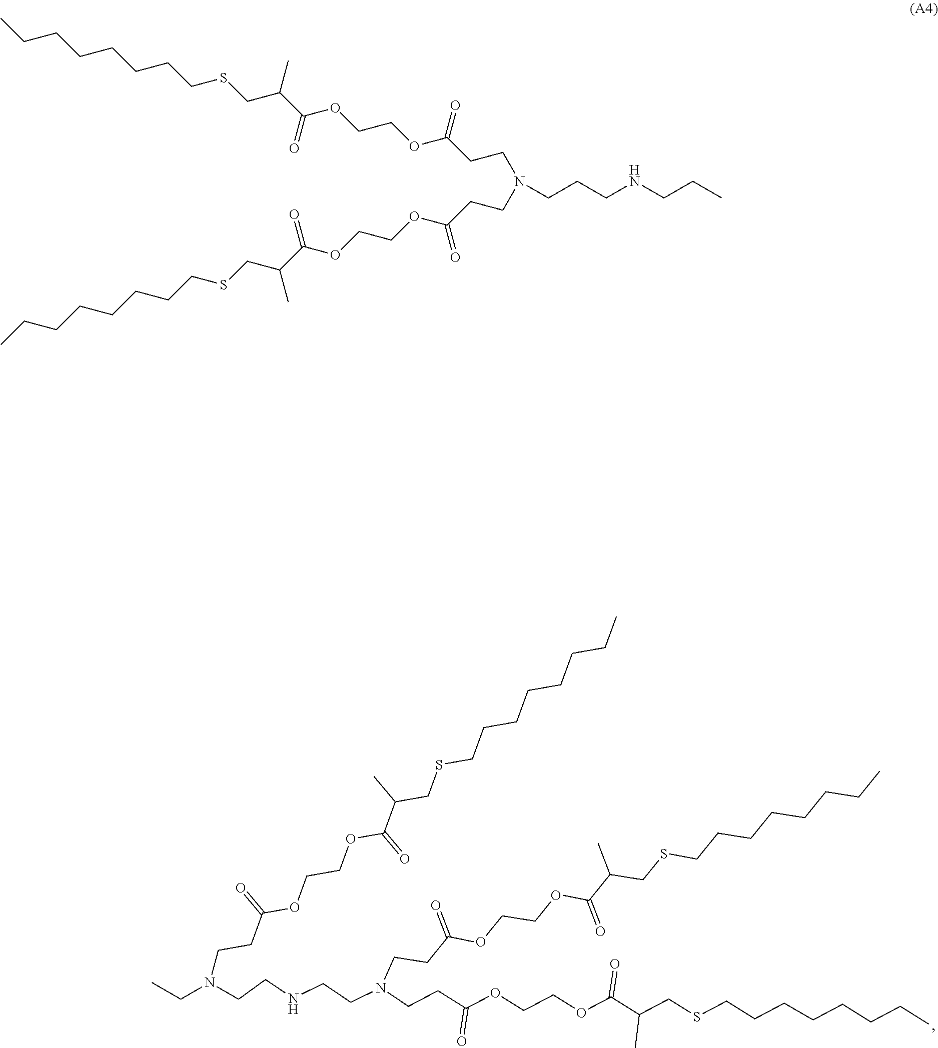

or a pharmaceutically acceptable salt thereof.

In some embodiments, the lipid nanoparticle comprises a lipid having the structure:

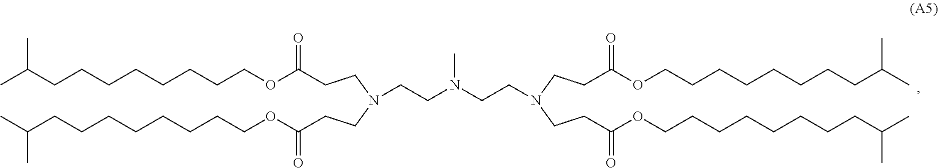 [SMILES: CC(C)CCCCCCCCOC(=O)CCN(CCC(=O)OCCCCCCCCC(C)C)CCN(C)CCN(CCC(=O)OCCCCCCCCC(C)C)CCC(=O)OCCCCCCCCC(C)C]

or a pharmaceutically acceptable salt thereof.

In some embodiments, the lipid nanoparticle comprises a lipid having the structure:

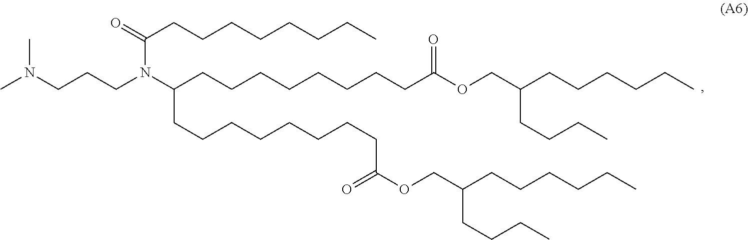 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

or a pharmaceutically acceptable salt thereof.

In some embodiments, the lipid nanoparticle comprises a lipid having the structure:

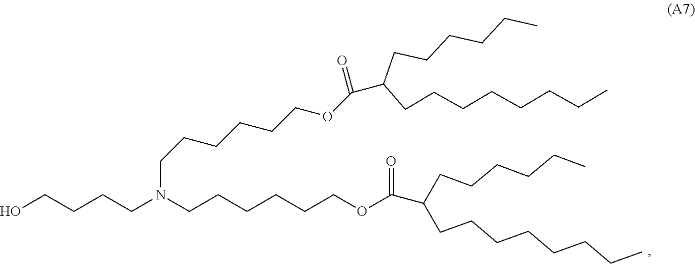 [SMILES: CCCCCCCCC(CCCCCC)C(=O)OCCCCCCN(CCCCO)CCCCCCOC(=O)C(CCCCCC)CCCCCCCC]

or a pharmaceutically acceptable salt thereof.

In some embodiments, the lipid nanoparticle comprises a lipid having the structure:

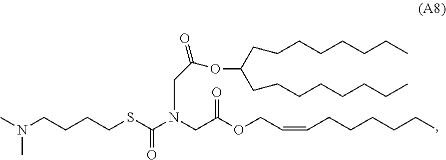

or a pharmaceutically acceptable salt thereof.

In some embodiments, the lipid nanoparticle comprises a lipid having the structure:

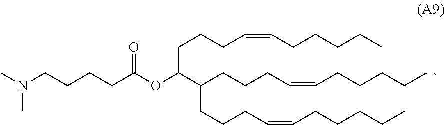 [SMILES: CCCCC/C=C\CCCC(CCC/C=C\CCCCC)C(CCC/C=C\CCCCC)OC(=O)CCCCN(C)C]

or a pharmaceutically acceptable salt thereof.

In some embodiments, the lipid nanoparticle comprises a lipid having the structure:

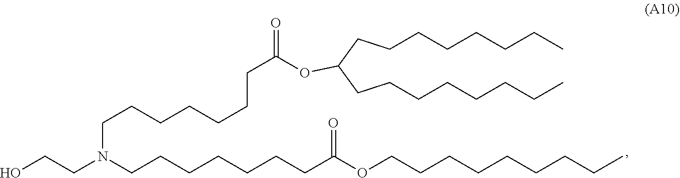 [SMILES: CCCCCCCCCOC(=O)CCCCCCCN(CCO)CCCCCCCC(=O)OC(CCCCCCCC)CCCCCCCC]

or a pharmaceutically acceptable salt thereof.

In some embodiments, the lipid nanoparticle comprises a lipid having the structure:

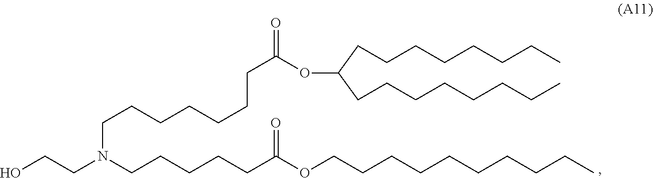 [SMILES: CCCCCCCCCCOC(=O)CCCCCN(CCO)CCCCCCCC(=O)OC(CCCCCCCC)CCCCCCCC]

or a pharmaceutically acceptable salt thereof.

### Non-Cationic Lipids

In certain embodiments, the lipid nanoparticles described herein comprise one or more non-cationic lipids. Non-cationic lipids may be phospholipids.

In some embodiments, the lipid nanoparticle comprises 5-25 mol % non-cationic lipid. For example, the lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, the lipid nanoparticle comprises 5 mol %, 10 mol %, 15 mol %, 20 mol %, or 25 mol % non-cationic lipid.

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In some embodiments, the lipid nanoparticle comprises 5-15 mol %, 5-10 mol %, or 10-15 mol % DSPC. For example, the lipid nanoparticle may comprise 5, 6, 7, 8, 9, 10, 11, 12, 13, 14, or 15 mol % DSPC.

In certain embodiments, the lipid composition of the lipid nanoparticle composition disclosed herein can comprise one or more phospholipids, for example, one or more saturated or (poly) unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

### Formula (HI)

In certain embodiments, a phospholipid is an analog or variant of DSPC. In certain embodiments, a phospholipid is a compound of Formula (HI):

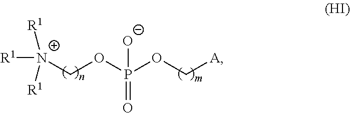

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

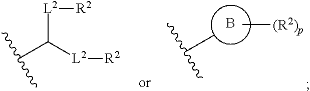

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or —NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N}) S(O), S(O)N(R^{N}), N(R^{N}) S(O)N(R^{N}), OS(O)N(R^{N}), N(R^{N}) S(O)O, S(O)₂, N(R^{N}) S(O)₂, S(O)₂N(R^{N}), —N(R^{N}) S(O)₂N(R^{N}), OS(O)₂N(R^{N}), or N(R^{N}) S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound is not of the formula:

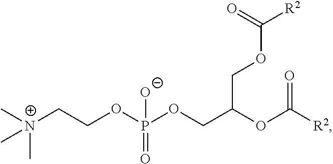

wherein each instance of R² is independently unsubstituted alkyl, unsubstituted alkenyl, or unsubstituted alkynyl.

In some embodiments, the phospholipids may be one or more of the phospholipids described in International Patent Application No. PCT/US2018/037922.

In some embodiments, the lipid nanoparticle comprises a molar ratio of 5-25% non-cationic lipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% non-cationic lipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% phospholipid lipid.

### Structural Lipids

The lipid composition of a pharmaceutical composition disclosed herein can comprise one or more structural lipids. As used herein, the term “structural lipid” includes sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises 30-45 mol % sterol, optionally 35-40 mol %, for example, 30-31 mol %, 31-32 mol %, 32-33 mol %, 33-34 mol %, 34-35 mol %, 35-36 mol %, 36-37 mol %, 37-38 mol %, 38-39 mol %, or 39-40 mol %. In some embodiments, the lipid nanoparticle comprises 25-55 mol % sterol. For example, the lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid nanoparticle comprises 25 mol %, 30 mol %, 35 mol %, 40 mol %, 45 mol %, 50 mol %, or 55 mol % sterol.

In some embodiments, the lipid nanoparticle comprises 35-40 mol % cholesterol. For example, the lipid nanoparticle may comprise 35, 35.5, 36, 36.5, 37, 37.5, 38, 38.5, 39, 39.5, or 40 mol % cholesterol.

### Polyethylene Glycol (PEG)-Lipids

The lipid composition of a pharmaceutical composition disclosed herein can comprise one or more polyethylene glycol (PEG) lipids.

As used herein, the term “PEG-lipid” or “PEG-modified lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines, and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino (polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG, and/or PEG-DPG.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In some embodiments, the PEG-lipid is PEG₂ₖ-DMG.

In some embodiments, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Patent Application Publ. No. WO 2015/130584 A2, which are incorporated herein by reference in their entirety.

In general, some of the other lipid components (e.g., PEG lipids) of various formulae described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments, the PEG-modified lipids are a modified form of PEG DMG. PEG-DMG has the following structure:

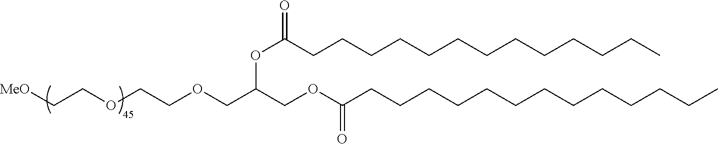

In some embodiments, PEG lipids can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment.

### Formula (PI)

In certain embodiments, a PEG lipid is a compound of Formula (PI):

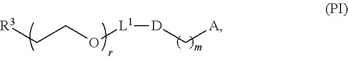

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R_{N}), NRC(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10;
A is of the formula:

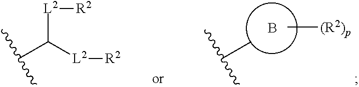

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{d}C(O)O, or —NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR)N(R^{N}), C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N}) S(O), S(O)N(R^{N}), N(R^{N}) S(O)N(R^{N}), OS(O)N(R^{N}), N(R)S(O)O, S(O)₂, N(R^{N}) S(O)₂, S(O)₂N(R^{N}), —N(R^{N}) S(O)₂N(R^{N}), OS(O)₂N(R^{N}), or N(R^{N}) S(O)₂O;
each instance of R^{a} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (PI) is a PEG-OH lipid (i.e., R³ is —OR^{O}, and R^{O} is hydrogen). In certain embodiments, the compound of Formula (PI) is of Formula (PI-OH):

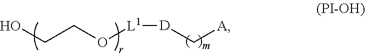

or a salt thereof.

### Formula (PII)

In certain embodiments, a PEG lipid is a PEGylated fatty acid. In certain embodiments, a PEG lipid is a compound of Formula (PII). In some embodiments, compounds of Formula (PII) have the following formula:

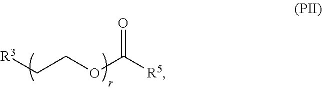

or a salts thereof, wherein:
R³ is —OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), —NR^{N}C(O)N(R^{N}), C(O)O, OC(O), OC(O)O, OC(O)N(R^{N}), NR^{N}C(O)O, C(O)S, SC(O), C(═NR^{N}), —C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N}) S(O), S(O)N(R^{N}), —N(R^{N}) S(O)N(R^{N}), OS(O)N(R^{N}), N(R^{N}) S(O)O, S(O)₂, N(R^{N}) S(O)₂, S(O)₂N(R^{N}), N(R^{N}) S(O)₂N(R^{N}), OS(O)₂N(R^{N}), or N(R^{N}) S(O)₂O; and
each instance of R^{N} is independently hydrogen, optionally substituted alkyl, or a nitrogen protecting group.

In certain embodiments, the compound of Formula (PII) is of Formula (PII-OH):

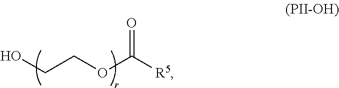

or a salt thereof. In some embodiments, r is 40-50.

In yet other embodiments, the compound of Formula (PII) is:

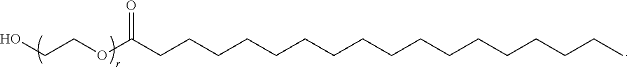

or a salt thereof.

In some embodiments, the compound of Formula (PII) is

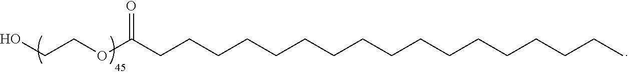

In some embodiments, the lipid composition of the pharmaceutical compositions disclosed herein does not comprise a PEG-lipid.

In some embodiments, the PEG-lipids may be one or more of the PEG lipids described in U.S. Application No. U.S. Ser. No. 15/674,872.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises 1-5% PEG-modified lipid, optionally 1-3 mol %, for example 1.5 to 2.5 mol %, 1-2 mol %, 2-3 mol %, 3-4 mol %, or 4-5 mol %. In some embodiments, the lipid nanoparticle comprises 0.5-15 mol % PEG-modified lipid. For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %. In some embodiments, the lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

Some embodiments comprise adding PEG to a composition comprising an LNP encapsulating a nucleic acid (e.g., which already includes PEG in the amounts listed above). Some embodiments comprise adding about 0.5 mol % or more PEG to an LNP composition, such as about 1 mol %, about 1.5 mol %, about 2 mol %, about 2.5 mol %, about 3 mol %, about 3.5 mol %, about 4 mol %, about 5 mol %, or more after formation of an LNP composition (e.g., which already contains PEG in amount listed elsewhere herein).

In some embodiments, the lipid nanoparticle comprises 20-60 mol % ionizable amino lipid, 5-25 mol % non-cationic lipid, 25-55 mol % sterol, and 0.5-15 mol % PEG-modified lipid.

In some embodiments, a LNP of the disclosure comprises an ionizable amino lipid of Compound 1, wherein the non-cationic lipid is DSPC, the structural lipid that is cholesterol, and the PEG lipid is DMG-PEG.

In some embodiments, a LNP of the disclosure comprises an ionizable amino lipid of Compound 2, wherein the non-cationic lipid is DSPC, the structural lipid that is cholesterol, and the PEG lipid is DMG-PEG.

In some embodiments, a LNP comprises an ionizable amino lipid of any of Formula (AIII),

(AIV), or (AV), a phospholipid comprising DSPC, a structural lipid, and a PEG lipid comprising PEG-DMG.

In some embodiments, a LNP comprises an ionizable amino lipid of any of Formula (AIII), (AIV), or (AV), a phospholipid comprising DSPC, a structural lipid, and a PEG lipid comprising a compound having Formula (PII).

In some embodiments, a LNP comprises an ionizable amino lipid of Formula (AIII), (AIV), or (AV), a phospholipid comprising a compound having Formula (HI), a structural lipid, and the PEG lipid comprising a compound having Formula (PI) or (PII).

In some embodiments, a LNP comprises an ionizable amino lipid of Formula (AIII), (AIV), or (AV), a phospholipid comprising a compound having Formula (HI), a structural lipid, and the PEG lipid comprising a compound having Formula (PI) or (PII).

In some embodiments, a LNP comprises an ionizable amino lipid of Formula (AIII), (AIV), or (AV), a phospholipid having Formula (HI), a structural lipid, and a PEG lipid comprising a compound having Formula (PII).

In some embodiments, the lipid nanoparticle comprises 49 mol % ionizable amino lipid, 10 mol % DSPC, 38.5 mol % cholesterol, and 2.5 mol % DMG-PEG.

In some embodiments, the lipid nanoparticle comprises 49 mol % ionizable amino lipid, 11 mol % DSPC, 38.5 mol % cholesterol, and 1.5 mol % DMG-PEG.

In some embodiments, the lipid nanoparticle comprises 48 mol % ionizable amino lipid, 11 mol % DSPC, 38.5 mol % cholesterol, and 2.5 mol % DMG-PEG.

In some embodiments, a LNP comprises an N:P ratio of from about 2:1 to about 30:1.

In some embodiments, a LNP comprises an N:P ratio of about 6:1.

In some embodiments, a LNP comprises an N:P ratio of about 3:1, 4:1, or 5:1.

In some embodiments, a LNP comprises a wt/wt ratio of the ionizable amino lipid component to the RNA of from about 10:1 to about 100:1.

In some embodiments, a LNP comprises a wt/wt ratio of the ionizable amino lipid component to the RNA of about 20:1.

In some embodiments, a LNP comprises a wt/wt ratio of the ionizable amino lipid component to the RNA of about 10:1.

Some embodiments comprise a composition having one or more LNPs having a diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. Some embodiments comprise a composition having a mean LNP diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. In some embodiments, the composition has a mean LNP diameter from about 30 nm to about 150 nm, or a mean diameter from about 60 nm to about 120 nm.

A LNP may comprise one or more types of lipids, including but not limited to amino lipids (e.g., ionizable amino lipids), neutral lipids, non-cationic lipids, charged lipids, PEG-modified lipids, phospholipids, structural lipids and sterols. In some embodiments, a LNP may further comprise one or more cargo molecules, including but not limited to nucleic acids (e.g., mRNA, plasmid DNA, DNA or RNA oligonucleotides, siRNA, shRNA, snRNA, snoRNA, lncRNA, etc.), small molecules, proteins and peptides.

In some embodiments, the composition comprises a liposome. A liposome is a lipid particle comprising lipids arranged into one or more concentric lipid bilayers around a central region. The central region of a liposome may comprise an aqueous solution, suspension, or other aqueous composition.

In some embodiments, a lipid nanoparticle may comprise two or more components (e.g., amino lipid and nucleic acid, PEG-lipid, phospholipid, structural lipid). For instance, a lipid nanoparticle may comprise an amino lipid and a nucleic acid. Compositions comprising the lipid nanoparticles, such as those described herein, may be used for a wide variety of applications, including the stealth delivery of therapeutic payloads with minimal adverse innate immune response.

Effective in vivo delivery of nucleic acids represents a continuing medical challenge. Exogenous nucleic acids (i.e., originating from outside of a cell or organism) are readily degraded in the body, e.g., by the immune system. Accordingly, effective delivery of nucleic acids to cells often requires the use of a particulate carrier (e.g., lipid nanoparticles). The particulate carrier should be formulated to have minimal particle aggregation, be relatively stable prior to intracellular delivery, effectively deliver nucleic acids intracellularly, and illicit no or minimal immune response. To achieve minimal particle aggregation and pre-delivery stability, many conventional particulate carriers have relied on the presence and/or concentration of certain components (e.g., PEG-lipid). However, it has been discovered that certain components may decrease the stability of encapsulated nucleic acids (e.g., mRNA molecules). The reduced stability may limit the broad applicability of the particulate carriers. As such, there remains a need for methods by which to improve the stability of nucleic acid (e.g., mRNA) encapsulated within lipid nanoparticles.

In some embodiments, the lipid nanoparticles comprise one or more of ionizable molecules, polynucleotides, and optional components, such as structural lipids, sterols, neutral lipids, phospholipids and a molecule capable of reducing particle aggregation (e.g., polyethylene glycol (PEG), PEG-modified lipid), such as those described above.

In some embodiments, a LNP described herein may include one or more ionizable molecules (e.g., amino lipids or ionizable lipids). The ionizable molecule may comprise a charged group and may have a certain pKa. In certain embodiments, the pKa of the ionizable molecule may be greater than or equal to about 6, greater than or equal to about 6.2, greater than or equal to about 6.5, greater than or equal to about 6.8, greater than or equal to about 7, greater than or equal to about 7.2, greater than or equal to about 7.5, greater than or equal to about 7.8, greater than or equal to about 8. In some embodiments, the pKa of the ionizable molecule may be less than or equal to about 10, less than or equal to about 9.8, less than or equal to about 9.5, less than or equal to about 9.2, less than or equal to about 9.0, less than or equal to about 8.8, or less than or equal to about 8.5. Combinations of the above referenced ranges are also possible (e.g., greater than or equal to 6 and less than or equal to about 8.5). Other ranges are also possible. In embodiments in which more than one type of ionizable molecule are present in a particle, each type of ionizable molecule may independently have a pKa in one or more of the ranges described above.

In general, an ionizable molecule comprises one or more charged groups. In some embodiments, an ionizable molecule may be positively charged or negatively charged. For instance, an ionizable molecule may be positively charged. For example, an ionizable molecule may comprise an amine group. As used herein, the term “ionizable molecule” has its ordinary meaning in the art and may refer to a molecule or matrix comprising one or more charged moiety. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In particular embodiments, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule and/or matrix may be selected as desired.

In some cases, an ionizable molecule (e.g., an amino lipid or ionizable lipid) may include one or more precursor moieties that can be converted to charged moieties. For instance, the ionizable molecule may include a neutral moiety that can be hydrolyzed to form a charged moiety, such as those described above. As a non-limiting specific example, the molecule or matrix may include an amide, which can be hydrolyzed to form an amine, respectively. Those of ordinary skill in the art will be able to determine whether a given chemical moiety carries a formal electronic charge (for example, by inspection, pH titration, ionic conductivity measurements, etc.), and/or whether a given chemical moiety can be reacted (e.g., hydrolyzed) to form a chemical moiety that carries a formal electronic charge.

The ionizable molecule (e.g., amino lipid or ionizable lipid) may have any suitable molecular weight. In certain embodiments, the molecular weight of an ionizable molecule is less than or equal to about 2,500 g/mol, less than or equal to about 2,000 g/mol, less than or equal to about 1,500 g/mol, less than or equal to about 1,250 g/mol, less than or equal to about 1,000 g/mol, less than or equal to about 900 g/mol, less than or equal to about 800 g/mol, less than or equal to about 700 g/mol, less than or equal to about 600 g/mol, less than or equal to about 500 g/mol, less than or equal to about 400 g/mol, less than or equal to about 300 g/mol, less than or equal to about 200 g/mol, or less than or equal to about 100 g/mol. In some instances, the molecular weight of an ionizable molecule is greater than or equal to about 100 g/mol, greater than or equal to about 200 g/mol, greater than or equal to about 300 g/mol, greater than or equal to about 400 g/mol, greater than or equal to about 500 g/mol, greater than or equal to about 600 g/mol, greater than or equal to about 700 g/mol, greater than or equal to about 1000 g/mol, greater than or equal to about 1,250 g/mol, greater than or equal to about 1,500 g/mol, greater than or equal to about 1,750 g/mol, greater than or equal to about 2,000 g/mol, or greater than or equal to about 2,250 g/mol. Combinations of the above ranges (e.g., at least about 200 g/mol and less than or equal to about 2,500 g/mol) are also possible. In embodiments in which more than one type of ionizable molecules are present in a particle, each type of ionizable molecule may independently have a molecular weight in one or more of the ranges described above.

In some embodiments, the percentage (e.g., by weight, or by mole) of a single type of ionizable molecule (e.g., amino lipid or ionizable lipid) and/or of all the ionizable molecules within a particle may be greater than or equal to about 15%, greater than or equal to about 16%, greater than or equal to about 17%, greater than or equal to about 18%, greater than or equal to about 19%, greater than or equal to about 20%, greater than or equal to about 21%, greater than or equal to about 22%, greater than or equal to about 23%, greater than or equal to about 24%, greater than or equal to about 25%, greater than or equal to about 30%, greater than or equal to about 35%, greater than or equal to about 40%, greater than or equal to about 42%, greater than or equal to about 45%, greater than or equal to about 48%, greater than or equal to about 50%, greater than or equal to about 52%, greater than or equal to about 55%, greater than or equal to about 58%, greater than or equal to about 60%, greater than or equal to about 62%, greater than or equal to about 65%, or greater than or equal to about 68%. In some instances, the percentage (e.g., by weight, or by mole) may be less than or equal to about 70%, less than or equal to about 68%, less than or equal to about 65%, less than or equal to about 62%, less than or equal to about 60%, less than or equal to about 58%, less than or equal to about 55%, less than or equal to about 52%, less than or equal to about 50%, or less than or equal to about 48%. Combinations of the above referenced ranges are also possible (e.g., greater than or equal to 20% and less than or equal to about 60%, greater than or equal to 40% and less than or equal to about 55%, etc.). In embodiments in which more than one type of ionizable molecule is present in a particle, each type of ionizable molecule may independently have a percentage (e.g., by weight, or by mole) in one or more of the ranges described above. The percentage (e.g., by weight, or by mole) may be determined by extracting the ionizable molecule(s) from the dried particles using, e.g., organic solvents, and measuring the quantity of the agent using high pressure liquid chromatography (i.e., HPLC), liquid chromatography-mass spectrometry (LC-MS), nuclear magnetic resonance (NMR), or mass spectrometry (MS). Those of ordinary skill in the art would be knowledgeable of techniques to determine the quantity of a component using the above-referenced techniques. For example, HPLC may be used to quantify the amount of a component, by, e.g., comparing the area under the curve of a HPLC chromatogram to a standard curve.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

According to the disclosures herein, a lipid composition may comprise one or more lipids as described herein. Such lipids may include those useful in the preparation of lipid nanoparticle formulations as described above or as known in the art.

### Stabilizing Compounds

Some embodiments of the compositions described herein are stabilized pharmaceutical compositions. Various non-viral delivery systems, including nanoparticle formulations, present attractive opportunities to overcome many challenges associated with mRNA delivery. Lipid nanoparticles (LNPs) have drawn particular attention in recent years as various LNP formulations have shown promise in a variety of pharmaceutical applications. However, lipids have been shown to degrade nucleic acids, including mRNA, and lipid nanoparticle formulations undergo rapid loss of purity when stored as refrigerated liquids. Moreover, the storage stability of mRNA encapsulated within LNPs is lower than that of unencapsulated mRNA.

A class of compounds has been found to stabilize nucleic acids within a lipid carrier such as an LNP, an unexpected and unprecedented discovery which enables applications including extended refrigerated liquid shelf-life, extended in-use periods at room temperature, and extended in-use stability at physiological temperatures up to higher temperatures such as 40° C. Such stabilizing compounds solve a critical problem, as current manufacturing processes and formulations experience a 5-10% purity loss during LNP formation and processing that is typical with current large-scale LNP production.

In some embodiments, the stabilized pharmaceutical composition comprises a nucleic acid formulation comprising a nucleic acid and a stabilizing compound (e.g., a compound of Formula (I), of Formula (II), or a tautomer or solvate thereof). In some embodiments, the stabilized pharmaceutical composition comprises a nucleic acid formulation comprising a nucleic acid and a lipid, and a compound of Formula (I):

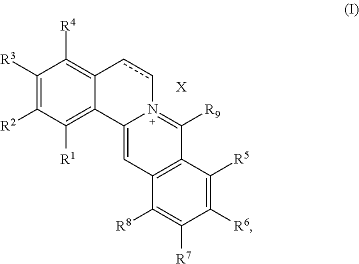

or a tautomer or solvate thereof, wherein:
[symbol] is a single bond or a double bond;
R¹ is H; R² is OCH₃, or together with R³ is OCH₂O; R³ is OCH₃, or together with R² is OCH₂O; R⁴ is H; R⁵ is H or OCH₃; R⁶ is OCH₃; R⁷ is H or OCH₃; R⁸ is H; R⁹ is H or CH₃; and X is a pharmaceutically acceptable anion, e.g., a halide such as chloride.

In some embodiments, the compound of Formula (I) has the structure of:

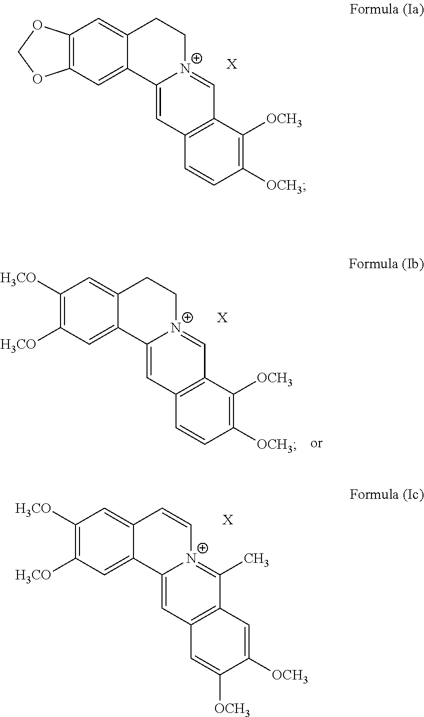

or a tautomer or solvate thereof.

In some embodiments, the stabilized pharmaceutical composition comprises a nucleic acid formulation comprising a nucleic acid and a lipid, and a compound of Formula (II):

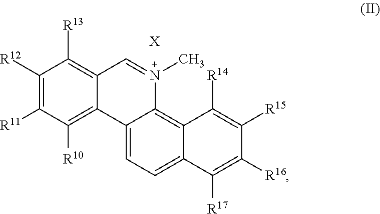

or a tautomer or solvate thereof, wherein:
R¹⁰ is H; R¹¹ is H; R¹² together with R¹³ is OCH₂O; R¹⁴ is H; R¹⁵ together with R¹⁶ is OCH₂O; R¹⁷ is H; and X is a pharmaceutically acceptable anion, e.g., a halide such as chloride.

In some embodiments, the compound of Formula (II) has the structure of:

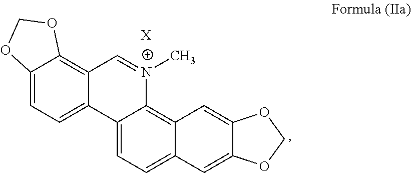

or a tautomer or solvate thereof.

Stabilizing compounds of Formulas (I), (Ia), (Ib), (Ic), (II), and (IIa) are described in International Patent Application No. PCT/US2022/025967, which is incorporated by reference herein in its entirety.

In some embodiments, the nucleic acid formulation comprises lipid nanoparticles. In some embodiments, the nucleic acid is mRNA.

In some embodiments, the stabilizing compound (“the compound”) has a purity of at least 70%, 80%, 90%, 95%, or 99%. In some embodiments, the compound contains fewer than 100 ppm of elemental metals. In some embodiments, the stabilized pharmaceutical composition (“the composition”) comprises a pharmaceutically acceptable metal chelator, e.g., EDTA (ethylenediaminetetraacetic acid) or DTPA (diethylenetriaminepentaacetic acid).

In some embodiments, the composition is an aqueous solution. In some embodiments, the compound is present at a concentration between about 0.1 mM and about 10 mM in the aqueous solution. In some embodiments, the aqueous solution has a pH of or about 5 to 8, including pH of about 5, 5.5, 6, 6.5, 7, 7.5, or 8. In some embodiments, the aqueous solution does not comprise NaCl. In some embodiments, the aqueous solution comprises NaCl in a concentration of or about 150 mM. In some embodiments, the aqueous solution comprises a phosphate buffer, a tris buffer, an acetate buffer, a histidine buffer, or a citrate buffer.

In some embodiments, microbial growth in the composition is inhibited by the compound.

In some embodiments, the composition is characterized as having a mRNA purity level of greater than 60%, greater than 70%, greater than 80%, or greater than 90% main peak mRNA purity after at least thirty days of storage. In some embodiments, the composition comprises a mRNA purity level of greater than 50% main peak mRNA purity after at least six months of storage. In some embodiments, the storage is at room temperature.

In some embodiments, the composition comprises a lipid nanoparticle encapsulating a mRNA, and the composition comprises less than 50%, less than 60%, less than 70%, less than 80%, less than 90%, or less than 95% RNA fragments after at least thirty days of storage. In some embodiments, the storage temperature is greater than room temperature. In some embodiments, the storage temperature is about 4° C.

In some embodiments, the compound interacts with the nucleic acid comprised within a lipid nanostructure (e.g., a lipid nanoparticle, liposome, or lipoplex), e.g., via pi-pi stacking and/or by changing backbone helicity of the nucleic acid. In some embodiments, the compound intercalates with a nucleic acid. In some embodiments, the compound binds with a nucleic acid, e.g., reversible binding, and/or binding to the stranded regions of the nucleic acid. In some embodiments, the compound self-associates, binds to nucleic acid ribose contacts, and/or binds to nucleic acid base contacts. In some embodiments, the compound does not substantially bind to nucleic acid phosphate contacts. In some embodiments, the positive charge of the compound contributes to nucleic acid binding. In some embodiments, the interacts with the nucleic acid with a binding affinity defined by an equilibrium dissociation constant of less than 10⁻³ M (e.g., less than 10⁻⁴ M, less than 10⁻⁵ M, less than 10⁻⁵ M, less than 10⁻⁷ M, less than 10⁻⁸ M, or less than 10⁻⁹ M).

In some embodiments, the compound interacts with a nucleic acid and provides shielding from solvent, e.g., water. In some embodiments, the compound shields ribose from solvent more than the compound shields the phosphate groups of the nucleic acid. In some embodiments, the solvent exposure is measured by the solvent accessible surface area (SASA). In some embodiments, a stabilizing compound decreases the solvent accessible area of ribose to about 5-10 nm². In some embodiments, a stabilizing compound decreases the solvent accessible area of ribose to about 6-8 nm². In some embodiments, a stabilizing compound decreases the solvent accessible area of phosphate to about 9-12 nm². In some embodiments, a stabilizing compound decreases the solvent accessible area of phosphate to about 10⁻¹¹ nm².

In some embodiments, a nucleic acid that is conformationally stabilized by the compound exhibits thermal unfolding temperatures (measured by circular dichroism or DSC, for example) that are higher than in the absence of the compound. In some embodiments, the compound confers increased stability, e.g., thermal stability, to the nucleic acid in a folded structure, e.g., relative to its unfolded or less folded or more linear form. In some embodiments, the compound causes compaction of the nucleic acid upon interaction with the nucleic acid. In some embodiments, the compound causes a decrease in the hydrodynamic radius of the nucleic acid molecule upon interaction with the nucleic acid. In some embodiments, a stabilizing compound causes compaction or a decrease in the hydrodynamic radius of a nucleic acid molecule by 5%, 10%, 15%, 20%, 25%, 30%, 35%, 40%, 45%, 50%, 55%, 60%, or more. In some embodiments, a stabilizing compound causes compaction or a decrease in the hydrodynamic radius of a nucleic acid molecule when the compound is in a concentration of 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, 50 μM, 60 μM, 70 μM, 80 μM, 90 μM, or 100 μM.

mRNA-Lipid Adducts

It has been determined that certain ionizable lipids are susceptible to the formation of lipid-polynucleotide adducts. In particular, ionizable lipids that comprise a tertiary amine group may decompose into one or both of a secondary amine and a reactive aldehyde species capable of interacting with polynucleotides (such as mRNA) to form an ionizable lipid-polynucleotide adduct impurity that can be detected by reverse phase ion pair chromatography (RP-IP HPLC). For example, oxidation of the tertiary amine may lead to N-oxide formation that can undergo acid/base-catalyzed hydrolysis at the amine to generate aldehydes and secondary amines which may form adducts with mRNA. Thus, in some aspects, the ionizable lipid-polynucleotide adduct impurity is an aldehyde-mRNA adduct impurity.

It also has been determined that such adducts may disrupt mRNA translation and impact the activity of lipid nanoparticle (LNP) formulated mRNA products. Thus, it can be advantageous to prepare and use LNP compositions with a reduced content of ionizable lipid-polynucleotide adduct impurity, such as wherein less than about 20%, less than about 10%, less than about 5%, or less than about 1%, of the mRNA is in the form of ionizable lipid-polynucleotide adduct impurity, as may be measured by RP-IP HPLC. Thus, in accordance with some aspects, an LNP composition is provided wherein less than about 10%, less than about 5%, or less than about 1%, of the mRNA is in the form of ionizable lipid-polynucleotide adduct impurity, including less than 10%, less than 5%, or less than 1%, as may be measured by RP-IP HPLC.

In some aspects, an amount of lipid aldehydes in the composition is less than about 50 ppm, including less than 50 ppm. Additionally or alternatively, in some aspects, an amount of N-oxide compounds in the composition is less than about 50 ppm, including less than 50 ppm. Additionally or alternatively, in some aspects, an amount of transition metals, such as Fe, in the composition is less than about 50 ppm, including less than 50 ppm. Additionally or alternatively, in some aspects, an amount of alkyl halide compounds in the composition is less than about 50 ppm, including less than 50 ppm. Additionally or alternatively, in some aspects, an amount of anhydride compounds in the composition is less than about 50 ppm, including less than 50 ppm. Additionally or alternatively, in some aspects, an amount of ketone compounds in the composition is less than about 50 ppm, including less than 50 ppm. Additionally or alternatively, in some aspects, an amount of conjugated diene compounds in the composition is less than about 50 ppm, including less than 50 ppm.

In some aspects, the composition is stable against the formation of ionizable lipid-polynucleotide adduct impurity. In some aspects, an amount of ionizable lipid-polynucleotide adduct impurity in the composition increases at an average rate of less than about 2% per day when stored at a temperature of about 25° C. or below, including at an average rate of less than 2% per day. In some aspects, an amount of ionizable lipid-polynucleotide adduct impurity in the composition increases at an average rate of less than about 0.5% per day when stored at a temperature of about 5° C. or below, including at an average rate of less than 0.5% per day. In some aspects, an amount of ionizable lipid-polynucleotide adduct impurity in the composition increases at an average rate of less than about 0.5% per day when stored at a refrigerated temperature, optionally wherein the refrigerated temperature is about 5° C.

Lipid vehicle (e.g., LNP) compositions with a reduced content of ionizable lipid-polynucleotide adduct impurity can be prepared by methods that inhibit formation of one or both of N-oxides and aldehydes. Such methods may comprise treating a composition comprising an ionizable lipid comprising a tertiary amine group to inhibit formation of one or both of N-oxides and aldehydes, such as by treating the composition with a reducing agent; treating the composition with a chelating agent; adjusting the pH of the composition; adjusting the temperature of the composition;

and adjusting the buffer in the composition. Such methods may comprise, prior to combining the ionizable lipid with a polynucleotide, one or more of treating the ionizable lipid with a scavenging agent; treating the ionizable lipid with a reductive treatment agent; treating the ionizable lipid with a reducing agent; treating the ionizable lipid with a chelating agent; treating the polynucleotide with a reducing agent; and treating the polynucleotide with a chelating agent.

In accordance with any of the foregoing, the scavenging agent, reductive treatment agent, and/or reducing agent may be an agent that reacts with aldehyde, ketone, anhydride and/or diene compounds. A scavenging agent may comprise one or more selected from (O-(2,3,4,5,6-Pentafluorobenzyl) hydroxylamine hydrochloride) (PFBHA), methoxyamine (e.g., methoxyamine hydrochloride), benzyloxyamine (e.g., benzyloxyamine hydrochloride), ethoxyamine (e.g., ethoxyamine hydrochloride), 4-[2-(aminooxy)ethyl]morpholine dihydrochloride, butoxyamine (e.g., tert-butoxyamine hydrochloride), 4-Dimethylaminopyridine (DMAP), 1,4-diazabicyclo[2.2.2]octane (DABCO), Triethylamine (TEA), Piperidine 4-carboxylate (BPPC), and combinations thereof. A reductive treatment agent may comprise a boron compound (e.g., sodium borohydride and/or bis(pinacolato)diboron). A reductive treatment agent may comprise a boron compound, such as one or both of sodium borohydride and bis(pinacolato)diboron). A chelating agent may comprise immobilized iminodiacetic acid. A reducing agent may comprise an immobilized reducing agent, such as immobilized diphenylphosphine on silica (Si-DPP), immobilized thiol on agarose (Ag-Thiol), immobilized cysteine on silica (Si-Cysteine), immobilized thiol on silica (Si-Thiol), or a combination thereof. A reducing agent may comprise a free reducing agent, such as potassium metabisulfite, sodium thioglycolate, tris(2-carboxyethyl) phosphine (TCEP), sodium thiosulfate, N-acetyl cysteine, glutathione, dithiothreitol (DTT), cystamine, dithioerythritol (DTE), dichlorodiphenyltrichloroethane (DDT), homocysteine, lipoic acid, or a combination thereof.

In accordance with any of the foregoing, the pH may be, or adjusted to be, a pH of from about 7 to about 9.

In accordance with any of the foregoing, a buffer may be selected from sodium phosphate, sodium citrate, sodium succinate, histidine, histidine-HCl, sodium malate, sodium carbonate, and TRIS (tris(hydroxymethyl)aminomethane). In accordance with any of the foregoing, a buffer may be TRIS and may be, or adjusted to be, from about 20 mM to about 150 mM TRIS.

In accordance with any of the foregoing, the temperature of the composition may be, or adjusted to be, 25° C. or less.

The composition may also comprise a free reducing agent or antioxidant.

### Formulations

Cancer vaccines (e.g., nucleic acid cancer vaccines such as mRNA cancer vaccines) may be formulated or administered in combination with one or more pharmaceutically-acceptable excipients. As a non-limiting set of examples, cancer vaccines can be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with cancer vaccines (e.g., for transplantation into a subject), hyaluronidase, nanoparticle mimics and combinations thereof.

In some embodiments, vaccine compositions comprise at least one additional active substance, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety for this purpose).

In some embodiments, cancer vaccines are administered to humans, human patients or subjects. The phrase “active ingredient” generally refers to the cancer vaccines or the nucleic acids contained therein, for example, RNA (e.g., mRNA) encoding antigenic polypeptides.

Formulations of the vaccine compositions described herein may be prepared by any method known or hereafter developed in the art of pharmacology. In general, such preparatory methods include the step of bringing the active ingredient (e.g., nucleic acids such as mRNA) into association with an excipient and/or one or more other accessory ingredients, and then, if necessary and/or desirable, dividing, shaping and/or packaging the product into a desired single- or multi-dose unit. The formulation of any of the compositions disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Patent Application Publication No. 2005/0222064. Carbohydrates can include simple sugars (e.g., glucose) and polysaccharides (e.g., glycogen and derivatives and analogs thereof).

A polymer can be included in and/or used to encapsulate or partially encapsulate a pharmaceutical composition disclosed herein (e.g., a pharmaceutical composition in lipid nanoparticle form). A polymer can be biodegradable and/or biocompatible. A polymer can be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates.

In some embodiments, the compositions disclosed herein may be formulated with lipid particles, e.g., lipid nanoparticles (LNP). Accordingly, the present disclosure also provides nanoparticle compositions comprising (i) a lipid composition comprising a delivery agent, and (ii) a nucleic acid (e.g., mRNA) encoding one or more peptide epitopes. In such nanoparticle composition, the lipid composition disclosed herein can encapsulate the nucleic acid encoding one or more peptide epitopes.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In one embodiment, a lipid nanoparticle comprises an ionizable lipid, a structural lipid, a phospholipid, and mRNA. In some embodiments, the LNP comprises an ionizable lipid, a PEG-modified lipid, a phospholipid, a structural lipid, and mRNA.

The ratio between the lipid composition and the cancer vaccine may be from about 10:1 to about 60:1 (wt/wt). In some embodiments, the ratio between the lipid composition and the nucleic acid may be about 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1 or 60:1 (wt/wt). In some embodiments, the wt/wt ratio of the lipid composition to the cancer vaccine is about 20:1 or about 15:1.

In some embodiments, the cancer vaccine (e.g., the nucleic acid cancer vaccine) may be comprised in lipid nanoparticles such that the lipid:polynucleotide weight ratio is 5:1, 10:1, 15:1, 20:1, 25:1, 30:1, 35:1, 40:1, 45:1, 50:1, 55:1, 60:1 or 70:1, or a range or any of these ratios such as, but not limited to, 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 5:1 to about 70:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 10:1 to about 70:1, from about 15:1 to about 20:1, from about 15:1 to about 25:1, from about 15:1 to about 30:1, from about 15:1 to about 35:1, from about 15:1 to about 40:1, from about 15:1 to about 45:1, from about 15:1 to about 50:1, from about 15:1 to about 55:1, from about 15:1 to about 60:1 or from about 15:1 to about 70:1.

In some embodiments, the cancer vaccine (e.g., the nucleic acid cancer vaccine) may be comprised in lipid nanoparticles in a concentration from approximately 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids lead them to form liposomes, vesicles, or membranes in aqueous media.

In some embodiments, a lipid nanoparticle (LNP) may comprise an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid”. In certain embodiments, an ionizable lipid molecule may comprise an amine group, and can be referred to as an ionizable amino lipids. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In particular embodiments, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired. Ionizable lipids can also be the compounds disclosed in International Patent Application Publication Nos.: WO2017075531, WO2015199952, WO2013086354, or WO2013116126, or selected from formulae CLI-CLXXXXII of U.S. Pat. No. 7,404,969; each of which is hereby incorporated by reference in its entirety for this purpose.

It should be understood that the terms “charged” or “charged moiety” does not refer to a “partial negative charge” or “partial positive charge” on a molecule. The terms “partial negative charge” and “partial positive charge” are given their ordinary meanings in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the ionizable lipid is an ionizable amino lipid, sometimes referred to in the art as an “ionizable cationic lipid”. In some embodiments, the ionizable amino lipid may have a positively charged hydrophilic head and a hydrophobic tail that are connected via a linker structure. In addition to these, an ionizable lipid may also be a lipid including a cyclic amine group.

Vaccines (e.g., nucleic acid vaccines) are typically formulated into lipid nanoparticles. In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises a molar ratio of 20-60% ionizable amino lipid. For example, the lipid nanoparticle may comprise a molar ratio of 20-50%, 20-40%, 20-30%, 30-60%, 30-50%, 30-40%, 40-60%, 40-50%, or 50-60% ionizable amino lipid. In some embodiments, the lipid nanoparticle comprises a molar ratio of 20%, 30%, 40%, 50, or 60% ionizable amino lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or 25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol. In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable amino lipid, 5-25% non-cationic lipid, 25-55% sterol, and 0.5-15% PEG-modified lipid.

In some embodiments, an ionizable amino lipid of the disclosure comprises a compound of Formula (I):

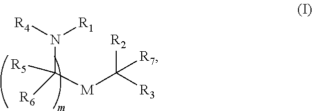

or a salt or isomer thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′) C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₈ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

In some embodiments, a subset of compounds of Formula (I) includes those in which when R₄ is —(CH₂)ₙQ, —(CH₂)ₙCHQR, —CHQR, or —CQ(R)₂, then (i) Q is not-N(R)₂ when n is 1, 2, 3, 4 or 5, or (ii) Q is not 5, 6, or 7-membered heterocycloalkyl when n is 1 or 2.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′) C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
   or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′) C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
   or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′) C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
   or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′) C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
   or salts or isomers thereof.

In some embodiments, another subset of compounds of Formula (I) includes those in which
R₇ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′) C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13, or salts or isomers thereof.

In some embodiments, a subset of compounds of Formula (I) includes those of Formula (IA):

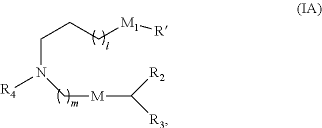

or a salt or isomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁-14 alkyl, and C₂₋₁₄ alkenyl.

In some embodiments, a subset of compounds of Formula (I) includes those of Formula (II):

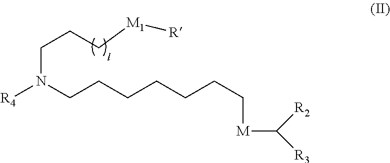

or a salt or isomer thereof, wherein 1 is selected from 1, 2, 3, 4, and 5; M₁ is a bond or M′; R₄ is unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(—CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (IIa), (IIb), (IIc), or (IIe):

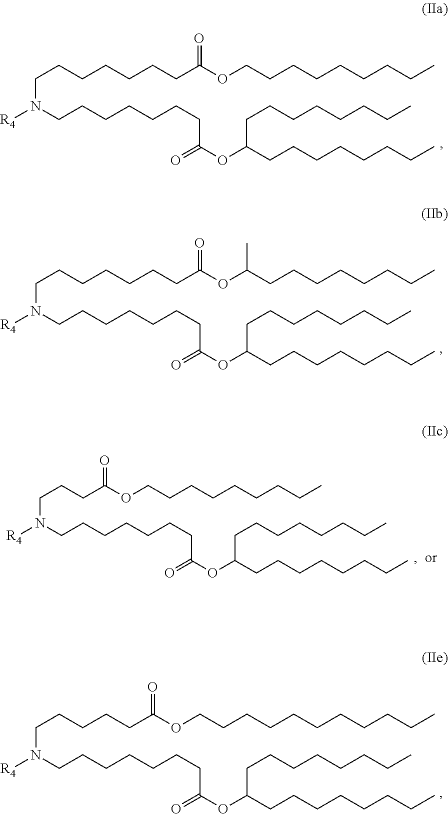

or a salt or isomer thereof, wherein R₄ is as described herein.

In some embodiments, a subset of compounds of Formula (I) includes those of Formula (IId):

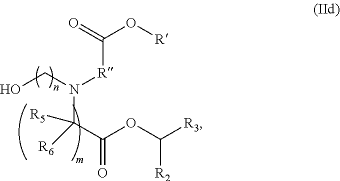

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable cationic lipid of the disclosure comprises a compound having structure:

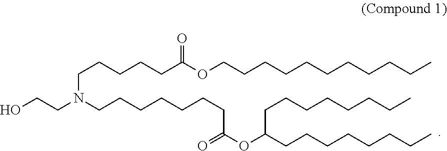 [SMILES: CCCCCCCCCCCOC(=O)CCCCCN(CCO)CCCCCCCC(=O)OC(CCCCCCCC)CCCCCCCC]

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid of the disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, a sterol of the disclosure comprises cholesterol, fecosterol, sitosterol, ergosterol, campesterol, stigmasterol, brassicasterol, tomatidine, ursolic acid, alpha-tocopherol, and mixtures thereof.

In some embodiments, a LNP of the disclosure comprises an ionizable amino lipid of Compound 1, wherein the non-cationic lipid is DSPC, the structural lipid is cholesterol, and the PEG lipid is PEG-DMG.

In some embodiments, a LNP of the disclosure comprises an N:P ratio of from about 2:1 to about 30:1.

In some embodiments, a LNP of the disclosure comprises an N:P ratio of about 6:1.

In some embodiments, a LNP of the disclosure comprises an N:P ratio of about 3:1.

In some embodiments, a LNP of the disclosure comprises a wt/wt ratio of the ionizable cationic lipid component to the RNA of from about 10:1 to about 100:1.

In some embodiments, a LNP of the disclosure comprises a wt/wt ratio of the ionizable cationic lipid component to the RNA of about 20:1.

In some embodiments, a LNP of the disclosure comprises a wt/wt ratio of the ionizable cationic lipid component to the RNA of about 10:1.

In some embodiments, a LNP of the disclosure has a mean diameter from about 50 nm to about 150 nm.

In some embodiments, a LNP of the disclosure has a mean diameter from about 70 nm to about 120 nm.

In some embodiments, the lipid may be a cleavable lipid such as those described in International Patent Application Publication No. WO2012170889, herein incorporated by reference in its entirety for this purpose. In some embodiments, the lipid may be synthesized by methods known in the art and/or as described in International Patent Application Publication No. WO2013086354; the contents of which are herein incorporated by reference in their entirety for this purpose.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide. As used herein, “size” or “mean size” in the context of nanoparticle compositions refers to the mean diameter of a nanoparticle composition.

Relative amounts of the active ingredient (e.g., the nucleic acid cancer vaccine), the pharmaceutically acceptable excipient, and/or any additional ingredients in a vaccine composition may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the package containing the pharmaceutical product contains 0.1 mg to 1 mg of nucleic acid (e.g., mRNA). In some embodiments, the package containing the pharmaceutical product contains 0.35 mg of nucleic acid (e.g., mRNA). In some embodiments, the concentration of the nucleic acid (e.g., mRNA) is 1 mg/mL.

In some embodiments, the nucleic acid (e.g., mRNA) vaccine compositions may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see e.g., the range of unit doses described in International Patent Application Publication No. WO2013078199, herein incorporated by reference in its entirety). In some embodiments, the nucleic acid (e.g., mRNA) vaccine is administered at a dosage level sufficient to deliver 0.0100 mg, 0.025 mg, 0.040 mg, 0.050 mg, 0.075 mg, 0.100 mg, 0.125 mg, 0.130 mg, 0.150 mg, 0.175 mg, 0.200 mg, 0.225 mg, 0.250 mg, 0.275 mg, 0.300 mg, 0.325 mg, 0.350 mg, 0.375 mg, 0.390 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. In some embodiments, the nucleic acid (e.g., mRNA) vaccine is administered at a dosage level sufficient to deliver between 10 μg and 400 μg of the mRNA vaccine to the subject. In some embodiments, the nucleic acid (e.g., mRNA) vaccine is administered at a dosage level sufficient to deliver at least 0.033 mg, at least 0.040 mg, at least 0.1 mg, at least 0.13 mg, at least 0.2 mg, at least 0.39 mg, at least 0.4 mg, or at least 1.0 mg to the subject.

In some embodiments, the nucleic acid (e.g., mRNA) vaccine is administered at a dosage level sufficient to deliver at least 1.0 mg, at least 1.2 mg, at least 1.4 mg, at least 1.6 mg, at least 1.8 mg, or at least 2.0 mg, at least to the subject. In some embodiments, the nucleic acid (e.g., mRNA) vaccine is administered at a dosage level sufficient to deliver at least 2.0 mg, at least 2.2 mg, at least 2.4 mg, at least 2.6 mg, at least 2.8 mg, or at least 3.0 mg, at least to the subject. In some embodiments, the nucleic acid (e.g., mRNA) vaccine is administered at a dosage level sufficient to deliver at least 3.0 mg, at least 3.2 mg, at least 3.4 mg, at least 3.6 mg, at least 3.8 mg, or at least 4.0 mg, at least to the subject. In some embodiments, the nucleic acid (e.g., mRNA) vaccine is administered at a dosage level sufficient to deliver at least 4.0 mg, at least 4.2 mg, at least 4.4 mg, at least 4.6 mg, at least 42.8 mg, or at least 5.0 mg, at least to the subject.

The desired dosage may be delivered three times a day, two times a day, once a day, every other day, every third day, every week, every two weeks, every three weeks, every four weeks, every 2 months, every three months, every 6 months, etc. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In some embodiments, the nucleic acid (e.g., mRNA) vaccine compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg. In some embodiments, the nucleic acid (e.g., mRNA) vaccine compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, the nucleic acid (e.g., mRNA) vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.0100 mg, 0.025 mg, 0.040 mg, 0.050 mg, 0.075 mg, 0.100 mg, 0.125 mg, 0.130 mg, 0.150 mg, 0.175 mg, 0.200 mg, 0.225 mg, 0.250 mg, 0.275 mg, 0.300 mg, 0.325 mg, 0.350 mg, 0.375 mg, 0.390 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, a nucleic acid (e.g., mRNA) vaccine composition may be administered three or four times, or more. In some embodiments, the mRNA vaccine composition is administered once a day every three weeks.

In some embodiments, the nucleic acid (e.g., mRNA) vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.010 mg, 0.025 mg, 0.100 mg or 0.400 mg.

In some embodiments the nucleic acid (e.g., mRNA) vaccine for use in a method of vaccinating a subject is administered the subject a single dosage of between 10 μg/kg and 400 μg/kg of the nucleic acid vaccine in an effective amount to vaccinate the subject. In some embodiments the RNA vaccine for use in a method of vaccinating a subject is administered the subject a single dosage of between 10 μg and 400 μg of the nucleic acid vaccine in an effective amount to vaccinate the subject.

### EXAMPLES

### Example 1

This example describes efficacy and safety results of a trial of a combination of mRNA-1, a personalized cancer vaccine, with pembrolizumab versus pembrolizumab standard of care after complete resection of high-risk melanoma.

mRNA-1 is a novel mRNA-based personalized cancer vaccine, which encodes up to 34 patient specific tumor neoantigens. It was evaluated whether mRNA-1 could synergize with adjuvant pembrolizumab to improve recurrence free survival (RFS) in patients with resected stages IIIB/IIIC/IIID and IV melanoma.

Eligible patients with completely resected, high-risk cutaneous melanoma were randomly assigned 2:1 (stratified by stage) to receive mRNA-1 in combination with pembrolizumab or pembrolizumab alone. mRNA-1 (1 mg) was administered intramuscularly every 3 weeks for a total of 9 doses and pembrolizumab (200 mg) intravenously was given every 3 weeks for up to 18 cycles. Safety was a secondary endpoint. RFS in the overall intention-to-treat population was the primary end point.

The study was designed with 800% power to detect a hazard ratio (HR) of 0.5 with an overall 1-sided type I error of 0.1 when a total of 40 RFS events were observed. The primary analysis for RFS was specified to occur after all patients completed a minimum of 12 months on study and at least 40 RFS events were observed.

107 patients received the combination of mRNA-1 with pembrolizumab and 50 patients were treated with pembrolizumab monotherapy. Recurrence or death was reported in 24 of 107 patients (22.4%) in the combination arm and in 20 of 50 patients (40%) in the monotherapy arm, at a median follow-up of 101 and 105 weeks, respectively. 18-month RFS rates (95% CI) were 78.6% (69.0%, 85.6%) vs 62.2% (46.9%, 74.3%) in the combination and monotherapy arms, respectively. The combination showed protocol defined statistical significance and a clinically meaningful improvement in RFS compared to pembrolizumab, with a reduction in the risk of recurrence or death by 44% (HR=0.561; 95% CI: (0.309, 1.017); stratified log-rank test 1-sided p-value of 0.0266). The majority of treatment related adverse events were Grade 1/2. The number of patients reporting treatment related Grade ≥3 adverse events was generally similar between the arms (25% vs 18%, respectively). The most common mRNA-1 related Grade 3 event was fatigue. No Grade 4 or Grade 5 events related to mRNA-1 were reported. No potentiation of immune-mediated adverse events was observed with the addition of mRNA-1 to pembrolizumab.

Conclusions: mRNA-1 in combination with pembrolizumab as adjuvant therapy for resected high-risk melanoma significantly prolonged RFS compared to pembrolizumab without an increase in clinically meaningful adverse events. These results are the first to demonstrate improvement of RFS over adjuvant standard of care PD-1 blockade in resected high-risk melanoma and provide the first randomized evidence that a personalized neoantigen approach is potentially beneficial for cancer patients. A phase 3 study will be initiated in patients with melanoma.

### Example 2

This example describes results of a study evaluating the efficacy of a combination of mRNA-1, a personalized cancer vaccine, with pembrolizumab. The combination improved recurrence free survival in adjuvant melanoma patients irrespective of tumor mutational burden.

An open-label randomized Phase 2 trial met its primary endpoint of prolonged recurrence free survival (RFS) with adjuvant therapy of mRNA-1 combined with pembrolizumab compared to pembrolizumab alone in patients with high-risk stage III/IV melanoma (HR=0.561; 95% CI: (0.309,1.017)). Tumor immunogenicity provides a favorable landscape for inflammatory processes associated with clinical benefit to checkpoint inhibitors (ICI) and tumor mutational burden (TMB) can be an independent predictor of treatment outcomes in patients treated with ICI therapy. mRNA-1 is a novel mRNA-based personalized cancer vaccine which encodes up to 34 patient-specific tumor neoantigens. Here we report analyses of baseline melanoma biopsies from the trial to explore the novel mechanism of action hypothesized to augment endogenous anti-tumor responses and generate immunity to additional tumor neoantigens.

Paraffin-embedded formalin-fixed baseline tumor core biopsies underwent whole exome and whole transcriptome sequencing. TMB was defined as the number of non-synonymous mutations with an allele frequency ≥5% and the established threshold for TMB-high with pembrolizumab of 175 per exome (10 mutations/megabase) was utilized for the analysis. The distribution of TMB expression in baseline tumor samples across study arms and their association with the primary RFS endpoint was evaluated. The association of other markers of inflamed tumors (e.g. gene expression profile (GEP) and PD-L1 expression) with RFS was also assessed.

Pembrolizumab monotherapy provided stronger efficacy in the TMB high compared to the TMB low group. The RFS benefit observed with the mRNA-1 and pembrolizumab combination compared to pembrolizumab monotherapy was maintained with a similar treatment effect magnitude in both high (HR=0.65; 95% CI: (0.3, 1.5)) and low (HR=0.59; 95% CI: (0.3, 1.4)) TMB subpopulations. A similar effect of other markers of tumor immunity and contexture on clinical activity of the mRNA-1 and pembrolizumab combination treatment was observed.

Conclusions: these results indicate that mRNA-1 demonstrates improvements in RFS irrespective of TMB status when administered in combination with pembrolizumab over pembrolizumab monotherapy in patients with resected high-risk cutaneous melanoma. The novel mechanism of action of mRNA-1 both deepens (TMB high) the activity of pembrolizumab and broadens (TMB low) the population of patients that can benefit from immune therapy.

### Example 3

This example describes the association between clinical benefit from personalized cancer vaccine (PCV) combination treatment with pembrolizumab and various biomarkers associated with cancer immunotherapy.

PCV can improve breadth and strength of responses to pembrolizumab by both increasing endogenous T cell responses and inducing T cell responses to non-endogenous neoantigens.

In this study, recurrence-free survival was evaluated in adjuvant resected melanoma patients at a high risk of recurrence following PCV administration in combination with pembrolizumab (n=107) compared to pembrolizumab monotherapy (n=50). PCV contained up to 34 neoantigens per patient. The results were analyzed in relation to biomarkers associated with response to pembrolizumab treatment.

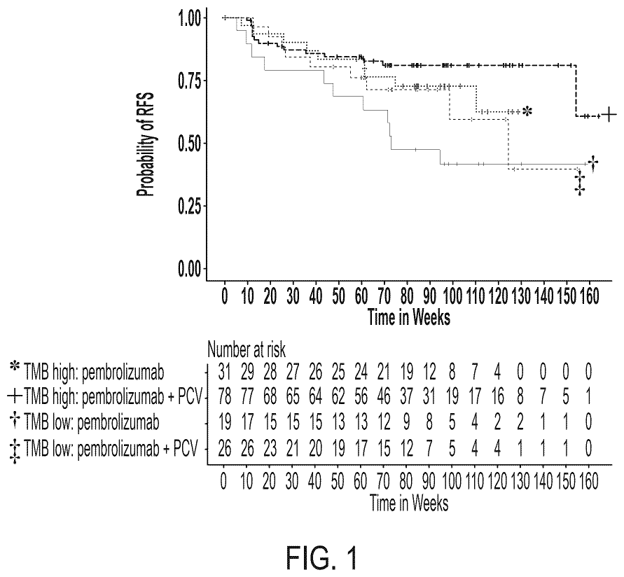

Clinical benefit was observed in PCV+pembrolizumab treatment group relative to pembrolizumab alone treatment group. Patient data was then grouped according to patient TMB value, into TMB-high and TMB-low groups. Combination treatment with PCV and pembrolizumab improved the clinical benefit over pembrolizumab alone in TMB-low populations, and PCV increased the neoantigen-specific T cell numbers on average regardless of baseline TMB. A clinical benefit of PCV+pembrolizumab compared to pembrolizumab-only treatment was observed in both TMB-high and TMB-low populations (FIG. 1). Disease stage did not confound the results, as the distribution was balanced between the study arms. A similar clinical benefit was also observed in both TMB-high and TMB-low populations when TMB outliers (>2000 and <10) were excluded, indicating that TMB outliers did not contribute to the clinical benefit observed in the total dataset.

Several additional tumor biomarkers were also investigated, including T cell-inflamed gene expression score (GEP score), cytotoxicity (CYT) score, and PD-L1 (CD274) expression. The association between these biomarkers and clinical benefit of pembrolizumab+PCV combination relative to pembrolizumab alone was assessed, to determine whether pembrolizumab+PCV increases the clinical benefit in populations already responsive to pembrolizumab monotherapy and/or broadens the clinical benefit into new patient populations.

Formalin-fixed paraffin embedded (FFPE) baseline biopsies and patient-matched normal whole blood samples were subjected to whole exome sequencing. TMB values were determined as the number of non-synonymous mutations per exome (identified by whole-exome sequencing) with an allele frequency of at least 5%, and a cutoff of 175 was used to differentiate between TMB-high and TMB-low populations.

FFPE baseline biopsies were subjected to RNA sequencing, and gene expression data was subsequently log-transformed and quantile normalized. GEP scores were determined as an average of the expression of 18 genes (CXCR6, TIGIT, CD27, CD274 (PD-L1), PDCD1LG2 (PD-L2), LAG3, NKG7, PSMB10, CMKLR1, CD8A, IDO1, CCL5, CXCL9, HLA-DQA1, CD276 (B7-H3), HLA-DRB1, STAT1, and HLA-E). The median GEP score for all of the samples was used as a cutoff to differentiate between GEP-high and GEP-low populations. CYT scores were determined as an average expression of GZMB and PERF1. The median CYT score for all of the samples was used as a cutoff to differentiate between CYT-high and CYT-low populations. PD-L1 expression values were determined as normalized gene expression. The median PD-L1 expression value was used as a cutoff to differentiate between PD-L1-high and PD-L1-low populations.

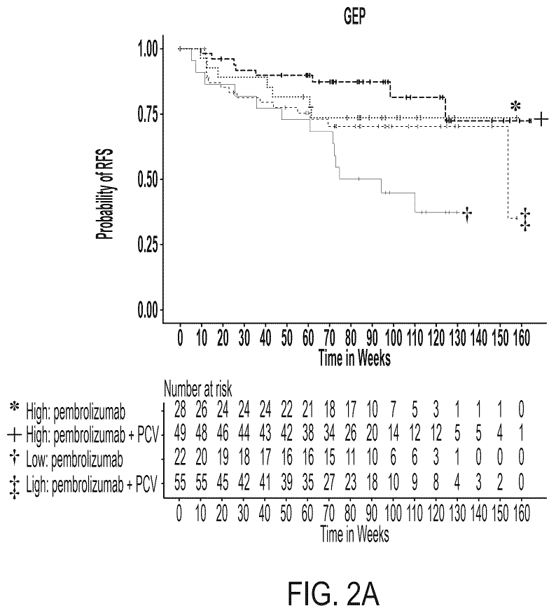

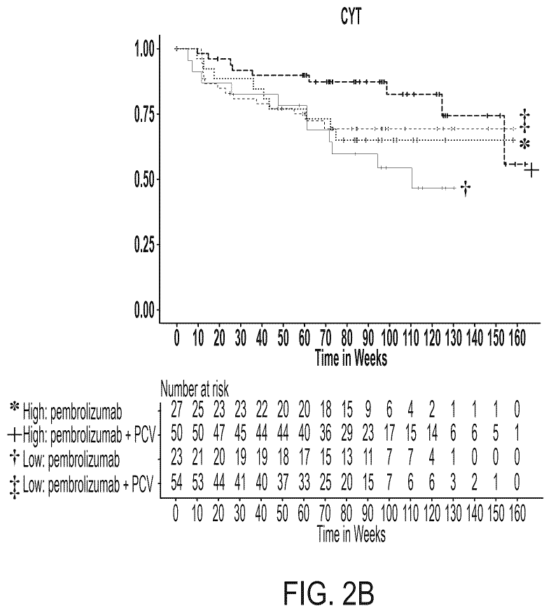

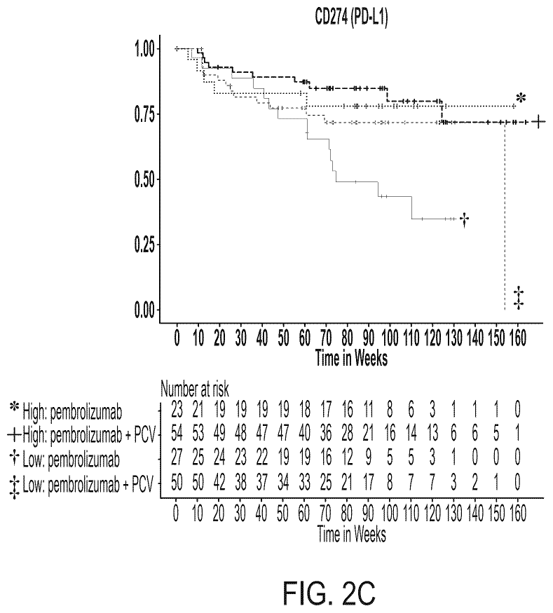

A clinical benefit of PCV+pembrolizumab was observed in GEP-high (FIG. 2A), CYT-high (FIG. 2B), and PD-L1-high (FIG. 2C) populations. These results indicated that the combination of pembrolizumab with PCV deepens the therapeutic effect of pembrolizumab in patient populations already predicted to respond to pembrolizumab. Furthermore, a clinical benefit of PCV+pembrolizumab was also observed in GEP-low (FIG. 2A), CYT-low (FIG. 2B), and PD-L1-low (FIG. 2C) populations, indicating that the combination of pembrolizumab with PCV increases the patient populations that can experience therapeutic effect of the immune checkpoint inhibitor.

The results of this study demonstrate that the magnitude of the treatment benefit of PCV+pembrolizumab compared to treatment with pembrolizumab monotherapy was maintained in both high and low biomarker subpopulations. The results of this study suggest that in biomarker high populations, PCV deepens the response to pembrolizumab, and in biomarker low populations, PCV broadens the population benefiting from pembrolizumab.

### Example 4

KEYNOTE-942 evaluated whether mRNA-1, a novel mRNA-based personalized cancer vaccine, improves relapse-free survival when combined with pembrolizumab in patients with resected melanoma.

Patients with completely resected stage IIIB, IIIC, IIID or IV cutaneous melanoma were randomly assigned 2:1 (stratified by stage) to receive mRNA-1 with pembrolizumab or pembrolizumab alone. mRNA-1 (1 mg) was administered intramuscularly every 3 weeks for up to 9 doses. Pembrolizumab (200 mg) was administered intravenously every 3 weeks for up to 18 doses. Recurrence-free survival was the primary end point.

157 patients were randomly assigned to the combination (107 patients) or pembrolizumab (50 patients) arms; median follow-up was 23 and 24 months, respectively. The combination was associated with longer recurrence-free survival than pembrolizumab (hazard ratio for recurrence or death, 0.561 (95% CI, 0.309 to 1.017); one sided P=0.027. Fewer recurrence/death events occurred in the combination than the control arm (22.4% [24/107] vs 40.0% [20/50]); 18-month recurrence-free survival rate was 78.6% (95% CI, 69.0 to 85.6) versus 62.2% (95% CI, 46.9 to 74.3), respectively. Combination therapy was also associated with longer distant metastasis-free survival (DMFS) than monotherapy (HR for recurrence or death, 0.347 [95% CI, 0.145-0.828]; two-sided P=0.013). Most treatment-related adverse events were grade 1 or 2. Grade ≥3 treatment-related adverse events occurred in 34.6% and 36.0% of patients in the combination and control arms, respectively. There were no mRNA-1 related grade 4 or 5 events.

mRNA-1 combined with pembrolizumab prolonged recurrence-free survival and DMFS versus pembrolizumab alone in patients with high-risk (stages IIIB-IIID or IV) resected melanoma with a manageable safety profile.

Neoantigens arise from cancer-specific mutations expressed on the cell surface that potentially stimulate antitumor T-cell responses. Tumor mutational burden is associated with neoantigen load and may be a predictive biomarker for response to immunotherapy. Most neoantigens are unique to each patient's cancer, but no individual neoantigen peptide sequence has been predictive of tumor response. Previous studies of adjuvant vaccines in resected melanoma and other cancers failed to show benefit or suggested a detrimental effect of vaccine therapy.

Disease recurrence for stage IIB-IV melanoma following complete resection is common despite treatment with standard-of-care adjuvant therapy, which includes checkpoint inhibitors and combination BRAF/MEK inhibitors for BRAF mutant melanomas. Personalized tumor-informed cancer vaccines may enhance the activity of checkpoint inhibitors and thereby potentially improve clinical benefit by increasing endogenous neoantigen T-cell responses, inducing T-cell responses to new neoantigens, and epitope spreading to other antigens.

mRNA-1, an mRNA-based personalized cancer vaccine comprising an mRNA encoding up to 34 antigens encapsulated in a lipid-nanoparticle, is tailored to a patient's tumor. Algorithmically derived and encoded neoantigens in mRNA-1 are endogenously translated and enter the cellular antigen presentation pathway. Preliminary data indicated that mRNA-1 induced robust and antigen-specific T-cell responses to several neoantigens encoded in the mRNA sequence. An open-label, randomized, phase 2 study of adjuvant mRNA-1 plus pembrolizumab versus pembrolizumab was conducted in patients with completely resected high-risk cutaneous melanoma to assess if the combination improved recurrence-free survival.

### Patients

Eligible patients were aged ≥18 years with resectable cutaneous melanoma stage IIIB, IIIC, IIID, or IV; patients with stage IIIB disease were eligible only if relapse occurred within 3 months of prior surgery. Patients had to have a complete surgical resection ≤13 weeks before the first dose of pembrolizumab and to be disease-free at study entry. Additionally, eligible patients had an Eastern Cooperative Oncology Group (ECOG) performance status score of 0-1.

### Vaccine Manufacture

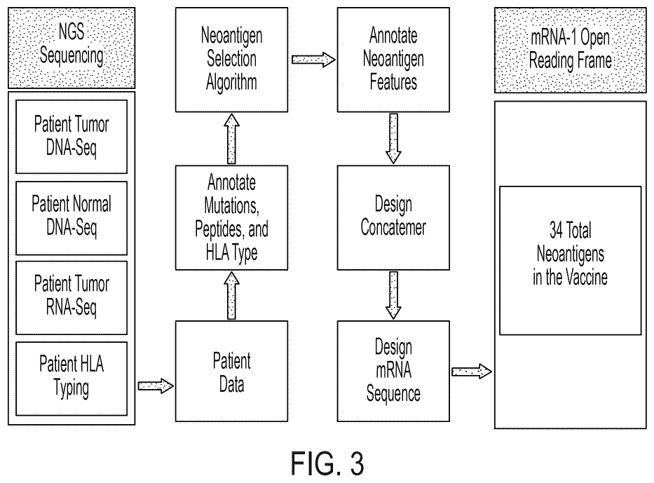

Each mRNA-1 was produced using an automated bioinformatics system for neoantigen prediction and vaccine design in an integrated manufacturing process (FIG. 3).

Patient samples were analyzed by next-generation sequencing. Whole exome sequencing data were generated from patient tumor and blood samples using the Illumina Novaseq™ Platform and results from each blood sample were used to determine the patient's human leukocyte antigen type per the guidelines of the American Association for Histocompatibility and Immunogenetics. The transcriptome was determined by RNA sequencing. The automated mRNA-1 bioinformatics system used patient-specific human leukocyte antigen typing, whole exome sequencing, and RNA sequencing results to determine amino acid sequences for up to 34 selected neoantigens and incorporated the top candidates into a concatemeric mRNA-1 sequence. The mRNA-1 sequence was then converted to multiple DNA nucleotide sequences optimized for ease of manufacturing, transferred electronically to production for the manufacture of each patient-specific mRNA-1 and formulated in lipid nanoparticles. Upon completion of manufacturing and testing, mRNA-1 was shipped in a single patient chain of custody to the clinical site for administration to the specific patient.

### Trial Design and Interventions

In this open-label, phase 2 study (NCT03897881), patients with completely resected high risk cutaneous melanoma were randomly assigned 2:1 to receive mRNA-1 plus pembrolizumab (combination arm) or pembrolizumab (control arm). Randomization was stratified according to stage (stage IIIB, IIIC, IIID, or IV), performed centrally at enrollment via an interactive web response system based on a block randomization technique.

Random allocation occurred prior to commencement of pembrolizumab. Limited manual allocation was permitted based on manufacturing availability and prespecified in the protocol; every effort was made to limit this and ensure a 2:1 ratio between treatment arms. Patients assigned to the combination arm received 200 mg intravenous pembrolizumab (typically 2 cycles of 3 weeks each) during mRNA-1 manufacture. The combination treatment period commenced upon availability of mRNA-1, which was administered intramuscularly in alternating limbs at a dose of 1 mg of mRNA concurrently with the next scheduled dose of pembrolizumab for synchronous dosing in 3-week cycles. Pembrolizumab was administered for up to 18 doses over 1 year. Patients in both treatment arms continued pembrolizumab until disease recurrence, unacceptable toxicity, or completion of 18 cycles (˜1 year), whichever was earlier. After completion of study treatment, all patients were followed until death, withdrawal of consent, loss to follow-up, or 3 years after their first dose of pembrolizumab, whichever occurred earlier.

The primary end point was investigator-assessed recurrence-free survival (RFS; defined as the time from first dose of pembrolizumab until the date of first recurrence [local, regional, or distant metastasis], a new primary melanoma, or death from any cause) in the intention-to-treat population. For patients without any events, RFS was censored at the latest disease evaluation before new anti-cancer therapy (including surgery) initiation or new primary cancer diagnosis (non-melanoma), if any. Secondary endpoints included safety and tolerability and investigator-assessed distant metastasis-free survival (DMFS; defined as the time from first dose of pembrolizumab until the date of first distant recurrence or death from any cause). The DMFS was hierarchically tested following the positive primary endpoint of RFS.

A study was conducted to examine the secondary efficacy endpoint of distant metastasis free survival. Patients in the mRNA-1 trial disclosed herein with resected, high-risk Stage IIIB/C/D and IV cutaneous melanoma received mRNA-1 in combination with pembrolizumab or pembrolizumab alone. The secondary endpoint of distant metastasis-free survival (DMFS) was pre-specified for testing following positive RFS. All tests were performed at 1-sided alpha=0.1 level.

The 157 patients were treated as noted above. There was a statistically and clinically significant improvement in DMFS for the combination treatment versus pembrolizumab monotherapy (HR=0.347; 95% CI: (0.145, 0.828); 1-sided p-value 0.0063). Distant recurrence or death was reported in 9 out of 107 patients (8.4%) and 12 out of 50 patients (24%), with 18-month DMFS rates (95% CI) were 91.8% (84.2%, 95.8%) vs 76.8% (61.0%, 86.8%) in the combination and monotherapy arm, respectively. Similar results were observed in the per-protocol population and were generally consistent with the overall ITT population, with a markedly lower event rate for the combination versus the monotherapy therapy arm (8/103 (7.8%) vs. 12/49 (24.5%); HR=0.311; 95% CI: (0.126, 0.766); 1-sided p-value 0.0036).

Thus, mRNA-1 in combination with pembrolizumab as adjuvant therapy for resected high-risk melanoma significantly prolonged DMFS compared to pembrolizumab. These results provide further evidence that the personalized neoantigen approach is beneficial for cancer patients.

Across multiple cancer types and disease settings, detection of minimal residual disease (MRD) by circulating tumor DNA (ctDNA) assays in plasma identifies patients at higher risk of relapse and progression, and can monitor presence of disease throughout treatment. ctDNA analyses has been performed in the clinical study to identify the association between minimal residual disease and the probability of recurrence.

In the human clinical study baseline tumor core biopsies were taken and matched whole blood was subjected to whole exome sequencing (WES). A personalized amplicon-based NGS assay by Inivata (RaDaR®) was used to identify and prioritize up to 48 patient-specific somatic variants to analyze ctDNA in longitudinal plasma samples for MRD detection. This method for ctDNA analysis is distinct from most prior studies in melanoma that report use of ddPCR for single mutations or fixed-gene panels. The association of MRD with RFS was evaluated with Kaplan Meier analyses and assessed with hazard ratio (95% CI) in ctDNApos and ctDNAneg subgroups and across study arms.

Of patients enrolled in this study with evaluable ctDNA, 88% (110/125) were ctDNAneg at start of treatment. Significantly longer RFS was observed in patients who were ctDNAneg compared to those with ctDNApos at baseline across study arms (RFS event rate of 20.9% (23/110) versus 80.0% (12/15); HR=0.150; 95% CI: 0.073, 0.306). Within the mRNA-1 and pembrolizumab combination arm, significantly longer RFS was observed in patients with ctDNAneg compared to ctDNApos samples (RFS event rate of 10.4% (8/77) versus 76.9% (10/13); HR=0.087; 95% CI: 0.034, 0.222). This trend was also observed in the pembrolizumab arm (RFS event rate of 45.5% (15/33) versus 100% (2/2); HR=0.008; 95% CI: 0.001, 0.088) study arm.

MRD detection by plasma ctDNA assay at the start of adjuvant melanoma treatment is uncommon but is associated with shorter RFS. This study demonstrated that treatment with the combination of mRNA-1 and pembrolizumab was associated with prolonged RFS compared to pembrolizumab monotherapy in patients with high risk resectable melanoma, irrespective of MRD status.

### Assessments

All patients were assessed by physical exam, blood tests and radiological imaging (computed tomography or magnetic resonance imaging) at baseline, on-study (every 12 weeks from first dose of pembrolizumab for 12 months), and during follow-up (every 12 weeks for 12-24 months, then every 26 weeks from 24-36 months). Recurrence was histologically confirmed whenever possible. For patients without any event, follow-up was censored at the latest disease evaluation.

Adverse events were collected and categorized by severity according to the National Cancer Institute Common Terminology Criteria for Adverse Events, version 5.0. Immune-mediated adverse events were programmatically determined from a predefined list of Medical Dictionary for Regulatory Activities (MedDRA) terms, which was updated in accordance with each new version of MedDRA. In the combination arm, adverse events and serious adverse events were followed for 100 days after the last dose of mRNA-1 or until the start of new anticancer therapy. In both treatment arms, adverse events were followed for 30 days (90 days for serious adverse events) after the last dose of pembrolizumab. Relatedness to treatment was determined by the investigator.

### Trial Oversight

The trial protocol and amendments were approved by the relevant independent review committees/independent ethics committees at each institution. The trial was conducted in accordance with the Declaration of Helsinki and with Good Clinical Practice. All patients provided written informed consent.

### Statistical Analysis

Approximately 150 patients were planned to be randomly assigned in a 2:1 ratio to the combination arm or the control arm. A total of 40 recurrence-free survival events (recurrence or death) were required to provide approximately 80% power to detect a HR of 0.5 with an overall 1-sided a of 0.10.

Efficacy analyses were performed for the intention-to-treat population, which included all randomized patients. A per-protocol population excluding patients never treated, patients who received treatment different from their final assignment, and ineligible patients with metastasis at baseline was also used for supportive efficacy analyses. All safety analyses were performed on the safety population, defined as all randomly assigned patients who received at least 1 dose of treatment.

Primary analysis of recurrence-free survival was compared using the log-rank test stratified by disease stage. If the result was positive in the intention-to-treat population, a hierarchical testing approach was applied to the secondary endpoint of DMFS and the stratified log rank test was used for comparison. Hazard ratios and 95% CIs in the intention-to-treat and per-protocol populations were estimated using a stratified Cox proportional hazards model with the Efron method of tie handling with treatment group as a covariate; medians with 95% CIs and rates at 12 and 18 months were estimated using the Kaplan-Meier method. Safety was summarized using descriptive statistics. For exploratory purposes, the predictive importance of several factors with regard to the differences in recurrence-free survival were investigated. Subgroup analyses were performed with hazard ratios and 95% CIs calculated based on an unstratified Cox proportional hazards model.

### Tumor Mutational Burden and Pd-L1 Assessments

An exploratory end point was assessment of tumor mutational burden and programmed death ligand 1 (PD-L1) status as potential predictive biomarkers of response. Tumor mutational burden, defined in this Example as the number of non-synonymous mutations ≥5% allele frequency, was determined by whole exome sequencing of tumor and matched normal whole blood samples from all patients using the Illumina Novaseq™ platform. The threshold for high tumor mutational burden was 175 mutations per exome (10 mutations per megabase). PD-L1 expression levels were assessed by immunohistochemistry staining of tumor (22C3 antibody, Agilent/Dako), and combined positivity of membranous PD-L1 on tumor cells and tumor-associated immune cells was scored on a melanoma-specific scale of 0-5. Tumors scoring ≥2, representing ≥1% PD-L1 positivity, were considered PD-L1 positive. PD-L1 status was assessed only for patients with sufficient baseline biopsy samples.

### Immunogenicity Assessments

Immunogenicity was measured in peripheral blood mononuclear cells (PBMCs) from leukapheresis collected at baseline (P1D1) and 8 days after the fourth combination treatment cycle (P6D8). T-cell responses to vaccine neoantigens pools and to individual neoantigens in this study were analyzed by IFNγ ELISpot directly ex vivo.

### Patients and Trial Regimen

157 patients were enrolled in the clinical trial, with 107 assigned to the combination arm and 50 to the control arm. One patient assigned to the combination arm received pembrolizumab only because their mRNA-1 could not be produced as a result of poor tissue quality leading to poor NGS. mRNA-1 was successfully prepared for all other patients in the combination arm (>99%); median number of vaccine neoantigens was 34 (range, 9-34) (Table 1).

TABLE 1
Neoantigen number included in vaccines

| Number of | Number of vaccines | |
|---|---|---|
| antigens | (%) (n = 104) | |
| 34 | 95 | (91%) |
| 32 | 2 | (2%) |
| 27 | 1 | (1%) |
| 22 | 2 | (2%) |
| 20 | 1 | (1%) |
| 15 | 1 | (1%) |
| 10 | 1 | (1%) |
| 9 | 1 | (1%) |

Between February 2021 and April 2021, the manufacturing and testing required to release and distribute the mRNA-1273 COVID-19 vaccine to over a billion people, limited the available resources for manufacturing mRNA-1. Because of this constraint, 9 patients who were randomly assigned to the combination arm were manually reallocated to the control arm. Manual reallocation was performed prior to tissue collection, next-generation sequencing, and treatment initiation. Baseline characteristics were balanced between treatment arms, and the study population was representative of patients with high-risk resected melanoma (Table 2).

TABLE 2
Demographic and Clinical Characteristics
of the Patients at Baseline

| | |
|---|---|
| mRNA-1 + | |
| pembrolizumab | Pembrolizumab |
| (n = 107) | (n = 50) |

Sex, n (%)

| | | | | |
|---|---|---|---|---|
| Male | 70 | (65.4) | 31 | (62.0) |
| Female | 37 | (34.6) | 19 | (38.0) |

Age (years)

| | | | | |
|---|---|---|---|---|
| Median (range) | 63 | (26-83) | 61.5 | (24-89) |
| Mean (SD) | 61.3 | (13.50) | 59.4 | (14.25) |

Age Group, n (%)

| | | | | |
|---|---|---|---|---|
| <65 years | 59 | (55.1) | 28 | (56.0) |
| ≥65 years | 48 | (44.9) | 22 | (44.0) |

ECOG performance status score

| | | | | |
|---|---|---|---|---|
| 0 | 90 | (84.1) | 40 | (80.0) |
| 1 | 15 | (14.0) | 9 | (18.0) |
| Missing^{a} | 2 | (1.9) | 1 | (2.0) |

Disease Stage at Randomization, n (%)^{b}

| | | | | |
|---|---|---|---|---|
| Stage IIIC | 89 | (83.2) | 42 | (84.0) |
| Stage IIID | 2 | (1.9) | 2 | (4.0) |
| Stage IV | 16 | (15.0) | 6 | (12.0) |

Number of Prior Cancer-related Surgeries, n (%)

| | | | | |
|---|---|---|---|---|
| 1 | 41 | (38.3) | 24 | (48.0) |
| 2 | 36 | (33.6) | 18 | (36.0) |
| ≥3 | 30 | (28.0) | 8 | (16.0) |
| Lactate Dehydrogenase | 189.5 | (118-528) | 185.5 | (113-1180) |
| (U/L), Median (range)^{c} | | | | |
| Lactate Dehydrogenase, | 5 | (4.7) | 3 | (6.0) |
| >upper limit of normal, n (%) | | | | |
| Lymph node dissection, n | 34 | (31.8) | 15 | (30.0) |
| (%) | | | | |

PD-L1 Status, n (%)

| | | | | |
|---|---|---|---|---|
| Positive | 69 | (64.5) | 27 | (54.0) |
| Negative | 13 | (12.1) | 5 | (10.0) |
| Indeterminate^{d} | 25 | (23.4) | 18 | (36.0) |

BRAF V600 status, n (%)^{e}

| | | | | |
|---|---|---|---|---|
| V600K or V600E mutation | 41 | (38.3) | 20 | (40.0) |
| WT | 66 | (61.7) | 30 | (60.0) |

Tumor Mutational Burden, n (%)^{c}

| | | | | |
|---|---|---|---|---|
| <10 mutations/Mb | 26 | (24.3) | 19 | (38.0) |
| ≥10 mutations/Mb | 79 | (73.8) | 30 | (60.0) |

Time from most recent surgery of curative intent to
first dose of pembrolizumab, weeks^{c}

| | | | | |
|---|---|---|---|---|
| Median (range) | 10.9 | (0.7, 13.9) | 10.1 | (3.0, 19.6) |
| Mean (SD) | 10.1 | (2.51) | 9.9 | (3.14) |

ECOG, Eastern Cooperative Oncology Group; PD-L1, programmed cell death ligand 1.
^{a}Three patients were not treated and therefore had no baseline ECOG score.
^{b}According to the 8th edition of the American Joint Committee on Cancer staging manual.
^{c}Available for 154 patients.
^{d}Patients for whom there was no sample to send for PD-LI evaluation or for whom sample quality or quantity was too low to perform the assay.
^{e}BRAF status determined by WES on baseline tumor samples. WT refers to V at position 600 on BRAF gene.

The median number of mRNA-1 doses was 9.0 (range, 1-9). The median number of pembrolizumab doses was 18.0 (range, 2-18) and 18.0 (range, 1-18) in the combination and control arms, respectively. In the combination arm, mRNA-1 treatment was initiated in >80% of patients during pembrolizumab cycle 3 (range, 3-5). Median duration of follow-up was 23 months (range, 14-39) in the combination arm and 24 months (range, 21-42) in the control arm.

Among 107 patients randomly assigned to the combination arm, the most common reason for discontinuation of either mRNA-1 or pembrolizumab was adverse events (15.0% and 25.2%). For the 50 patients randomly assigned to the control arm, the most common reason for discontinuation of pembrolizumab was disease recurrence (20.0%). A total of 69 patients (64.5%) completed mRNA-1 treatment in the combination arm; 64 patients (59.8%) in the combination arm and 28 patients (56.0%) in the control arm completed pembrolizumab treatment.

### Efficacy

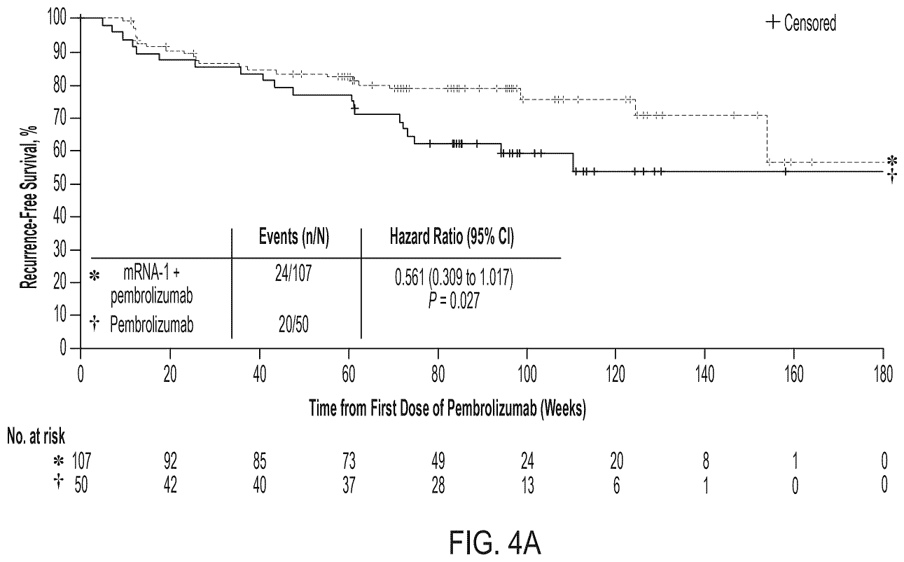

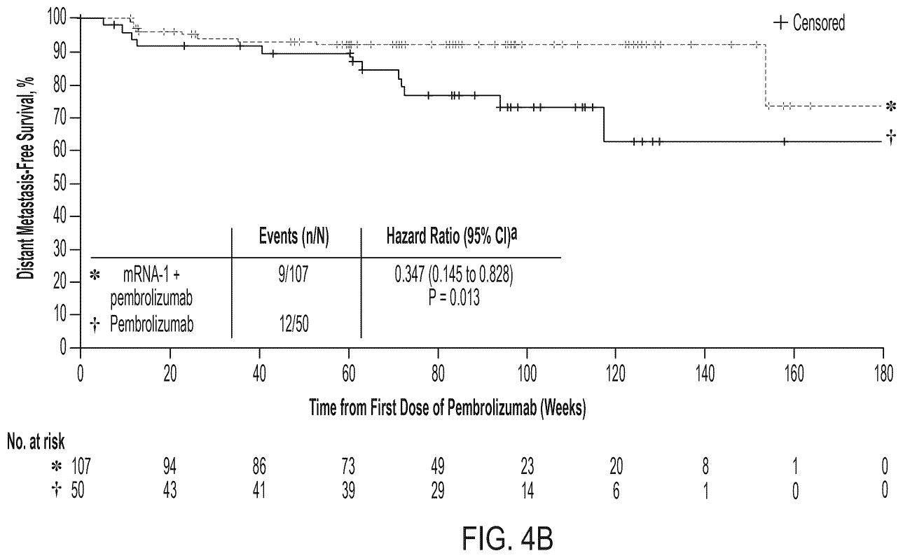

In the intention-to-treat population, recurrence-free survival (RFS) was significantly longer in the combination than the control arm (hazard ratio for recurrence or death, 0.561 [95% CI, 0.309 to 1.017]; one sided P-0.027; two-sided P=0.053; FIG. 4A). The 12-month rate of recurrence-free survival was 83.4% (95% CI, 74.7 to 89.3) and 77.1% (95% CI, 62.5 to 86.6) in the combination and control arms, respectively. At 18 months, the rates of recurrence-free survival were 78.6% (95% CI, 69.0 to 85.6) and 62.2% (95% CI, 46.9 to 74.3) in the combination and control arms, respectively. Supportive analysis of recurrence-free survival demonstrated a piecewise hazard ratio of 0.885 (95% CI, 0.378 to 2.070) within the first 40 weeks and 0.331 (95% CI, 0.135 to 0.815) beyond 40 weeks.

DMFS was significantly longer in the combination arm versus the control arm (HR for distant recurrence or death, 0.347 [95% CI, 0.145-0.828]; two-sided P=0.013; FIG. 4B). At 18 months, the rates of distant RFS were 91.8% (95% CI, 84.2-95.8) and 76.8% (95% CI, 61.0-86.8). At the time of the database cutoff, distant recurrence or death occurred in 9 patients (8.4%) and 12 patients (24.0%) in the combination and pembrolizumab monotherapy arms, respectively.

At the time of database cutoff, 44 events (recurrences or deaths) were reported in the intention-to-treat population with a minimum follow-up of 13.5 months. Recurrence or death occurred in 24 patients (22.4%) and 20 patients (40.0%) in the combination and control arms. RFS events (local, regional, or distant metastatic recurrence; new primary melanoma; or death from any cause) in the intention-to-treat population were: (i) 14 (13.1% of patients who experienced an RFS event) local/regional recurrence, 7 (6.5%) distant recurrence, and 3 (2.8%) other in the mRNA-1+pembrolizumab combination treatment arm; and (ii) 9 (18%) local/regional recurrence, 10 (20%) distant recurrence, and 1 (2%) other in the pembrolizumab control arm. The 4 total “other” RFS events were new primary cutaneous melanoma or death (n=1, from sepsis, unrelated to mRNA-1 or pembrolizumab). Distant recurrence location in each group are shown in Table 3 below.

TABLE 3
Distant recurrence location

| | mRNA-1 + | | | |
|---|---|---|---|---|
| | pembrolizumab | | Pembrolizumab | |
| | n = 107 | | n = 50 | |
| Patients with distant | 9 | (8.4) | 12 | (24.0) |
| recurrence or death, n (%) | | | | |

Site of distant recurrence, n (%)

| | | | | |
|---|---|---|---|---|
| Lymph node | 2 | (1.9) | 4 | (8.0) |
| Lung | 2 | (1.9) | 4 | (8.0) |
| Liver | 3 | (2.8) | 1 | (2.0) |
| Bone | 1 | (0.9) | 2 | (4.0) |
| Brain | 1 | (0.9) | 3 | (6.0) |

| | | | |
|---|---|---|---|
| Skin | 0 | 4 | (8.0) |

| | | | | |
|---|---|---|---|---|
| Colon | 1 | (0.9) | 1 | (2.0) |

| | | | |
|---|---|---|---|
| Spleen | 0 | 1 | (2.0) |

| Soft tissue | 1 | (0.9) | 0 | |
|---|---|---|---|---|
| Other site | 2 | (1.9) | 1 | (2.0) |

| | | | |
|---|---|---|---|
| Death not due to melanoma | 1 | (0.9)^{a} | 0 |

^{a}Sepsis, unrelated to mRNA-1 or pembrolizumab.

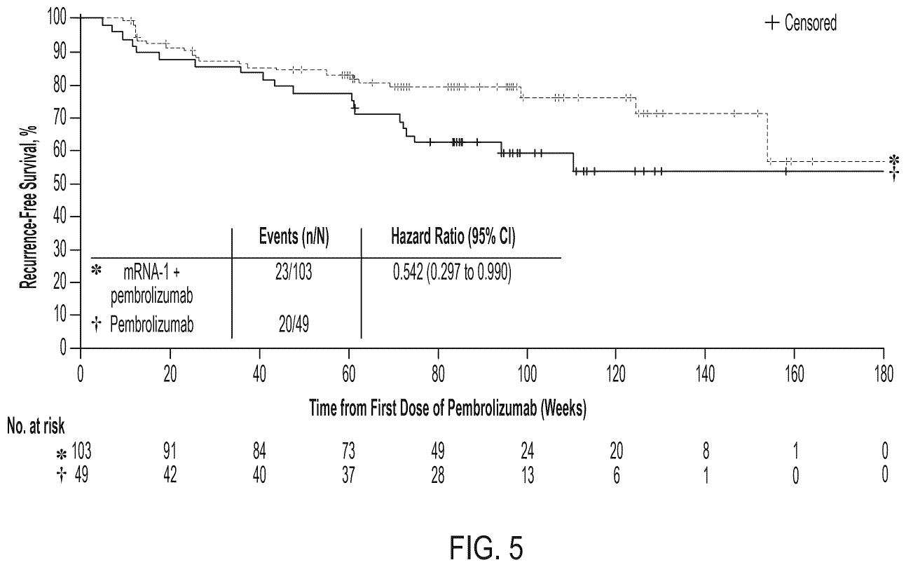

Results in the per-protocol population were consistent with the intention-to-treat population (hazard ratio for recurrence or death, 0.542 [95% CI, 0.297 to 0.990]; descriptive P=0.02; FIG. 5).

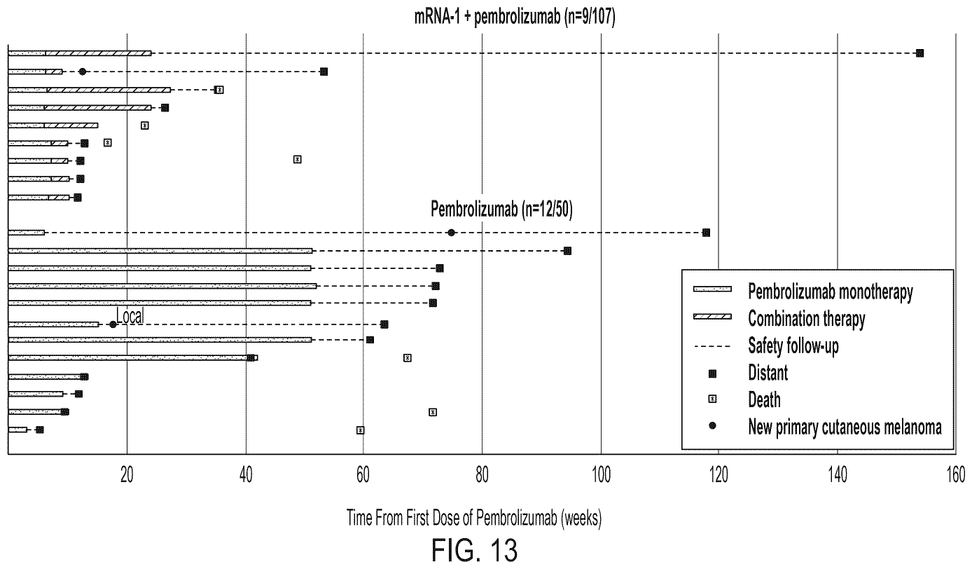

Timelines of DMFS during and after treatment completion for the 21 patients who experienced distant recurrence and/or death during and after treatment are shown in FIG. 13. Reasons for death were disease progression (n=2), unrelated adverse event (n=1, without distant recurrence), and unknown (n=1) in the combination treatment arm, and disease progression (n=3) in the pembrolizumab monotherapy arm.

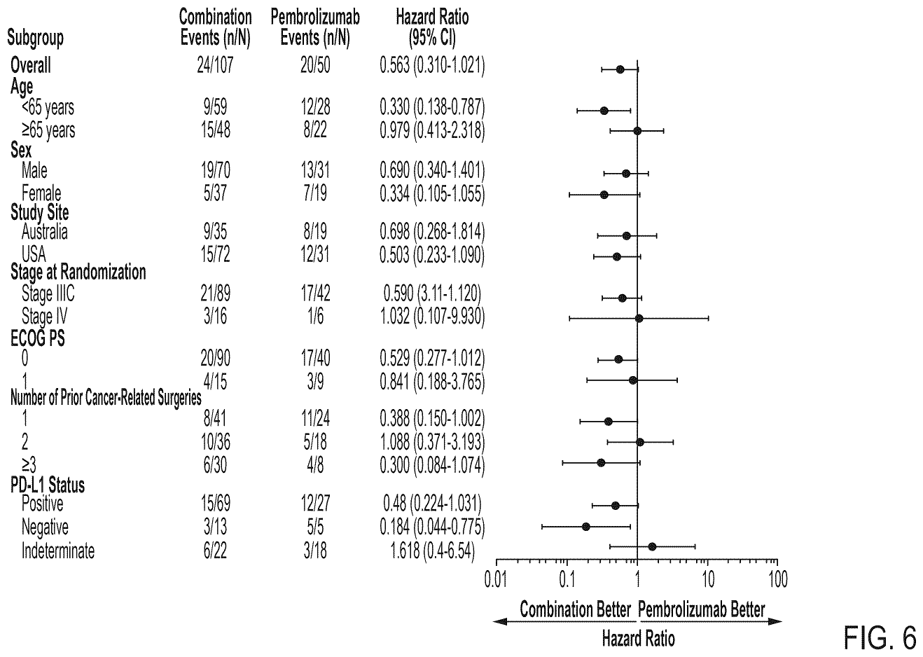

A subgroup analysis including only patients randomized before the manufacturing reprioritization confirmed that manual allocation of 9 patients did not affect the results (Table 4). In addition, the recurrence-free survival benefit was generally consistent across most predefined subgroups (FIG. 6) with point estimate hazard ratios less than 1, regardless of tumor mutational burden or PD-L1 status.

TABLE 4
Sensitivity Analysis of Recurrence-Free Survival
in the Intention-to-Treat Population.^{a}

| | mRNA-1 + | |
|---|---|---|
| | pembrolizumab | Pembrolizumab |
| | n = 107 | n = 50 |
| Events, n (%) | 24 (22.4) | 20 (40.0) |

Piecewise hazard ratio group 1 (95% CI)

| | |
|---|---|
| ≤40 weeks | 0.885 (0.378 to 2.070) |
| >40 weeks | 0.331 (0.135 to 0.815) |

Piecewise hazard ratio group 2 (95% CI)

| | |
|---|---|
| ≤52 weeks | 0.689 (0.323 to 1.473) |
| >52 weeks | 0.393 (0.145 to 1.063) |

^{a}The piecewise hazard ratio and 95% confidence interval for mRNA-1 plus pembrolizumab versus pembrolizumab is estimated using a stratified time-dependent Cox model with effects for period-by-treatment interaction, stratified by disease stage (stages IIIB or IIIC or IIID vs stage IV) used for randomization.

### Safety

The safety analysis population included 154 patients, 104 in the combination arm and 50 in the control arm. Treatment-related adverse events occurred in 145 patients (94.2%); 104 patients (100%) in the combination arm, and 41 patients (82.0%) in the control arm. Treatment-related adverse events were grade 1 or 2 in 78 patients (75.0%) in the combination arm and 32 patients (64.0%) in the control arm.

mRNA-1-related events were grade 1 or 2 in 82.7% of patients; the median time to resolution was 3 days. Grade 3 mRNA-1-related adverse events were reported in 11.5% of patients; the most common was fatigue (4.8%). No grade 4 or 5 mRNA-1-related adverse events occurred (Table 5). Adverse events led to discontinuation of mRNA-1 in 15.4% of patients. The most common adverse event of any grade attributed either to mRNA-1 alone or to both mRNA-1 and pembrolizumab (mRNA-1-related events) were fatigue (60.6%), injection site pain (55.8%), and chills (50.0%). Injection site reactions occurred in 69.2% of patients, most frequently during the initial treatment cycle.

In the combination arm, adverse events related to either pembrolizumab alone or to both pembrolizumab and mRNA-1 were grade 1 or 2 in 74.0% and grade ≥3 in 23.1% of patients, none were grade 5 (Table 5). The most common pembrolizumab-related adverse events were fatigue (69.2%), diarrhea (29.8%), and pruritus (28.8%). In the control arm, adverse events related to pembrolizumab were grade 1 or 2 in 64.0% and grade ≥3 in 18.0% of patients; none were grade 5. The most frequent pembrolizumab-related adverse events in the control arm were fatigue (40.0%), pruritus (20.0%), hypothyroidism (16.0%), and rash (16.0%). Adverse events led to discontinuation of pembrolizumab in 25.0% and 18.0% in the combination and control arms, respectively. The incidence of pembrolizumab-related serious adverse events in the combination and control arms was 14.4% and 10.0%, respectively.

TABLE 5
Adverse Events Occurring in >20% of Patients in the Combination Arma.

| | mRNA-1+pembrolizumab | | | | Pembrolizumab | | | |
|---|---|---|---|---|---|---|---|---|
| | (n = 104) | | | | (n = 50) | | | |
| | Any grade | | Grade ≥3 | | Any grade | | Grade ≥3b | |
| Any | 104 | (100.0) | 36 | (34.6) | 47 | (94.0) | 18 | (36.0) |
| Any treatment- | 104 | (100.0) | 26 | (25.0) | 41 | (82.0) | 9 | (18.0) |
| related AE | | | | | | | | |

| | | | | | | |
|---|---|---|---|---|---|---|
| Serious AE^{†} | 15 | (14.4) | — | 5 | (10.0) | — |

All cause immune-related AEs

| Immune-mediated | 37 | (35.6) | 11 | (10.6)^{f} | 18 | (36.0) | 7 | (14.0) |
|---|---|---|---|---|---|---|---|---|
| AEs | | | | | | | | |
| Adrenal | 5 | (4.8) | 4 | (3.8) | 2 | (4.0) | 1 | (2.0) |
| insufficiency | | | | | | | | |
| Colitis | 6 | (5.8) | 3 | (2.9) | 2 | (4.0) | 0 | |
| Hepatitis | 1 | (1.0) | 0 | | 1 | (2.0) | 1 | (2.0) |
| Hyperthyroidism | 6 | (5.8) | 0 | | 3 | (6.0) | 1 | (2.0) |
| Hypophysitis | 1 | (1.0) | 0 | | 0 | | 0 | |
| Hypothyroidism | 21 | (20.2) | 0 | | 8 | (16.0) | 0 | |
| Myositis | 0 | | 0 | | 1 | (2.0) | 1 | (2.0) |
| Nephritis | 3 | (2.9) | 3 | (2.9) | 1 | (2.0) | 0 | |
| Pancreatitis | 0 | | 0 | | 1 | (2.0) | 1 | (2.0) |
| Pneumonitis | 4 | (3.8) | 1 | (1.0) | 0 | | 0 | |
| Sarcoidosis | 0 | | 0 | | 1 | (2.0) | 0 | |
| Severe skin reaction | 3 | (2.9) | 0 | | 1 | (2.0) | 0 | |
| Thyroiditis | 0 | | 0 | | 2 | (4.0) | 1 | (2.0) |
| Type 1 diabetes | 0 | | 0 | | 1 | (2.0) | 1 | (2.0) |
| mellitus | | | | | | | | |

mRNA-1 or combination treatment-related adverse events^{c}

| Any | 98 | (94.2) | 12 | (11.5)^{d} | — | — |
|---|---|---|---|---|---|---|
| Fatigue | 63 | (60.6) | 5 | (4.8) | — | — |
| Injection site pain | 58 | (55.8) | 0 | | — | — |
| Chills | 52 | (50.0) | 0 | | — | — |
| Pyrexia | 50 | (48.1) | 1 | (1.0) | — | — |
| Headache | 33 | (31.7) | 0 | | — | — |
| Injection site | 33 | (31.7) | 0 | | — | — |
| erythema | | | | | | |
| Influenza-like illness | 32 | (30.8) | 0 | | — | — |
| Nausea | 26 | (25.0) | 0 | | — | — |
| Myalgia | 22 | (21.2) | 1 | (1.0) | — | — |

Pembrolizumab or combination treatment-related adverse events^{c}

| | | | | | | | | |
|---|---|---|---|---|---|---|---|---|
| Any | 101 | (97.1) | 24 | (23.1)^{b} | 41 | (82.0) | 9 | (18.0)^{e} |

| | | | | | | | |
|---|---|---|---|---|---|---|---|
| Fatigue | 72 | (69.2) | 6 | (5.8) | 20 | (40.0) | 0 |
| Diarrhea | 31 | (29.8) | 2 | (1.9) | 5 | (10.0) | 0 |

| | | | | | | |
|---|---|---|---|---|---|---|
| Pruritus | 30 | (28.8) | 0 | 10 | (20.0) | 0 |
| Nausea | 23 | (22.1) | 0 | 5 | (10.0) | 0 |
| Chills | 22 | (21.2) | 0 | 1 | (2.0) | 0 |

| | | | | | |
|---|---|---|---|---|---|
| Pyrexia | 22 | (21.2) | 0 | 0 | 0 |

Values are n (%).
ALT, alanine aminotransferase.
Grading per National Cancer Institute Common Terminology Criteria for Adverse Events version 5.0.
^{a}A summary of Investigator-attributed adverse events with a start date on or after the date study treatment began (treatment-emergent adverse events). Treatment-emergent adverse events related to treatment include events attributed by the Investigator to mRNA-1 or pembrolizumab. mRNA-1-related treatment-emergent adverse events include events attributed by the Investigator to mRNA-1 alone as well as events attributed to both mRNA-1 and pembrolizumab. Pembrolizumab-related treatment-emergent adverse events include events attributed by the Investigator to pembrolizumab alone and events attributed to both mRNA-1 and pembrolizumab.
^{b}No grade 4 or 5 mRNA-1-related events occurred in the combination therapy arm.
^{c}Includes grade 4 increased ALT, adrenal insufficiency, and enterocolitis (n = 1 each; 2.9% total).
^{d}Includes grade 4 increased blood creatinine phosphokinase (n = 1; 2.0%).
^{e}No deaths were related to mRNA-1 or pembrolizumab.
^{f}Includes grade 4 adrenal insufficiency and colitis (n = 1 each); no grade 5 events occurred.
^{g}No grade 4 or 5 events occurred.
^{†}Serious AEs included grade 1 fever, attributed to mRNA-1, and grade 3 muscular weakness and grade 3 autoimmune nephritis, attributed to both mRNA-1 and pembrolizumab.

Immune-mediated adverse events occurred in 35.6% in the combination arm and 36.0% in the control arm. Most immune-mediated adverse events were grade 1 or 2; grade ≥3 events occurred in 10.6% and 14.0% in the combination and control arm, respectively.

### Tumor Mutational Burden

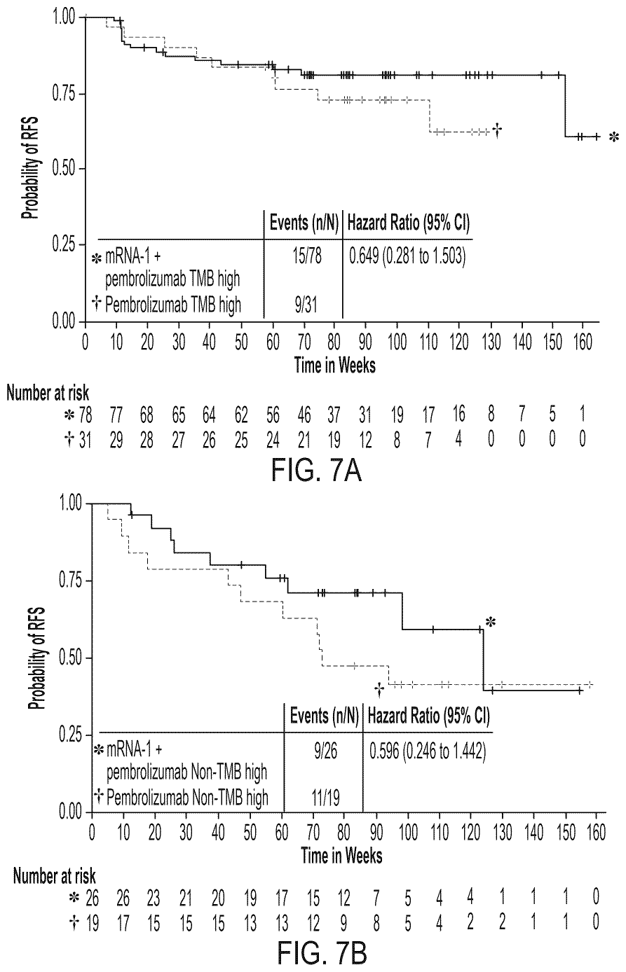

Tumor mutational burden data were available for 154 patients (98.1%), including 104 in the combination arm and 50 in the control arm. Of the intention-to-treat population, 75.0% and 62.0% of patients in the combination and control arms, respectively, had high tumor mutational burden. Recurrence-free survival was longer in the combination than in the control arm regardless of tumor mutational burden (FIGS. 7A and 7B). The hazard ratio for recurrence-free survival showed a similar effect of combination regardless of tumor mutational burden (high: hazard ratio 0.649 [95% CI, 0.281 to 1.503]; non-high: hazard ratio 0.596 [95% CI, 0.246 to 1.442]). Recurrence-free survival was longer in high versus non-high tumor mutational burden patient subgroups in both treatment arms. Similar trends were noted in PD-L1 positive and negative subgroups (FIG. 6).

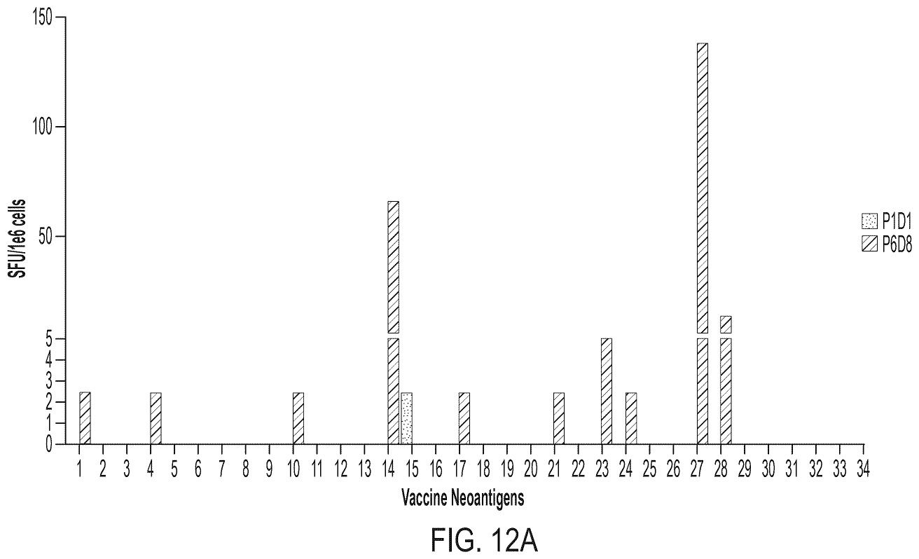

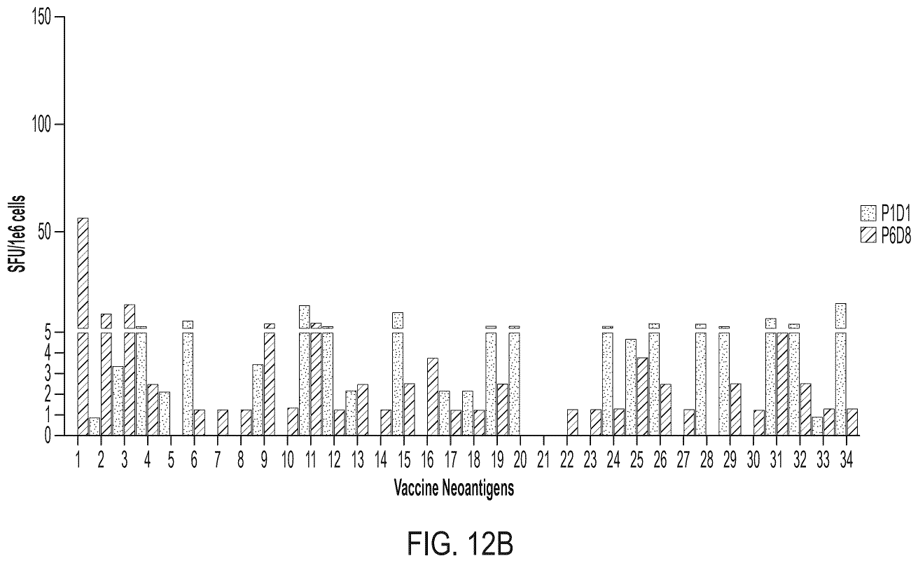

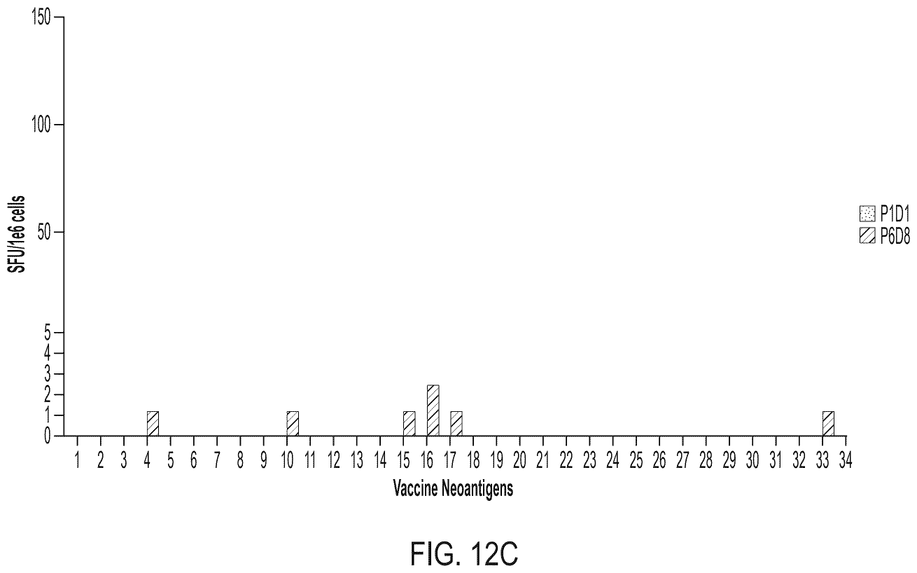

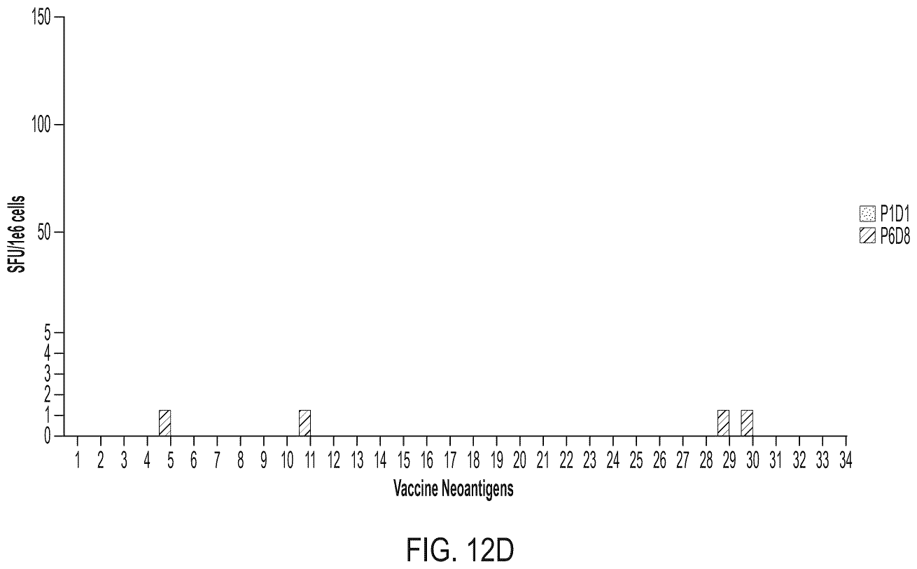

Immune responses in the pembrolizumab monotherapy arm were tested for neoantigens that were predicted to be immunogenic. Samples from two patients treated with the combination of mRNA-1 and pembrolizumab therapy exhibited broad presence of mRNA-1 neoantigen-specific T-cells, both at baseline and on study, demonstrating successful selection of immunogenic antigens for inclusion in mRNA-1 and the ability of combination treatment to induce neoantigen-specific T-cell responses that were not detectable at baseline and increase endogenous T-cell responses to neoantigens (FIG. 12A and FIG. 12B). In contrast, samples from two patients treated with pembrolizumab monotherapy overall had very low responses to vaccine neoantigens (FIG. 12C and FIG. 12D).

In the primary analysis of this randomized, phase 2 trial in patients with resected, high-risk melanoma, the risk of recurrence or death was significantly lower per protocol in patients treated with combination mRNA-1 and pembrolizumab versus pembrolizumab alone (hazard ratio 0.561 [95% CI, 0.309 to 1.017]; one sided P=0.027; two sided P=0.053), recurrence or death was markedly lower in the combination versus control arm (22.4% vs 40.0%). In the present study, the 18-month recurrence-free survival rate was higher in the combination (78.6%) versus the control arm (62.2%), suggesting clinical benefit with the addition of mRNA-1 to pembrolizumab. DMFS was also evaluated as an overall survival surrogate and was strongly correlated; combination therapy was associated with a longer DMFS than pembrolizumab monotherapy (HR for recurrence or death, 0.347 [95% CI, 0.145-0.828]; two-sided P=0.013), suggesting that mRNA-1 combined with pembrolizumab provides a clinically meaningful benefit. The 18-month recurrence-free survival rate in the control arm (62.2%) was also consistent with prior studies of pembrolizumab and nivolumab in this setting.

Longer recurrence-free survival with mRNA-1 plus pembrolizumab over standard-of-care pembrolizumab was observed across baseline prognostic subgroups, although interpretation may be limited by the small sample.

RFS curves for mRNA-1 combination and control arms began separating after ˜40 weeks. Improved HR after 40 weeks suggests recurrent dosing to induce repeat neoantigen exposure may be required to stimulate a robust antitumor immune response with neoantigen-specific T cells.

A key challenge in developing effective personalized cancer vaccines is identifying tumor neoantigens that induce a clinically beneficial immune response. Neoantigen burden varies considerably among tumors and individuals; some may be less immunogenic and evade or even suppress the host immune system. The impact of tumor heterogenicity, immune fitness, and the minimum number of neoantigens necessary to elicit a clinically meaningful immune response is currently unknown. mRNA technology for personalized cancer vaccines confers advantages over other approaches, as mRNA can encode several dozen neoantigens, has a rapid turnaround time, and does not incorporate into the host cell, which may carry unintended off target effects. The mRNA-1 combination and pembrolizumab monotherapy recurrence-free survival estimation curves started to separate after approximately 40 weeks. The delayed separation of the curve with therapeutic vaccines has been well described in the literature. As emphasized by the improved hazard ratio after 40 weeks, repeat dosing to enable repeat neoantigen exposure may be required to induce a robust antitumor immune response with neoantigen specific T cells.

The safety profile of mRNA-1 plus pembrolizumab was manageable. Most treatment-related adverse events were grade 1 or 2, and rates of grade ≥3 and serious adverse events were similar between treatment arms. The most common mRNA-1-related adverse events were flu-like symptoms (fatigue, chills, pyrexia, headache) and local injection site reactions (injection site pain, erythema), which are considered related to the mechanism of action, were generally self-limited, and decreased in incidence in subsequent dosing cycles. There was no evidence of potentiation of immune-mediated adverse events with mRNA-1 plus pembrolizumab; incidence of these events were similar in each arm (˜36%).

As prespecified in the protocol, a limited number of manual allocations were permitted for flexibility in manufacturing, ensuring the arms would be rebalanced to ensure equipoise. Early in the study, one patient was manually reallocated to the control arm because the quality of their tissue sample was unsuitable for the next-generation sequencing required for mRNA-1 manufacture. In conclusion, this phase 2 trial shows prolonged recurrence-free survival with mRNA-1 plus pembrolizumab over standard-of-care pembrolizumab in resected cutaneous high-risk melanoma, with a manageable safety profile.

### Example 5

This example describes results of a study evaluating the efficacy of a combination of mRNA-1, a personalized cancer vaccine, with pembrolizumab, across tumor mutational burden and biomarker subgroups.

The inflamed tumor microenvironment provides a favorable milieu associated with clinical benefit with immune checkpoint inhibitors. Tumor mutational burden (TMB), gene-expression profile (GEP) score, programmed death ligand-1 (PD-L1) expression, and circulating tumor DNA (ctDNA) are biomarkers associated with clinical responses to pembrolizumab that may function by promoting endogenous responses to tumor neoantigens. Higher TMB provides additional opportunities for tumor-antigen recognition by T cells, suggesting a potential benefit for T cell-based immunotherapies. Higher GEP score and PD-L1 expression are characteristic of an inflamed tumor microenvironment that is indicative of T cell infiltration and function, which are critical for the efficacy of T cell-based immunotherapies.

The objectives of the present study were: (1) to assess the distribution of TMB, tumor inflammation signature (TIS; a gene expression profile similar to GEP), PD-L1 expression, and ctDNA in baseline tumors in both study arms (mRNA-1 and pembrolizumab versus pembrolizumab monotherapy) in patients; (2) to explore the prognostic impact of TMB, TIS, PD-L1 expression, and ctDNA within the study arms; and (3) to assess recurrence-free survival (RFS) in biomarker-high and -low subgroups across the study arms, and define whether the mRNA-1 and pembrolizumab combination deepens the clinical benefit in the populations already responsive to pembrolizumab monotherapy and/or extends clinical benefit into new patient populations.

Biomarker assessments were conducted on formalin-fixed paraffin-embedded (FFPE) tumor core biopsies provided at study screening and utilized for the design of mRNA-1 for each patient.

To assess TMB, macrodissected FFPE biopsies and matched normal whole-blood samples were subjected to whole-exome sequencing (WES) to identify patient-specific somatic mutations. TMB in this Example represents the number of nonsynonymous tumor mutations with an allele frequency ≥5%. The TMB-high threshold utilized for analysis in this Example was 175/exome (10 mutations/megabase as measured by FoundationOne® CDx).

To assess TIS, macrodissected FFPE biopsies underwent RNA sequencing to define the tumor transcriptome. Gene-expression data were log base 2 transformed (offset by 0.01) and quantile normalized. TIS was computed as the weighted average of 18 genes included in the GEP score (CXCR6, TIGIT, CD27, CD274 (PD-L1), PDCD1LG2 (PD-L2), LAG3, NKG7, PSMB10, CMKLR1, CD8A, IDO1, CCL5, CXCL9, HLA-DQA1, CD276 (B7-H3), HLA-DRB1, STAT1, and HLA-E). TIS cutoff of 4.56 was used in this Example, based on median values across the combination (vaccine+pembrolizumab) treatment population.

To assess PD-L1, available FFPE biopsies were stained by immunohistochemistry for PD-L1 (22C₃ pharmDx; Agilent/Dako; Santa Clara, CA, USA), and the combined positivity score (CPS) across tumor cells and infiltrating immune cells was used to evaluate PD-L1 expression. PD-L1 positivity was considered as a CPS score of ≥1.

ctDNA was assessed in liquid biopsies (whole-blood samples). ctDNA was not evaluable at baseline for 20.4% (32/157) of patients due to unavailability of the sample at baseline (mRNA-1+pembrolizumab: n=15; pembrolizumab monotherapy: n=14) or insufficient number of variants identified by whole-exome sequencing (mRNA-1+pembrolizumab: n=2; pembrolizumab monotherapy: n=1). The majority of ctDNA-evaluable patients were ctDNA-negative at baseline (88.0% [110/125]; 85.6% [77/90] of the mRNA-1+pembrolizumab group and 94.3% [33/35] of the pembrolizumab monotherapy group). The proportion of ctDNA-positive patients at baseline was slightly higher in the mRNA-1+pembrolizumab arm (14.4% [13/90]) than in the pembrolizumab monotherapy arm (5.7% [2/35]). Baseline disease characteristics and tumor biomarker levels (TMB, TIS, and PD-L1) were generally similar between the ctDNA-evaluable population (n=125) and the overall intention-to-treat (ITT) population (N=157; Table 6).

TABLE 6
Characteristics of study groups by ctDNA status

| ctDNA status | | |
|---|---|---|
| Evaluable | Non-evaluable | ITT |
| (n = 125) | (n = 32) | (N = 157) |

Sex, n (%)

| | | | | | | |
|---|---|---|---|---|---|---|
| Female | 40 | (32.0) | 16 | (50.0) | 56 | (35.7) |
| Male | 85 | (68.0) | 16 | (50.0) | 101 | (64.3) |

Age, years

| | | | | | | |
|---|---|---|---|---|---|---|
| Mean (SD) | 60.9 | (13.3) | 60.2 | (15.5) | 60.7 | (13.7) |
| Median (range) | 63.0 | (26.0, 83.0) | 60.5 | (24.0, 89.0) | 62.0 | (24.0, 89.0) |
| <65, n (%) | 68 | (54.4) | 19 | (59.4) | 87 | (55.4) |
| ≥65, n (%) | 57 | (45.6) | 13 | (40.6) | 70 | (44.6) |

Race, n (%)

| | | | | | | |
|---|---|---|---|---|---|---|
| White | 119 | (95.2) | 32 | (100) | 151 | (96.2) |
| Not reported | 6 | (4.8) | 0 | | 6 | (3.8) |

ECOG PS score, n (%)

| | | | | | | |
|---|---|---|---|---|---|---|
| 0 | 105 | (84.0) | 25 | (78.1) | 130 | (82.8) |
| 1 | 20 | (16.0) | 4 | (12.5) | 24 | (15.3) |

| | | | | | |
|---|---|---|---|---|---|
| Missing | 0 | 3 | (9.4) | 3 | (1.9) |

Disease stage at randomization, n (%)

| Stage IIIC | 103 | (82.4) | 28 | (87.5) | 131 | (83.4) |
|---|---|---|---|---|---|---|
| Stage IIID | 4 | (3.2) | 0 | | 4 | (2.5) |
| Stage IV | 18 | (14.4) | 4 | (12.5) | 22 | (14.0) |

TMB^{a}

| | | | | | | |
|---|---|---|---|---|---|---|
| Mean (SD) | 878 | (1210) | 561 | (634) | 818 | (1130) |
| Median (range) | 400 | (4.00, 7600) | 361 | (2.00, 2020) | 400 | (2.00, 7600) |

| | | | | | |
|---|---|---|---|---|---|
| Missing, n (%) | 0 | 3 | (9.4) | 3 | (1.9) |

TIS

| | | | | | | |
|---|---|---|---|---|---|---|
| Mean (SD) | 4.44 | (1.23) | 4.69 | (1.22) | 4.49 | (1.23) |
| Median (range) | 4.54 | (0.77, 7.14) | 4.95 | (1.83, 6.56) | 4.56 | (0.77, 7.14) |

| | | | | | |
|---|---|---|---|---|---|
| Missing, n (%) | 0 | 3 | (9.4) | 3 | (1.9) |

PD-L1 Combined Positivity Score (CPS) ≥1, n (%)

| | | | | | | |
|---|---|---|---|---|---|---|
| Positive | 79 | (63.2) | 17 | (53.1) | 96 | (61.1) |
| Negative | 15 | (12.0) | 3 | (9.4) | 18 | (11.5) |
| Missing | 31 | (24.8) | 12 | (37.5) | 43 | (27.4) |

^{a}TMB in this Table = the number of nonsynonymous tumor mutations with an allele frequency ≥5%.

Biomarker associations with RFS were evaluated with Kaplan-Meier analyses and assessed with HRs (95% CIs) based on an unstratified Cox proportional hazards model. RFS was defined as the time from first dose of pembrolizumab until the date of first recurrence (local, regional, or distant metastasis), a new primary melanoma, or death from any cause. Data were analyzed by assigned treatment arms and are reported in this Example for biomarker-evaluable patients.

Baseline characteristics of the patients were generally balanced between both study arms across most biomarker subgroups, as shown in Table 7 below.

TABLE 7
Baseline characteristics of TBM, TIS, and PD-L1 expression subgroups
TMB

| Non-High | High |
|---|---|
| mRNA-1 + | mRNA-1 + |

| | | | |
|---|---|---|---|
| pembrolizumab | Pembrolizumab | pembrolizumab | Pembrolizumab |
| (n = 26) | (n = 19) | (n = 79) | (n = 30) |

Sex, n (%)

| | | | | | | | | |
|---|---|---|---|---|---|---|---|---|
| Female | 9 | (34.6) | 7 | (36.8) | 28 | (35.4) | 11 | (36.7) |
| Male | 17 | (65.4) | 12 | (63.2) | 51 | (64.6) | 19 | (63.3) |

Age, years

| | | | | | | | | |
|---|---|---|---|---|---|---|---|---|
| Mean (SD) | 54.7 | (14.0) | 54.4 | (16.3) | 63.8 | (12.5) | 62.2 | (12.2) |
| Median (range) | 56.5 | (29.0, 81.0) | 54.0 | (24.0, 76.0) | 66.0 | (26.0, 83.0) | 62.5 | (40.0, 89.0) |
| <65, n (%) | 21 | (80.8) | 12 | (63.2) | 36 | (45.6) | 16 | (53.3) |
| ≥65, n (%) | 5 | (19.2) | 7 | (36.8) | 43 | (54.4) | 14 | (46.7) |

ECOG PS score, n (%)

| | | | | | | | | |
|---|---|---|---|---|---|---|---|---|
| 0 | 23 | (88.5) | 16 | (84.2) | 67 | (84.8) | 24 | (80.0) |
| 1 | 3 | (11.5) | 3 | (15.8) | 12 | (15.2) | 6 | (20.0) |

Disease stage at randomization, n (%)^{a}

| Stage IIIC | 24 | (92.3) | 16 | (84.2) | 63 | (79.7) | 25 | (83.3) |
|---|---|---|---|---|---|---|---|---|
| Stage IIID | 0 | | 2 | (10.5) | 2 | (2.5) | 0 | |
| Stage IV | 2 | (7.7) | 1 | (5.3) | 14 | (17.7) | 5 | (16.7) |

Number of prior cancer-related surgeries, n (%)^{b}

| | | | | | | | | |
|---|---|---|---|---|---|---|---|---|
| 1 | 11 | (42.3) | 15 | (78.9) | 29 | (36.7) | 9 | (30.0) |
| 2 | 9 | (34.6) | 1 | (5.3) | 26 | (32.9) | 17 | (56.7) |
| >3 | 6 | (23.1) | 3 | (15.8) | 24 | (30.4) | 4 | (13.3) |

LDH, U/L

| | | | | | | | | |
|---|---|---|---|---|---|---|---|---|
| Mean (SD) | 233 | (115) | 189 | (73.9) | 208 | (74.2) | 244 | (201) |
| Median (range) | 189 | (134, 528) | 182 | (113, 454) | 189 | (118, 456) | 187 | (130, 1180) |

| | | | | | | |
|---|---|---|---|---|---|---|
| >ULN, n (%) | 0 | 0 | 5 | (6.3) | 3 | (10.0) |

TIS

| Low | High |
|---|---|
| mRNA-1 + | mRNA-1 + |

| | | | |
|---|---|---|---|
| pembrolizumab | Pembrolizumab | pembrolizumab | Pembrolizumab |
| (n = 55) | (n = 22) | (n = 50) | (n = 27) |

Sex, n (%)

| | | | | | | | | |
|---|---|---|---|---|---|---|---|---|
| Female | 18 | (32.7) | 10 | (45.5) | 19 | (38.0) | 8 | (29.6) |
| Male | 37 | (67.3) | 12 | (54.5) | 31 | (62.0) | 19 | (70.4) |

Age, years

| | | | | | | | | |
|---|---|---|---|---|---|---|---|---|
| Mean (SD) | 63.0 | (12.7) | 58.7 | (16.4) | 60.0 | (14.2) | 59.6 | (12.6) |
| Median (range) | 64.0 | (29.0, 83.0) | 61.5 | (24.0, 89.0) | 61.5 | (26.0, 81.0) | 60.0 | (26.0, 84.0) |
| <65, n (%) | 28 | (50.9) | 13 | (59.1) | 29 | (58.0) | 15 | (55.6) |
| ≥65, n (%) | 27 | (49.1) | 9 | (40.9) | 21 | (42.0) | 12 | (44.4) |

ECOG PS score, n (%)

| | | | | | | | | |
|---|---|---|---|---|---|---|---|---|
| 0 | 48 | (87.3) | 17 | (77.3) | 42 | (84.0) | 23 | (85.2) |
| 1 | 7 | (12.7) | 5 | (22.7) | 8 | (16.0) | 4 | (14.8) |

Disease stage at randomization, n (%)^{a}

| Stage IIIC | 50 | (90.9) | 19 | (86.4) | 37 | (74.0) | 22 | (81.5) |
|---|---|---|---|---|---|---|---|---|
| Stage IIID | 0 | | 1 | (4.5) | 2 | (4.0) | 1 | (3.7) |
| Stage IV | 5 | (9.1) | 2 | (9.1) | 11 | (22.0) | 4 | (14.8) |

Number of prior cancer-related surgeries, n (%)^{b}

| | | | | | | | | |
|---|---|---|---|---|---|---|---|---|
| 1 | 20 | (36.4) | 15 | (68.2) | 20 | (40.0) | 9 | (33.3) |
| 2 | 19 | (34.5) | 5 | (22.7) | 16 | (32.0) | 13 | (48.1) |
| ≥3 | 16 | (29.1) | 2 | (9.1) | 14 | (28.0) | 5 | (18.5) |

LDH, U/L

| | | | | | | | | |
|---|---|---|---|---|---|---|---|---|
| Mean (SD) | 197 | (74.2) | 227 | (124) | 234 | (94.6) | 219 | (194) |
| Median (range) | 180 | (118, 528) | 184 | (113, 590) | 194 | (134, 518) | 185 | (130, 1180) |

| | | | | | | | |
|---|---|---|---|---|---|---|---|
| >ULN, n (%) | 1 | (1.8) | 0 | 4 | (8.0) | 3 | (11.1) |

PD-L1 expression

| Negative | Positive |
|---|---|
| mRNA-1 + | mRNA-1 + |

| | | | |
|---|---|---|---|
| pembrolizumab | Pembrolizumab | pembrolizumab | Pembrolizumab |
| (n = 13) | (n = 5) | (n = 69) | (n = 27) |

Sex, n (%)

| | | | | | | | | |
|---|---|---|---|---|---|---|---|---|
| Female | 7 | (53.8) | 3 | (60.0) | 24 | (34.8) | 8 | (29.6) |
| Male | 6 | (46.2) | 2 | (40.0) | 45 | (65.2) | 19 | (70.4) |

Age, years

| | | | | | | | | |
|---|---|---|---|---|---|---|---|---|
| Mean (SD) | 67.5 | (13.8) | 64.6 | (14.2) | 60.0 | (13.5) | 59.0 | (14.5) |
| Median (range) | 73.0 | (30.0, 81.0) | 61.0 | (52.0, 89.0) | 63.0 | (26.0, 81.0) | 62.0 | (24.0, 82.0) |
| <65, n (%) | 4 | (30.8) | 4 | (80.0) | 41 | (59.4) | 14 | (51.9) |
| ≥65, n (%) | 9 | (69.2) | 1 | (20.0) | 28 | (40.6) | 13 | (48.1) |

ECOG PS score, n (%)

| | | | | | | | | |
|---|---|---|---|---|---|---|---|---|
| 0 | 10 | (76.9) | 4 | (80.0) | 61 | (88.4) | 22 | (81.5) |
| 1 | 3 | (23.1) | 1 | (20.0) | 8 | (11.6) | 5 | (18.5) |

Disease stage at randomization, n (% )^{a}

| Stage IIIC | 12 | (92.3) | 3 | (60.0) | 52 | (75.4) | 23 | (85.2) |
|---|---|---|---|---|---|---|---|---|
| Stage IIID | 0 | | 1 | (20.0) | 2 | (2.9) | 1 | (3.7) |
| Stage IV | 1 | (7.7) | 1 | (20.0) | 15 | (21.7) | 3 | (11.1) |

Number of prior cancer-related surgeries, n (%)^{b}

| | | | | | | | | |
|---|---|---|---|---|---|---|---|---|
| 1 | 7 | (53.8) | 2 | (40.0) | 21 | (30.4) | 16 | (59.3) |
| 2 | 3 | (23.1) | 2 | (40.0) | 25 | (36.2) | 8 | (29.6) |
| ≥3 | 3 | (23.1) | 1 | (20.0) | 23 | (33.3) | 3 | (11.1) |

LDH, U/L

| | | | | | | | | |
|---|---|---|---|---|---|---|---|---|
| Mean (SD) | 274 | (120) | 338 | (196) | 202 | (73.2) | 191 | (62.4) |
| Median (range) | 191 | (163, 438) | 370 | (122, 590) | 186 | (118, 528) | 185 | (113, 454) |

ctDNA Status

| ctDNA-positive | ctDNA-negative |
|---|---|
| mRNA-1 + | mRNA-1 + |

| | | | |
|---|---|---|---|
| pembrolizumab | Pembrolizumab | pembrolizumab | Pembrolizumab |
| (n = 13) | (n = 2) | (n = 77) | (n = 33) |

Sex, n (%)

| Female | 4 | (30.8) | 0 | | 26 | (33.8) | 10 | (30.3) |
|---|---|---|---|---|---|---|---|---|
| Male | 9 | (69.2) | 2 | (100) | 51 | (66.2) | 23 | (69.7) |

Age, years

| | | | | | | | | |
|---|---|---|---|---|---|---|---|---|
| Mean (SD) | 65.0 | (11.4) | 67.5 | (10.6) | 61.2 | (13.9) | 58.0 | (12.4) |
| Median (range) | 67.0 | (46.0, 81.0) | 67.5 | (60.0, 75.0) | 63.0 | (26.0, 83.0) | 61.0 | (26.0, 76.0) |
| <65, n (%) | 6 | (46.2) | 1 | (50.0) | 42 | (54.5) | 19 | (57.6) |
| ≥65, n (%) | 7 | (53.8) | 1 | (50.0) | 35 | (45.5) | 14 | (42.4) |

Race, n (%)

| | | | | | | | | |
|---|---|---|---|---|---|---|---|---|
| White | 13 | (100) | 2 | (100) | 73 | (94.8) | 31 | (93.9) |

| | | | | | | |
|---|---|---|---|---|---|---|
| Not reported | 0 | 0 | 4 | (5.2) | 2 | (6.1) |

ECOG PS score, n (%)

| | | | | | | | | |
|---|---|---|---|---|---|---|---|---|
| 0 | 9 | (69.2) | 1 | (50.0) | 67 | (87.0) | 28 | (84.8) |
| 1 | 4 | (30.8) | 1 | (50.0) | 10 | (13.0) | 5 | (15.2) |

Disease stage at randomization, n (%)^{a}

| | | | | | | | | |
|---|---|---|---|---|---|---|---|---|
| Stage IIIC | 11 | (84.6) | 2 | (100) | 63 | (81.8) | 27 | (81.8) |

| | | | | | | | |
|---|---|---|---|---|---|---|---|
| Stage IIID | 1 | (7.7) | 0 | 1 | (1.3) | 2 | (6.1) |
| Stage IV | 1 | (7.7) | 0 | 13 | (16.9) | 4 | (12.1) |

TMB^{c}

| | | | | | | | | |
|---|---|---|---|---|---|---|---|---|
| Mean (SD) | 608 | (727) | 79.0 | (83.4) | 1060 | (1340) | 619 | (963) |
| Median (range) | 235 | (70.0, 2210) | 79.0 | (20.0, 138) | 563 | (17.0, 7600) | 264 | (4.00, 4700) |

TIS

| | | | | | | | | |
|---|---|---|---|---|---|---|---|---|
| Mean (SD) | 4.08 | (1.21) | 4.34 | (2.67) | 4.48 | (1.25) | 4.49 | (1.16) |
| Median (range) | 4.02 | (2.07, 6.71) | 4.34 | (2.45, 6.23) | 4.59 | (0.77, 7.14) | 4.57 | (2.13, 6.88) |

PD-L1 CPS ≥ 1, n (%)

| | | | | | | | | |
|---|---|---|---|---|---|---|---|---|
| Positive | 9 | (69.2) | 2 | (100) | 49 | (63.6) | 19 | (57.6) |

| | | | | | | | |
|---|---|---|---|---|---|---|---|
| Negative | 2 | (15.4) | 0 | 9 | (11.7) | 4 | (12.1) |
| Missing | 2 | (15.4) | 0 | 19 | (24.7) | 10 | (30.3) |

ECOG PS, Eastern Cooperative Oncology Group performance status; LDH, lactate dehydrogenase; PD-L1, programmed death ligand-1; SD, standard deviation; TIS, tumor inflammation signature; TMB, tumor mutational burden; ULN, upper limit of normal.
^{a}According to the 8th edition of the American Joint Committee on Cancer staging manual.
^{b}Duration from the most recent surgery to the first dose of pembrolizumab is balanced across study arms and biomarker subgroups, with a mean range between approximately 9 and 11 weeks.
^{c}TMB in this Table = number of nonsynonymous tumor mutations with an allele frequency ≥5%.

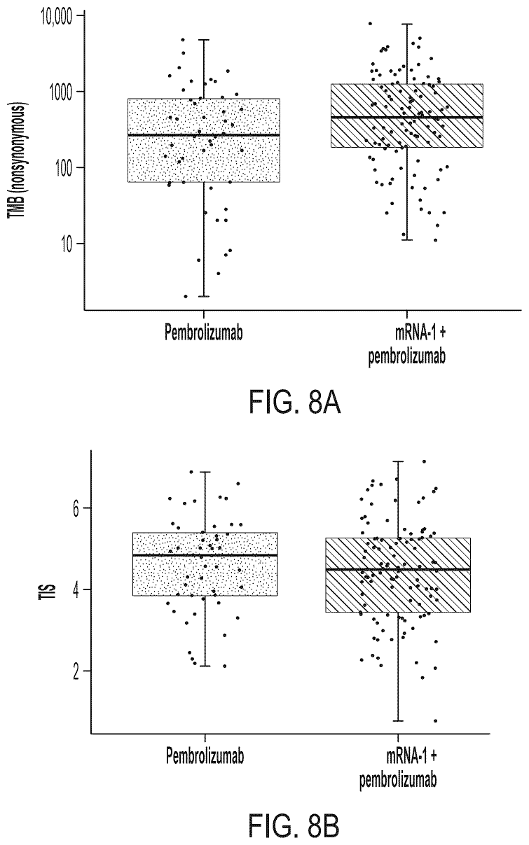

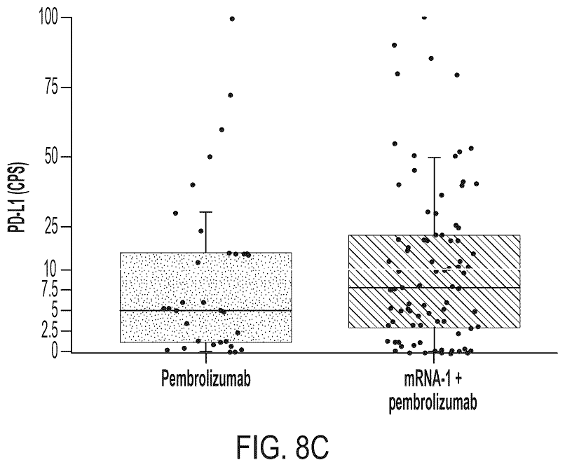

There was a larger subgroup of TMB-high patients in the mRNA-1 and pembrolizumab arm (79/105 [75%]) compared to the pembrolizumab monotherapy arm (30/49 [61%]), as shown in FIG. 8A. The distribution of TIS (FIG. 8B) and PD-L1 expression (FIG. 8C) in baseline tumor biopsies was balanced between study arms. TMB and TIS were evaluable for 154/157 (98.1%) patients. Randomized patients who were never treated in the study and not evaluable for TMB and TIS subgroup analyses (n=3) were not included in the analyses shown in FIGS. 8A and 8B. PD-L1 was evaluable for 114/157 (72.6%) patients, of which 18/114 (15.8%) were PD-L1 negative. Randomized patients who were not evaluable for PD-L1 (n=43) included those with insufficient sample quantity or quality to perform the assay.

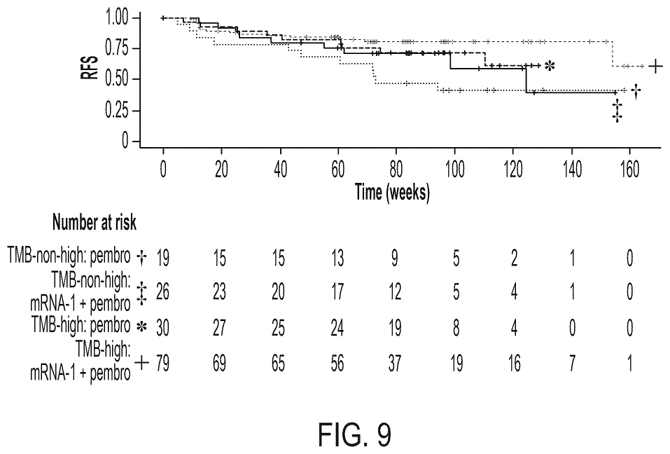

The increased RFS observed with the mRNA-1 and pembrolizumab combination compared to pembrolizumab monotherapy was maintained with a similar magnitude of treatment effect across both high (HR: 0.652; 95% CI: 0.284, 1.494) and non-high (HR: 0.586; 95% CI: 0.243, 1.425) TMB subgroups (FIG. 9 and Table 8). Improved RFS was observed in TMB-high compared to TMB-non-high patient subgroups in the pembrolizumab monotherapy arm. Increased RFS benefit in the TMB-high compared to the TMB-nonhigh subgroup was also observed in the mRNA-1 and pembrolizumab combination arm.

TABLE 8
RFS by treatment arm stratified by TMB-high and TMB-non-high subgroups

| | mRNA-1 + | | | | |
|---|---|---|---|---|---|
| | pembro vs pembro | mRNA-1 + pembro | | Pembro | |
| | HR (95% CI) | #events/n (%) | | #events/n (%) | |
| TMB-high | 0.652 (0.284, 1.494) | 15/79 | (19.0) | 9/30 | (30.0) |
| TMB-non-high | 0.586 (0.243, 1.415) | 9/26 | (34.6) | 11/19 | (57.9) |

| | TMB-high vs TMB- | | | | |
|---|---|---|---|---|---|
| | non-high | TMB-high | | TMB-non-high | |
| | HR (95% CI) | #events/n (%) | | #events/n (%) | |
| mRNA-1 + pembro | 0.536 (0.234, 1.225) | 15/79 | (19.0) | 9/26 | (34.6) |
| Pembro | 0.482 (0.199, 1.166) | 9/30 | (30.0) | 11/19 | (57.9) |

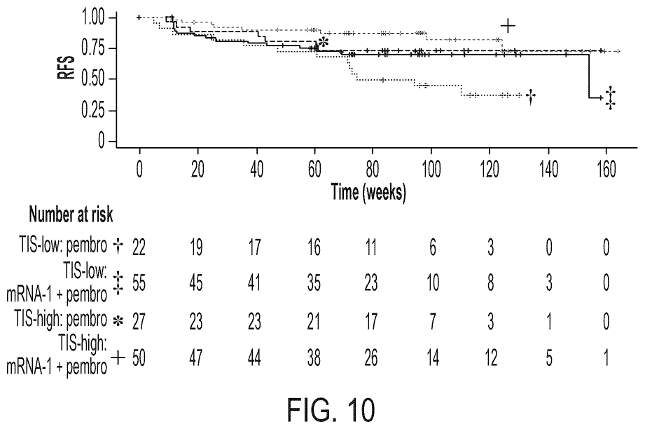

The RFS benefit of mRNA-1 and pembrolizumab was observed in both TIS-high and TIS-low subgroups (FIG. 10 and Table 9). Improved RFS was observed in TIS-high compared to TIS-low subgroups in the pembrolizumab monotherapy arm. The increased RFS benefit in the TIS-high subgroup was also observed in the mRNA-1 and pembrolizumab combination arm.

TABLE 9
RFS by treatment arm stratified by TIS-high and TIS-low subgroups

| | mRNA-1 + | | | | |
|---|---|---|---|---|---|
| | pembro vs pembro | mRNA-1 + pembro | | Pembro | |
| | HR (95% CI) | #events/n (%) | | #events/n (%) | |
| TIS-high | 0.576 (0.209, 1.591) | 8/50 | (16.0) | 7/27 | (25.9) |
| TIS-low | 0.528 (0.253, 1.101) | 16/55 | (29.1) | 13/22 | (59.1) |

| | TIS-high vs TIS-low | TIS-high | TIS-low |
|---|---|---|---|
| | HR (95% CI) | #events/n (%) | #events/n (%) |
| mRNA-1 + pembro | 0.459 (0.196, 1.074) | 8/50 (16.0) | 16/55 (29.1) |
| Pembro | 0.420 (0.167, 1.056) | 7/27 (25.9) | 13/22 (59.1) |

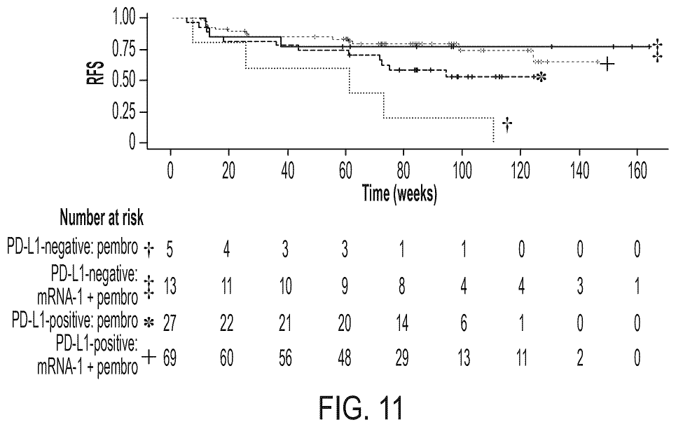

The RFS benefit of mRNA-1 and pembrolizumab compared to pembrolizumab monotherapy was observed in the baseline PD-L1-positive subgroup (FIG. 11 and Table 10). A similar trend was observed for patients with PD-L1-negative baseline tumors; however, the smaller sample size limits the interpretation of these results.

TABLE 10
RFS by treatment arm stratified by PD-L1-positive and PD-L1-negative subgroups

| | mRNA-1 + | | | | |
|---|---|---|---|---|---|
| | pembro vs pembro | mRNA-1 + pembro | | Pembro | |
| | HR (95% CI) | #events/n (%) | | #events/n (%) | |
| PD-L1-positive | 0.485 (0.226, 1.039) | 15/69 | (21.7) | 12/27 | (44.4) |
| PD-L1-negative | 0.162 (0.038, 0.685) | 3/13 | (23.1) | 5/5 | (100) |
| | PD-L1-positive vs | | | | |
| | PD-L1-negative | PD-L1-positive | | PD-L1-negative | |
| | HR (95% CI) | #events/n (%) | | #events/n (%) | |
| mRNA-1 + pembro | 1.085 (0.312, 3.768) | 15/69 | (21.7) | 3/13 | (23.1) |
| Pembro | 0.361 (0.127, 1.029) | 12/27 | (44.4) | 5/5 | (100) |

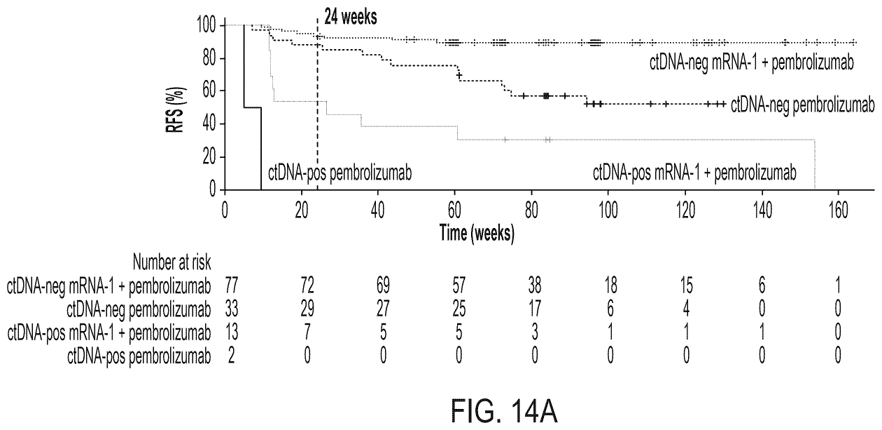

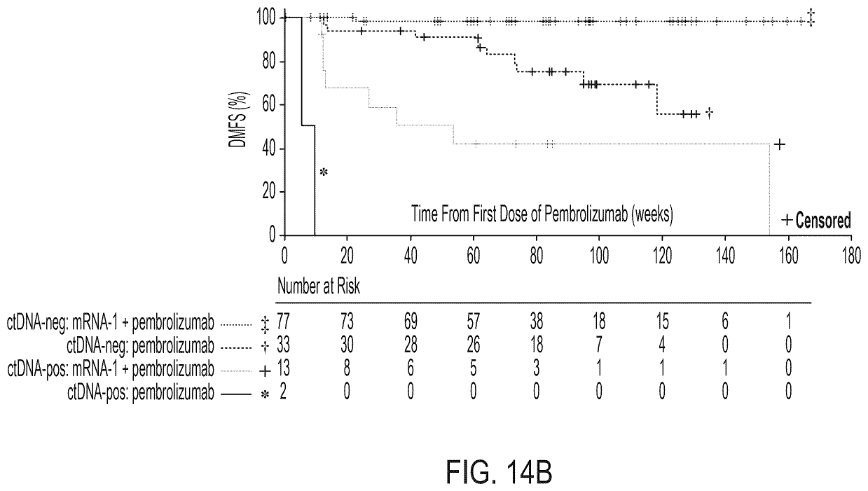

In ctDNA-negative patients at baseline, substantial RFS and DMFS benefits with mRNA-1+pembrolizumab versus pembrolizumab monotherapy were observed (FIGS. 14A and 14B and Table 11). A similar trend was observed for patients in the ctDNA-positive subgroup. In ctDNA-negative patients (n=110), the RFS hazard ratio was 0.254 (95% CI: 0.106, 0.607) with adjustment by TMB versus 0.225 (95% CI: 0.095, 0.531) without adjustment by TMB, suggesting that the RFS treatment effect was robust and not driven by TMB.

TABLE 11
RFS and DMFS by treatment arm stratified by
ctDNA-positive and ctDNA-negative subgroups
RFS

| | mRNA-1 + | | | | |
|---|---|---|---|---|---|
| | pembro vs pembro | mRNA-1 + pembro | | Pembro | |
| | HR (95% CI) | events, n/N (%) | | events, n/N (%) | |
| ctDNA-pos | Non-evaluable | 10/13 | (76.9) | 2/2 | (100) |
| ctDNA-neg | 0.225 (0.095, 0.531) | 8/77 | (10.4) | 15/33 | (45.5) |

DMFS

| | mRNA-1 + | | | | |
|---|---|---|---|---|---|
| | pembro vs pembro | mRNA-1 + pembro | | Pembro | |
| | HR (95% CI) | events, n/N (%) | | events, n/N (%) | |
| ctDNA-pos | Non-evaluable | 8/13 | (61.5) | 2/2 | (100) |
| ctDNA-neg | 0.048 (0.006, 0.380) | 1/77 | (1.3) | 9/33 | (27.3) |

Assessments of the association of various biomarkers with RFS across study arms suggested that ctDNA status at baseline had notable negative prognostic value (RFS HR by ctDNA status: 0.149; 95% CI: 0.073, 0.306) in patients with resected high-risk melanoma.

In the present study in patients with resected high-risk stage III/IV melanoma, across study arms, higher TMB and TIS values were associated with better RFS compared to lower values.

Irrespective of TMB status, the results indicate that targeting an individual patient's unique tumor mutations with mRNA-1 demonstrates improved RFS when administered in combination with pembrolizumab compared to pembrolizumab monotherapy. The imbalance in the TMB-high subpopulation of the mRNA-1 and pembrolizumab combination arm is unlikely to have impacted the clinical benefit observed in this study arm over pembrolizumab monotherapy, as the trends in magnitude of added RFS benefit in TMB-high and TMB-non-high subpopulations are similar.

The novel mechanism of action of mRNA-1 and increased RFS with mRNA-1 and pembrolizumab observed in the TMB-high and TMB-non-high populations, TIS-high and TIS-low populations, and in the PD-L1-positive population suggest that mRNA-1 may both deepen the activity of pembrolizumab (biomarker-high populations) and broaden the population of patients who can benefit from immune therapy (biomarker-low populations).

### Example 6

This Example describes a study of adjuvant mRNA-1 or placebo with pembrolizumab in patients with high-risk stage II-IV melanoma.

Pembrolizumab, an anti-PD-1 antibody, is approved as adjuvant therapy for stage IIB-C and stage III melanoma by American Joint Committee on Cancer (AJCC) Cancer Staging Manual 8th edition, following complete resection. Adjuvant pembrolizumab has improved recurrence-free survival (RFS) and distant metastasis-free survival (DMFS) in patients with high-risk melanoma, but many patients experience disease recurrence. mRNA-1 is an individualized neoantigen therapy that showed improved RFS and DMFS when used in combination with pembrolizumab compared with pembrolizumab alone in patients with stage III/IV melanoma in the randomized phase 2b KEYNOTE-942 study.

A randomized, double-blind, phase 3 study is conducted to evaluate the efficacy and safety of adjuvant pembrolizumab+mRNA-1 versus pembrolizumab+placebo in patients with resected high-risk stage II-IV melanoma. Eligible patients are ≥18 years of age, with surgically resected stage IIB or IIC (pathologic or clinical), III, or IV cutaneous melanoma per AJCC 8th edition, and have an Eastern Cooperative Oncology Group performance status of 0 or 1. Patients have not received any prior systemic therapy, and no more than 13 weeks have elapsed between last surgical resection and first dose of pembrolizumab. Patients with ocular or mucosal melanoma and past or current in-transit metastases or satellitosis are excluded. All patients provide a blood sample and a FFPE tumor sample for sequencing. Patients are stratified by risk (IIB, IIC, IIIA, and IIIB vs IIIC/D and IV) and age (<65 years vs ≥65 years). Approximately 1089 patients are randomly assigned 2:1 to receive pembrolizumab (400 mg) intravenously every 6 weeks with either (i) mRNA-1 (1 mg) or (ii) placebo intramuscularly every 3 weeks for 9 doses or until disease recurrence, unacceptable toxicity, or withdrawal.

The primary end point is RFS by investigator review. Secondary end points are DMFS by investigator review, overall survival, safety and tolerability, and quality of life. Hazard ratios and 95% CIs are estimated using a stratified Cox regression model with the Efron method of handling ties. Between-treatment differences are evaluated using a stratified log-rank test.

### Example 7

This example describes results of analysis of clinical data emerging from a phase 2 trial evaluating the efficacy of a combination of mRNA-1, an individualized neoantigen therapy (INT), with pembrolizumab.

In the phase 2 Keynote-942 trial, the combination of mRNA-1 with pembrolizumab demonstrated enhanced clinical efficacy in patients with resected high-risk stage IIIB-IV melanoma, meeting the primary and secondary objectives of extended recurrence-free survival (RFS) and distant metastasis-free survival (DMFS) when compared to pembrolizumab alone treatment (see, e.g., Examples 1-5). Minimal residual disease (MRD) detection by plasma circulating tumor DNA (ctDNA) at the start of treatment was associated with shorter RFS. This Example investigates ctDNA longitudinal dynamics and associated clinical outcomes. RFS by ctDNA and BRAF status were also investigated.

Eligible patients were assigned 2:1 (stratified by stage) to receive mRNA-1 in combination with pembrolizumab or pembrolizumab alone. RaDaR® (Inivata) was used to measure ctDNA in plasma samples collected during treatment and follow-up. BRAF mutation status was derived from whole exome sequencing using baseline tumor tissue. Longitudinal ctDNA patterns were classified into two categories: Disease Control (DC) and No Disease Control (NDC). Classification into the DC category indicates that MRD (ctDNA-positive from treatment start) or molecular recurrence (MR, ctDNA-negative at treatment initiation with subsequent ctDNA-positive detection) were resolved during treatment (e.g., ctDNA-positive detection from treatment start or during the course of treatment resolved such that the patient later became ctDNA-negative), and classification into the NDC category indicates that MRD or MR were not resolved (e.g., ctDNA-positive detection from treatment start or during the course of treatment remained positive following treatment). Kaplan-Meier analysis was used to estimate survival curves for RFS and DMFS, and the Cox proportional hazards regression was used for HR estimation.

Across treatment arms, longitudinal ctDNA characteristics were associated with different recurrence outcomes. Disease recurrence was observed in 63% (10/16) of patients classified into the DC category and 86% (12/14) of patients classified into NDC category. Local recurrence was more common in DC patients, and distant events were more common in NDC patients. This trend was further supported by a stronger separation in DMFS compared to RFS between the DC and NDC populations. DC patterns were observed in 88% (14/16) of patients treated with combination (INT+pembrolizumab) and 12% (2/16) of patients treated with pembrolizumab monotherapy. A trend for enhanced efficacy of the combination was observed across BRAF WT and mutant subpopulations (HR=0.808; 95% CI (0.366, 1.784) vs HR=0.332; 95% CI (0.130, 0.850) respectively), with ctDNA status influencing response characteristics. Overall, ctDNA status demonstrated a strong prognostic value across various status definitions, with the detection of ctDNA in a patient sample associated with poorer outcomes. This included detection of ctDNA at treatment initiation, within 90 days from treatment initiation, within 180 days from treatment initiation, and within all available timepoints.

In addition, some ctDNA-positive longitudinal patterns displayed less clinical recurrence. Move favorable patterns, including cdDNA-positivity but with disease control following treatment, were observed primarily in the combination treatment group, and more frequently resulted in no detected disease recurrence; similarly, no disease recurrence occurred predominantly in the combination treatment group. By contrast, less favorable patterns, including ctDNA-positivity without disease control following treatment, were observed primarily in the pembrolizumab monotherapy treatment group, and more frequently resulted in disease recurrence.

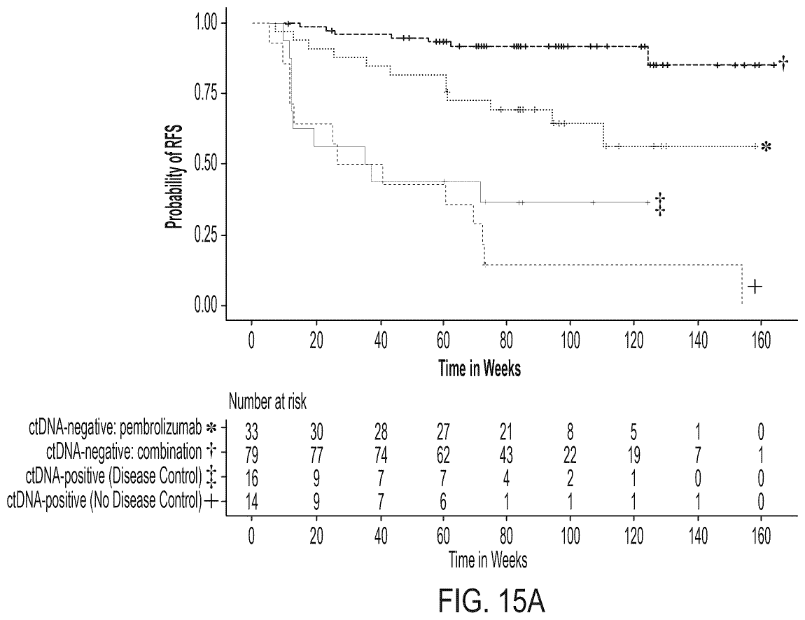

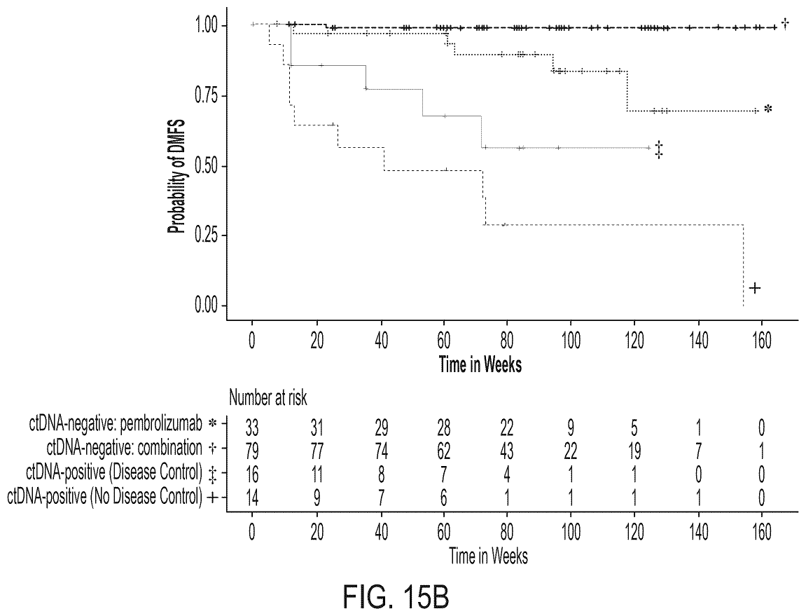

A further comparison of recurrence-free survival (RFS) and distant metastasis-free survival (DMFS) in DC and NDC categorized patients demonstrated that favorable response patterns correlated with DMFS. RFS (FIG. 15A) and DMFS (FIG. 15B) were evaluated in ctDNA-negative patients in both pembrolizumab monotherapy and mRNA-1+pembrolizumab combination therapy treatment groups, in addition to ctDNA-positive patients classified into the DC and NDC categories. Comparing patients with DC patterns with those with NDC patterns via RFS and DMFS endpoint analyses demonstrated that DMFS showed a larger separation between the two classes of ctDNA-positive patients than did RFS. These data suggest that longitudinal ctDNA patterns may both capture treatment effects and predict overall survival.

Additional analyses of RFS and DMFS data were conducted for based on ctDNA status. ctDNA-negative patients (patients who were never ctDNA positive) were compared to ctDNA positive patients who showed molecular responder (MR) phenotypes, and ctDNA positive patients who showed molecular non-responder (MNR) phenotypes. Patients in whom ctDNA was detected at some point during the course of the study (e.g., at the start, or sometime during the course of treatment) but in whom ctDNA was not detected at the final time point were categorized as having an MR phenotype. Patients in whom ctDNA was detected at some point during the course of the study (e.g., at the start, or sometime during the course of treatment) and who were ctDNA positive at the final time point were categorized as having an MNR phenotype.

Recurrence events (defined as the first recurrence [local, regional, or distant metastasis], a new primary melanoma, or death from any cause) were detected in 19/112 (17%) of ctDNA negative patients; in 8/14 (57%) of ctDNA positive MR patients, and in 15/16 (94%) of ctDNA positive MNR patients. Higher distant recurrence rates were observed in MNR patients than in MR patients, suggesting that ctDNA may be prognostic. Of patients in whom recurrence was detected, local/regional/new primary melanoma recurrence events were detected in 15/19 (79%) of ctDNA negative patients; in 5/8 (62.5%) of ctDNA positive MR patients, and in 6/15 (40%) of ctDNA positive MNR patients; distant metastatic recurrence events (defined as any site beyond regional draining nodes or cutaneous >2 cm beyond tumor bed) were detected in 3/19 (16%) of ctDNA negative patients; in 3/8 (37.5%) of ctDNA positive MR patients, and in 9/15 (60%) of ctDNA positive MNR patients; and death occurred in 1/19 (5%) of ctDNA negative patients (death was attributed to sepsis, unrelated to mRNA-1 or pembrolizumab); in 0/8 (0%) of ctDNA positive MR patients, and in 0/15 (0%) of ctDNA positive MNR patients.

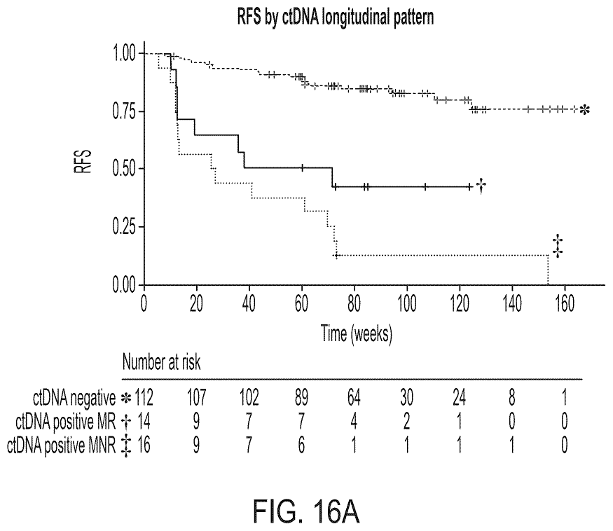

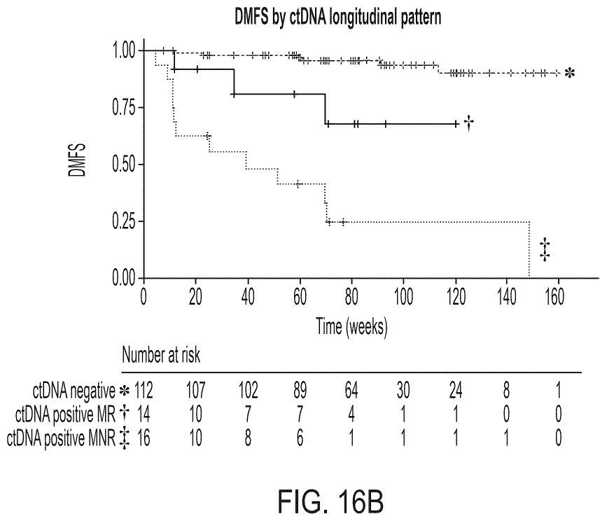

Kaplan-Meier curves for patients stratified by ctDNA status (ctDNA negative, ctDNA positive with MR phenotype, or ctDNA positive with MNR phenotype) are shown in FIG. 16A (RFS) and FIG. 16B (DMFS). The hazard ratio for RFS (MR vs MNR) was 0.535 (0.224-1.278 95% CI), and for DMFS (MR vs MNR) was 0.274 (0.076-0.984 95% CI).

These findings underscore the potential impact of ctDNA dynamics on treatment outcomes in patients with high-risk resectable melanoma and suggest INT in combination with pembrolizumab can clear MRD and reverse molecular recurrence in patients.

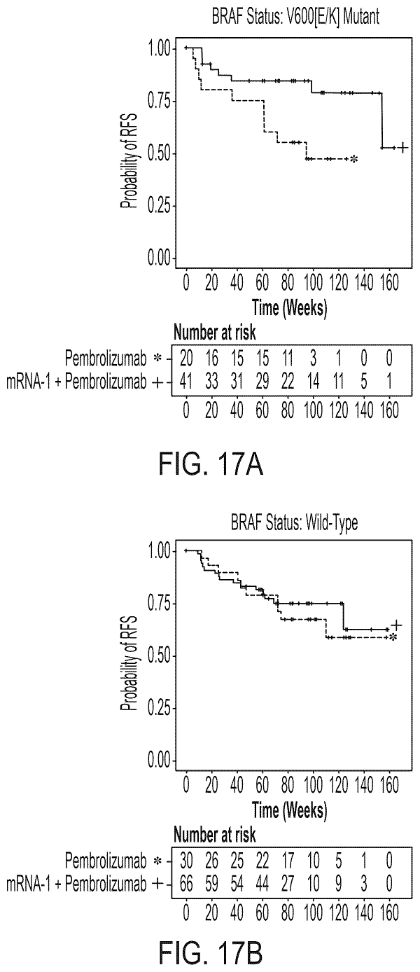

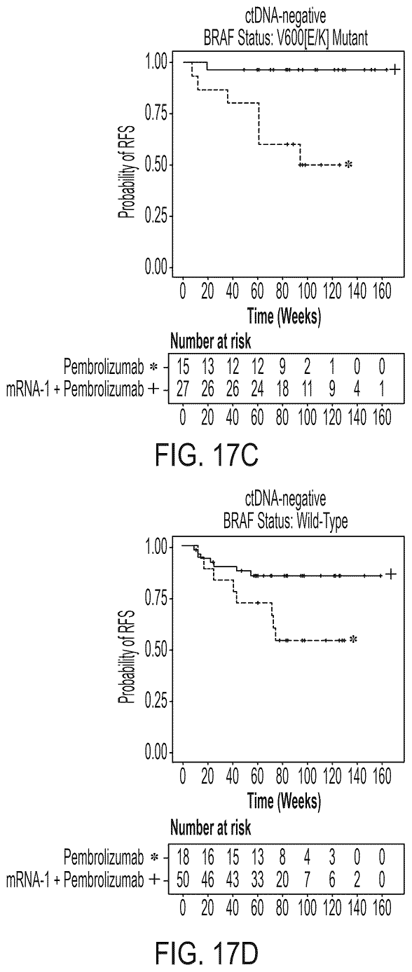

Additional analyses of the clinical data were performed to investigate effects of BRAF mutant status on outcomes. BRAF V600 E/K mutants were equally distributed across the two treatment arms (pembrolizumab: V600 [E/K] 20/50 (40%), wild-type 30/50 (60%); combination: V600 [E/K] 41/107 (38.3%), wild-type 66/107 (61.7%)). Combination treatment with mRNA-1 and pembrolizumab improved RFS in patients with either BRAF wild-type or BRAF V600 [E/K] mutated tumors, and the added benefit of the combination treatment compared to the pembrolizumab monotherapy was stronger in patients with BRAF mutated tumors than in patients with BRAF wild-type tumors (FIG. 17A and FIG. 17B; Table 12). Consistent with previous reports, BRAF mutant status did not impact RFS in the pembrolizumab monotherapy treatment group.

TABLE 12

| BRAF | | Events/N | Median Survival, | HR | |
|---|---|---|---|---|---|
| Status | Arm | (%) | weeks (95% CI) | (95% CI) | P |
| V600[E/K] | Pembro | 10/20 | 94.3 | | |
| mutant | | (50.0%) | (61.0, NA) | | |
| | mRNA-1 + | 8/41 | NA | 0.332 | 0.013 |
| | Pembro | (19.5%) | (153.9, NA) | (0.13, 0.85) | |
| Wild Type | Pembro | 10/30 | NA | | |
| | | (33.3%) | (110.3, NA) | | |
| | mRNA-1 + | 16/66 | NA | 0.808 | 0.604 |
| | Pembro | (24.2%) | (124.3, NA) | (0.366, 1.784) | |

Baseline patient characteristics in the BRAF V600 [E/K] mutant and BRAF wild-type subgroups included some imbalances (Table 13). In the BRAF V600 [E/K] mutant subpopulation (38.9% of patients), the baseline disease characteristics were relatively balanced, with only a slightly higher percentage of patients with high TIS in the combination therapy treatment arm. In the BRAF wild-type subpopulation (61.1% of patients), while there were no ctDNA-positive patients at baseline in the pembrolizumab monotherapy treatment arm, 12.1% of the patients in the mRNA-1+pembrolizumab combination treatment arm were ctDNA-positive at baseline. In addition, there was a higher proportion of patients with PD-L1 CPS≥1 in the combination arm than in the monotherapy arm (66.7% vs 46.7%), mean and median TMB were higher in the combination arm relative to the monotherapy arm, and a lower proportion of patients in the combination therapy arm exhibited low TIS relative to the monotherapy arm.

TABLE 13
Baseline characteristics of BRAF status subgroups

| V600[E/K] Mutant BRAF | | Wild-Type BRAF | |
|---|---|---|---|
| Pembro | Combination | Pembro | Combination |
| (N = 20) | (N = 41) | (N = 30) | (N = 66) |

SEX

| | | | | | | | | |
|---|---|---|---|---|---|---|---|---|
| F | 8 | (40.0%) | 15 | (36.6%) | 11 | (36.7%) | 22 | (33.3%) |
| M | 12 | (60.0%) | 26 | (63.4%) | 19 | (63.3%) | 44 | (66.7%) |

AGE

| | | | | | | | | |
|---|---|---|---|---|---|---|---|---|
| Mean (SD) | 55.1 | (14.0) | 57.8 | (12.2) | 62.3 | (13.9) | 63.6 | (13.9) |

| | | | | |
|---|---|---|---|---|
| Median | 59.0 | 58.0 | 65.5 | 65.0 |
| [Min, Max] | [24.0, 75.0] | [30.0, 80.0] | [32 .0, 89.0] | [26.0, 83.0] |

RACE

| | | | | | | | | |
|---|---|---|---|---|---|---|---|---|
| White | 20 | (100%) | 40 | (97.6%) | 28 | (93.3%) | 63 | (95.5%) |
| Not Reported | 0 | (0%) | 1 | (2.4%) | 2 | (6.7%) | 3 | (4.5%) |

ECOG

| | | | | | | | | |
|---|---|---|---|---|---|---|---|---|
| 0 | 17 | (85.0%) | 34 | (82.9%) | 23 | (76.7%) | 56 | (84.8%) |
| 1 | 3 | (15.0%) | 5 | (12.2%) | 6 | (20.0%) | 10 | (15.2%) |
| No Data | 0 | (0%) | 2 | (4.9%) | 1 | (3.3%) | 0 | (0%) |

STAGE

| | | | | | | | | |
|---|---|---|---|---|---|---|---|---|
| IIIC | 18 | (90.0%) | 37 | (90.2%) | 24 | (80.0%) | 52 | (78.8%) |
| IV | 2 | (10.0%) | 4 | (9.8%) | 4 | (13.3%) | 12 | (18.2%) |
| IIID | 0 | (0%) | 0 | (0%) | 2 | (6.7%) | 2 | (3.0%) |

No. of Prior Cancer Related Surgeries

| | | | | | | | | |
|---|---|---|---|---|---|---|---|---|
| >=3 | 3 | (15.0%) | 8 | (19.5%) | 5 | (16.7%) | 22 | (33.3%) |
| 1 | 12 | (60.0%) | 19 | (46.3%) | 12 | (40.0%) | 22 | (33.3%) |
| 2 | 5 | (25.0%) | 14 | (34.1%) | 13 | (43.3%) | 22 | (33.3%) |

TIS

| Mean (SD) | 4.31 | (1.28) | 4.48 | (1.23) | 4.81 | (1.08) | 4.40 | (1.27) |
|---|---|---|---|---|---|---|---|---|
| Median | 4.30 | | 4.50 | | 5.01 | | 4.47 | |
| [Min, Max] | [2.19, 6.23] | | [2.13, 6.56] | | [2.13, 6.88] | | [0.772, 7.14] | |
| No Data | 0 | (0%) | 2 | (4.9%) | 1 | (3.3%) | 0 | (0%) |

TMB

| Mean (SD) | 369 | (358) | 412 | (442) | 776 | (1080) | 1210 | (1420) |
|---|---|---|---|---|---|---|---|---|
| Median | 235 | | 273 | | 361 | | 737 | |
| [Min, Max] | [20.0, 1330] | | [11.0, 2100] | | [2.00, 4700] | | [13.0, 7600] | |
| No Data | 0 | (0%) | 2 | (4.9%) | 1 | (3.3%) | 0 | (0%) |

TMB (log10)

| Mean (SD) | 2.33 | (0.513) | 2.38 | (0.500) | 2.29 | (0.951) | 2.73 | (0.666) |
|---|---|---|---|---|---|---|---|---|
| Median | 2.37 | | 2.44 | | 2.56 | | 2.87 | |
| [Min, Max] | [1.30, 3.12] | | [1.04, 3.32] | | [0.301, 3.67] | | [1.11, 3.88] | |
| No Data | 0 | (0%) | 2 | (4.9%) | 1 | (3.3%) | 0 | (0%) |

PD-L1 CPS >= 1

| | | | | | | | | |
|---|---|---|---|---|---|---|---|---|
| Negative | 2 | (10.0%) | 5 | (12.2%) | 3 | (10.0%) | 8 | (12.1%) |
| Positive | 13 | (65.0%) | 25 | (61.0%) | 14 | (46.7%) | 44 | (66.7%) |
| No Data | 5 | (25.0%) | 11 | (26.8%) | 13 | (43.3%) | 14 | (21.2%) |

ctDNA Status

| | | | | | | | | |
|---|---|---|---|---|---|---|---|---|
| Not Detected | 15 | (75.0%) | 27 | (65.9%) | 18 | (60.0%) | 50 | (75.8%) |
| Detected | 2 | (10.0%) | 5 | (12.2%) | 0 | (0%) | 8 | (12.1%) |
| No Data | 3 | (15.0%) | 9 | (22.0%) | 12 | (40.0%) | 8 | (12.1%) |

Further analysis of BRAF mutant and wild-type subgroups demonstrated that the treatment benefit of mRNA-1 combination treatment with pembrolizumab relative to pembrolizumab monotherapy was maintained in ctDNA-negative patients (110/157 total patients) in both BRAF V600 [E/K] mutant and BRAF wild-type subgroups (FIG. 17C and FIG. 17D; Table 14). At baseline, BRAF mutational status were similar between the ctDNA-negative patients in the pembrolizumab monotherapy treatment group and the combination treatment group (pembrolizumab: V600 [E/K] 15/33 (45.5%), wild-type 18/33 (54.5%); combination: V600 [E/K] 27/77 (35.1%), wild-type 50/77 (64.9%)).

TABLE 14

| BRAF | | Events/N | Median Survival, | HR | |
|---|---|---|---|---|---|
| Status | Arm | (%) | weeks (95% CI) | (95% CI) | P |
| V600[E/K] | Pembro | 7/15 | 94.3 | | |
| mutant | | (46.7%) | (61.0, NA) | | |
| | mRNA-1 + | 1/27 | NA | 0.069 | 0.001 |
| | Pembro | (3.7%) | (NA, NA) | (0.009, 0.563) | |
| Wild Type | Pembro | 8/18 | NA | | |
| | | (44.4%) | (72.1, NA) | | |
| | mRNA-1 + | 7/50 | NA | 0.334 | 0.025 |
| | Pembro | (14.0%) | (NA, NA) | (0.121, 0.923) | |

These data show that combination treatment of mRNA-1 with pembrolizumab improves outcomes in patients with BRAF wild-type and BRAF V600 [E/K] mutant tumors when compared to pembrolizumab treatment alone.

Further analysis of a particular patient responsive to combination treatment of mRNA-1 with pembrolizumab was conducted. The study characterized T cell responses and the change in target lesion size in the patient. Target lesion size was monitored over the course of the study, from the initiation of treatment with pembrolizumab, through combination treatment with pembrolizumab and mRNA-1, and through the following pembrolizumab monotherapy. T cell responsiveness to neoantigens was also tested in samples collected in each of the time windows mentioned in the previous sentence, and at 100 days of follow-up (100 days following the end of treatment).

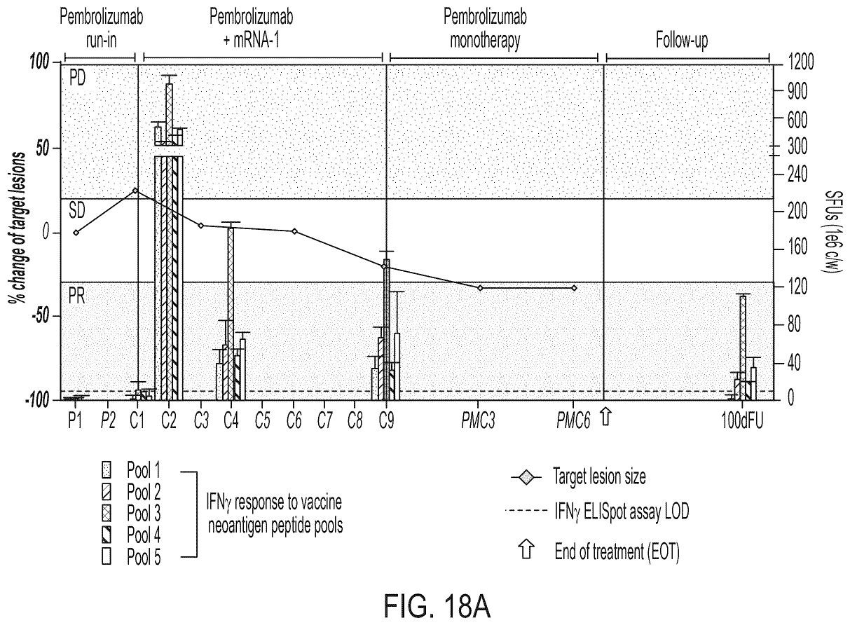

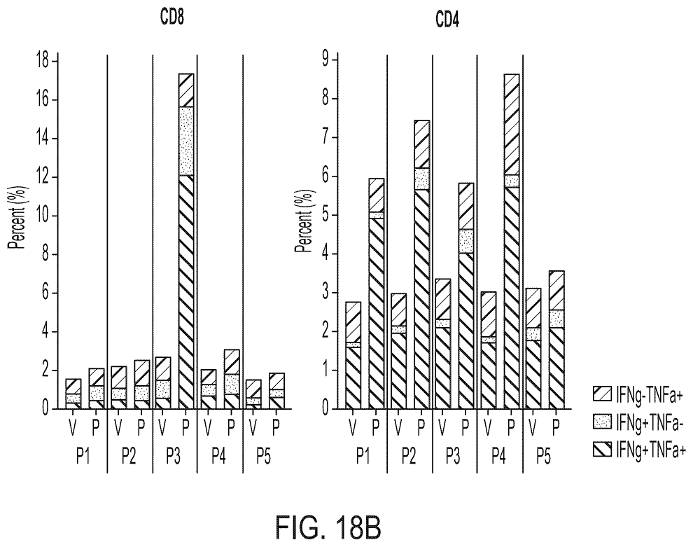

The neoantigens of the subject's vaccine were split into five (5) pools, and T cell response to each vaccine pool was measured ex vivo using IFNγ ELISpot assays, using samples collected at five distinct timepoints: at pembrolizumab run-in administration 1 (“P1”), at combination treatment administration 1 (“C1”), at combination treatment administration 2 (“C2”), at combination treatment administration 4 (“C4”), at combination treatment administration 9 (“C9” the final combination treatment administration for this subject), and at 100 days follow-up (“100dFU”, 100 days following the end of treatment). Target lesion size was also measured at each pembrolizumab run-in administration (“P1” and “P2”), at combination treatment administrations 1, 3, 6, and 8 (“C1”, “C3”, “C6”, and “C9”, respectively), and at both post-combination pembrolizumab monotherapy administrations (“PMC3” and “PMC6”). The results demonstrated that the peak of T cell responses to vaccine neoantigen pools was concurrent with tumor regression amounting to a clinical response. In addition, T cell responses to all 5 neoantigen pools were only detected following mRNA-1 administration (not during the pembrolizumab run-in period), and T cell responses to 4 of the 5 neoantigen pools were still measurable 35 weeks after the last mRNA-1 administration. These data are shown in FIG. 18A.

Neoantigen pool-specific T cells were also analyzed for their phenotype in sample collected on the fourth administration of the combination treatment (“C4”). T cell samples were expanded in the presence of a neoantigen peptide pool (one of five), and then were restimulated with either vehicle (“V”) or the same neoantigen pool (“P”) that they were expanded in. Following expansion and restimulation, CD8+ and CD4+ T cells were characterized for their phenotypes, by identifying IFNγ TNFα⁺ cells, IFNγ⁺TNFα⁺ cells, and IFNγ⁺TNFα⁺ cells. The results shown in FIG. 18B demonstrate that both class I and class II polyfunctional neoantigen pool-specific T cells were detected. ctDNA clearance was also observed in this patient during concurrent pembrolizumab and mRNA-1 treatment.

### Example 8

This example describes T cell responses to individualized neoantigen therapy mRNA-1 as monotherapy or in combination with pembrolizumab.

T-cell targeting of mutation-derived epitopes (neoantigens) has been demonstrated to drive anti-tumor responses. Developing therapies against such neoantigens either as monotherapy or in combination with a checkpoint inhibitor (CPI) may elicit greater anti-tumor responses than CPIs alone. The combination of the novel individual neoantigen therapy (INT) mRNA-1 and pembrolizumab improved recurrence free survival and demonstrated a manageable safety profile compared with pembrolizumab monotherapy in patients with resected high-risk stage III/IV cutaneous melanoma in a randomized Phase 2 study (see, e.g., Examples 1-5). This Example describes results from a first-in-human Phase 1 study of mRNA-1±pembrolizumab to characterize the mechanism of action in patients with resected non-small cell lung cancer (NSCLC) or resected cutaneous melanoma.

In patients with resected stage IIIB-IV melanoma, mRNA-1+pembrolizumab showed clinically meaningful benefit versus pembrolizumab alone. This Example in part reports mechanism of action (MoA) characterization from a first-in-human phase 1 study of mRNA-1+pembrolizumab. Patients with resected NSCLC (Part A; 1 mg mRNA-1, n=4) or resected cutaneous melanoma (Part D; 1 mg mRNA-1 and 200 mg pembrolizumab, n=12) were included. Safety, tolerability, and immunogenicity were assessed. All patients experienced ≥1 treatment-emergent adverse event (AE); there were no grade 4/5 AEs or dose-limiting toxicities. In both Parts, 1 mg mRNA-1 induced consistent T cell responses and strengthened pre-existing low T cell responses to targeted neoantigens. Following combination therapy, sustained mRNA-1-induced neoantigen-specific T cell responses and expansion of cytotoxic CD8 and CD4 T cells were observed. These results demonstrate immunogenicity of mRNA-1 in patients with resected NSCLC or melanoma, support the hypothesized MoA, and show the potential of mRNA technology in oncology.

Baseline tumor core biopsies and matched whole blood from patients with resected NSCLC (cohort A, treated with 1 mg mRNA-1) and resected cutaneous melanoma (cohort D, treated with 1 mg mRNA-1 and 200 mg pembrolizumab), were subjected to whole exome sequencing. T cell immunogenicity was used to examine the immune response to mRNA-1 via antigen-specific T cell assays in peripheral blood mononuclear cells. T cell responses to INT neoantigen pools or to individual neoantigens were analyzed directly ex vivo using IFNγ Enzyme-linked ImmunoSpot at longitudinal study timepoints. Characterization of neoantigen-specific responding CD4 and/or CD8 T cells was enabled through restimulation of expanded cells followed by intracellular cytokine staining.

Four patients were assessed in cohort A and 12 patients in cohort D. There was consistent induction of T cell responses with 1 mg mRNA-1 in both cohorts and strengthening of pre-existing T cell responses to targeted neoantigens. Longitudinal immunogenicity analysis showed sustained mRNA-1-induced neoantigen-specific T cell responses. Pre-existing neoantigen responses observed in patients prior to treatment or following pembrolizumab run-in were increased in the combination treatment compared with monotherapy. Polyfunctional neoantigen specific T cells were induced in all patients tested. Combination treatment increased Granzyme B expression of effector memory T cells and circulating CD45RA+ effector-type T cells compared with monotherapy. Patients who demonstrated robust T cell responses also remained on the study longer, whereas patients with weaker T cell responses progressed.

Advances in cancer immunotherapy have resulted in the approval of several immune checkpoint inhibitors (CPIs) that provide substantial clinical benefit for many patients. Clinical response to anti-programmed cell death (PD-1) therapy is correlated with the magnitude of reinvigoration of exhausted tumor-specific T cells relative to pretreatment tumor burden. However, more than half of patients who receive CPI therapy do not respond, and evidence suggests that an immunosuppressive tumor microenvironment (TME) and insufficient T cell priming within the TME contribute to this unresponsiveness. This leaves an unmet need for novel treatment approaches that mobilize T cells.

Somatic mutations within the tumor's genome result in the creation of tumor-specific antigens that are not present within the normal proteome. These neoantigens can be recognized by the immune system and are ideal targets for personalized immunotherapeutic interventions that aim to mobilize neoantigen specific T cells against the tumor. Initial studies using synthetic peptides to target neoantigen epitopes successfully induced tumor-specific T cell responses and enhanced anti-tumor efficacy in preclinical models (see, e.g., Castle, et al. “Exploiting the mutanome for tumor vaccination” Cancer Res. 72(5): 1081-91 (2012), and Yadav M., et al. “Predicting immunogenic tumour mutations by combining mass spectrometry and exome sequencing” Nature 515(7528): 572-76(2014)). In patients with unresectable or metastatic solid tumors, a peptide-based neoantigen vaccine in combination with a CPI augmented de novo neoantigen-specific T cell induction and expansion of pre-existing responses (Carreno, et al. “Cancer immunotherapy. A dendritic cell vaccine increases the breadth and diversity of melanoma neoantigen-specific T cells” Science 348(6236):803-8 (2015); Ott, et al. “A Phase Ib Trial of Personalized Neoantigen Therapy Plus Anti-PD-1 in Patients with Advanced Melanoma, Non-small Cell Lung Cancer, or Bladder Cancer” Cell 183(2):347-362.e24 (2020)). However, the first generation neoantigen vaccines based on synthetic peptides were limited by the time consuming and costly processing and manufacturing process.

More recent advances in the mRNA platform have resulted in faster manufacturing times, greater flexibility in immunogen design, and robust immunogenicity. Building on these advances, mRNA-1 is an mRNA based individualized neoantigen therapy (INT) that encodes up to 34 patient-specific immunogenic tumor neoantigens derived using an algorithm based on whole exome sequencing (WES) and RNA-sequencing (RNA-seq) of patient tumor and blood samples. While unprotected mRNA is rapidly degraded in biological fluids, unlikely to persist in tissues, unable to integrate into genomic DNA, and unlikely to enter the nucleus via the route administered, mRNA-1 is formulated within a novel lipid nanoparticle (LNP) and delivered intramuscularly. The patient-specific neoantigens are endogenously translated and enter the cellular antigen processing and presentation pathway, inducing robust T cell responses.

In an open-label, randomized, phase 2 study, mRNA-1 in combination with pembrolizumab demonstrated a clinically meaningful benefit in recurrence-free survival (RFS) and distant metastasis-free survival (DMFS) versus pembrolizumab monotherapy, with a manageable safety profile, in patients with high risk resected stage IIIB-IV melanoma (See, e.g., Examples 1-5). Exploratory analyses in patients with evaluable baseline circulating tumor DNA (ctDNA) revealed a trend of prolonged RFS with mRNA-1 in combination with pembrolizumab compared with mRNA-1 monotherapy, regardless of ctDNA status (positive or negative). To inform the mechanism of action of mRNA-1 and the validity of the neoantigen selection algorithm, the immunogenicity of mRNA-1 was assessed in a first-in-human phase 1 study of patients with resected non-small-cell lung cancer (NSCLC) treated with mRNA-1 monotherapy or patients with resected cutaneous melanoma treated with mRNA-1 in combination with pembrolizumab in an adjuvant setting.

### Study Design and Patients

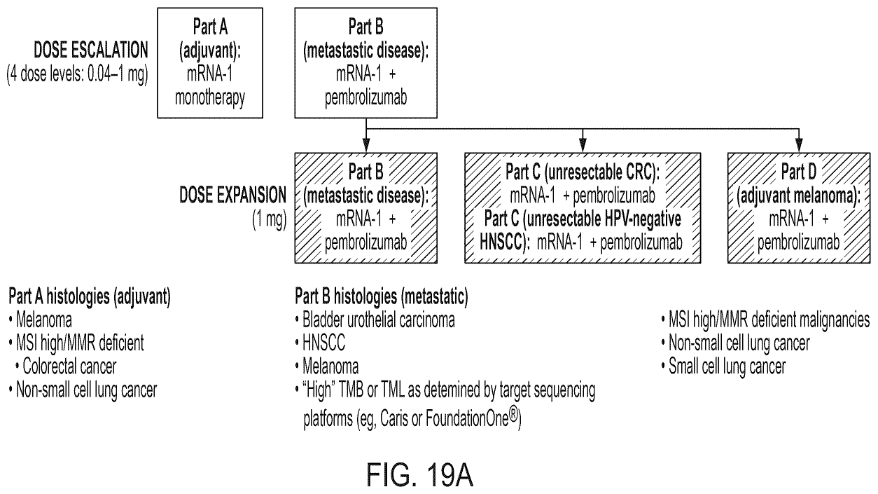

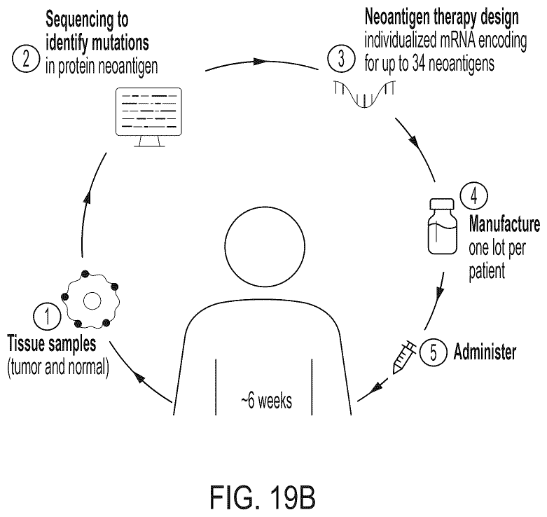

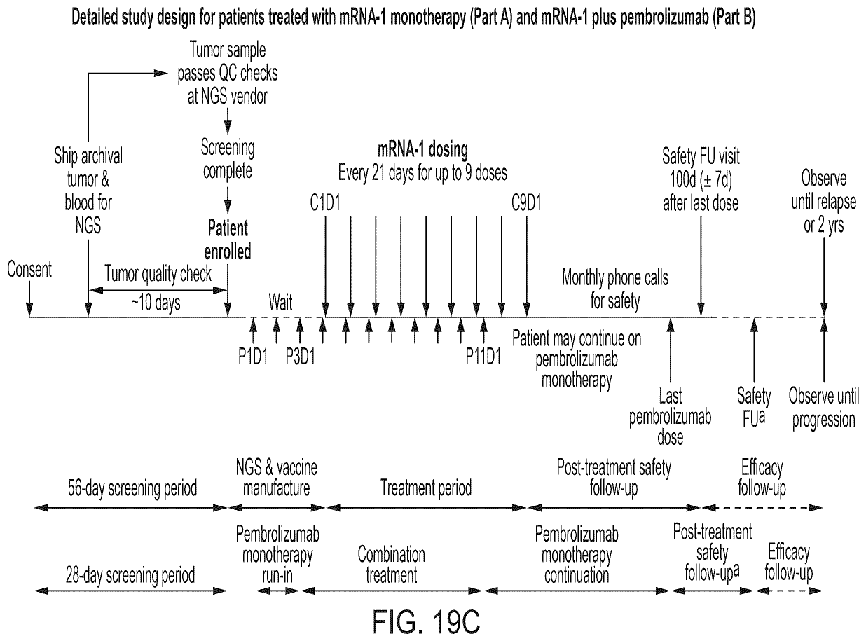

A multicenter dose escalation, multi-part, open-label, phase 1 trial investigated the safety, tolerability, and immunogenicity of mRNA-1 monotherapy in patients with resected solid tumors, including NSCLC, in the adjuvant setting (Part A) and mRNA-1 in combination with pembrolizumab in patients with completely resected stage II-IV cutaneous melanoma in the adjuvant setting (combination therapy; Part D) (FIGS. 19A, 19B, 19C). Eligible patients were aged ≥18 years with Eastern Cooperative Oncology Group performance status (ECOG PS) 0 or 1 who were clinically disease-free at study entry and were allowed to complete any standard of care adjuvant therapy. Prior PD-1/PD-L1 treatment was permitted. Patients were required to provide an archived tumor sample from a paraffin tissue block of unstained slides suitable for next-generation sequencing (NGS).

### INT Manufacturing

To manufacture INT mRNA-1 for each patient, DNA obtained from blood and tumor tissue samples was sequenced using WES and RNA-seq to identify tumor-specific mutations (FIG. 19B). The identified mutations were assessed to predict which were most likely to generate an anti-tumor immune response in the patient, and the selected neoantigens were incorporated into mRNA-1. Each patient-specific mRNA-1 was formulated in LNPs (comprising ionizable cationic lipid (Compound 1), neutral lipid (DSPC), sterol (cholesterol), and PEG-modified lipid (PEG-DMG)) and administered as an intramuscular injection.

### Treatment

During the study dose escalation phase, patients enrolled in Part A received mRNA-1 as a monotherapy every 3 weeks (Q3W) for up to 9 cycles at a fixed dose of 0.04 mg, 0.13 mg, 0.39 mg, or 1 mg (FIG. 19C). The 1 mg dose was selected for further dose expansion as the recommended dose in the phase 2 study; therefore, patients from Part A who received the 1 mg dose were included in the present analysis. Patients enrolled in Part D received pembrolizumab 200 mg Q3W for up to 3 cycles, followed by 1 mg mRNA-1 Q3W and pembrolizumab 200 mg Q3W for up to 9 cycles (combination therapy). Patients could then continue pembrolizumab monotherapy at the same dose for up to 18 total cycles (˜1 year) or until disease relapse or unacceptable toxicity, whichever was sooner.

### Procedures

Tumor imaging of the chest, abdomen, pelvis, head, and neck was preferentially performed using computed tomography (CT) or, if contraindicated, magnetic resonance imaging (MRI) at screening to confirm disease-free status. MRI was used for imaging of the brain and on-study imaging was performed per local standard of care. Any date of relapse within 2 years of the first dose of mRNA-1 was recorded in the case report form.

Adverse events (AEs), dose limiting toxicities, laboratory test abnormalities, electrocardiogram abnormalities, and vital sign abnormalities were graded according to the National Cancer Institute Common Terminology Criteria for Adverse Events version 5. AEs were collected through the time of informed consent through the last safety follow-up visit (100 days after the last dose of mRNA-1 or 30 days following the last dose of mRNA-1 if the participant initiated new anticancer therapy, and 30 days after the last dose of pembrolizumab). Follow-up phone calls for relapse observation occurred every 3 months after the last dose of mRNA-1 (Part A) or pembrolizumab (Part D) for up to 2 years after the first dose of mRNA-1, or until disease relapse, withdrawal from the study/new therapy, or loss to follow-up or death, whichever occurred first. Any-cause AEs were defined as any untoward medical occurrence in a patient enrolled into the study regardless of the causal relationship to the study drug. Serious AEs (SAEs) were defined as any AE that was life-threatening, medically significant, or resulted in a disability, inpatient or prolonged hospitalization, or death. Treatment-emergent AEs (TEAE) were defined as any new event or previous event that worsened in either intensity or frequency after exposure to either mRNA-1 or pembrolizumab. Causality between the TEAE and exposure to study drug was determined by the investigator (mRNA-1- or pembrolizumab-related TEAEs).

### Processing of Patient Tumor Biopsies

Core needle biopsies were fixed in formalin and embedded in paraffin. 5-μm slides were cut and macrodissected by a pathologist.

### Patient Tumor Biopsy WES and Whole Transcriptome Sequencing Analyses and Comparison to the Cancer Genome Atlas (TCGA) Reference Cohorts

To assess the tumor inflammation score (TIS) (an unweighted T cell-inflamed GEP score, using an unweighted average of the 18 genes as discussed above), cytolytic (CYT) activity score, and PD-L1 mRNA expression, RNA was extracted from macrodissected paraffin-embedded formalin-fixed baseline core biopsies and subjected to RNA sequencing. Gene expression data from all patients were log transformed (offset by 0.01) and quantile normalized. TIS score was then computed as an average of 18 genes (Ayers, et al. “IFN-γ-related mRNA profile predicts clinical response to PD-1 blockade” J Clin Invest. 127(8):2930-2940 (2017)) and CYT scores as an average of 2 genes (granzyme B and perforin). For PD-L1, normalized gene expression was used. To compute tumor mutational burden (TMB), DNA was extracted from macrodissected paraffin-embedded formalin-fixed baseline core biopsies and from a matched normal whole blood sample from the same patient. DNA from both samples was subjected to WES and duplicate exomes from tumor and matched normal samples were analyzed for single nucleotide variants. TMB in this Example represents the number of non-synonymous mutations with an allele frequency ≥5%.

The RNA_seq RSEM data for the tumor samples (n=443) from the TCGA-skin cutaneous melanoma (SKCM) cohort (portal.gdc.cancer.gov/projects/TCGA-SKCM) were used as a reference cohort in this study. In order to compare with the INT normalized gene expression data, the TCGA-SKCM RNA_seq data were normalized using the same approach that was implemented for the INT RNA_seq data. Furthermore, the ComBat method built in R package sva was applied to adjust for batch effects between the normalized gene expression data from patients in Part D and the TCGA-SKCM cohorts.

### Peripheral Blood Mononuclear Cell (PBMC) Processing and Storage

Apheresis samples were collected from all patients in collection bags and processed within 24 hours. PBMCs were isolated according to standardized protocols. Briefly, the apheresis product was diluted 1:1 with phosphate-buffered saline (PBS) and PBMCs were isolated using FICOLL® (MilliporeSigma, 17-1440-03) separation system. PBMCs were counted and frozen at 2×10⁷ cells/ml in Cryostor (STEMCELL™ Technologies) using controlled-rate freezing in a −80° C. freezer and were moved the next day to liquid nitrogen until further processing.

### Immunogenicity Analyses

T cell immunogenicity was used to examine the immune response to mRNA-1 via antigen-specific T cell assays in PBMCs from blood samples. Patients in Parts A and D also underwent apheresis at screening and at cycle 4 day 8 (C4D8) to provide sufficient T cell PBMC samples for in depth characterization of T cell immunogenicity and phenotypes.

To analyze individual patient responses to INT neoantigens, mRNA-1 neoantigen peptides were pooled for each individual patient. For patients who received mRNA-1 encoding 20 neoantigens, four pools, each containing 5 neoantigens, were prepared. For patients who received mRNA-1 encoding 34 neoantigens, five pools, each containing 6 or 7 neoantigens, were prepared. For each neoantigen, one 25-mer peptide, four overlapping peptides, and 1 minimal peptide were included. Individual patient T cell responses to INT neoantigen pools in this study were analyzed directly ex vivo using IFNγ Enzyme-linked ImmunoSpot (ELISpot) at longitudinal study timepoints. T cell responses to individual neoantigens were assessed following ex vivo expansion at pre-treatment and at C4D8. Characterization of neoantigen-specific responding CD4 and/or CD8 T cells was enabled through restimulation of expanded cells followed by intracellular cytokine staining.

### Ex Vivo IFNγ ELISpot

PBMCs were thawed with and rested at a concentration of 2×10⁶ cells/mL at 37° C. and 5% CO₂ in AIM V™ media (ThermoFisher, 0870112DK) supplemented with 2 mM GlutaMAX™ (Gibco®, 35050061), 1% sodium pyruvate (Gibco, 11360-070), 1× non-essential amino acids (Gibco 11140-050), and 0.01% 2-mercaptoethanol (Gibco, 21985-023). ELISpot plates pre-coated with antibodies specific for IFNγ (Mabtech, 3420-3 PT) were washed with PBS and blocked with AIM V™ for 2 h. Rested PBMCs were seeded at 4×10⁵ per well in duplicates and stimulated with peptides for 16-20 h. Peptides specific for each patient were synthesized by GenScript Biotech and reconstituted at 2 mg/ml per peptide in sterile dimethyl sulfoxide (DMSO, Sigma, D8418) or ultrapure distilled water (Invitrogen, 10977015) and stored at −20° C. Peptide pools corresponding to individual neoantigens consisted of the full length neoantigen sequence, a minimal peptide with strongest predicted human leukocyte antigen (HLA) class I binding affinity (9-10 amino acids [aa] in length) and 4-5 overlapping peptides (OLP, 15aa in length). Peptide pools corresponding to neoantigen pools consisted of all peptides for all individual neoantigens in that pool (up to 35 peptides per pool). Neoantigens were pooled in the order of appearance in the vaccine. Phytohemagglutinin (PHA, Sigma Aldrich, L8902) was included as a positive control and a combination of water and DMSO at concentrations used for peptide reconstitution for individual patients was included as a negative control. Spot development was enabled by addition of a biotin-conjugated anti-IFNγ antibody (Mabtech, 3420-6-1000) followed by incubation with VECTASTAIN® avidin peroxidase complex (Vector labs, PK-6100) and 3-Amino-9-Ethylcarbazole (AEC) substrate (BD Bioscience, 551951). ELISpot plates were allowed to dry. Briefly, plates were scanned using KS ELISpot Reader (Zeiss) and analyzed using the KS ELISpot software version 4.9.16.

Responses were presented as spot forming unit (SFU) per 1×10⁶ cells, which was calculated as the mean difference between the numbers of spots in wells stimulated with each peptide pool and the average number of spots+2 standard deviations from the corresponding control wells.

### Expansion of Neoantigen Specific T Cells

Neoantigen specific T cells were expanded from patient PBMCs immediately after thaw (Day 0) for 12-14 days with peptide pools corresponding either to individual neoantigens or neoantigen pools. Individual peptides were used at a final concentration of 2 ug/ml in complete media consisting of RPMI 1640 (Gibco, A10491-01) supplemented with 10% HI FBS (ThermoFisher, 16140071), 2 mM Glutamax (Gibco, 35050061), 1×NEAA (Gibco 11140-050), 25000U Pen/Strep (Gibco 150-70-063) and HEPES (Gibco, 15630-080). PBMCs were seeded at 2e6 cells/ml and cultured in 24-well plates. recombinant human (rh) IL-21 (Peprotech, 200-21) was added to the culture media on day 1. Cells were fed on days 2, 5, 7 and 9 by replacing half of the culture media with fresh expansion media supplemented with rhIL-7 (Peprotech, 200-07) and rhIL-15 (Peprotech, 200-15).

### Post Expansion Intracellular Staining and Identification of CD4 and CD8 Responses

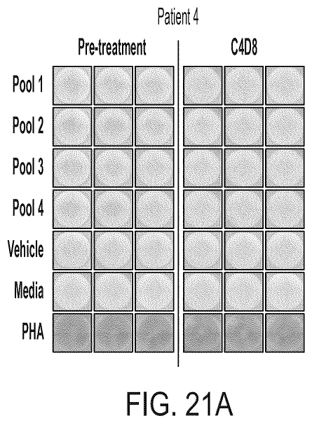

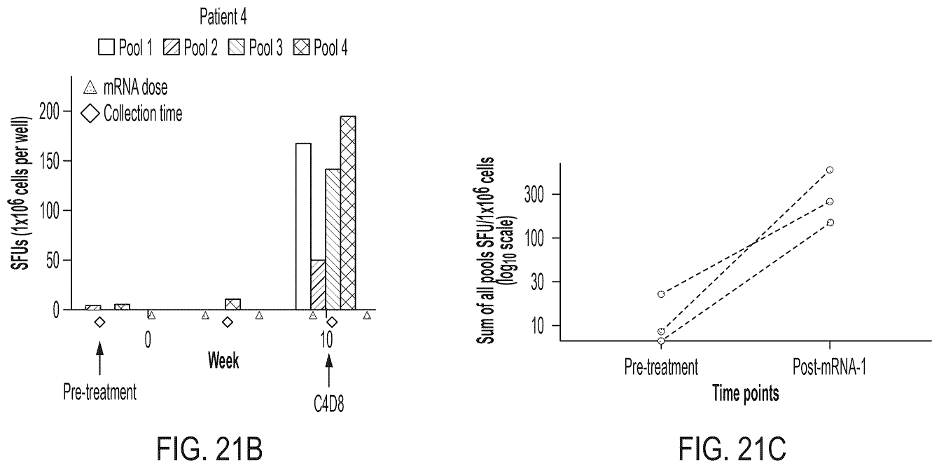

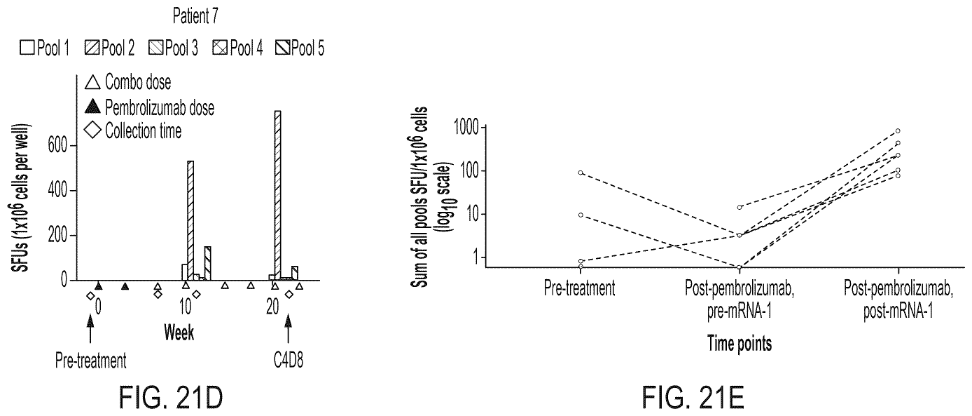

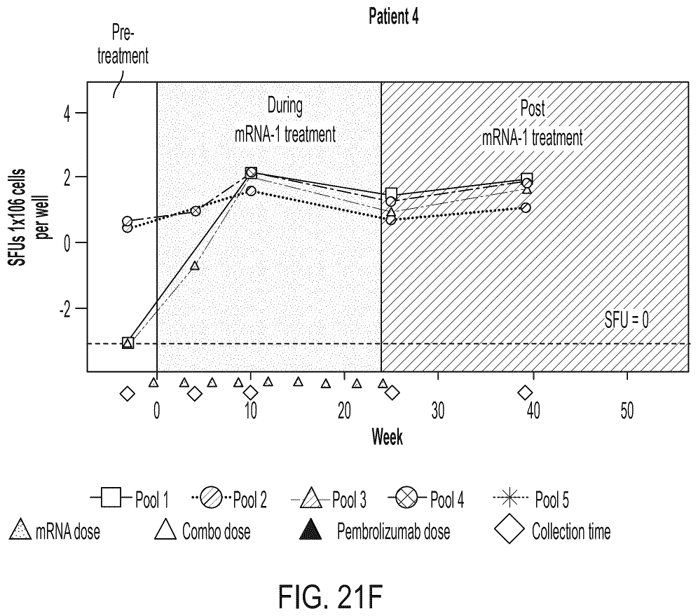

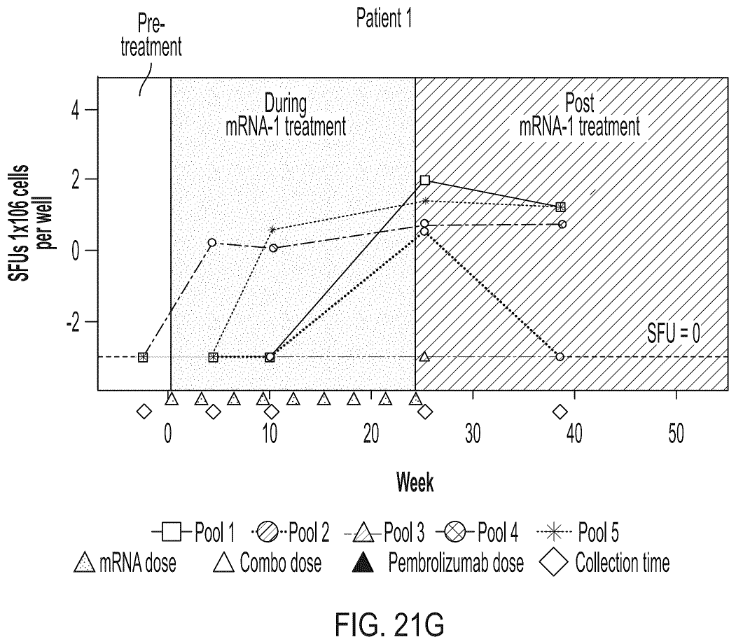

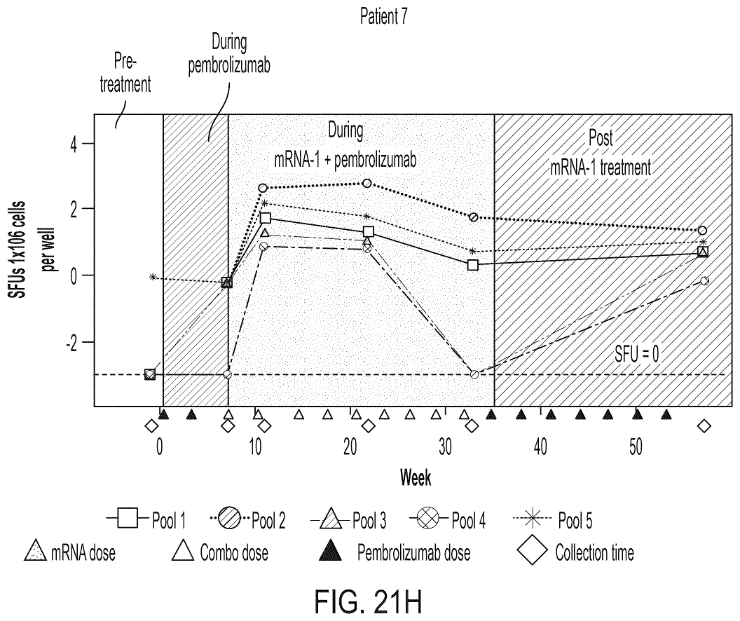

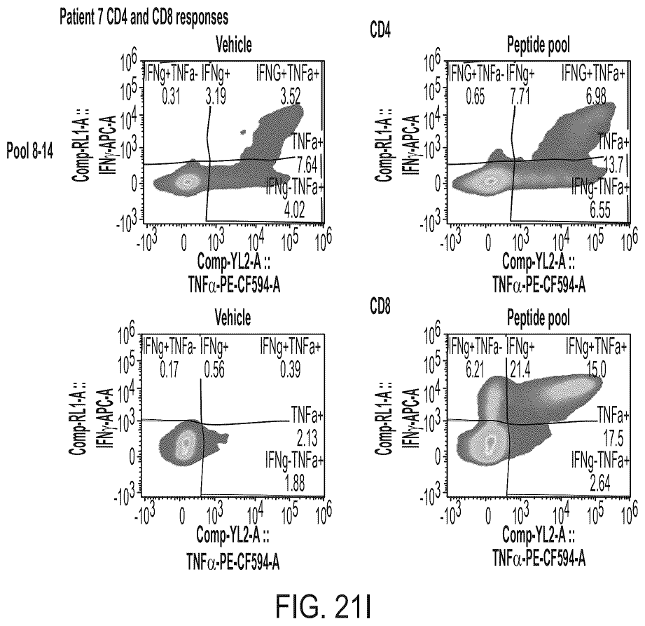

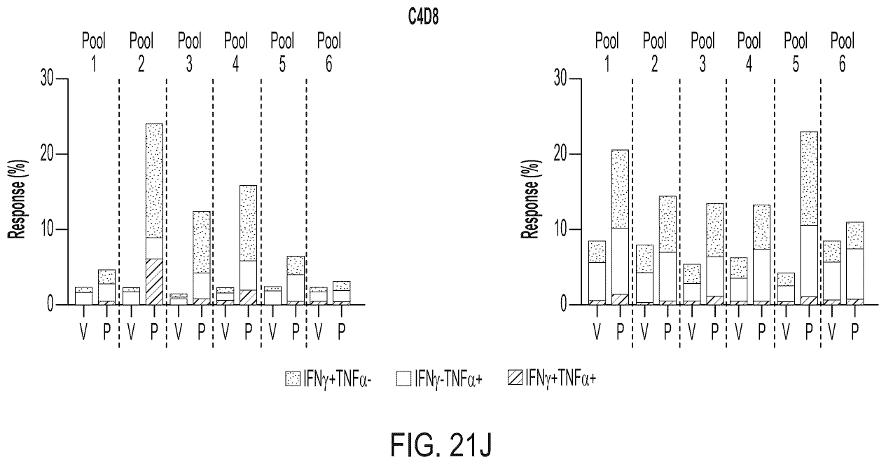

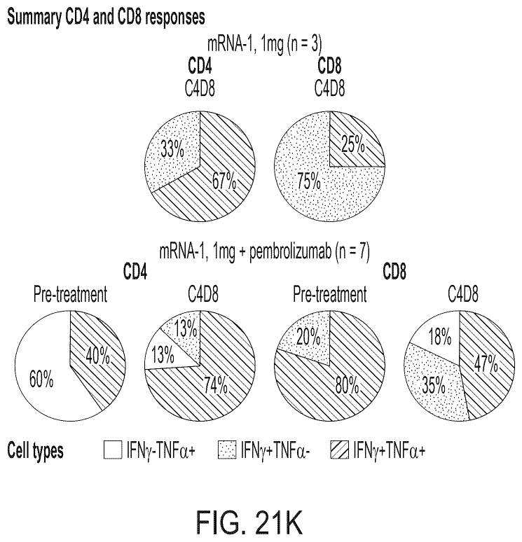

Autologous pre-treatment PBMCs were used as antigen-presenting cells for restimulation of expanded T cells. On the day prior to restimulation, pre-treatment PBMCs were thawed, and pulsed overnight separately with peptide pools corresponding to individual neoantigens or neoantigen pools, or with vehicle (combination of water and DMSO at concentrations used for peptide reconstitution for corresponding peptide pools). The next day, autologous PBMCs were stained with 0.1 uM carboxyfluorescein succinimidyl ester (CFSE, Thermofisher C34554). Expanded T cells were stimulated for 5 hours in separate wells with CFSE-stained peptide or vehicle pulsed autologous PBMCs at a ratio of 1:1, or phorbol 12-myristate 13-acetate (PMA)/ionomycin cell stimulation cocktail (Biolegend 423301) in the presence of Protein Transport Inhibitor cocktail (BD Biosciences, 554724) and Brefeldin A (BD Biosciences, 555029). One replicate was used for each condition and for each fluorescence minus one (FMO) gating control. Following incubation, cells were stained with live/dead stain (Thermofisher, L34975), followed by the anti-human surface marker antibodies (Table 15) and fixation and permeabilization with Cytofix/Cytoperm reagents according to manufacturer recommendations (BD, 554722). Following permeabilization, cells were stained with the anti-human antibodies directed at intracellular cytokines listed in Table 15. Data was acquired on Attune flow cytometer (ThermoFisher) and analyzed with FlowJo™ software. An example gating strategy is shown in FIG. 21I. For calling of CD4 and/or CD8 responses, distance of peptide stimulation data points to mean of variance of vehicle datapoints for individual CD4 and CD8 subpopulations was used to generate a p value. Fold change of peptide to vehicle wells for individual subpopulations was plotted against p-values. All plotted datapoints above p=0.01 and a fold change of 2 were considered positive calls for the subpopulation considered.

TABLE 15
Cellular markers used for post expansion intracellular
staining and identification of CD4 and CD8 responses

| | Extracellular or | | | |
|---|---|---|---|---|
| Marker | Intracellular | Fluorochrome | Clone | Source |
| CD3 | Extracellular | BV421 | UCHT1 | Biolegend |
| CD4 | Extracellular | PE-Cy7 | RPA-T4 | Biolegend |
| CD8 | Extracellular | Percp-Cy5.5 | RPA-T8 | BD |
| CD14 | Extracellular | PE | 63D3 | Biolegend |
| CD56 | Extracellular | efluor506 | TULY56 | BD |
| IFNγ | Intracellular | APC | 4S.B3 | BD |
| TNFα | Intracellular | PE-CF594 | MAb11 | Invitrogen |

### Ex Vivo Immune Phenotyping

Patient PBMCs were thawed in complete RPMI media (supplemented as described above). Cells were plated at 1×10⁶ cells/well in triplicates for each sample condition and in one replicate for fluorescence minus one (FMO) gating controls. Cells were incubated with Fc receptor blocking solution (Biolegend, 422302) followed by Live dead stain (Thermofisher, L34975). Cells were stained for extracellular markers (Table 16) and incubated for 30 minutes at 4° C. in the dark. Following nuclear fixation for 30-60 minutes at 4° C. with Transcription factor staining buffer set (eBioscience, 00-5523-00), cells were stained with intracellular markers (Table 16) for 1 hour at room temperature. Isotype controls were used to complete FMO controls. Data was acquired on Attune flow cytometer (ThermoFisher) and analyzed with FlowJo™ software. An example gating strategy is shown in FIG. 21I.

TABLE 16
Cellular markers used for ex vivo immune phenotyping

| | Extracellular or | | | |
|---|---|---|---|---|
| Marker | Intracellular | Fluorochrome | Clone | Source |
| PD-1 | Extracellular | BV421 | EH12.2H7 | Biolegend |
| CD127 | Extracellular | BV510 | A019D5A | Biolegend |
| GD TCR | Extracellular | BV605 | 11F2 | BD |
| TIM-3 | Extracellular | BV650 | TIM-3 F38-362 | Biolegend |
| CD3 | Extracellular | BV711 | Clone SP34-2 | BD |
| Tbet | Intracellular | BV786 | 564141 | BD |
| CD8 | Extracellular | FITC | MHCD08014 | Invitrogen |
| CD4 | Extracellular | PerCP-Cy5.5 | 344608 | Biolegend |
| GATA3 | Intracellular | PE | 16E10A23 | Biolegend |
| FOXP3 | Intracellular | PE Dazzle | 206D | Biolegend |
| Grzm B | Intracellular | PE-Cy7 | QA16A02 | Biolegend |
| RORyT | Intracellular | APC | AFKJS-9 | Invitrogen |
| CD14 | Extracellular | AF-700 | 63D3 | Biolegend |
| CD56 | Extracellular | AF-700 | HIB19 | Biolegend |
| CD20 | Extracellular | AF-700 | 2H7 | Biolegend |
| CD19 | Extracellular | AF-700 | 5.1H11 | Biolegend |
| CCR7 | Extracellular | BV605 | G043H7 | Biolegend |
| CD45RA | Extracellular | BV785 | HI100 | Biolegend |
| TCF1/7 | Intracellular | PE | S33966 | BD |
| CD45RO | Extracellular | APC | UCHL1 | Biolegend |

### Statistical Analysis

Safety analyses were performed on the full analysis set (FAS), which included all patients who received at least 1 dose of mRNA-1 or pembrolizumab. Safety was analyzed according to the final treatment received. Immunogenicity was descriptively analyzed in patients who received at least one dose of mRNA-1, and had samples collected at baseline (pre-treatment) and at least one post-treatment time point.

Continuous variables were summarized using, median, maximum value, and minimum value; categorical variables were summarized using frequency counts and percentages. All statistical analyses were performed using SAS, version 9.4.

### Patients

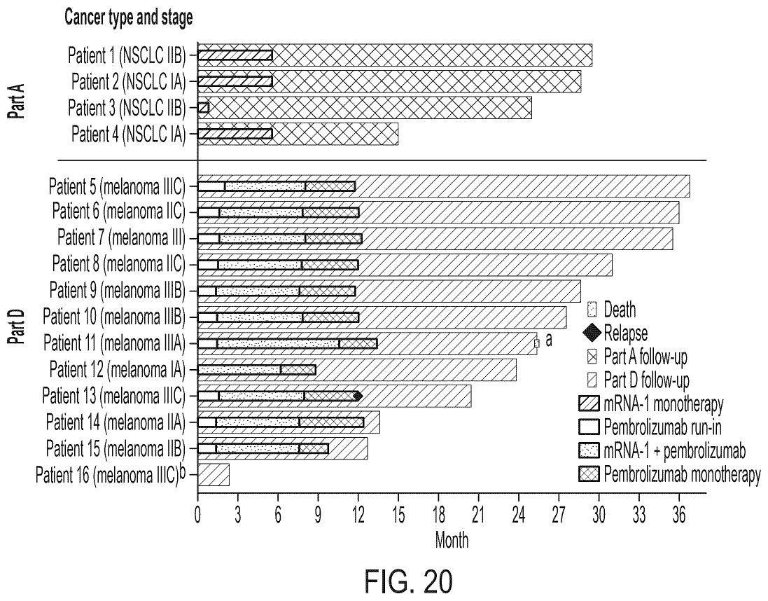

The analysis included 4 patients with NSCLC from Part A and 12 patients with melanoma from Part D of the phase study who received mRNA-1 therapy (FIG. 19A, FIG. 20). The median (range) age was 67.0 (36-78) years (Table 17). Seven patients (43.8%) had ECOG PS scores of 0; 5 patients (31.3%) had previously received adjuvant systemic therapy, and 15 patients (93.8%) had ≥1 prior cancer-related surgery. At primary diagnosis, three patients (18.8%) had stage I disease, 6 patients (37.5%) had stage II disease, and 7 patients (43.8%) had stage III disease (FIG. 20). At the time of database cutoff (May 4, 2023), 12 patients (75.0%) had completed the study, and 4 patients (25.0%) had discontinued the study. Reasons for study discontinuation were loss to follow-up (n=2), death (n=1), and increased liver enzymes (n=1). One patient experienced recurrence during the follow-up period after receiving all doses of mRNA-1 and pembrolizumab.

TABLE 17
Patient demographics pre-treatment

| | Part A | | Part D | | Total | |
|---|---|---|---|---|---|---|
| | (n = 4) | | (n = 12) | | (N = 16) | |
| Sex | | | | | | |
| Male | 2 | (50.0) | 8 | (66.7) | 10 | (62.5) |
| Female | 2 | (50.0) | 4 | (33.3) | 6 | (37.5) |
| Age | | | | | | |
| Median (range), years | 70.5 | (67-76) | 67.0 | (36-78) | 67.0 | (36-78) |
| ≥65 years | 4 | (100.0) | 8 | (66.7) | 12 | (75.0) |
| BMI, median (range), | 35.5 | (20.7-38.5) | 35.8 | (21.3-43.1) | 35.8 | (20.7-43.1) |
| kg/m² | | | | | | |
| Race | | | | | | |
| White | 2 | (50.0) | 12 | (100.0) | 14 | (87.5) |
| Not reported | 2 | (50.0) | 0 | (0) | 2 | (12.5) |
| Ethnicity | | | | | | |
| Hispanic or Latino | 1 | (25.0) | 0 | (0) | 1 | (6.3) |
| Not Hispanic or | 3 | (75.0) | 12 | (100.0) | 15 | (93.8) |
| Latino | | | | | | |
| ECOG PS score | | | | | | |
| 0 | 1 | (25.0) | 6 | (50) | 7 | (43.8) |
| 1 | 3 | (75.0) | 6 | (50) | 9 | (56.3) |
| Tumor type | | | | | | |
| Melanoma | 0 | (0) | 12 | (100.0) | 12 | (75.0) |
| Non-small cell lung | 4 | (100.0) | 0 | (0) | 4 | (25.0) |
| cancer | | | | | | |
| Disease stage at | | | | | | |
| primary diagnosis | | | | | | |
| IA | 2 | (50.0) | 1 | (8.3) | 3 | (18.8) |
| IIA | 0 | (0) | 1 | (8.3) | 1 | (6.3) |
| IIB | 2 | (50.0) | 1 | (8.3) | 3 | (18.8) |
| IIC | 0 | (0) | 2 | (16.7) | 2 | (12.5) |
| III | 0 | (0) | 1 | (8.3) | 1 | (6.3) |
| IIIA | 0 | (0) | 1 | (8.3) | 1 | (6.3) |
| IIIB | 0 | (0) | 2 | (16.7) | 2 | (12.5) |
| IIIC | 0 | (0) | 3 | (25.0) | 3 | (18.8) |
| Prior immuno- | 0 | (0.0) | 2 | (16.7) | 2 | (12.5) |
| oncology therapy | | | | | | |
| Number of prior | | | | | | |
| systemic therapies | | | | | | |
| 0 | 1 | (25.0) | 10 | (83.3) | 11 | (68.8) |
| 1 | 3 | (75.0) | 2 | (16.7) | 5 | (31.3) |
| Prior adjuvant | 3 | (75.0) | 2 | (16.7) | 5 | (31.3) |
| systemic therapy | | | | | | |
| Type of prior | | | | | | |
| systemic therapy | | | | | | |
| Cisplatin | 3 | (75.0) | 0 | (0) | 3 | (18.8) |
| Gemcitabine | 1 | (25.0) | 0 | (0) | 1 | (6.3) |
| Nivolumab | 0 | (0) | 1 | (8.3) | 1 | (6.3) |
| Pembrolizumab | 0 | (0) | 1 | (8.3) | 1 | (6.3) |
| Pemetrexed disodium | 2 | (50.0) | 0 | (0) | 2 | (12.5) |
| heptahydrate | | | | | | |
| Number of prior | | | | | | |
| cancer-related | | | | | | |
| surgeries | | | | | | |
| 1 | 2 | (50.0) | 4 | (33.3) | 6 | (37.5) |
| 2 | 1 | (25.0) | 6 | (50.0) | 7 | (43.8) |
| 3 | 1 | (25.0) | 0 | (0) | 1 | (6.3) |
| 4 | 0 | (0) | 0 | (0) | 0 | (0) |
| 5 | 0 | (0) | 1 | (8.3) | 1 | (6.3) |
| Missing | 0 | (0) | 1 | (8.3) | 1 | (6.3) |
| Prior distant | 1 | (25.0) | 4 | (33.3) | 5 | (31.3) |

metastasis

^{a}Death due to disease progression and not related to mRNA-1 or pembrolizumab.
^{b}Discontinued treatment after the initial pembrolizumab monotherapy lead-in (during the manufacture of mRNA-1).
Data are n (%) unless otherwise stated.
BMI, body mass index; ECOG PS, Eastern Cooperative Oncology Group Performance score.

The median (range) number of mRNA-1 doses received was 9 (2-9) for Part A and 9 (0-9) for Part D. All patients in Part D received pembrolizumab doses; however, one patient received 1 dose of pembrolizumab during the run-in phase but discontinued treatment due to aspartate aminotransferase (AST) elevation and thus did not receive mRNA-1. For the 11 Part D patients that received both mRNA-1 and pembrolizumab, the median (range) number of pembrolizumab doses was 16 (13-16) during the treatment phase.

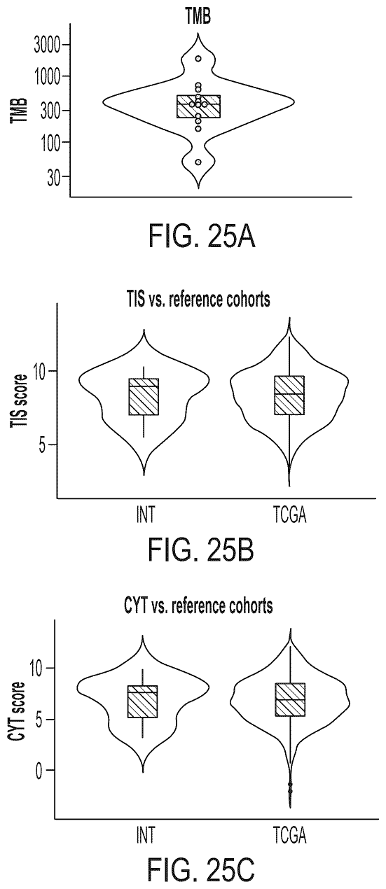

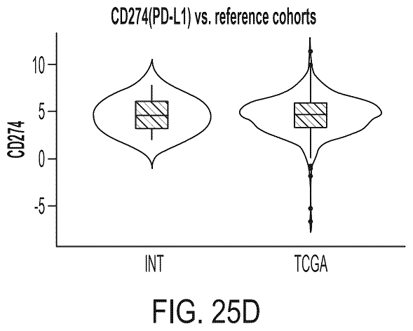

Patients with melanoma had tumors comparable to the broader melanoma population in terms of tumor mutational burden, tumor inflammation score, and CD274 mRNA expression (FIGS. 25A, 25B, 25C, 25D).

### Safety

There were no grade 4 or 5 AEs, and no dose-limiting toxicities with mRNA-1 treatment. Of the 15 patients who received a dose of mRNA-1, all experienced at least 1 any grade TEAE (Table 18 below). The most frequent TEAEs of any grade across Parts A and D were fatigue (n=10; 66.7%), pyrexia (n=9; 60.0%), and injection site pain (n=6; 40.0%).

In Part A, all 4 patients experienced TEAEs with the most common mRNA-1-related events as assessed by the investigator being pyrexia (n=2, 50.0%) and influenza-like illness (n=2, 50.0%). The events did not result in a delay or discontinuation of study treatment. The events considered related by the investigator were transient with all events resolving after median (range) duration of 4 (1-43) days with the exception of grade 1 fatigue. One elderly patient with NSCLC (Part A) had TEAEs meeting serious criteria (grade 1 pyrexia and musculoskeletal pain).

In Part D, among patients who received both mRNA-1 and pembrolizumab, 10/11 (90.9%) patients experienced TEAEs considered related to mRNA-1 by the investigator with the most common events being pyrexia (n=7, 63.6%), fatigue (n=6, 54.5%), injection site pain (n=5, 45.5%), and chills (n=5, 45.5%). The events were mostly transient with most resolving after a median (range) duration of 3 (1-176) days and none resulted in a delay or discontinuation of study treatment. mRNA-1-related events were mostly grade 1 or 2 with one patient having grade 3 fatigue attributed to both mRNA-1 and pembrolizumab. One patient had TEAEs meeting serious criteria related to treatment, an elderly patient with grade 2 pyrexia and asthenia, attributed to both mRNA-1 and pembrolizumab. Pembrolizumab-related TEAEs were mostly grade 1 or 2. The most common TEAEs considered related to pembrolizumab treatment by the investigator were fatigue (n=6, 54.5%), chills (n=3, 27.3%), pyrexia (n=3, 27.3%), and rash (n=3, 27.3%).

TABLE 18
Safety summary for patients who received a dose of mRNA-1

| | | | Part D | | | |
|---|---|---|---|---|---|---|
| | Part A | | mRNA-1 1 mg + | | | |
| Patients with ≥1 treatment- | mRNA-1 1 mg | | pembrolizumab | | Total | |
| emergent AE | (n = 4) | | (n = 11^{a}) | | (N = 15) | |
| Any-cause, any grade | 4 | (100.0) | 11 | (100.0) | 15 | (100.0) |
| Any cause, grade 3^{b} | 1 | (25.0) | 4 | (36.4) | 5 | (33.3) |
| Any cause, serious | 1 | (25.0) | 2 | (18.2) | 3 | (20.0) |
| mRNA-1-related^{c} | 4 | (100.0) | 10 | (90.9) | 14 | (93.3) |
| mRNA-1-related grade 3^{b} | 0 | (0) | 1 | (9.1) | 1 | (6.7) |
| mRNA-1-related serious | 1 | (25.0) | 1 | (9.1) | 2 | (13.3) |

| | Any | | Grade | Any | | Grade | | Any | | Grade | |
|---|---|---|---|---|---|---|---|---|---|---|---|
| Treatment-related AEs | grade | | 3 | grade | | 3 | | grade | | 3 | |
| mRNA-1-related^{c} | | | | | | | | | | | |
| Pyrexia | 2 | (50.0) | 0 (0) | 7 | (63.6) | 0 | (0) | 9 | (60.0) | 0 | (0) |
| Fatigue | 1 | (25.0) | 0 (0) | 6 | (54.5) | 1 | (9.1) | 7 | (46.7) | 1 | (6.7) |
| Injection site pain | 1 | (25.0) | 0 (0) | 5 | (45.5) | 0 | (0) | 6 | (40.0) | 0 | (0) |
| Chills | 0 | (0) | 0 (0) | 5 | (45.5) | 0 | (0) | 5 | (33.3) | 0 | (0) |
| Influenza-like illness | 2 | (50.0) | 0 (0) | 2 | (18.2) | 0 | (0) | 4 | (26.7) | 0 | (0) |
| Arthralgia | 0 | (0) | 0 (0) | 2 | (18.2) | 0 | (0) | 2 | (13.3) | 0 | (0) |
| Hyperhidrosis | 0 | (0) | 0 (0) | 2 | (18.2) | 0 | (0) | 2 | (13.3) | 0 | (0) |
| Injection site rash | 1 | (25.0) | 0 (0) | 1 | (9.1) | 0 | (0) | 2 | (13.3) | 0 | (0) |
| Myalgia | 1 | (25.0) | 0 (0) | 1 | (9.1) | 0 | (0) | 2 | (13.3) | 0 | (0) |
| Nausea | 0 | (0) | 0 (0) | 2 | (18.2) | 0 | (0) | 2 | (13.3) | 0 | (0) |
| Asthenia | 0 | (0) | 0 (0) | 1 | (9.1) | 0 | (0) | 1 | (6.7) | 0 | (0) |
| Headache | 0 | (0) | 0 (0) | 1 | (9.1) | 0 | (0) | 1 | (6.7) | 0 | (0) |
| Injection site erythema | 1 | (25.) | 0 (0) | 0 | (0) | 0 | (0) | 1 | (6.7) | 0 | (0) |
| Injection site warmth | 1 | (25.0) | 0 (0) | 0 | (0) | 0 | (0) | 1 | (6.7) | 0 | (0) |
| Lymph node pain | 0 | (0) | 0 (0) | 1 | (9.1) | 0 | (0) | 1 | (6.7) | 0 | (0) |
| Musculoskeletal pain | 1 | (25.0) | 0 (0) | 0 | (0) | 0 | (0) | 1 | (6.7) | 0 | (0) |
| Pain | 0 | (0) | 0 (0) | 1 | (9.1) | 0 | (0) | 1 | (6.7) | 0 | (0) |
| Pain in extremity | 1 | (25.0) | 0 (0) | 0 | (0) | 0 | (0) | 1 | (6.7) | 0 | (0) |
| Peripheral sensory neuropathy | 0 | (0) | 0 (0) | 1 | (9.1) | 0 | (0) | 1 | (6.7) | 0 | (0) |
| Pruritus | 0 | (0) | 0 (0) | 1 | (9.1) | 0 | (0) | 1 | (6.7) | 0 | (0) |
| Vomiting | 1 | (25.0) | 0 (0) | 0 | (0) | 0 | (0) | 1 | (6.7) | 0 | (0) |

| Pembrolizumb-related^{d} | | | | | | | | |
|---|---|---|---|---|---|---|---|---|
| Fatigue | NA | NA | 6 | (54.5) | 1 | (9.1) | NA | NA |
| Chills | NA | NA | 3 | (27.3) | 0 | (0) | NA | NA |
| Pyrexia | NA | NA | 3 | (27.3) | 0 | (0) | NA | NA |
| Rash | NA | NA | 3 | (27.3) | 0 | (0) | NA | NA |
| Hyperhidrosis | NA | NA | 2 | (18.2) | 0 | (0) | NA | NA |
| Influenza-like illness | NA | NA | 2 | (18.2) | 0 | (0) | NA | NA |
| Myalgia | NA | NA | 2 | (18.2) | 0 | (0) | NA | NA |
| Nausea | NA | NA | 2 | (18.2) | 0 | (0) | NA | NA |
| Arthralgia | NA | NA | 1 | (9.1) | 0 | (0) | NA | NA |
| Increased aspartate | NA | NA | 0 | (0) | 0 | (0) | NA | NA |
| aminotransferase^{a} | | | | | | | | |
| Asthenia | NA | NA | 1 | (9.1) | 0 | (0) | NA | NA |
| Decreased appetite | NA | NA | 1 | (9.1) | 0 | (0) | NA | NA |
| Diarrhea | NA | NA | 1 | (9.1) | 0 | (0) | NA | NA |
| Increased gamma- | NA | NA | 1 | (9.1) | 0 | (0) | NA | NA |
| glutamyltransferase | | | | | | | | |
| Hypothyroidism | NA | NA | 1 | (9.1) | 0 | (0) | NA | NA |
| Injection site pain | NA | NA | 1 | (9.1) | 0 | (0) | NA | NA |
| Insomnia | NA | NA | 1 | (9.1) | 0 | (0) | NA | NA |
| Peripheral sensory neuropathy | NA | NA | 1 | (9.1) | 0 | (0) | NA | NA |
| Pneumonitis | NA | NA | 1 | (9.1) | 0 | (0) | NA | NA |
| Pruritus | NA | NA | 1 | (9.1) | 0 | (0) | NA | NA |

^{a}One patient did not receive mRNA-1; patient discontinued during pembrolizumab lead-in at run-in because of pembrolizumab-related grade 3 increased aspartate aminotransferase.
^{b}No grade 4 or 5 AEs were reported.
^{c}Determined by the investigator to be related to mRNA-1 treatment.
^{d}Not all patients received pembrolizumab, therefore, some pembrolizumab-related AEs were designated as NA.
Values are n (%).
AE, adverse event; INT, individualized neoantigen therapy; n, number of patients; NA, not applicable.

### Immunogenicity:

mRNA-1 Induces Durable and Polyfunctional Neoantigen-Specific CD4 and CD8 T Cell Responses

To evaluate the ability of mRNA-1 to elicit antigen-specific immune responses and to assess the durability of these responses, ELISpot analysis was performed after ex vivo restimulation of PBMCs collected from patients following mRNA-1 monotherapy (Part A) or combination therapy (Part D). Of the 4 patients in Part A, blood samples were available for 3 patients to assess the immunogenicity of mRNA-1 monotherapy; exemplary samples for Patient 4 are shown in FIGS. 21A and 21B. Analysis of longitudinal peripheral blood samples available for the 3 patients in Part A indicated variable breadth, strength, and kinetics of T cell responses to neoantigen pools across patients. Low or absent neoantigen-specific T cell responses were observed prior to INT treatment. INT-specific robust T cell responses were observed in all 3 patients after administration of mRNA-1 (FIG. 21C).

Of the 12 patients assigned to combination therapy, 7 (58%) had available blood samples to evaluate mRNA-1-specific immunogenicity; an exemplary sample is shown in FIG. 21D. INT-specific T cell responses were detected in all 7 patients (e.g., FIG. 21E). Prior to treatment with pembrolizumab, pre-existing T cell responses to neoantigens included in mRNA-1 were low (<32 spot-forming units [SFU]/1×10⁶ cells; n=3), not observed (n=3), or not analyzed (n=1). T cell responses to the neoantigens included in mRNA-1 post-pembrolizumab run-in remained low (<10 SFU/1×10⁶ cells; n=3) or were not observed (n=4). Most of the immune responses were observed following combination therapy.

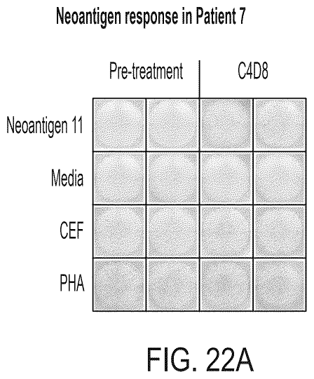

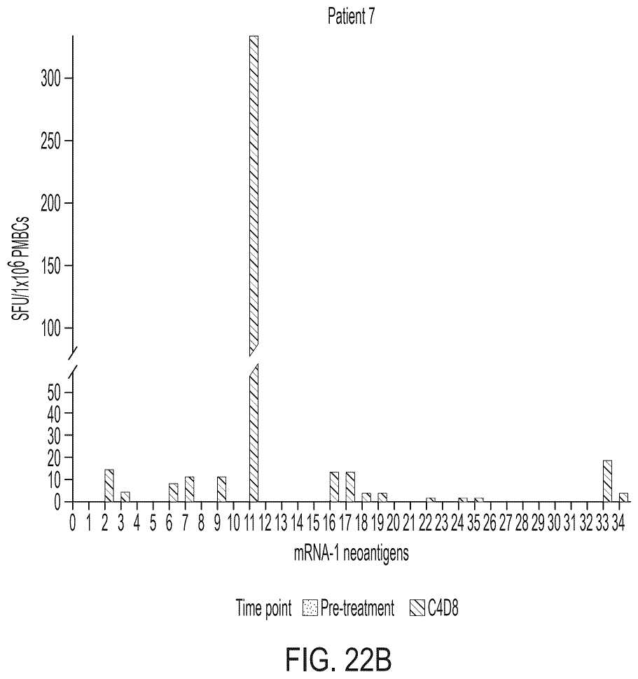

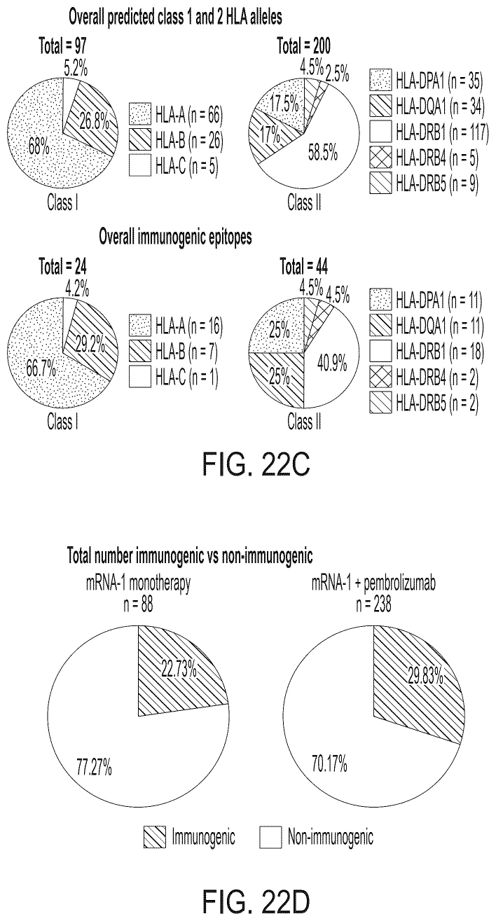

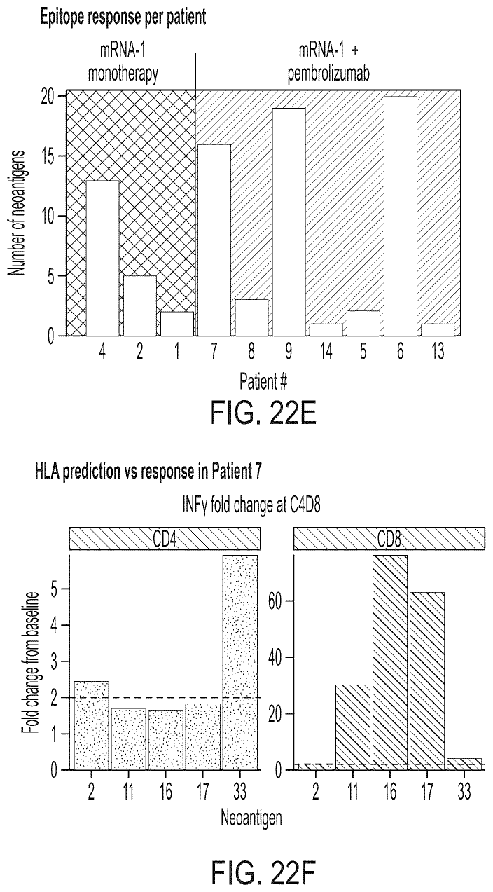

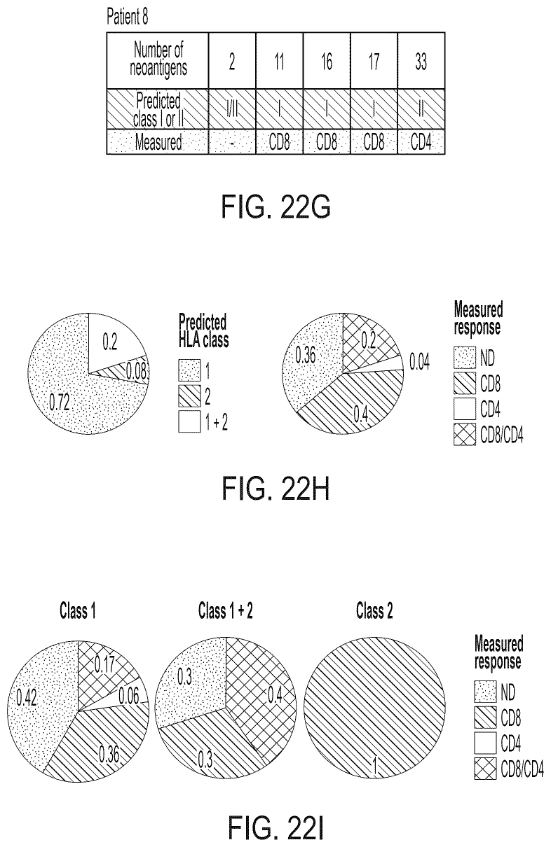

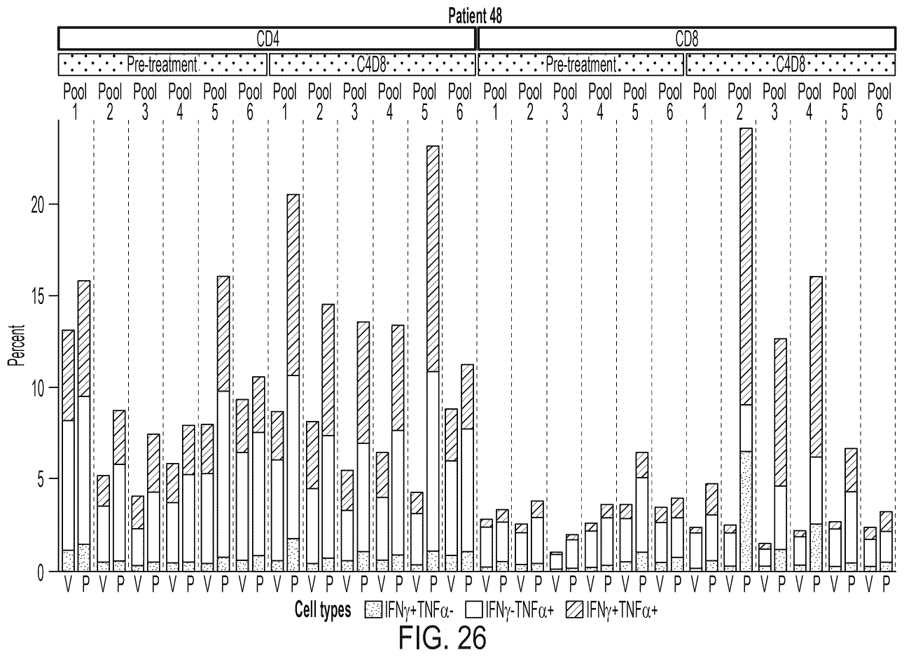

To assess persistence of INT-specific immune responses, samples were collected and evaluated 100 days post-treatment. Two patients with NSCLC (patients 1 and 4) and one patient with melanoma (patient 7) had samples available for immunogenicity analyses at 100 days post-treatment; neoantigen-specific responses to the majority of neoantigen pools were observed, indicating durability of responses (FIG. 22F, 22G, 22H). mRNA-1 induced both CD4 and CD8 T cell neoantigen-specific responses (FIG. 22I, 22J, 22K; FIG. 26).

mRNA-1 Encodes Predicted Immunogenic Neoantigens Recognized by Both CD4 and CD8 T Cells

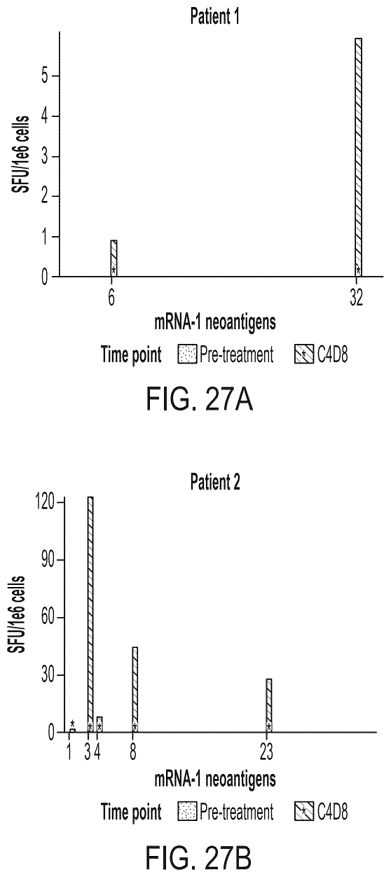

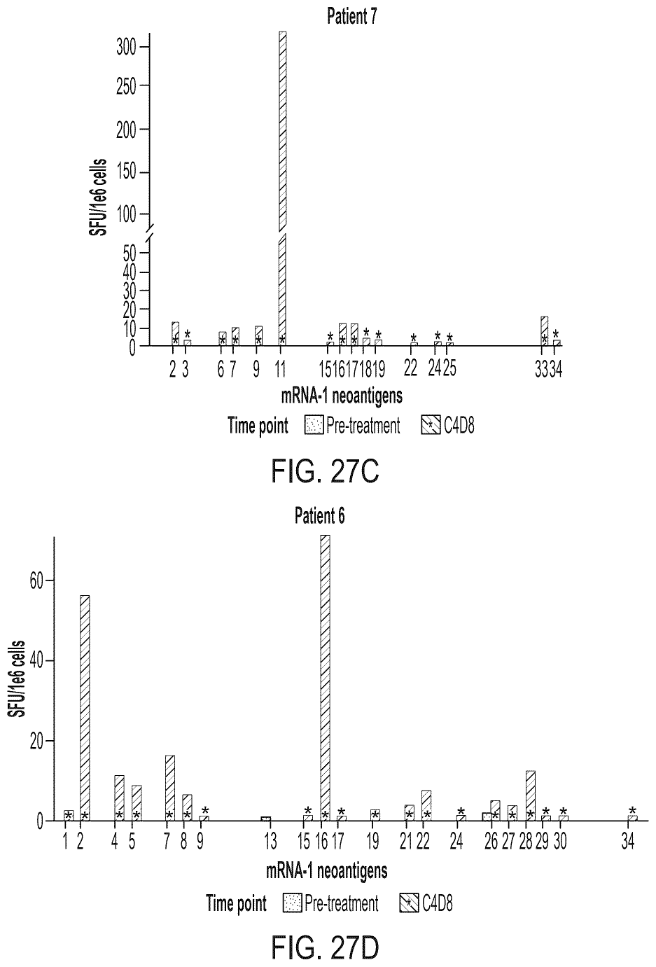

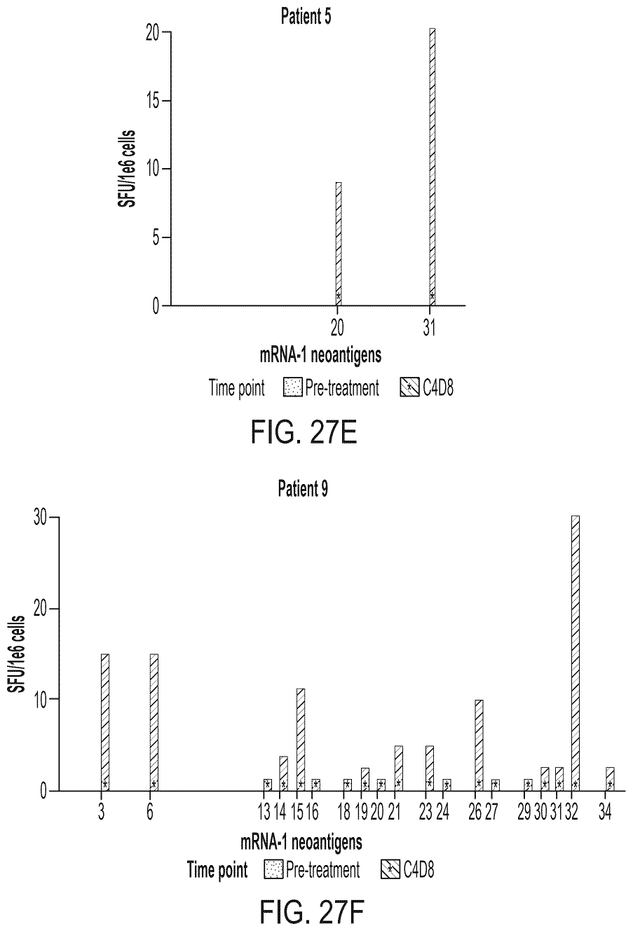

After establishing the ability of mRNA-1 to illicit T cell responses in all patients tested, the neoantigen-specific responses were deconvoluted to further characterize their quality at the epitope level. At C4D8, all evaluable patients exhibited ex-vivo T cell-specific responses to individual deconvoluted mRNA-1 neoantigens (FIG. 22A, 22B; FIGS. 27A, 27B, 27C, 27D, 27E, 27F). Enrolled patients included a heterogenous diversity of human leukocyte antigen (HLA) allele haplotypes that were representative of a North American population. In-silico predicted neoantigens encoded by mRNA-1 included both class I and class II HLA alleles (FIG. 22C).

Overall, 22.7% and 29.8% of the total number of neoantigens included in mRNA-1 were immunogenic with mRNA-1 monotherapy and combination therapy, respectively (FIG. 22D). mRNA-1 induced a breadth of neoantigen-specific T cell responses. The median number of neoantigen responses was 4 across all 10 patients (Parts A and D), with a range of 1-20 responses to individual neoantigens, with five patients exhibiting responses to ≥5 individual neoantigens (FIG. 22E).

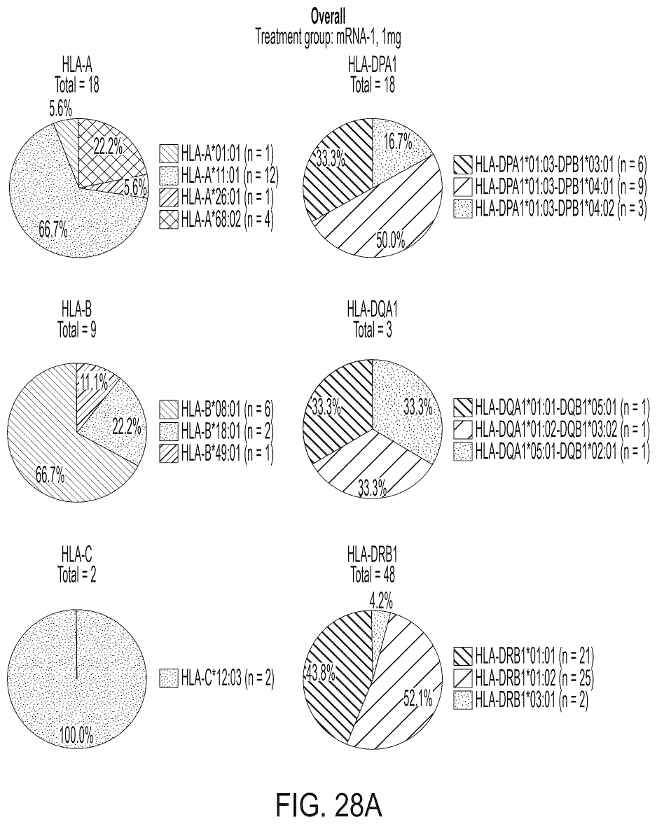

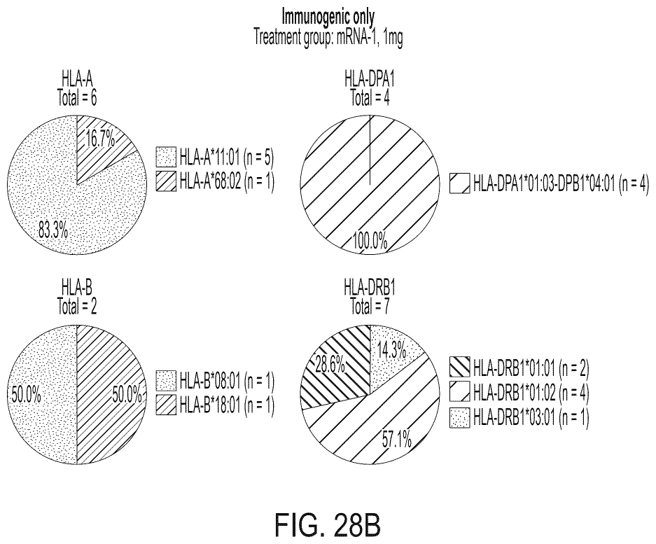

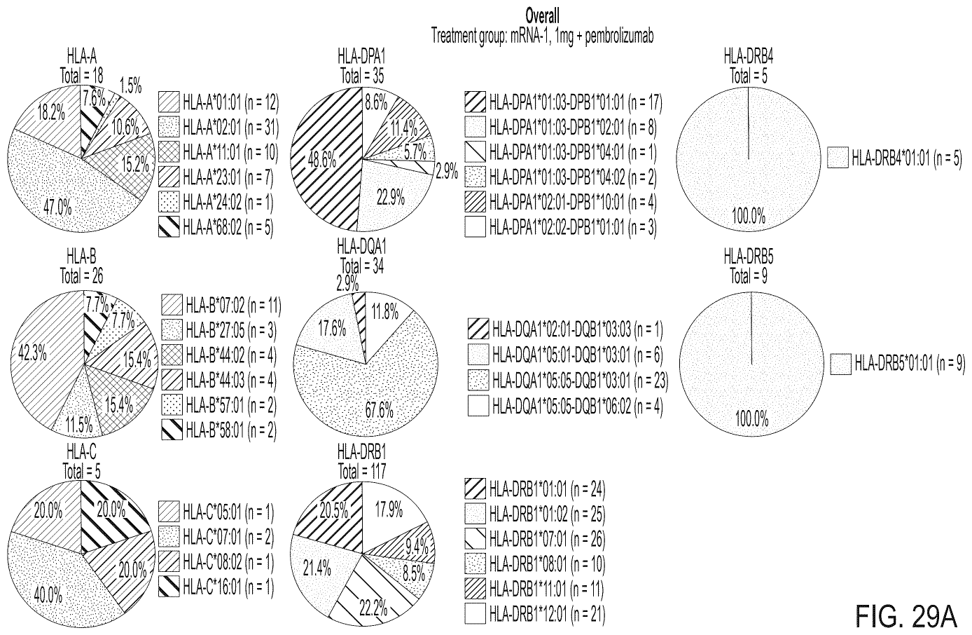

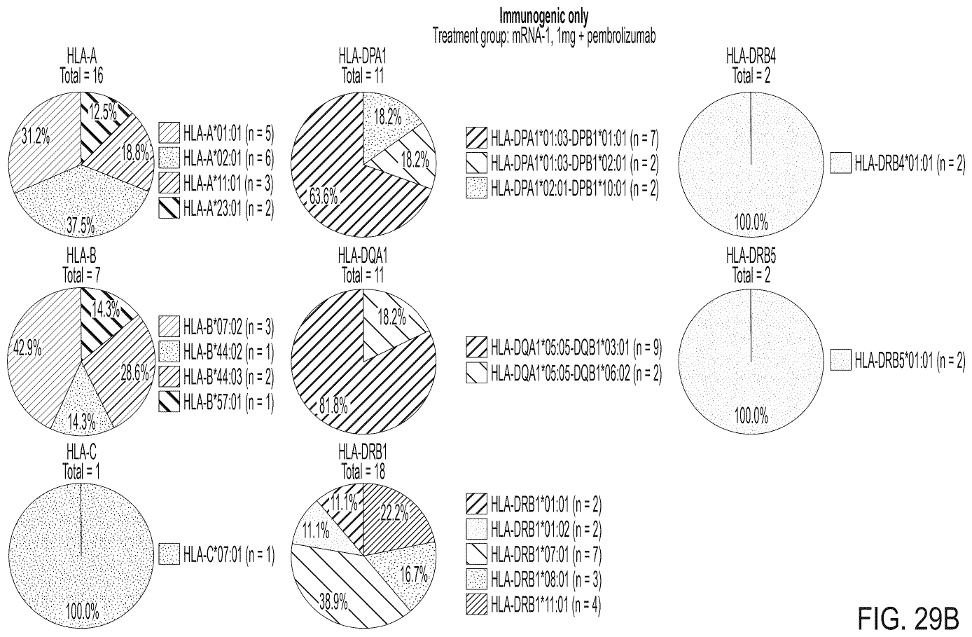

In order to evaluate the capacity of mRNA-1 to drive neoantigen-specific responses from both CD8 and CD4 T cells, individual neoantigens that were considered immunogenic by IFNγ ELISpot were used to expand patient PBMCs ex vivo. In silico-predicted neoantigens were verified to drive either MHC class I (CD8 T cell antigens) or class II (CD4 T cell antigens) molecules, or both (FIGS. 28A, 28B, 29A, 29B). Five individual neoantigens were expanded from one patient with melanoma. Neoantigen 2 was predicted to bind to MHC class I and class II; however, in vitro expansion did not drive measurable responses above those at the pre-treatment timepoint (FIG. 22F, 22G). Neoantigens 11, 16, and 17 were predicted to bind to MHC class I molecules and were validated to generate CD8 T cell responses. Neoantigen 33 was predicted to bind to MHC class II molecules; measured IFNγ responses were driven by CD4 T cells.

Across the 10 patients evaluable for immunogenicity analyses, MHC class I antigens were selected 72% of the time, class II antigens were selected 8% of the time, and class I and II antigens were selected 20% of the time (FIG. 22H). Responses to individual neoantigens were detected after in vitro expansion. Across all individual neoantigens, 6% of neoantigen-specific CD8+ responses were detected, whereas 24% of neoantigen-specific CD4+ responses were detected (FIG. 22I).

mRNA-1 Refines the T Cell Response to Neoantigens

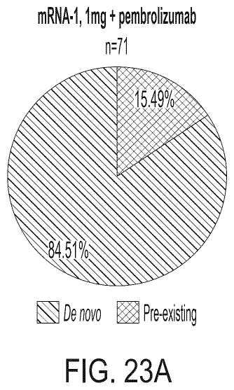

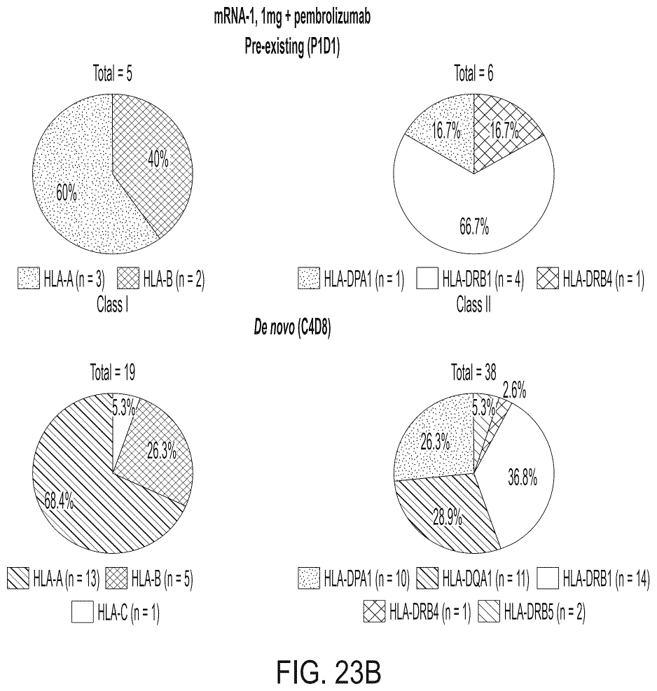

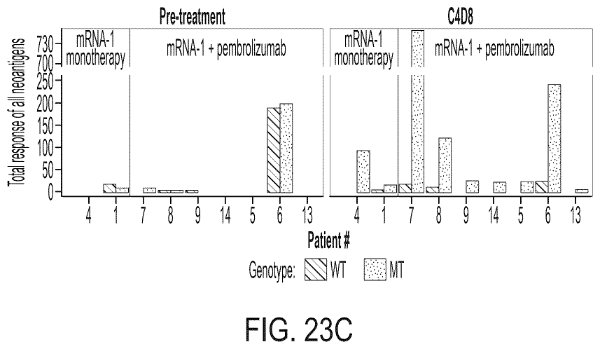

In order to assess whether mRNA-1 enhances pre-existing T cell responses and generates de novo T cell responses, a subanalysis of the in-silico predicted neoantigens was performed, and results were compared with the experimentally validated immunogenic responses. A total of 238 in-silico-predicted neoantigens were assessed in the 7 patients evaluable for immunogenicity analyses in Part D, of which 29.8% were immunogenic (FIG. 22D); 84.5% (71 neoantigens) solicited de novo responses (FIG. 23A). Pre-existing neoantigen responses (15%) were rarely seen prior to mRNA-1 treatment or after pembrolizumab treatment. Additionally, both pre-existing and de novo neoantigen-specific T cells were detected against peptides binding to a variety of HLA alleles, including class I and class II molecules (FIG. 23B). De novo responses to mRNA-1 neoantigens (not observed pre-treatment) were detected in 6/7 patients. The magnitude of pre-existing neoantigen responses observed in patients prior to treatment or following pembrolizumab run-in were further increased with combination therapy. In one patient with melanoma, a reduction in pre-existing responses to all mRNA-1 neoantigen pools was observed after pembrolizumab run-in; combination therapy increased the measured antigen-specific responses detected at C4D8 beyond what was initially measured prior to pembrolizumab run-in.

Pre-existing responses were predominately specific for the mutant antigen pre-treatment (FIG. 23C). A marginal response to the wildtype peptide was seen in one patient with NSCLC. Strong pre-existing responses to both the wildtype and mutant antigens were observed in one patient with melanoma. All responses detected post-treatment (C4D8) were against the mutated antigen with minimal or no cross-reactivity to the wildtype peptide.

Combination of mRNA-1 and Pembrolizumab Generates Activated T Cells with Cytotoxic Potential

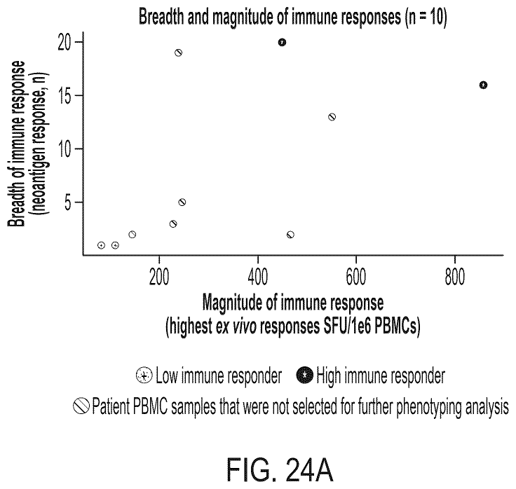

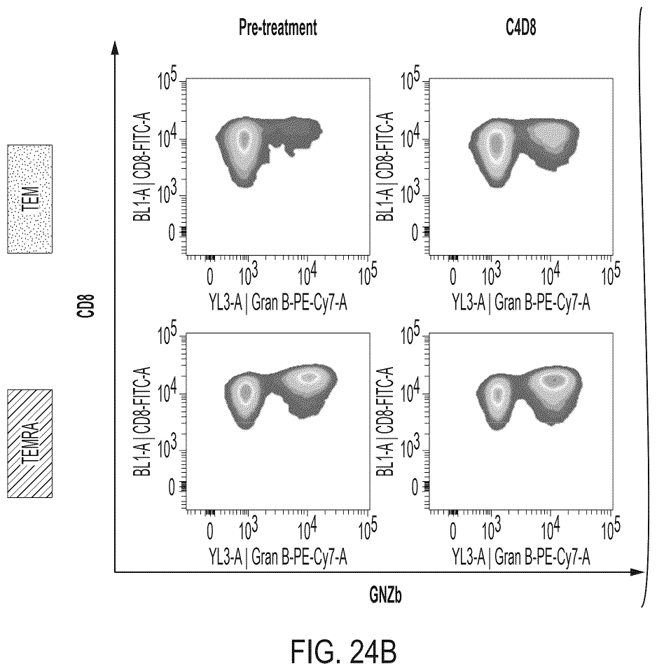

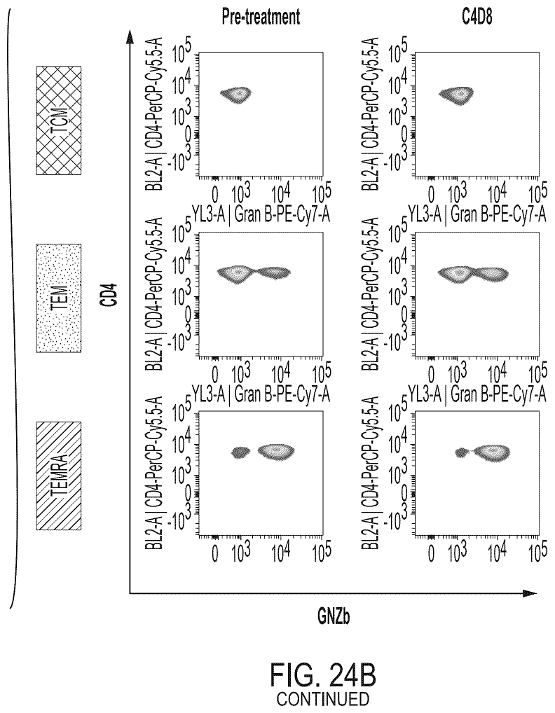

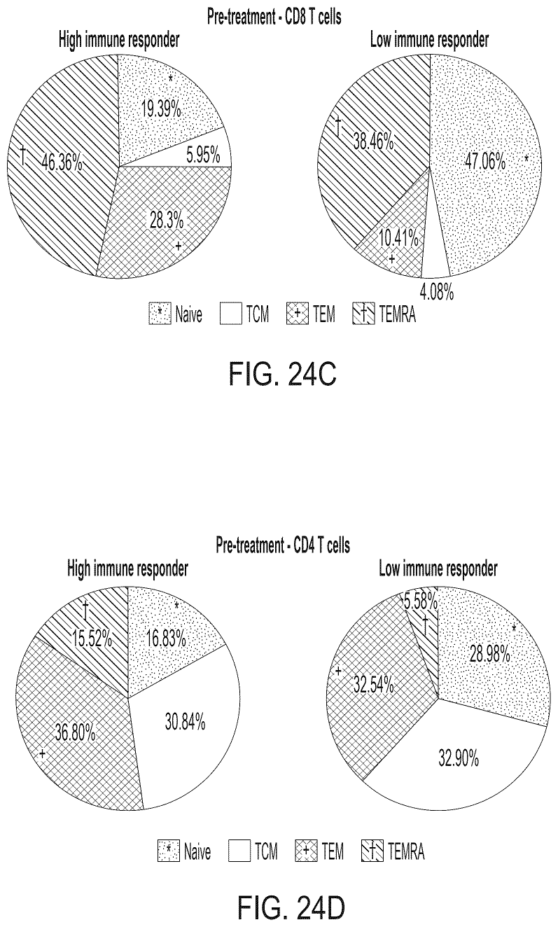

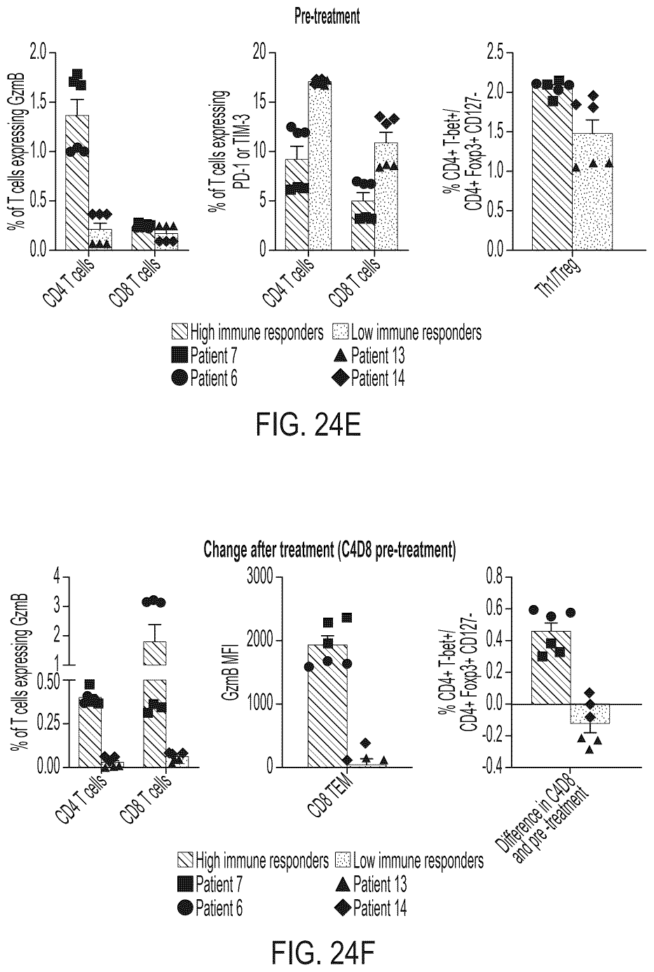

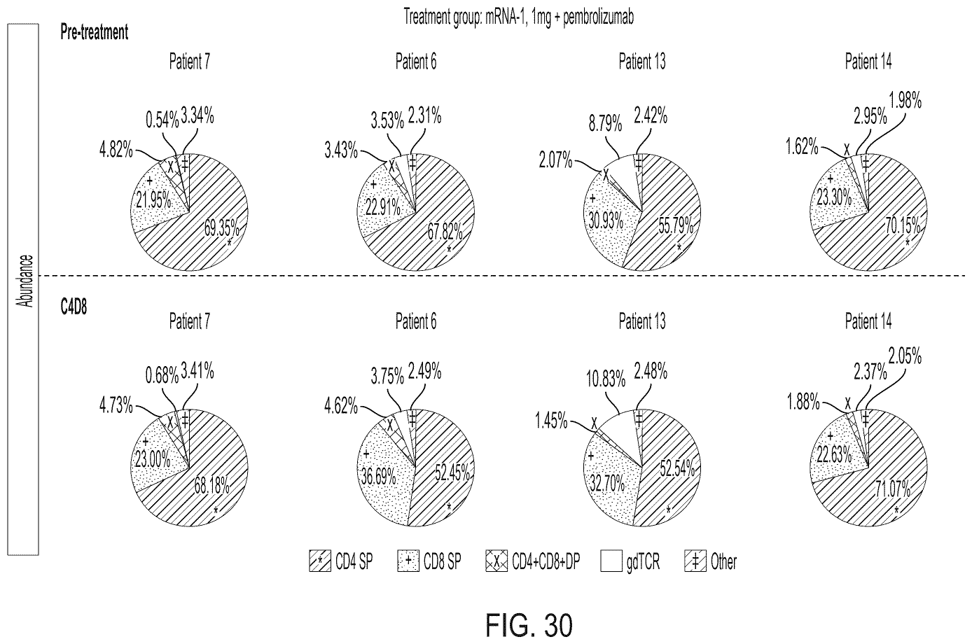

To better understand the underlying mechanisms driving T cell reactivity to mRNA-1, bulk PBMCs of two high immune responders (patients 6 and 7) and two low immune responders (patients 13 and 14) were further immunophenotyped based on the breadth and magnitude of neoantigen specific responses after combination treatment (FIG. 24A). Circulating T cells were phenotyped directly ex vivo at pre-treatment and C4D8 (FIGS. 24B, 24C, 24D, 24E, 24F). The proportion of CD4 and CD8 T cells remained consistent between pre-treatment and post-treatment for 3/4 evaluable patients (FIG. 30), with the exception of one high immune responder, in which a decrease in the frequency of CD4 T cells and an increase in CD8 T cells after treatment was observed.

Prior to treatment, the two high immune responders were characterized by a more pro-inflammatory state, with a higher proportion of effector memory CD8 T cells, Th1 T cells, and terminal CD4 effectors with cytotoxic potential as measured by granzyme B (FIGS. 24C, 24D). In contrast, low immune responders had an increased frequency of naïve memory cells but lower frequency of effector memory and terminally differentiated effector T cells (FIGS. 24C, 24D). Additionally, they had a higher proportion of inhibitory receptor (PD1+ or TIM-3+) expressing CD4 and CD8 T cells, and the CD4 T cells had little to no expression of granzyme B.

Notably, the majority of granzyme B-positive T cells across the 4 patients were CD8 T cells; however the high immune responders had a substantial proportion of granzyme B-expressing CD4 T cells (patient 7: 47%, patient 6: 33%) that were largely absent in the low immune responders (<6% each for patients 13 and 14). The low immune responders had a greater proportion of granzyme B-expressing gamma delta T cells (patient 13, 33%; patient 14, 30%) compared with only 1% in patient 7 and 10% in patient 6 (FIG. 30).

From pre-treatment to post-treatment, the proportion of effector CD8 T cell subsets (central memory, effector, and terminal effectors) were mostly unchanged, with the exception of one patient who had a decrease in naïve CD8 T cells (25% to 13%) and an increase in effector memory T cells (23% to 34%). Comparatively, combination treatment led to a shift in naïve CD4 T cells to effector CD4 T cells for the high immune responders; however, naïve CD4 T cells remained relatively high for the low immune responders. Interestingly, there was a high proportion of central memory CD4 T cells in all 4 patients; a decline in central memory CD4 T cells in favor of terminally differentiated T cells (TEMRA) was observed in one high immune responder, while a measurable increase in central memory CD4 T cells from 12% to 34% was observed in the other high immune responder.

Interestingly, a high proportion of naïve T cells was observed pre-treatment for the two low immune responders and remained prevalent after combination treatment, indicative of a more immunosuppressive environment or defective T cell priming. Combination treatment most notably drove an increase in the abundance of activated effector CD8 T cells with cytotoxic potential associated with granzyme B expression (FIGS. 24E, 24F). The two high immune responders had the greatest increase in frequency of granzyme B expression from pre-treatment to C4D8; however, the low immune responders also had an increased proportion of granzyme B effector CD8 T cells following combination treatment.

Following combination treatment, the per-cell expression of granzyme B in CD8 effectors increased in the high immune responders but was unchanged in the low immune responders (FIGS. 24E, 24F). Cytotoxic CD4 T cells were detected pre-treatment and further expanded after combination treatment in 3/4 patients (2 high immune responders and 1 low immune responder); however no cytotoxic CD4 responses were detected in patient 14 (low immune responder) either before or after treatment. Notably, no change in the abundance of regulatory T cells was observed after combination treatment (FIG. 30); however, the ratio of Th1/Treg CD4 T cells increased post-treatment in the high immune responders and decreased in the low immune responders.

The results of the first-in-human phase 1 study described herein inform the safety, immunogenicity, and MoA for mRNA-1 in patients with resected NSCLC (Part A) and for mRNA-1 plus pembrolizumab in patients with resected melanoma (Part D). Results presented here demonstrate the safety profile of mRNA-1, with no dose-limiting toxicities, and sustained and robust T cell immunogenicity; disease recurrence was limited to one patient. These findings are supportive of the proposed MoA of mRNA-1 and the Phase 2 clinical findings, and are further indicative of the potential benefit of an INT approach in the clinical setting.

All patients described in this Example experienced at least one TEAE, the events being mostly grade 1/2, self-limited, and manageable. No dose-limiting toxicities, study discontinuations related to mRNA-1, or grade 4/5 TEAEs were reported. Pyrexia, fatigue, and injection site reactions were the most common TEAEs with mRNA-1, aligning with the established safety profile of the Covid-19 vaccine, mRNA-1273 (Baden, et al. “Efficacy and Safety of the mRNA-1273 SARS-COV-2 Vaccine” N Engl J Med. 384(5):403-416 (2021)). The safety profile of the combination treatment was consistent with that of pembrolizumab monotherapy, suggesting that the combination of mRNA-1 and pembrolizumab reflects adverse effects associated with the individual treatments.

Although CPI therapy in the adjuvant setting has shown promise with immunologically active and high mutational burden tumors, nearly half of patients with melanoma do not respond to adjuvant anti-PD-1 therapy, underscoring a need for additional interventions in CPI resistant tumors. CPIs act on pre-existing T cells that are suboptimally primed in an immunosuppressive environment. Treatment with mRNA-1 monotherapy or combination therapy generated de novo neoantigen-specific T cell responses, indicating a benefit with mRNA-1-specific T cell priming compared to endogenous priming within the TME. Administration of mRNA-1 generated de novo T cell responses against the individualized neoantigens in both Parts A and D. Longitudinal immunogenicity analyses showed sustained T cell response to targeted neoantigens collected at 30 weeks after treatment initiation in Part D. Administration of mRNA-1 alone or with pembrolizumab induced both CD4 and CD8 T cells, in which expression of IFNγ and TNFα increased over time. Although the sample size in Part A was small, it is worth nothing that all 3 patients showed mRNA-1-specific T cell responses, which provides added rationale to support clinical trials of mRNA-1 in other cancers, such as NSCLC.

In patients who received combination therapy, robust T cell activation was observed, best characterized by an expansion of cytotoxic (granzyme B+) CD4 and CD8 T cells. T cell responses were low both at pre-treatment and after pembrolizumab run-in, suggesting that 1) treatment with pembrolizumab monotherapy did not expand neoantigen-specific T cell clones against neoantigens encoded in mRNA-1 and 2) mRNA-1 contributes to the observed increases in T cell response. These observations suggest that mRNA-1 and pembrolizumab are complementary to one another, with mRNA-1 capable of expanding de novo T cell clones which are unique compared to detected T cell clones with pembrolizumab alone.

Prior studies evaluating INTs in the clinic with synthetic long peptide or mRNA-lipoplex based therapies have demonstrated the capacity to expand neoantigen-specific T cells, however those studies reported a bias toward CD4 T cell responses, irrespective of in silico MHC-I binding predictions (Ott, et al. 2020, supra). In the present study, circulating neoantigen-specific CD4 and CD8 T cells were detected after combination therapy, with 60% of neoantigen reactive T cells being CD8. The observed T cell phenotypes are likely driven by a combination of the in silico neoantigen prediction algorithm, the intramuscular route of administration, cytosolic delivery of neoantigens into the MHC class I pathway, and an immunostimulatory capacity of modified mRNA encapsulated lipid nanoparticles.

In high immune responders versus low immune responders to combination therapy, stronger neoantigen-specific immunogenicity was associated with larger Th1, but smaller Treg populations pre-treatment. In melanoma and other cancer types it has been shown that elevated Th1 and effector memory T cell responses to melanoma and lung and hepatocellular carcinomas are correlated with positive clinical outcomes, whereas Treg responses to multiple solid tumor types are correlated with low clinical success. Based on the literature and the results of this study, it seems that patients who are high immune responders and who have a larger Th1 population may have better clinical success with mRNA-1 compared with low immune responders who have a smaller Th1 population. A high proportion of naïve T cells was also observed in the two low responders who received mRNA-1 plus pembrolizumab, suggesting a more immunosuppressive environment or defective T cell priming. Of note, one of the low immune responders in this analysis experienced disease recurrence in the study.

CPIs and other immunotherapies used for cancer can reverse CD8 effector T cell exhaustion following antigen exposure. The current findings show that mRNA-1 drives expansion of CD8 effector T cells and suggest that the MoA of mRNA-1 may include destruction of tumor cells by reversal of T cell exhaustion and enhanced T cell recognition of tumors. Furthermore, when combined with pembrolizumab, mRNA-1 increased granzyme B expression in peripheral CD4 and CD8 effector memory T cells and TEMRA cells ex vivo compared with pre-treatment, suggesting an immunological memory response after combination therapy. Future work will further investigate the MoA of mRNA-1 alone or in combination with pembrolizumab.

Individualized cancer vaccines have shown promise as adjuvant monotherapy in several solid tumor types, with some demonstrating a potential correlation between immunogenic induction and clinical response. Current data on individualized cancer vaccines and neoantigen therapies are limited; however, new insights continue to emerge. mRNA-1 is specific to each patient's tumor mutational profile and antigen-presenting molecules, including HLA type, thereby presenting a novel, individualized, addition to the oncology treatment landscape. Results of this study further support the validity and performance of the algorithm for mRNA-1, which can predict and select neoantigens with pre-existing tumor infiltrating lymphocyte reactivities with high accuracy.

These results show the INT mRNA-1 as monotherapy or in combination with pembrolizumab was immunogenic in patients with resected NSCLC or resected cutaneous melanoma and support the mechanism of action hypothesized for mRNA-1. They also demonstrate the robustness of the neoantigen selection algorithm and underscore potential clinical benefit when combined with a CPI.

### Example 9

A Phase 3, randomized, placebo- and active-controlled, parallel-group, multicenter, double-blind safety and efficacy study of adjuvant mRNA-1 plus pembrolizumab versus adjuvant placebo plus pembrolizumab in participants with completely resected Stage II, IIIA, IIIB (N2) NSCLC per American Joint Committee on Cancer Eighth Edition guidelines will be performed. Participants must have received at least 1 dose of adjuvant chemotherapy (platinum doublet) prior to full screening.

All participants must provide a blood sample and a formalin-fixed, paraffin embedded (FFPE) tumor sample as soon as possible after consent. A Limited Screening consent will be available for participants considering the study before or around the time of their surgery. Both the blood and FFPE samples are required for Next Generation Sequencing (NGS) and for design of the participant's mRNA-1, which will only be made for those participants randomly assigned to the combination arm (Arm A). In this study, randomization will only occur after the blood and FFPE tumor samples have successfully completed NGS, and the participant satisfies eligibility requirements. Eligible participants will be randomly assigned in a 1:1 ratio to receive treatment with either mRNA-1 plus pembrolizumab or placebo plus pembrolizumab. Randomization will be stratified according to the participants' histology (squamous versus nonsquamous); PD-L1 expression (TPS <1% versus 1 to 49% versus ≥50%); disease Stage (II versus III per AJCC Eighth Edition); and geographic location (North America/Western Europe/Australia versus Rest of World). Participants should begin pembrolizumab treatment as soon as possible (within 24 weeks after their surgery of curative intent).

For all participants:
The combination treatment period will begin once a participant's mRNA-1 or placebo is available. The start of placebo will be randomly adjusted to maintain the study blind.
Typically, the first dose of either mRNA-1 or placebo will be administered with the second dose of pembrolizumab (Day 1 of Cycle 2).
   The first dose of either mRNA-1 or placebo may begin as soon as Day 22 of Cycle 1.
   The first dose of either mRNA-1 or placebo may be started up to the time of the fourth dose (Day 1 of Cycle 4) of pembrolizumab (in consideration of timing for manufacturing of mRNA-1).

If mRNA-1 cannot be provided, the participant should continue in the study and receive placebo to maintain the study blind. Participants will receive treatment for up to 9 doses of either mRNA-1 or placebo every 3 weeks (q3w) plus 9 cycles of pembrolizumab every 6 weeks (q6w) until any of the criteria for discontinuation of study intervention are met. Crossover from one intervention arm to the other is not permitted. Participants will undergo imaging. Participants will be followed after discontinuation of study intervention for disease recurrence, distant metastasis, and survival.

The primary endpoint of the study is disease-free survival (DFS) and overall survival (OS) is a secondary endpoint.

Adverse Events (AEs) will be monitored throughout the study and graded in severity according to the guidelines outlined in the National Cancer Institute (NCI) Common Terminology Criteria for Adverse Events (CTCAE) v5.0. Each participant will be monitored for AEs and Serious Adverse Events (SAEs).
1. An individual is eligible for inclusion in the study if the individual meets all of the following criteria: Has surgically resected and histologically confirmed diagnosis of Stage II, IIIA, IIIB (with nodal involvement) squamous or nonsquamous NSCLC per AJCC Eighth Edition guidelines. A complete resection will have resection margins confirmed to be clear on microscopy and nodal sampling.
2. Confirmation that epidermal growth factor receptor (EGFR)-directed therapy is not indicated as primary therapy (historical documentation of absence of tumor-activating EGFR mutations or as determined by either a local or the central laboratory). If participant's tumor has a predominantly squamous histology, molecular testing for EGFR mutation is not required.
3. Has no evidence of disease (NED) at the time of providing documented consent for the main study. Disease-free status must be confirmed based on baseline radiological assessment as documented by contrast-enhanced chest/upper abdomen computed tomography (CT) or magnetic resonance imaging (MRI), and positron emission tomography (PET) scan per standard of care (SOC) within 28 days prior to randomization and contrast-enhanced brain CT/MRI within 28 days prior to randomization.
4. Has received at least one dose of adjuvant treatment with standard of care platinum doublet chemotherapy up to 4 cycles.
5. No more than 24 weeks have elapsed between surgical resection of curative intent and the first dose of pembrolizumab. Treatment should start only after adequate wound healing from the surgical procedure as assessed by the investigator. If there is a delay of ≤2 weeks exceeding 24 weeks after surgery due to unforeseen circumstances, the eligibility should be discussed with the Sponsor and the decision documented. Participants must have recovered from surgery and any postoperative complications before the first dose of pembrolizumab.
6. Has an FFPE tumor sample available (from their recent surgery) that is suitable for PD-L1 testing and the NGS required for this study.
7. Has an Eastern Cooperative Oncology Group Performance Status (ECOG PS) of 0 or 1 within 7 days before the first dose of pembrolizumab.
8. Is an individual of any sex/gender, from 18 years of age inclusive, at the time of providing the full informed consent.
9. For male participants, no contraception measures are required for participants capable of producing sperm; however, exposure to prior medication, including chemotherapy, and contraception requirements need to be reviewed.
10. A participant assigned female sex at birth is eligible to participate if not pregnant or breastfeeding, and at least one of the following conditions applies:
   a. Is not a person of childbearing potential (POCBP), OR
   b. Is a POCBP and:
      i. Uses a contraceptive method that is highly effective (with a failure rate of <1% per year), or is abstinent from penile-vaginal intercourse as their preferred and usual lifestyle (abstinent on a long-term and persistent basis) during the intervention period and for at least 120 days after the last dose of study intervention. The investigator should evaluate the potential for contraceptive method failure (i.e., noncompliance, recently initiated) in relationship to the first dose of study intervention. Contraceptive use by POCBPs should be consistent with local regulations regarding the methods of contraception for those participating in clinical studies. If the contraception requirements in the local label for any of the study interventions are more stringent than the requirements above, the local label requirements are to be followed. Exposure to prior medication, including chemotherapy, and contraception requirements need to be reviewed.
      ii. Has a negative highly sensitive pregnancy test (urine or serum) as required by local regulations within 24 hours (for a urine test) or 72 hours (for a serum test) before the first dose of study intervention. If a urine test cannot be confirmed as negative (e.g., an ambiguous result), a serum pregnancy test is required. In such cases, the participant must be excluded from participation if the serum pregnancy result is positive.
      iii. Abstains from breastfeeding during the study intervention period and for at least 120 days after study intervention mRNA-1/placebo or pembrolizumab.
      iv. Medical history, menstrual history, and recent sexual activity has been reviewed by the investigator to decrease the risk for inclusion of a POCBP with an early undetected pregnancy.
11. Participants who have AEs due to previous anticancer therapies must have recovered to ≤Grade 1 or baseline. Participants with endocrine-related Aes who are adequately treated with hormone replacement or participants who have ≤Grade 2 neuropathy are eligible.
12. Adequate organ function as defined in Table 19. Specimens must be collected within 7 days before the start of study intervention.

TABLE 19
Adequate Organ Function Laboratory Values

| | |
|---|---|
| System | Laboratory Value |

Hematological

| | |
|---|---|
| Absolute neutrophil count (ANC) | ≥1500/μL |
| Platelets | ≥100 000/μL |
| Hemoglobin | ≥9.0 g/dL or ≥5.6 mmol/L^{a} |

Renal

| | |
|---|---|
| Measured or calculated creatinine | ≥30 mL/min |
| clearance^{b} | |

Hepatic

| | |
|---|---|
| Total bilirubin | ≤1.5 × ULN OR direct bilirubin ≤ ULN for |
| | participants with total bilirubin levels > |
| | 1.5 × ULN |
| AST (SGOT) and ALT (SGPT) | ≤2.5 × ULN (≤5 × ULN for participants with |
| | liver metastases) |

Coagulation

| | |
|---|---|
| International normalized ratio (INR) | ≤1.5 × ULN unless participant is receiving |
| OR prothrombin time (PT) | anticoagulant therapy as long as PT or PTT |
| Activated partial thromboplastin time | is within therapeutic range |
| (aPTT) | of intended use of anticoagulants |

ALT (SGPT) = alanine aminotransferase (serum glutamic pyruvic transaminase); AST (SGOT) = aspartate aminotransferase (serum glutamic oxaloacetic transaminase); CrCl = creatinine clearance; ULN = upper limit of normal.
^{a}Criteria must be met without erythropoietin dependency and without packed red blood cell (pRBC) transfusion within last 2 weeks.
^{b}CrCl using Cockcroft-Gault CrCl formula: [(140 − age [years]) × weight (kg)]/[72 × serum Cr (mg/dL)] × F where F = 0.85 for females and F = 1 for males.

13. Participants who are hepatitis B surface antigen (HBsAg) positive are eligible if they have received hepatitis B virus (HBV) antiviral therapy for at least 4 weeks, and have undetectable HBV viral load prior to randomization. Participants should remain on antiviral therapy throughout study intervention and follow local guidelines for HBV antiviral therapy post completion of study intervention.
14. Participants with history of hepatitis C virus (HCV) infection are eligible if HCV viral load is undetectable at screening. Participants must have completed curative antiviral therapy at least 4 weeks prior to randomization.
15. Human immunodeficiency virus (HIV)-infected participants must have well controlled HIV on antiretroviral therapy (ART), defined as:
   a. Having a CD4+ T-cell count ≥350 cells/mm3 at the time of screening.
   b. Having achieved and maintained virologic suppression defined as confirmed HIV RNA level below 50 or the LLOQ (below the limit of detection) using the locally available assay at the time of screening and for at least 12 weeks before screening.
   c. Have not had any AIDS-defining opportunistic infections within the past 12 months.
   d. Have been on a stable ART regimen, without changes in drugs or dose modification, for at least 4 weeks before randomization and agree to continue ART throughout the study.

An individual must be excluded from the study if the individual meets any of the following criteria:
1. Diagnosis of SCLC or, for mixed tumors, presence of small cell elements, or has a neuroendocrine tumor with large cell components or a sarcomatoid carcinoma.
2. HIV-infected participants with a history of Kaposi's sarcoma and/or Multicentric Castleman's Disease.
3. Received prior neoadjuvant therapy for their current NSCLC diagnosis.
4. Received or is a candidate to receive radiotherapy for their current NSCLC diagnosis.
5. Received prior treatment with another personalized cancer vaccine.
6. Received prior therapy with an anti-PD-1, anti-PD-L1, or anti-PD-L2 agent, or with an agent directed to another stimulatory or coinhibitory T-cell receptor (e.g., CTLA-4, OX-40, CD137).
7. Received prior systemic anticancer therapy including investigational agents within 4 weeks before randomization.
8. Received a live or live-attenuated vaccine within 30 days before the first dose of study intervention. Administration of killed vaccines are allowed.
9. Has received an investigational agent or has used an investigational device within 4 weeks prior to study intervention administration.
10. Diagnosis of immunodeficiency or is receiving chronic systemic steroid therapy (in dosing exceeding 10 mg daily of prednisone equivalent) or any other form of immunosuppressive therapy within 7 days prior the first dose of study medication.
11. Known additional malignancy that is progressing or has required active treatment within the past 5 years. Participants with basal cell carcinoma of the skin, squamous cell carcinoma of the skin, or carcinoma in situ, excluding carcinoma in situ of the bladder, that have undergone potentially curative therapy are not excluded. Participants with low-risk early-stage prostate cancer (T1-T2a, Gleason score ≤6, and prostate specific antigen (PSA) <10 ng/mL) either treated with definitive intent or untreated in active surveillance with stable disease are not excluded. Participants receiving hormonal therapy must discontinue within 30 days prior to the first dose of study medication and for the duration of the study.
12. Severe hypersensitivity (≥Grade 3) to either mRNA-1 or pembrolizumab and/or any of its excipients.
13. Active autoimmune disease that has required systemic treatment in the past 2 years. Replacement therapy (e.g., thyroxine, insulin, or physiologic corticosteroid) is allowed.
14. History of (noninfectious) pneumonitis/interstitial lung disease that required steroids or has current pneumonitis/interstitial lung disease.
15. Active infection requiring systemic therapy.
16. Concurrent active Hepatitis B (defined as HBsAg positive and/or detectable HBV DNA) and Hepatitis C virus (defined as anti-HCV Ab positive and detectable HCV RNA) infection.
17. History or current evidence of any condition, therapy, laboratory abnormality, or other circumstance that might confound the results of the study or interfere with the participant's participation for the full duration of the study, such that it is not in the best interest of the participant to participate, in the opinion of the treating investigator.
18. Known psychiatric or substance abuse disorder that would interfere with the participant's ability to cooperate with the requirements of the study.
19. History of allogenic tissue/solid organ transplant.
20. Participants who have not adequately recovered from major surgery or have ongoing surgical complications.

### Example 10

A phase 2, randomized, double-blind, placebo- and active-controlled, parallel-group, multicenter, safety and efficacy study of mRNA-1 plus pembrolizumab versus placebo plus pembrolizumab in the adjuvant treatment of participants with RCC post nephrectomy will be performed. This study will enroll participants with RCC with clear cell or papillary histology that is intermediate-high risk, high risk, or M1 NED (M1 NED refers to participants who present not only with the primary kidney tumor, but also solid, isolated, soft tissue metastases that can be completely resected within 2 years from the time of nephrectomy, but ≤12 weeks before randomization).

Eligible participants will be randomly assigned in a 1:1 ratio to receive treatment with either mRNA-1 plus pembrolizumab or placebo plus pembrolizumab. Randomization will be stratified according to histology (clear cell versus papillary) and the participant's disease risk (intermediate-high risk versus high risk versus M1 NED). Participants should begin pembrolizumab treatment as soon as possible (within 12 weeks of their surgery of curative intent).

For all participants:
The combination treatment period will begin once a participant's mRNA-1 or placebo is available. The start of placebo will be randomly adjusted to maintain the study blind.
Typically, the first dose of either mRNA-1 or placebo will be administered with the second dose of pembrolizumab (Day 1 of Cycle 2).
The first dose of either mRNA-1 or placebo may begin as soon as Day 22 of Cycle 1.
The first dose of either mRNA-1 or placebo may be delayed until Day 22 of Cycle 2 or up until the third dose (Day 1 of Cycle 3) of pembrolizumab (in the event of any NGS or INT manufacture delays).
If either mRNA-1 or placebo cannot be provided by the time of Day 1 of Cycle 3 of pembrolizumab administration, the participant may continue in the study and receive placebo to maintain the study blind.
Delays in starting mRNA-1 or placebo beyond Day 1 of Cycle 3 for reasons unrelated to mRNA-1/placebo production may be allowed following Sponsor consultation.

Participants will receive treatment for up to 9 doses of either mRNA-1 or placebo q3w (once available at the next closest scheduled cycle day [either Day 1 or Day 22]) plus 9 cycles of pembrolizumab q6w until any of the criteria for discontinuation of study intervention are met.

Participants will be followed after discontinuation of study intervention for disease recurrence and survival.

The primary endpoint of the study is DFS.

AEs will be monitored throughout the study and graded in severity according to the guidelines outlined in the NCI CTCAE v5.0. Each participant will be monitored for AEs and SAEs.

An individual is eligible for inclusion in the study if the individual meets all of the following criteria:
1. Has histologically or cytologically confirmed diagnosis of RCC with clear cell or papillary histology per AJCC, with or without sarcomatoid features, that has not been treated with prior systemic therapy or radiotherapy.
2. Has intermediate-high-risk, high-risk, or M1 NED RCC as defined by the following pathological tumor-node metastasis and tumor grading:
   a. Intermediate-high-risk RCC:
      i. pT2 Gr4, N0, M0
      ii. pT3 Gr3/4, N0, M0
   b. High-risk RCC:
      i. pT4, N0, M0
      ii. pT any stage, N1, M0
   c. M1 NED RCC participants who present not only with the primary kidney tumor, but also solid, isolated, soft tissue metastases that can be completely resected at 1 of the following:
      i. the time of nephrectomy (synchronous), or
      ii. ≤2 years from nephrectomy (metachronous)
3. Has undergone complete resection of the primary tumor (partial or radical nephrectomy) and complete resection of solid, isolated, soft tissue metastatic lesion(s) in M1 NED participants.
4. Participants with microscopically positive soft tissue or vascular margins without gross residual disease are permitted.
5. Must have undergone a nephrectomy and/or metastasectomy ≤12 weeks prior to randomization. If there is a delay of ≤4 weeks exceeding 12 weeks due to unforeseen circumstances, the eligibility should be discussed with the Sponsor and the decision documented. Participants must have recovered from surgery and any post-operative complications before randomization.
6. Must be tumor-free before randomization as assessed by the investigator by either CT or MRI scan of the brain; and chest, abdomen, and pelvis (CAP) (≤28 days from randomization) and a bone scan (≤42 days from randomization).
7. Must provide FFPE tissue sample from:
   a. Nephrectomy for intermediate-high or high-risk disease
   b. Metastasectomy for M1 NED (nephrectomy if metastasectomy tissue not available).
8. Is an individual of any sex/gender, at least 18 years of age, at the time of providing the informed consent.
9. A participant assigned female sex at birth is eligible to participate if not pregnant or breastfeeding, and at least one of the following conditions applies:
   a. Is not a POCBP, or
   b. Is a POCBP and:
      i. Uses a contraceptive method that is highly effective (with a failure rate of <1% per year), or is abstinent from penile-vaginal intercourse as their preferred and usual lifestyle (abstinent on a long-term and persistent basis), during the intervention period and for at least 120 days after the last dose of study intervention. The investigator should evaluate the potential for contraceptive method failure (i.e., noncompliance, recently initiated) in relationship to the first dose of study intervention. Contraceptive use by POCBPs should be consistent with local regulations regarding the methods of contraception for those participating in clinical studies. If the contraception requirements in the local label for any of the study interventions are more stringent than the requirements above, the local label requirements are to be followed.
      ii. Has a negative highly sensitive pregnancy test (urine or serum) as required by local regulations within 24 hours (for a urine test) or 72 hours (for a serum test) before the first dose of study intervention. If a urine test cannot be confirmed as negative (e.g., an ambiguous result), a serum pregnancy test is required. In such cases, the participant must be excluded from participation if the serum pregnancy result is positive.
      iii. Abstains from breastfeeding during the study intervention period and for at least 120 days after study intervention.
      iv. Medical history, menstrual history, and recent sexual activity has been reviewed by the investigator to decrease the risk for inclusion of a POCBP with an early undetected pregnancy.
10. The participant (or legally acceptable representative) has provided documented informed consent for the study.
11. Adequate organ function as defined in the following table (Table 20). Specimens must be collected within 7 days before the start of study intervention.

TABLE 20

| | |
|---|---|
| System | Laboratory Value |

Hematological

| | |
|---|---|
| Absolute neutrophil count (ANC) | ≥1500/μL |
| Platelets | ≥100,000/μL |
| Hemoglobin | ≥9.0 g/dL or ≥5.6 mmol/L^{a} |

Renal

| | |
|---|---|
| Measured or calculated creatinine clearance^{b} | ≥30 mL/min |

Hepatic

| | |
|---|---|
| Total bilirubin | ≤1.5 × ULN OR direct bilirubin ≤ ULN for participants |
| | with total bilirubin levels > 1.5 × ULN |
| AST (SGOT) and ALT (SGPT) | ≤2.5 × ULN (≤5 × ULN for participants with liver |
| | metastases) |

Coagulation

| | |
|---|---|
| International normalized ratio (INR) OR | ≤1.5 × ULN unless participant is receiving anticoagulant |
| prothrombin time (PT) | therapy as long as PT or PTT is within therapeutic range |
| Activated partial thromboplastin time (aPTT) | of intended use of anticoagulants |

ALT (SGPT) = alanine aminotransferase (serum glutamic pyruvic transaminase); AST (SGOT) = aspartate aminotransferase (serum glutamic oxaloacetic transaminase); ULN = upper limit of normal.
^{a}Criteria must be met without erythropoietin dependency and without packed red blood cell (pRBC) transfusion within last 2 weeks.
^{b}CrCl using Cockcroft-Gault CrCl formula: [(140 − age [years]) × weight (kg)]/[72 × serum Cr (mg/dL)] × F where F = 0.85 for females and F = 1 for males. As an alternative, CrCl can be determined from a 24-hour urine collection.

12. Participants who are HBsAg positive are eligible if they have received HBV antiviral therapy for at least 4 weeks, and have undetectable HBV viral load prior to randomization. Participants should remain on antiviral therapy throughout study intervention and follow local guidelines for HBV antiviral therapy post completion of study intervention.
   a. Hepatitis B screening tests are not required unless:
      i. Known history of HBV infection
      ii. As mandated by local health authority
13. Participants with history of HCV infection are eligible if HCV viral load is undetectable at screening. Participants must have completed curative antiviral therapy at least 4 weeks prior to randomization.
   a. Hepatitis C screening tests are not required unless:
      i. Known history of HCV infection
      ii. As mandated by local health authority
14. HIV-infected participants must have well controlled HIV on ART, defined as:
   a. Having a CD4+ T-cell count ≥350 cells/mm3 at the time of screening.
   b. Having achieved and maintained virologic suppression defined as confirmed HIV RNA level below 50 or the LLOQ (below the limit of detection) using the locally available assay at the time of screening and for at least 12 weeks before screening.
   c. Have not had any AIDS-defining opportunistic infections within the past 12 months.
   d. Have been on a stable ART regimen, without changes in drugs or dose modification, for at least 4 weeks before randomization and agree to continue ART throughout the study.
   e. HIV screening tests are not required unless:
      i. Known history of HIV infection
      ii. As mandated by local health authority
15. Has an ECOG performance status of 0 or 1 within 7 days before randomization.

An individual must be excluded from the study if the individual meets any of the following criteria:
1. Has had a major surgery within 4 weeks prior to randomization. Nephrectomy and metastasectomy are allowed if participant has fully recovered from surgery.
2. Has residual thrombus post nephrectomy in the vena renalis or vena cava.
3. Received prior therapy with an anti-PD-1, anti-PD-L1, or anti-PD-L2 agent, or with an agent directed to another stimulatory or coinhibitory T-cell receptor (e.g., CTLA-4, OX-40, CD137).
4. Received prior systemic anticancer therapy including investigational agents within 4 weeks before randomization.
5. Received prior radiotherapy within 2 weeks of start of study intervention, or radiation-related toxicities, requiring corticosteroids. Two weeks or fewer of palliative radiotherapy for non-CNS disease is permitted. The last radiotherapy treatment must have been performed at least 7 days before the first dose of study intervention.
6. Received a live or live-attenuated vaccine within 30 days before the first dose of study intervention. Administration of killed vaccines is allowed.
7. Received prior treatment with a cancer vaccine.
8. Has received an investigational agent or has used an investigational device within 4 weeks prior to study intervention administration.
9. Diagnosis of immunodeficiency or is receiving chronic systemic steroid therapy (in dosing exceeding 10 mg daily of prednisone equivalent) or any other form of immunosuppressive therapy within 7 days prior to the first dose of study medication.
10. Known additional malignancy that is progressing or has required active treatment within the past 3 years. Participants with basal cell carcinoma of the skin, squamous cell carcinoma of the skin, or carcinoma in situ, excluding carcinoma in situ of the bladder, that have undergone potentially curative therapy are not excluded. Participants with low-risk early-stage prostate cancer (T1-T2a, Gleason score ≤6, and PSA <10 ng/mL) either treated with definitive intent or untreated in active surveillance with stable disease are not excluded.
11. History of brain or bone metastatic lesions.
12. Severe hypersensitivity (≥Grade 3) to either mRNA-1 or pembrolizumab and/or any of their excipients.
13. Active autoimmune disease that has required systemic treatment in the past 2 years. Replacement therapy (e.g., thyroxine, insulin, or physiologic corticosteroid) is allowed.
14. History of (noninfectious) pneumonitis/interstitial lung disease that required steroids or has current pneumonitis/interstitial lung disease.
15. Active infection requiring systemic therapy.
16. History or current evidence of any condition, therapy, laboratory abnormality, or other circumstance that might confound the results of the study or interfere with the participant's participation for the full duration of the study, such that it is not in the best interest of the participant to participate, in the opinion of the treating investigator.
17. Known psychiatric or substance abuse disorder that would interfere with the participant's ability to cooperate with the requirements of the study.
18. History of allogeneic tissue/solid organ transplant.
19. Participants who have not adequately recovered from major surgery or have ongoing surgical complications.

### Example 11

A Phase 2, randomized, placebo- and active-controlled, parallel-group, multicenter, double-blind safety and efficacy study of adjuvant mRNA-1 plus pembrolizumab (Arm A) versus adjuvant placebo plus pembrolizumab (Arm B) in participants with pathologic high-risk muscle-invasive urothelial carcinoma (MIUC) (e.g., ypT2-4a and/or ypN+ after neoadjuvant cisplatin-based chemotherapy; pT3-4a and/or pN+ participants without neoadjuvant cisplatin-based chemotherapy) after radical resection will be performed.

All participants must provide a blood sample and a FFPE tumor tissue sample as soon as possible after documented (limited or full) informed consent. Both the blood and FFPE tumor samples are required for NGS and for design of the mRNA-1, which will only be made for those participants randomly assigned to the combination arm (Experimental Arm A). All 9 doses of mRNA-1 are manufactured prior to the start of treatment with mRNA-1. Randomization will only occur after the blood and FFPE tumor tissue samples have passed the NGS QC check, and the participant satisfies all eligibility requirements. Archival tissue may be submitted if the tissue is of sufficient quantity and quality.

Participants will be randomly assigned in a 1:1 ratio to receive treatment with either mRNA-1 plus pembrolizumab or placebo plus pembrolizumab. Randomization will be stratified according to ctDNA status at screening (e.g., positive vs negative vs nonevaluable) and prior neoadjuvant chemotherapy (e.g., yes vs no). The ctDNA test that will be used in this study is a tumor-informed MRD test.

Given the prognostic significance of ctDNA for MIUC after radical resection, the presence or absence of ctDNA in the peripheral blood during screening will be used for stratification at the time of randomization in this study. Peripheral blood and tumor tissue will be collected for ctDNA evaluation during screening and peripheral blood thereafter at regular intervals during the course of the study. Analyses with ctDNA will include evaluation of the prognostic significance of ctDNA at screening, before the initiation of pembrolizumab and before the first dose of mRNA-1/placebo administration and at subsequent timepoints. At the interim and final efficacy analyses DFS will be assessed in the ctDNA-positive and ctDNA-negative subgroups.

The second stratification factor used for randomization is prior neoadjuvant chemotherapy (NAC) (yes vs no), given the prognostic significance of prior NAC for high-risk MIUC.

All participants will receive treatment for up to 9 doses of either mRNA-1 or placebo every 3 weeks (q3w) plus 9 cycles of pembrolizumab every 6 weeks (q6w) or until any of the criteria for discontinuation of study intervention is met. The administration schedule for pembrolizumab and mRNA-1/placebo is as follows:
Pembrolizumab is given on Day 1 of each 6-week cycle.
mRNA-1 production begins only after a participant is randomized to the experimental arm, and the earliest it will be available at a site for administration is Day 22 of Cycle 1. Hence, on Day 1 of Cycle 1, all participants on both the experimental arm and comparator arm will receive pembrolizumab monotherapy.
The combination treatment period will begin once a participant's mRNA-1 or placebo is available. The start of placebo will be randomly adjusted to maintain the study blind. The administration for the first dose of placebo will be staggered in interactive response technology (IRT) to mimic the anticipated administration of the first dose of mRNA-1.
Typically, the first dose of either mRNA-1 or placebo will be administered with the second dose of pembrolizumab (Day 1 of Cycle 2).
The first dose of either mRNA-1 or placebo may begin as soon as Day 22 of Cycle 1.
The first dose of either mRNA-1 or placebo may be delayed until Day 22 of Cycle 2 or up until the fourth dose (Day 1 of Cycle 4) of pembrolizumab (in consideration of timing for completion of NGS or manufacturing of mRNA-1).
Once mRNA-1/placebo is initiated, it will be administered on Day 1 and Day 22 of each 6-week cycle.
If mRNA-1 is not available at the sites for administration to a participant on the experimental arm by Day 1 of Cycle 4, then placebo will be initiated on that day, to maintain the study blind. If mRNA-1 becomes available subsequently (after Day 1 of Cycle 4), placebo will be discontinued and mRNA-1 will be initiated on Day 22 of the ongoing cycle, or Day 1 of the next cycle, whichever is sooner. The participant will receive a total maximum of 9 doses of mRNA-1/placebo, including both initial placebo and subsequent mRNA-1 doses.

This study will use DFS based on recurrence as assessed by the investigator as the primary endpoint. DFS is an acceptable measure of clinical benefit for a randomized study in MIUC that demonstrates superiority of a new antineoplastic therapy especially if the magnitude of the effect is large and the therapy has an acceptable risk/benefit profile.

The secondary efficacy objectives of this study are to evaluate OS and DMFS between the 2 treatment arms in this study. OS has been recognized as the gold standard for the demonstration of superiority of a new antineoplastic therapy in randomized clinical studies. For patients with MIUC, disease recurrence outside the urothelial tract is associated with worse prognosis than local recurrence within the urothelial tract which can often be managed with local curative therapy. The DMFS endpoint in this study provides an additional measure of efficacy, evaluating the clinical benefit of the experimental treatment compared with the comparator treatment in preventing disease recurrence outside the urothelial tract.

RECIST 1.1 principles will be used when assessing scans for efficacy measures.

AEs will be monitored throughout the study and graded in severity according to the guidelines outlined in the NCI CTCAE v5.0. Each participant will be monitored for AEs and SAEs.

An individual is eligible for inclusion in the study if the individual meets all of the following criteria:
1. The participant must have MIUC originating in the lower tract (bladder, urethra) or upper tract (renal pelvis, ureter).
2. Dominant histology must be urothelial carcinoma (UC). Histology will be determined locally.
   a. Participants with mixed histology are eligible provided the urothelial component is ≥50%.
   b. Participants whose tumors contain any neuroendocrine component are not eligible.
3. Participants must have undergone radical resection for MIUC ≤8 weeks before providing informed consent and ≤16 weeks before randomization. Radical resection refers to radical nephroureterectomy (for upper tract MIUC) or radical cystectomy with pelvic lymph node dissection per American Urological Association (AUA)/American Society of Clinical Oncology (ASCO)/American Society for Radiation Oncology (ASTRO)/Society of Urologic Oncology (SUO) guidelines. Partial cystectomy may be permitted upon Sponsor consultation.
   a. Participants with positive surgical margins for microscopic disease (R1) are eligible.
4. Participants must have high-risk pathologic disease (determined locally) after radical resection, as per 1 of 2 definitions:
   a. For participants who received cisplatin-based neoadjuvant chemotherapy: ypT2-4a and/or ypN+
   b. For participants who have not received cisplatin-based neoadjuvant chemotherapy: pT3-4a and/or pN+
5. Participants who have not received cisplatin-based neoadjuvant chemotherapy are eligible with 1 of following scenarios:
   a. Participant is cisplatin-ineligible per 1 or more of the following criteria:
      i. CrCl (using the Cockcroft-Gault formula): <60 mL/min and >30 mL/min
      ii. CTCAE v5.0 Grade 2 or higher audiometric hearing loss
      iii. CTCAE v5.0 Grade 2 or higher peripheral neuropathy
      iv. ECOG performance status 2
   b. Participant is cisplatin-eligible but declines adjuvant cisplatin-based chemotherapy.
6. Participants must provide an FFPE tumor tissue sample that is suitable for the NGS required for this study. Tissue from the radical resection is mandated for participants who received neoadjuvant chemotherapy followed by radical resection. For participants who did not receive neoadjuvant chemotherapy and underwent radical resection, tissue from radical resection is strongly preferred to ensure QC for NGS is successful, but tissue from transurethral resection of bladder tumor (TURBT) is allowed as long as tissue requirements are met.
7. Participants must provide blood samples as specified in the protocol, to enable mRNA-1 production, and ctDNA testing.
8. Participants must be disease-free (NOMO) with no evidence of disease per investigator assessment based on imaging studies within 4 weeks before randomization.
   a. Imaging must include CT or MRI of the chest, abdomen, and pelvis.
   b. For participants with upper tract disease and an intact bladder, a cystoscopy (with or without biopsy) must be performed within 4 weeks before randomization.
   c. If there is clinical suspicion of CNS disease at screening, brain scan is required within 4 weeks before randomization. MRI of the brain is preferred, however, CT scan will be acceptable.
   d. Bone scan may be performed during screening if clinically indicated.
9. Participants must provide blood samples as specified in the protocol, to enable mRNA-1 production, and ctDNA testing.
10. Prior treatment for non-muscle invasive bladder cancer (NMIBC) with intravesical instillation therapy such as Bacillus Calmette-Guérin (BCG) or intra-vesical chemotherapy is permitted.
11. Prior systemic neoadjuvant cisplatin-based chemotherapy is permitted for MIUC.
12. Is an individual of any sex/gender, from 18 of age inclusive, at the time of providing the informed consent.
13. No contraception measures are required for participants capable of producing sperm.
14. A participant assigned female sex at birth is eligible to participate if not pregnant or breastfeeding, and at least one of the following conditions applies:
   a. Is not a POCBP, or
   b. Is a POCBP and:
      i. Uses a contraceptive method that is highly effective (with a failure rate of <1% per year), or is abstinent from penile-vaginal intercourse as their preferred and usual lifestyle (abstinent on a long-term and persistent basis), during the intervention period and for at least 120 days after the last dose of study intervention. The investigator should evaluate the potential for contraceptive method failure (i.e., noncompliance, recently initiated) in relationship to the first dose of study intervention. Contraceptive use by POCBPs should be consistent with local regulations regarding the methods of contraception for those participating in clinical studies. If the contraception requirements in the local label for any of the study interventions are more stringent than the requirements above, the local label requirements are to be followed.
      ii. Has a negative highly sensitive pregnancy test (urine or serum) as required by local regulations within 24 hours (for a urine test) or 72 hours (for a serum test) before the first dose of study intervention. If a urine test cannot be confirmed as negative (e.g., an ambiguous result), a serum pregnancy test is required. In such cases, the participant must be excluded from participation if the serum pregnancy result is positive.
      iii. Abstains from breastfeeding during the study intervention period and for at least 120 days after study intervention with mRNA-1/placebo or pembrolizumab.
      iv. Medical history, menstrual history, and recent sexual activity has been reviewed by the investigator to decrease the risk for inclusion of a POCBP with an early undetected pregnancy.
15. The participant (or legally acceptable representative) has provided documented informed consent for the study.
16. An ECOG performance status of 0 to 2 assessed within 7 days before randomization.
17. Participants who have AEs due to previous anticancer therapies must have recovered to ≤Grade 1 or baseline. Participants with endocrine-related AEs who are adequately treated with hormone replacement or participants who have <Grade 2 neuropathy are eligible.
18. Adequate organ function as defined in Table 21. Specimens must be collected within 7 days before the start of study intervention.

TABLE 21
Adequate Organ Function Laboratory Values

| | |
|---|---|
| System | Laboratory Value |

Hematologic

| | |
|---|---|
| Absolute neutrophil count | ≥1500/μL |
| Platelets | ≥100,000/μL |
| Hemoglobin | ≥9.0 g/dL or ≥5.6 mmol/L^{a} |

Renal

| | |
|---|---|
| Creatinine OR measured or | ≤1.5 × ULN or ≥30 mL/min |
| calculated^{b} CrCl OR GFR | |

Hepatic

| | |
|---|---|
| Total bilirubin | ≤1.5 × ULN OR direct bilirubin ≤ ULN for |
| | participants with total bilirubin > 1.5 × ULN |
| AST (SGOT) and ALT (SGPT) | ≤2.5 × ULN |

Coagulation

| | |
|---|---|
| INR or PT | ≤1.5 × ULN unless the participant is receiving |
| aPTT | anticoagulants, as long as PT or aPTT is within the |
| | therapeutic range for intended use of anticoagulants |

ALT (SGPT) = alanine aminotransferase (serum glutamic-pyruvic transaminase); aPTT = activated partial thromboplastin time; AST (SGOT) = aspartate aminotransferase (serum glutamic-oxaloacetic transaminase); CrCl = creatinine clearance; GFR = glomerular filtration rate; INR = international normalized ratio; PT = prothrombin time; ULN = upper limit of normal.
^{a}This criterion must be met without erythropoietin dependency and without packed red blood cell transfusion within the last 2 weeks.
^{b}CrCl should be calculated or measured per institutional standard.

19. Participants who are HBsAg positive are eligible if they have received HBV antiviral therapy for at least 4 weeks, and have undetectable HBV viral load before randomization. Participants should remain on antiviral therapy throughout study intervention and follow local guidelines for HBV antiviral therapy post completion of study intervention. Hepatitis B screening tests are not required unless:
   a. Known history of HBV infection
   b. As mandated by local health authority
20. Participants with history of HCV infection are eligible if HCV viral load is undetectable at screening. Participants must have completed curative antiviral therapy at least 4 weeks before randomization. Hepatitis C screening tests are not required unless:
   a. Known history of HCV infection
   b. As mandated by local health authority
21. HIV-infected participants must have well controlled HIV on ART, defined as:
   a. Having a CD4+ T-cell count ≥350 cells/mm3 at the time of screening.
   b. Having achieved and maintained virologic suppression defined as confirmed HIV RNA level below 50 or the LLOQ (below the limit of detection) using the locally available assay at the time of screening and for at least 12 weeks before screening.
   c. Have not had any AIDS-defining opportunistic infections within the past 12 months.
   d. Have been on a stable ART regimen, without changes in drugs or dose modification, for at least 4 weeks before randomization and agree to continue ART throughout the study.

An individual must be excluded from the study if the individual meets any of the following criteria:
1. Received prior therapy with an anti-PD-1, anti-PD-L1, or anti-PD-L2 agent, or with an agent directed to another stimulatory or coinhibitory T-cell receptor (e.g., CTLA-4, OX-40, CD137). Exception includes participants who received anti-PD-1 or PD-L1 therapy for NMIBC with recurrence >12 months before study randomization.
2. Received prior systemic anticancer therapy including investigational agents in the adjuvant setting after radical surgery.
3. Received a live or live-attenuated vaccine within 30 days before the first dose of study intervention. Administration of killed vaccines are allowed.
4. Received therapy with hematopoietic growth factor such as granulocyte colony-stimulating factor (G-CSF) or granulocyte macrophage colony-stimulating factor (GM-CSF) within 14 days before randomization.
5. Received prior treatment with a cancer vaccine.
6. Prior neoadjuvant therapy, with the exception of neoadjuvant cisplatin-based chemotherapy.
7. Has received an investigational agent or has used an investigational device within 4 weeks prior to study intervention administration.
8. Diagnosis of immunodeficiency or is receiving chronic systemic steroid therapy (in dosing exceeding 10 mg daily of prednisone equivalent) or any other form of immunosuppressive therapy within 7 days prior the first dose of study medication.
9. Known additional malignancy that is progressing or has required active treatment ≤3 years prior to study randomization. Participants with basal cell carcinoma of the skin, squamous cell carcinoma of the skin, or carcinoma in situ, excluding carcinoma in situ of the bladder, that have undergone potentially curative therapy are not excluded. Participants with low-risk early-stage prostate cancer (T1-T2a, Gleason score ≤6, and PSA <10 ng/ml) either treated with definitive intent or untreated in active surveillance with stable disease are not excluded.
10. Severe hypersensitivity (≥Grade 3) to either mRNA-1 or pembrolizumab and/or any of their excipients.
11. Active autoimmune disease that has required systemic treatment in the past 2 years. Replacement therapy (e.g., thyroxine, insulin, or physiologic corticosteroid) is allowed.
12. History of (noninfectious) pneumonitis/interstitial lung disease that required steroids or has current pneumonitis/interstitial lung disease.
13. Active infection requiring systemic therapy.
14. HIV-infected participants with a history of Kaposi's sarcoma and/or Multicentric Castleman's Disease.
15. Concurrent active hepatitis B (defined as HBsAg positive and/or detectable HBV DNA) and hepatitis C virus (defined as anti-HCV Ab positive and detectable HCV RNA) infection. Hepatitis B and C screening tests are not required unless:
   a. Known history of HBV and HCV infection
   b. As mandated by local health authority
16. History or current evidence of any condition, therapy, laboratory abnormality, or other circumstance that might confound the results of the study or interfere with the participant's participation for the full duration of the study, such that it is not in the best interest of the participant to participate, in the opinion of the treating investigator.
17. Known psychiatric or substance abuse disorder that would interfere with the participant's ability to cooperate with the requirements of the study.
18. Had radiation therapy for UC after radical resection.
19. History of allogeneic tissue/solid organ transplant.
20. Participants who have not adequately recovered from major surgery or have ongoing surgical complications.

### Example 12

A Phase 2/3, adaptive, randomized, open-label, clinical study to evaluate neoadjuvant and adjuvant mRNA-1 in combination with pembrolizumab versus standard of care, and pembrolizumab monotherapy in participants with resectable locally advanced cutaneous squamous cell carcinoma (LA cSCC) will be performed.

Participants will be with resectable Stage II, Stage III, and Stage IV (M0) without distant metastases. Resectable cSCC is defined as cSCC that is amenable to achieve complete oncologic resection (R0 or R1) and is not expected to result in permanent significant functional loss or severe disfigurement. R0 is a complete surgical resection where all resection margins are confirmed to be negative by microscopy. R1 resection is when the tumor is resected leaving microscopic residual disease at the margin(s), which cannot be re-resected to clear margins. For participants with Stage II, the tumor must be ≥3 cm at the longest diameter in an aesthetic and/or organ-function threatening areas (e.g., ear, eye, mouth, cranial nerves).

Distant metastasis, which refers to cancer that has spread from the primary tumor and beyond local tissues and regional lymph nodes to distant organs or non-regional lymph nodes, is excluded. Participants must not have received any prior systemic anticancer therapy for their cSCC.

All participants must provide a blood sample and a FFPE tumor tissue sample as soon as possible after documented (limited or full) informed consent. Both the blood and FFPE tumor samples are required for NGS and for design of the mRNA-1, which will only be made for those participants randomly assigned to the combination arm (Experimental Arm A). All 9 doses of mRNA-1 are manufactured prior to the start of treatment with mRNA-1.

Participants will be randomly assigned to one of the following study intervention arms:
Experimental Arm A: mRNA-1 plus Pembrolizumab
Comparator Arm B: standard of care (SOC).
Experimental Arm C: Pembrolizumab

For participants in Experimental Arm A:
In order not to delay pembrolizumab administration in the Neoadjuvant Period, pembrolizumab will begin after randomization; however, treatment with mRNA-1 will start once a participant's mRNA-1 is available. In case the mRNA-1 is started earlier or delayed, then the second dose of mRNA-1 will occur 3 weeks (±3 days) from the first mRNA-1 dose.
Participants will receive treatment for up to 9 doses of mRNA-1 q3w (2 neoadjuvant doses+7 adjuvant doses are recommended) and up to 11 doses of pembrolizumab q6w (2 neoadjuvant doses+9 adjuvant doses) or until any of the criteria for discontinuation of study intervention are met. It is recommended that participants receive 2 doses of mRNA-1 and pembrolizumab in the Neoadjuvant Period.

Participants in Comparator Arm B will not receive neoadjuvant study intervention and will proceed directly to surgery as per local practice.

Participants in Experimental Arm C will receive treatment for up to 11 doses of pembrolizumab q6w (2 neoadjuvant doses+9 adjuvant doses) or until any of the criteria for discontinuation of study intervention are met.

Participants will undergo imaging and digital photography. Pre-surgery scans are required within 14 days prior to the date of surgery. Repeat scans are required if imaging is done >14 days prior to the date of surgery. For Comparator Arm B, the screening scans can serve as the pre-surgery scans if performed within 14 days prior to surgery. If the screening scans are done >14 days prior to the date of surgery for Comparator Arm B, then repeat pre-surgery scans are required.

For Experimental Arm A and Experimental Arm C:
Surgical resection will occur between 77 and 100 days following N1D1 (Day 1 of Cycle 1 in the Neoadjuvant Period). It is recommended that there is a minimum of 14 days from the last dose of neoadjuvant study intervention to surgery.
Participants who decline a biopsy or a warranted surgery, cannot undergo surgery due to any AE, or do not meet freedom from surgery (FFS) criteria will discontinue study intervention and imaging and will proceed directly to end of treatment (EOT) and continue with survival follow-up (SFU).

For Comparator Arm B:
Surgical resection will occur after the pre-surgery scans. Participants in Comparator Arm B who decline surgery, or cannot undergo surgery due to any AE, will discontinue study intervention and imaging, and then they will proceed directly to EOT and continue with SFU.

For All Arms:
Participants with a clinical CR with no residual tumor on clinical exam and by imaging and confirmed by negative biopsy, are allowed not to undergo surgical resection, and will continue to receive adjuvant study intervention without adjuvant radiation therapy (RT). These participants who have surgery omitted will be asked to undergo a biopsy. The biopsy site should be consistent with known prior sites of disease (e.g., prior nodal metastasis, prior primary site, or if no tumor was identified, then biopsy of the scar).
Participants that have a negative biopsy may skip surgery and adjuvant RT and will continue with adjuvant study intervention. For participants who meet the freedom from surgery (FFS) criteria in Arm A or Arm C, A1D1 should occur no later than 4 weeks after the confirmation negative biopsy. Participants in any arm that have a positive biopsy will be asked to undergo surgery.
Following surgical resection, for those participants with residual disease or microscopic positive margin involvement on pathology, re-resection should be performed unless medically contraindicated prior to starting adjuvant therapy. Post-surgery follow-up (FU) will occur anytime up to 3-6 weeks following surgical resection or re-resection, whichever is applicable. Participants who have surgery with subsequent pathological complete response (pCR) by local assessment are not allowed to have adjuvant RT and will proceed with adjuvant study intervention. Post-surgical complications will be assessed and graded according to NCI CTCAE v5.0.
After surgical resection (or re-resection) of the tumor, if margins are still positive on pathology, participants with R0/R1 disease may proceed with adjuvant RT prior to receiving adjuvant study intervention. RT must start within 4-8 weeks after surgery and must be completed prior to A1D1 (for Arm A and Arm C).
Adjuvant RT is permitted for participants with negative margins prior to the start of adjuvant study intervention. Participants will receive their assigned adjuvant therapy, starting at least 4 weeks and up to 12 weeks after surgery (or last dose of RT if the participant undergoes RT).
Tumor biopsy will be required at any disease recurrence, or suspicion of second primary tumor, unless there is an unacceptable safety risk associated with biopsy in a particular participant.
Participants will either complete study intervention or discontinue study intervention early due to unacceptable toxicity, event-free survival (EFS)/DMFS event, or any of the other discontinuation criteria. Participants who meet EFS event criteria before surgery or after surgery (e.g., participants with R2 disease) will not be allowed to receive adjuvant therapy, and they instead must proceed directly to EOT and continue with SFU; no further imaging will be performed.
If a participant in Experimental Arm A or Experimental Arm C discontinues neoadjuvant study intervention early due to unacceptable toxicity, they may still proceed to surgery. Participants who, undergo surgery or meet FFS criteria, and do not meet EFS/DMFS event criteria will be followed after discontinuation of study intervention during the Efficacy Follow-Up Phase.
AEs will be monitored throughout the study and graded in severity according to the guidelines outlined in the NCI CTCAE v5.0. Each participant will be monitored for AEs and SAEs.

An individual is eligible for inclusion in the study if the individual meets all of the following criteria:
1. The participant must have a histologically confirmed diagnosis of resectable cSCC as the primary site of malignancy (metastatic skin involvement from another primary cancer or from an unknown primary cancer is not permitted). Resectable cSCC is defined as cSCC that is amenable to achieve complete oncologic resection (R0 or R1) and is not expected to result in permanent significant functional loss or severe disfigurement. Participants for whom the primary site of SCC was an anogenital area (e.g., penis, scrotum, vulva, perianal region) are not eligible. Participants with tumors arising on cutaneous non-glabrous (hair-bearing) lip with extension onto vermillion (dry red lip) may be eligible after communication and approval from the Clinical Director. Participants for whom the primary site is the nose may be eligible after communication and approval from the CD if the primary site is skin, not nasal mucosa with outward extension to skin.
2. Has LA Stage II-IV (M0) cSCC without distant metastases. cSCC tumors arising in the head and neck will be staged according to AJCC Ed. 8 and cSCC tumors arising in non-head and neck locations will be staged according to UICC Ed. 7. For participants with Stage II cSCC lesion(s), their tumor(s) must be ≥3 cm at the longest diameter in aesthetic and/or organ-function threatening areas (e.g., ear, eye, mouth, cranial nerves). Stage II will be capped at 5% of total enrollment. Participants with the recurrent cSCC who were treated with prior definitive surgery with curative intent are allowed.
3. cSCC must be amenable to surgery (resectable) with curative intent.
4. Has an FFPE tumor sample available that is suitable for the Next-generation Sequencing (NGS) required for this study. The tumor sample must meet the following criteria:
   a. Meet the minimum standards for tissue quantity and quality as defined in the Procedures/Laboratory Manual for this study.
   b. Pass the required QC checks for NGS by the Sponsor's NGS vendor. Archival or newly obtained tumor tissue sample from the tumor biopsy or surgical resection of the primary tumor (in a participant with recurrent cSCC lesion), or lymph node resection, not previously irradiated can be provided. FNA and cytology samples are not acceptable. Newly obtained tumor tissue from a biopsy is preferred to archival tissue.
5. Is an individual of any sex/gender, at least 18 years of age, at the time of providing the full informed consent.
6. No contraception measures are required for participants capable of producing sperm if only mRNA-1 and pembrolizumab are administered. For participants exposed to adjuvant RT and capable of producing sperm, the participant agrees to the following during the intervention period and for at least the time needed to eliminate the study intervention after the last dose of study intervention. The length of time required to continue contraception for the study intervention is adjuvant RT, if performed: 90 days
   a. Refrains from donating sperm plus either:
      i. Abstains from penile-vaginal intercourse as their preferred and usual lifestyle (abstinent on a long-term and persistent basis) and agrees to remain abstinent or
   b. Uses contraception as detailed below unless confirmed to be azoospermic (vasectomized or secondary to medical cause, documented from the site personnel's review of the participant's medical records, medical examination, or medical history interview) as detailed below:
      i. Uses a penile/external condom when having penile-vaginal intercourse with a nonparticipant of childbearing potential who is not currently pregnant plus partner use of an additional contraceptive method, as a condom may break or leak. Note: Participants capable of producing ejaculate whose partner is pregnant or breastfeeding must agree to use a penile/external condom during each episode of sexual activity in which the partner is at risk of exposure via ejaculate.
      ii. Contraceptive use by participants capable of producing sperm should be consistent with local regulations regarding the methods of contraception for those participating in clinical studies.
7. A participant assigned female sex at birth is eligible to participate if not pregnant or breastfeeding, and at least one of the following conditions applies:
   a. Is not a POCBP or
   b. Is a POCBP and:
      i. Uses a contraceptive method that is highly effective (with a failure rate of <1% per year), with low user dependency, or is abstinent from penile-vaginal intercourse as their preferred and usual lifestyle (abstinent on a long-term and persistent basis). The participant agrees not to donate eggs (ova, oocytes) to others or freeze/store eggs during this period for the purpose of reproduction. The length of time required to continue contraception for each study intervention is:
         1. mRNA-1:15 days
         2. Pembrolizumab: 120 days
         3. Adjuvant RT, if performed: 90 days after last exposure
      ii. The investigator should evaluate the potential for contraceptive method failure (i.e., noncompliance, recently initiated) in relationship to the first dose of study intervention. Contraceptive use by POCBPs should be consistent with local regulations regarding the methods of contraception for those participating in clinical studies. If the contraception requirements in the local label for any of the study interventions are more stringent than the requirements above, the local label requirements are to be followed.
      iii. Has a negative highly sensitive pregnancy test (urine or serum) as required by local regulations within 24 hours (for a urine test) or 72 hours (for a serum test) before the first dose of study intervention. If a urine test cannot be confirmed as negative (e.g., an ambiguous result), a serum pregnancy test is required. In such cases, the participant must be excluded from participation if the serum pregnancy result is positive.
      iv. Abstains from breastfeeding during the study intervention period and for at least 15 days after study intervention with mRNA-1 or 120 days after study intervention with pembrolizumab.
      v. Medical history, menstrual history, and recent sexual activity has been reviewed by the investigator to decrease the risk for inclusion of a POCBP with an early undetected pregnancy.
8. The participant (or legally acceptable representative) has provided documented informed consent (full consent) for the study.
9. Measurable disease per RECIST 1.1 as assessed by the local site investigator/radiology. Lesions situated in a previously irradiated area are considered measurable if progression has been shown in such lesions. If an excisional or incisional biopsy has been performed, participants remain eligible for the study provided the residual disease is measurable per RECIST 1.1.
10. Has a life expectancy of >3 months per investigator assessment.
11. An ECOG performance status of 0 to 1 assessed within 14 days before randomization.
12. Adequate organ function as defined in the following table (Table 22). Specimens must be collected within 7 days before the start of randomization.

TABLE 22
Adequate Organ Function Laboratory Values

| | |
|---|---|
| System | Laboratory Value |

Hematological

| | |
|---|---|
| Absolute neutrophil count (ANC) | ≥1500/μL |
| Platelets | ≥100,000/μL |
| Hemoglobin | ≥9.0 g/dL or ≥5.6 mmol/L^{a} |

Renal

| | |
|---|---|
| Measured or calculated creatinine clearance^{b} | ≥30 mL/min |

Hepatic

| | |
|---|---|
| Total bilirubin | ≤1.5 × ULN OR direct bilirubin ≤ ULN for participants |
| | with total bilirubin levels > 1.5 × ULN |
| AST (SGOT) and ALT (SGPT) | ≤2.5 × ULN (≤5 × ULN for participants with liver |
| | metastases) |

Coagulation

| | |
|---|---|
| International normalized ratio (INR) OR | ≤1.5 × ULN unless participant is receiving anticoagulant |
| prothrombin time (PT) | therapy as long as PT or PTT is within therapeutic range |
| Activated partial thromboplastin time (aPTT) | of intended use of anticoagulants |

ALT (SGPT) = alanine aminotransferase (serum glutamic pyruvic transaminase); AST (SGOT) = aspartate aminotransferase (serum glutamic oxaloacetic transaminase); GFR = glomerular filtration rate; ULN = upper limit of normal.
^{a}Criteria must be met without erythropoietin dependency and without packed red blood cell (pRBC) transfusion within last 2 weeks.
^{b}CrCl using Cockcroft-Gault CrCl formula [(140 − age [yr]) × weight (kg)]/[72 × serum Cr (mg/dL)] × F where F = 0.85 for females and F = 1 for males

13. Participants who are HBsAg positive are eligible if they have received HBV antiviral therapy for at least 4 weeks, and have undetectable HBV viral load prior to randomization. Participants should remain on antiviral therapy throughout study intervention and follow local guidelines for HBV antiviral therapy post completion of study intervention. Hepatitis B screening tests are not required unless:
   a. Known history of HBV infection
   b. As mandated by local health authority
14. Participants with history of HCV infection are eligible if HCV viral load is undetectable at screening. Participants must have completed curative antiviral therapy at least 4 weeks prior to randomization. Hepatitis C screening tests are not required unless:
   a. Known history of HCV infection
   b. As mandated by local health authority
15. HIV-infected participants must have well controlled HIV on ART, defined as:
   a. Having a CD4+ T-cell count ≥350 cells/mm³ at the time of screening.
   b. Having achieved and maintained virologic suppression defined as confirmed HIV RNA level below 50 or the LLOQ (below the limit of detection) using the locally available assay at the time of screening and for at least 12 weeks before screening.
   c. Have not had any AIDS-defining opportunistic infections within the past 12 months.
   d. Have been on a stable ART regimen, without changes in drugs or dose modification, for at least 4 weeks before randomization and agree to continue ART throughout the study.

Individual must be excluded from the study if the individual meets any of the following criteria:
1. Has any other histologic type of skin cancer other than invasive cSCC as well as mixed histology, e.g., basal cell carcinoma that has not been definitively treated with surgery or radiation, Bowen's disease, MCC, or melanoma.
2. Distant metastatic disease (M1), visceral and/or distant nodal
3. Received prior therapy with an anti-PD-1, anti-PD-L1, or anti-PD-L2 agent, or with an agent directed to another stimulatory or coinhibitory T-cell receptor (e.g., CTLA-4, OX-40, CD137).
4. Received prior systemic anticancer therapy including investigational agents for cSCC before randomization.
5. Received prior radiotherapy within 2 weeks of start of study intervention, or has radiation-related toxicities, requiring corticosteroids.
6. Received a live or live-attenuated vaccine within 30 days before the first dose of study intervention. Administration of killed vaccines is allowed.
7. Received transfusion of blood products (including platelets or red blood cells) or administration of colony stimulating factors (including granulocyte colony-stimulating factor, granulocyte macrophage colony-stimulating factor, or recombinant erythropoietin) within 2 weeks of the screening blood sample (including the NGS blood sample).
8. Received prior treatment with another cancer vaccine.
9. Received prior radiotherapy to the index lesion (in-field lesion). Participant must have recovered from all radiation-related toxicities prior to randomization and not have had radiation pneumonitis.
10. Has received an investigational agent or has used an investigational device within 4 weeks prior to study intervention administration.
11. Diagnosis of immunodeficiency or is receiving chronic systemic steroid therapy (in dosing exceeding 10 mg daily of prednisone equivalent) or any other form of immunosuppressive therapy within 7 days prior the first dose of study medication.
12. Known additional malignancy that is progressing or has required active treatment within the past 2 years. Participants with basal cell carcinoma of the skin, squamous cell carcinoma of the skin, melanoma in situ, invasive melanoma <1 mm without ulceration or carcinoma in situ, excluding carcinoma in situ of the bladder, that have undergone potentially curative therapy are not excluded. Participants with low-risk early-stage prostate cancer (T1-T2a, Gleason score ≤6, and PSA <10 ng/mL) either treated with definitive intent or untreated in active surveillance with stable disease are not excluded. Participants with low-risk early-stage prostate cancer defined as below are not excluded: Stage T1c or T2a with a Gleason score ≤6 and a prostate-specific antigen (<10 ng/ml) either treated with definitive intent or untreated in active surveillance that has been stable for the past year prior to study allocation. Other known additional malignancy may be considered with Sponsor consultation. Other exceptions may be considered with Sponsor consultation.
13. History of chronic lymphocytic leukemia (CLL).
14. History of CNS metastases and/or carcinomatous meningitis.
15. Severe hypersensitivity (≥Grade 3) to either mRNA-1 or pembrolizumab and/or any of their excipients.
16. Active autoimmune disease that has required systemic treatment in the past 2 years. Replacement therapy (e.g., thyroxine, insulin, or physiologic corticosteroid) is allowed.
17. History of (noninfectious) pneumonitis/interstitial lung disease that required steroids or has current pneumonitis/interstitial lung disease.
18. Active infection requiring systemic therapy.
19. HIV-infected Multicentric Castleman's Disease. Hepatitis B and C screening tests are not required unless:
   a. Known history of HBV and HCV infection
   b. As mandated by local health authority
20. History or current evidence of any condition, therapy, laboratory abnormality, or other circumstance that might confound the results of the study or interfere with the participant's ability to cooperate with the requirements of the study, such that it is not in the best interest of the participant to participate, in the opinion of the treating investigator.
21. Myocardial infarction within 6 months of randomization.
22. History of allogeneic tissue/solid organ transplant.
23. Participants who have not adequately recovered from major surgery or have ongoing surgical complications.

### Example 13

This Example describes an example molecular sequence structure of mRNA-1. mRNA-1 in this Example has a sequence length of 1,233-2,925 nucleotides and incorporates the features in Table 23 below.

TABLE 23
Structural elements of exemplary mRNA-1 messenger RNA sequence

| mRNA Sequence Molecular | 419,900-969,354 Daltons | |
|---|---|---|
| Weight Range (free acid) | | |
| mRNA Sequence Molecular | 1,233-2,925 nucleotides | |
| Length Range | | |
| mRNA Sequence Elements | Cap + 5′ UTR: | 49 nucleotides |
| | ORF | ORF specific to an individual patient, with an |
| | | expected length of 960-2,652 nucleotides. |
| | | Below, a representative ORF sequence in |
| | | underlined text is provided, however this will |
| | | be unique for each patient. |
| | 3′ UTR: | 119 nucleotides |
| | PolyA tail: | 105 nucleotides |

Example mRNA-1 molecular sequence:

5′7MeGpppG2′OMe-

| | |
|---|---|
| AGGAAAUA AGAGAGAAAA GAAGAGUAAG AAGAAAUAUA AGAGCCACCA | 50 |
| UGUGGGCCGC CUGCACCAAC UUCAGCAGAA CCAGAAAGGA GAUCCUUCUU | 100 |
| UUCGCCGAGA UCAUCUUGUG CCUUGUGGCC CCUAACCAGG AGAGCGGCAU | 150 |
| GAAGACCGCC GACUUCCUCA GAGUGCUAAG CGGCCACUUA AUGCAGACCA | 200 |
| GAGAGGCCGA GCCUAAGGGC ACCAUCACCC UGGAGCUGAU CGAGCACAAC | 250 |
| GAGGCCUACA CCUGGACCAA CCCUACCGGC GGCCUGGCCG UGCUGGCCAA | 300 |
| GGAGCGUCCU CCUAACCUCA UUGAGUUCCU GGCCAGCUAC CUGCUGAAGA | 350 |
| ACAAGGGCGA GCAGUACUUC AAGAGAACCA UGCCUAGAAU CAGCACCCUG | 400 |
| AAGAAUCUGG AGGACCUGGU GACCGAGUAC CCUAGAGGCA UCUUCACCAA | 450 |
| GGAGGACGCC CUGAAGUUCG UGCAGCUGAA GCAGACCGGC AAGAUCACCG | 500 |
| AGAGCCCUGA GAAGACCGUG CUGACCCAGG AGGCCAUCAU CAUCGUGAAG | 550 |
| GGCGUGAGCC UGUCCUCCUA UCUCGAAGGC CAGGCCCCUG CCGUGGAGGU | 600 |
| GGCUCCAGCC GGCGCCUUCU ACAACCCUAG CUUCGAGGAC CACCAGACCC | 650 |
| UGUUGUCGGA CAGACUGAGC AACCACAUCA GCAGCCUGUU CUGUGAGGAU | 700 |
| CAGAUCUACA GAAUCGACCA CUACCUGGGC AGCACCGAAC ACAAUAAGGA | 750 |
| GUGCCUGAUC AACAUCUUCA AGUACAAGUU CAGCCUGGUG AUCAGCGGCC | 800 |
| UGACCGUGUG GAGCAUCAGA GUGACGAGCA CGGAGGAGUA CCUGCACCUG | 850 |
| AAGCCUGCCA GAUACAGAAG AGGCUUCAUC GAGCAGAGAA ACGUGAGCGG | 900 |
| CGGUUACCUG GUGCUGUGCA AGAUGAACUA CGCCACCAGA AUCGUGACAC | 950 |
| UGGAGUGCUC CUACCCUGAG ACUGAGGAGG AGGGCGAAGC AAUUCCUGUG | 1000 |
| AGAGACAGCU UCUACAGACU GGAGAAGAGA CUGUGGAAGC AGCAGAUGUA | 1050 |
| CACCAUCGCC AAGUUCUACU UCUUCACACU UAAGAGAGAG AGCAAGAGCG | 1100 |
| UGAGGCGUCU CUCAGAAGAA GAACUGCCUG CCAUCCUGAA GCUUAAGAAG | 1150 |
| AGUCUGCAGA AGCUGGUGAG CGACUGGAAC AGAGGCGCCC AGCUACGCAG | 1200 |
| GCCUAGACAC UCACACCUGA CCAGAGCCAG AAGAUGCCCU GGAGGCCUCC | 1250 |
| CUGGCCCUGC CAUGAACUAU GACAAGCUGA GCAGAAGCCU GUGCUACUAC | 1300 |
| UACGAGAAGG GCAUCAUGCA GAAGGUGGCC ACCAAGGCCU UCCUGGACGG | 1350 |
| CUUCAACGAA GUGGCCAGCC UUGAAUGGCU GAGAUACUUC GAUGAGAAGG | 1400 |
| AACUUAAGUC UGGUUAUCCA GACAUCGGCU UCCCUCUGUU CCUGCUGAGU | 1450 |
| AAGGGUUUCA UCAAGUCCAU CAGAGGCGUG GACAACCCUG GCGUGCACCC | 1500 |
| UGCCACCUUC GCCGCCUACA CUGCACCGGC CUACUACCCU UACGGCCAGU | 1550 |
| UCCAGGGCAG CUUCGUCCAG GCCACCCCUC ACAACAGAGU GAGUUACUUG | 1600 |
| AGAGCCUUCU GGAGAUGCUU CAGAACCGUG GGCACCGAGA AGUGCGAGGU | 1650 |
| GGUGAUCCAG CACUUCAACA GAAAGUACCU GAAGACCCCU CCUGGCAUCC | 1700 |
| CUGCCCCUAC CAAGCUGGAC GAGCUAAAGA GGCAGGAGGU GUCACAGCUC | 1750 |
| CGCAUGUUAC UUAAGGCCAA GAUGGACGCC GAGAAGGCCA GCAAUUUGCA | 1800 |
| GAAGAAUAAG ACAGCCAGCU UCACCACAAA GGAGAAGGAG ACGAAGCUGC | 1850 |
| CUCUGCUGCA GCUCGAAGAU UUGGACAGCC GAGUGCUUUA UUAUUUGGCC | 1900 |
| ACAAAGAACG GCAUCCUGUA CAGAGUGGGC GACCUGGGCU UACGGAGCGG | 1950 |
| CAGCUACAGC CUGCCUCGCA UUAUCCACAU CGCCAGAAGC CAGAACUUCA | 2000 |
| GAAAGAGCAA GUUCGGCAGC AGACACUCCG CAGAAUCCCA GAUUUUCAAG | 2050 |
| CACCUCCUUA AGAACCUUUU CAAGAUCUUC UGCAAGCAGU CUCAGCACAU | 2100 |
| GACCGAAGUG GUGAGACGGU GGCCUCACCA CGAGAGAUGC AGCGACAGCG | 2150 |
| ACGGCCUGAG CCACUACAGC AUCUGGGAGG AAGUAGUCGC CCGUAGCUUG | 2200 |
| GCACGGGUGU UAGUGUUCGA GGAUGACGUG AGAGGAGUCG GCAAGGGCAA | 2250 |
| GCGACGACCU CUGAACAACA ACUUCGAGAU CGCCCUUUCA CUGUCAAGAA | 2300 |
| ACAAGGUGUU CGACCCUGUG AUCGUGGAAG CUUUCUUAGC CUCACUAUUC | 2350 |
| AACAGGCUGU ACAUCUCUCA GGAAUCAGAC AAGGAUCACC UGAGACUGGU | 2400 |
| GGAGAGCGCC AAUGAGAAGG CUAAGCGGGA GGCCCUCCUG CGCGGUGUCG | 2450 |
| AACCAGGUCC UCAGGAGUUC UUCAAGGGCU ACUUAAAGGG AGAGCACAGC | 2500 |
| AGACUACUCU CUCUCUGGAG AGAGGUCGUG ACAUUCUCCC GCCAGGCCAG | 2550 |
| CUCUGACUCU GAUUCUAUAC UCUUCCUCAA GAGUGGUAUC UCCCUGGGCU | 2600 |
| CUCCUUUCCA CUGAUAAUAG GCUGGAGCCU CGGUGGCCAU GCUUCUUGCC | 2650 |
| CCUUGGGCCU CCCCCCAGCC CCUCCUCCCC UUCCUGCACC CGUACCCCCG | 2700 |
| UGGUCUUUGA AUAAAGUCUG AGUGGGCGGC AAAAAAAAAA AAAAAAAAAA | 2750 |
| AAAAAAAAAA AAAAAAAAAA AAAAAAAAAA AAAAAAAAAA AAAAAAAAAA | 2800 |
| AAAAAAAAAA AAAAAAAAAA AAAAAAAAAA UCUAG-OH3′ | 2835 |
| (SEQ ID NO: 41) | |

Abbreviations in exemplary mRNA-1 sequence above: A=adenosine-5′-monophosphate; C=cytidine-5′-monophosphate; G=guanosine-5′-monophosphate; Me=methyl; p=inorganic phosphate; U=N1-methylpseudouridine-5′-monophosphate (N1-Me-YMP)

The underlined ORF sequence above encodes a concatemer of 34 neoantigens, with around 75 nucleotides encoding each individual neoantigen (2562 total nucleotides). The entire mRNA sequence is 2835 nucleotides. The concatemer encoded by the ORF has an amino acid sequence of:

```
  (SEQ ID NO: 42)
    1 MWAACTNFSR TRKEILLFAE IILCLVAPNQ ESGMKTADFL RVLSGHLMQT
  51 REAEPKGTIT LELIEHNEAY TWINPTGGLA VLAKERPPNL IEFLASYLLK
101 NKGEQYFKRT MPRISTLKNL EDLVTEYPRG IFTKEDALKF VQLKQTGKIT
151 ESPEKTVLTQ EAIIIVKGVS LSSYLEGQAP AVEVAPAGAF YNPSFEDHQT
201 LLSDRLSNHI SSLFCEDQIY RIDHYLGSTE HNKECLINIF KYKFSLVISG
251 LTVWSIRVTS TEEYLHLKPA RYRRGFIEQR NVSGGYLVLC KMNYATRIVT
301 LECSYPETEE EGEAIPVRDS FYRLEKRLWK QQMYTIAKFY FFTLKRESKS
351 VRRLSEEELP AILKLKKSLQ KLVSDWNRGA QLRRPRHSHL TRARRCPGGL
401 PGPAMNYDKL SRSLCYYYEK GIMQKVATKA FLDGENEVAS LEWLRYFDEK
451 ELKSGYPDIG FPLELLSKGF IKSIRGVDNP GVHPATFAAY TAPAYYPYGQ
501 FQGSFVQATP HNRVSYLRAF WRCFRTVGTE KCEVVIQHEN RKYLKTPPGI
551 PAPTKLDELK RQEVSQLRML LKAKMDAEKA SNLQKNKTAS FTTKEKETKL
601 PLLQLEDLDS RVLYYLATKN GILYRVGDLG LRSGSYSLPR IIHIARSQNE
651 RKSKFGSRHS AESQIFKHLL KNLFKIFCKQ SQHMTEVVRR WPHHERCSDS
701 DGLSHYSIWE EVVARSLARV LVFEDDVRGV GKGKRRPLNN NFEIALSLSR
751 NKVFDPVIVE AFLASLENRL YISQESDKDH LRLVESANEK AKREALLRGV
801 EPGPQEFFKG YLKGEHSRLL SLWREVVTES RQASSDSDSI LFLKSGISLG
851 SPFH
```

### Additional Embodiments

1. A method of inducing an immune response to a tumor in a subject, the method comprising:
(a) identifying a subject, wherein the subject has one or more biomarkers or biomarker levels associated with responsiveness to an immune checkpoint inhibitor therapy;
(b) administering to the subject an effective amount of an immune checkpoint inhibitor; and
(c) administering to the subject an effective amount of a personalized cancer vaccine, wherein the personalized cancer vaccine comprises:
   (i) an mRNA having an open reading frame encoding at least two cancer antigen epitopes expressed in the tumor in the subject; and
   (ii) a lipid delivery vehicle, thereby inducing an immune response to the tumor in the subject.
      2. The method of embodiment 1, wherein the subject is determined to have the one or more biomarkers or biomarker levels by measuring the one or more biomarkers or biomarker levels in a biological sample collected from the subject.
      3. The method of embodiment 2, wherein step (a) further comprises comparing the measurement of the one or more biomarkers or biomarker levels to a set value or range.
      4. The method of any preceding embodiments, wherein the one or more biomarkers or biomarker levels comprise tumor mutational burden (TMB), T cell-inflamed gene expression profile (GEP) score, interferon-gamma (IFN-γ) signature score, immune gene signature score, T cell cytotoxicity activity (CYT) score, PD-L1 expression, minimal residual disease (MRD) level, level of γδ T cells, TCR clonotyping value (e.g., DE50 or Gini coefficient), and/or Th1 cell population level.
      5. The method of any preceding embodiment, wherein the one or more biomarkers comprise TMB, optionally wherein the set value of TMB is 7, 10, 20, 30, 50, 100, 175, or 300 non-synonymous mutations with an allele frequency of at least 5% per exome.
      6. The method of any preceding embodiment, wherein the one or more biomarkers comprise T cell-inflamed GEP score, optionally wherein the set value of T cell-inflamed GEP score is 2, 3, 4, 5, or 6.
      7. The method of any preceding embodiment, wherein the one or more biomarkers comprise IFN-γ signature score, optionally wherein the set value of IFN-γ signature score is 2, 3, 4, 5, or 6.
      8. The method of any preceding embodiment, wherein the one or more biomarkers comprise immune gene signature score, optionally wherein the sets value of immune gene signature score is 2, 3, 4, 5, or 6.
      9. The method of any preceding embodiment, wherein the one or more biomarkers comprise CYT score, optionally wherein the set value of CYT score is 2, 3, 4, 5, or 6.
      10. The method of any preceding embodiment, wherein the one or more biomarkers comprise PD-L1 expression, optionally wherein the set value of PD-L1 expression is 1, 2, 3, 4, 5, or 6 when normalized relative to one or more housekeeping genes.
      11. The method of any preceding embodiment, wherein the one or more biomarkers comprise MRD level, optionally wherein the set value of MRD level is 10,000 copies per mL, 5,000 copies per mL, 1,000 copies per mL, 500 copies per mL, 250 copies per mL, 125 copies per mL, 100 copies per mL, 75 copies per mL, 50 copies per mL, or 25 copies per mL of a mutated gene present in the tumor but not in healthy cells of the subject, in a biological sample comprising circulating tumor DNA (ctDNA).
      12. The method of embodiment 11, wherein the set value of MRD level is detectable ctDNA in a biological sample collected from the subject following primary treatment, optionally wherein the biological sample is a blood sample.
      13. The method of any preceding embodiment, wherein the one or more biomarkers comprise γδ T cells or a subset of γδ T cells (e.g., regulatory γδ T cells), optionally wherein the set value of γδ T cells or the subset of γδ T cells is 20%, 15%, 10%, 5%, 4%, 3%, 2%, or 1% of T lymphocytes in peripheral blood mononuclear cells in a biological sample collected from the subject.
      14. The method of any preceding embodiment, wherein the one or more biomarkers comprise TCR clonotyping value (e.g., DE50 or Gini coefficient).
      15. The method of any preceding embodiment, wherein the one or more biomarkers comprise Th1 cell population level, optionally wherein the set value of Th1 cell population is 5%, 10%, 15%, or 20% of T lymphocytes (e.g., CD4⁺ T lymphocytes) in a biological sample (e.g., in a blood sample, such as in peripheral blood mononuclear cells in a blood sample).
      16. A method of inducing an immune response to a tumor in a subject, the method comprising:
(a) selecting a subject identified as having or being likely to have low responsiveness to an immune checkpoint inhibitor therapy;
(b) administering to the subject an effective amount of an immune checkpoint inhibitor; and
(c) administering to the subject an effective amount of a personalized cancer vaccine, wherein the personalized cancer vaccine comprises:
   (i) an mRNA having an open reading frame encoding at least two cancer antigen epitopes expressed in the tumor in the subject; and
   (ii) a lipid particle, thereby inducing an immune response to the tumor in the subject.
      17. The method of embodiment 16, wherein the subject has one or more biomarkers or biomarker levels associated with low responsiveness to the immune checkpoint inhibitor therapy, and/or wherein the subject previously demonstrated low responsiveness to the immune checkpoint inhibitory therapy.
      18. The method of embodiment 17, wherein the subject is determined to have the one or more biomarkers or biomarker levels by a method comprising measurement of the one or more biomarkers or biomarker levels in a biological sample collected from the subject.
      19. The method of embodiment 17 or embodiment 18, wherein the one or more biomarkers or biomarker levels are selected from the group consisting of tumor mutational burden (TMB), PD-L1 expression, PD-L2 expression, T cell-inflamed gene expression (GEP) score, interferon-gamma (IFN-γ) signature score, immune gene signature score, T cell cytotoxicity activity (CYT) score, peripheral blood mononuclear cell (PBMC) content, TCR clonotyping value (e.g., DE50 or Gini coefficient), and Th1 cell population level.
      20. The method of any preceding embodiment, wherein metastasis of the tumor has not been detected in the subject prior to administration of the immune checkpoint inhibitor and/or the personalized cancer vaccine to the subject.
      21. A method of inducing an immune response to a tumor in a subject, the method comprising:
(a) selecting a subject having undetectable levels of metastatic tumor cells;
(b) administering to the subject an effective amount of an immune checkpoint inhibitor; and
(c) administering to the subject an effective amount of a personalized cancer vaccine, wherein the personalized cancer vaccine comprises:
   (i) an mRNA having an open reading frame encoding at least two cancer antigen epitopes expressed in the tumor in the subject; and
   (ii) a lipid delivery vehicle, thereby inducing an immune response to the tumor in the subject.
      22. The method of any preceding embodiment, wherein the lipid delivery vehicle comprises a lipid nanoparticle, a liposome, or a lipoplex.
      23. The method of any preceding embodiment, wherein the lipid delivery vehicle comprises a lipid nanoparticle comprising an ionizable cationic lipid, a neutral lipid, cholesterol, and a PEG-modified lipid.
      24 The method of any preceding embodiment, wherein the immune checkpoint inhibitor is an antibody or fragment thereof, optionally wherein the antibody or fragment thereof specifically binds to a molecule selected from the group consisting of PD-1, TIM-3, VISTA, A2AR, B7-H3, B7-H4, BTLA, CTLA-4, IDO, KIR and LAG3.
      25. The method of any preceding embodiment, wherein the immune checkpoint inhibitor is an anti-PD-1 antibody.
      26. The method of any preceding embodiment, wherein the immune checkpoint inhibitor and/or the personalized cancer vaccine is administered to the subject following surgical resection of a primary tumor from the subject.
      27 The method of any preceding embodiment, wherein the personalized cancer vaccine is administered to the subject 6 weeks or fewer following the administration of the immune checkpoint inhibitor to the subject.
      28. The method of any preceding embodiment, wherein the immune response to the tumor comprises an increase in a population of T cells specific to at least one of the cancer antigen epitopes in a biological sample collected from the subject, relative to the population of T cells in a comparable biological sample collected from the subject prior to induction of the immune response to the tumor.
      29. The method of embodiment 28, wherein the population of T cells is detectable in a pre-treatment biological sample collected from the subject prior to administration of the personalized cancer vaccine and/or the immune checkpoint inhibitor to the subject.
      30. The method of embodiment 28 or embodiment 29, wherein the biological sample comprises peripheral blood mononuclear cells.
      31. The method of any preceding embodiment, wherein a first T cell response to one of the cancer antigen epitopes is detectable in the subject following administration of the personalized cancer vaccine to the subject,
optionally wherein additional T cell responses to an additional one or more of the cancer antigen epitopes are detectable in the subject following administration of the personalized cancer vaccine to the subject.
   32. The method of embodiment 31, wherein the first T cell response is not detectable in the subject prior to administration of the personalized cancer vaccine to the subject,
optionally wherein the additional T cell responses are not detectable in the subject prior to administration of the personalized cancer vaccine to the subject.
   33. The method of embodiment 31, wherein the first T cell response is a CD4 T cell response or a CD8 T cell response.
   34. The method of any one of embodiments 31-33, wherein the first T cell response can be detected and/or quantified by collecting a biological sample comprising peripheral blood mononuclear cells (PBMCs) from the subject, stimulating the PBMCs with the cancer antigen epitopes, and subsequently measuring cytokine production by the PBMCs.
   35. The method of any preceding embodiment, wherein a preexisting T cell response to a first cancer antigen epitope of the cancer antigen epitopes is detectable in the subject prior to administration of the personalized cancer vaccine, and wherein the magnitude of the preexisting T cell response is increased following administration of the personalized cancer vaccine to the subject relative to the magnitude prior to administration of the personalized cancer vaccine.
   36. The method of embodiment 35, wherein the magnitude of the preexisting T cell response corresponds to a ratio of T cells responsive to the first cancer antigen epitope to a total number of T cells in a biological sample, or wherein the magnitude of the preexisting T cell response corresponds to an increased strength of response per cell to the first cancer antigen epitope.
   37 The method of embodiment 35 or embodiment 36, wherein the preexisting T cell response can be detected and/or quantified by collecting a biological sample comprising peripheral blood mononuclear cells (PBMCs) from the subject, stimulating the PBMCs with the cancer antigen epitopes, and subsequently measuring cytokine production by the PBMCs.
   38. The method of any preceding embodiment, wherein administration of the personalized cancer vaccine to the subject reduces the likelihood of progression or recurrence of the tumor in the subject and/or delays the progression or recurrence of the tumor in the subject.
   39. The method of embodiment 38, wherein the reduction in the likelihood of progression or recurrence of the tumor is greater in magnitude than a corresponding reduction in a subject not treated with the personalized cancer vaccine, and/or the delay in the progression or recurrence of the tumor is longer than a corresponding delay in a subject not treated with the personalized cancer vaccine.
   40. The method of any preceding embodiment, wherein the mRNA of the personalized cancer vaccine encodes 0, 1, 2, 3, 4, 5, 6, 7, 8, 9, 10, or more peptides corresponding to driver mutations.
   41. A method of preparing an optimized personalized cancer vaccine, the method comprising:
(a) identifying a plurality of neoantigens present in a tumor in a subject;
(b) selecting a first subset of the neoantigens;
(c) preparing a primary personalized cancer vaccine comprising:
   (i) an mRNA having an open reading frame encoding a first plurality of peptides, each peptide corresponding to a neoantigen of the first subset; and (ii) a lipid delivery vehicle;
(d) administering an effective amount of the primary personalized cancer vaccine to the subject;
(e) evaluating immune cell responses in the subject to the peptides encoded by the mRNA of the primary personalized cancer vaccine;
(f) selecting a second subset of the neoantigens identified in (a) based on the evaluating in (e), wherein the second subset is distinct from the first subset; and
(g) preparing an optimized personalized cancer vaccine comprising:
   (x) an optimized mRNA having an open reading frame encoding an optimized plurality of peptides comprising the second subset; and (y) a lipid delivery vehicle.
      42 The method of embodiment 41, wherein the selecting of (f) comprises removing one or more neoantigens of the first subset from the second subset, and/or adding a duplicate of one or more neoantigens of the first subset to the second subset.
      43. The method of embodiment 41 or embodiment 42, wherein the optimized plurality of peptides comprises two or more peptides each corresponding to an effective neoantigen, wherein the effective neoantigen stimulates an immune cell response in the subject, optionally wherein the two or more peptides have identical amino acid sequences.
      44. The method of any one of embodiments 41-43, wherein the optimized plurality of peptides does not comprise a peptide corresponding to an ineffective neoantigen, wherein the ineffective neoantigen does not stimulate an immune cell response in the subject.
      45. The method of any one of embodiments 41-44, wherein the lipid delivery vehicle comprises a lipid nanoparticle, a liposome, or a lipoplex.
      46. The method of any one of embodiments 41-45, wherein the lipid delivery vehicle comprises a lipid nanoparticle comprising an ionizable cationic lipid, a neutral lipid, cholesterol, and a PEG-modified lipid.
      47. The method of any one of embodiments 41-46, wherein prior to (e), an effective amount of an immune checkpoint inhibitor is administered to the subject.
      48. The method of embodiment 47, wherein the immune checkpoint inhibitor is an antibody or fragment thereof, optionally wherein the antibody or fragment thereof specifically binds to a molecule selected from the group consisting of PD-1, TIM-3, VISTA, A2AR, B7-H3, B7-H4, BTLA, CTLA-4, IDO, KIR and LAG3.
      49. The method of embodiment 47 or embodiment 48, wherein the immune checkpoint inhibitor is an anti-PD-1 antibody.
      50. A method of inducing an immune response to a tumor in a subject, the method comprising administering to the subject an effective amount of an optimized personalized cancer vaccine prepared according to the method of any one of embodiments 41-49.
      51. A composition comprising an optimized personalized cancer vaccine, wherein the optimized personalized cancer vaccine is prepared according to the method of any one of embodiments 41-49.
      52. The composition of embodiment 51, wherein the mRNA of the optimized personalized cancer vaccine encodes 0, 1, 2, 3, 4, 5, 6, 7, 8, 9, 10, or more peptides corresponding to driver mutations.
      53. A method of inducing an immune response to a tumor in a subject, the method comprising administering to the subject an effective amount of the composition of embodiment 51 or embodiment 52.
      54. The method of embodiment 53, further comprising administering to the subject an effective amount of an immune checkpoint inhibitor.
      55. A method of treating a cancer in a human subject, the method comprising:
(a) administering to a subject an effective amount of an immune checkpoint inhibitor; and
(b) administering to the subject an effective amount of a personalized cancer vaccine, wherein the personalized cancer vaccine comprises:
   (i) an mRNA having an open reading frame encoding at least two cancer antigen epitopes expressed in the tumor in the subject; and
   (ii) a lipid delivery vehicle, wherein the cancer is non-small cell lung cancer.
      56 The method of embodiment 55, wherein the tumor comprises resected stage II non-small cell lung cancer.
      57. The method of embodiment 55, wherein the tumor comprises resected stage III non-small cell lung cancer.
      58 The method of embodiment 55, wherein the tumor comprises resected stage IIIA non-small cell lung cancer.
      59. The method of embodiment 55, wherein the tumor comprises resected stage IIIB non-small cell lung cancer.
      60. The method of any one of embodiments 55-59, wherein prior to the administering of (a), the subject has received a treatment for the cancer.
      61. The method of embodiment 60, wherein the treatment is chemotherapy (e.g., adjuvant chemotherapy (e.g., platinum doublet)).
      62. A method of treating a cancer in a human subject, the method comprising:
(a) administering to a subject an effective amount of an immune checkpoint inhibitor; and
(b) administering to the subject an effective amount of a personalized cancer vaccine, wherein the personalized cancer vaccine comprises:
   (i) an mRNA having an open reading frame encoding at least two cancer antigen epitopes expressed in the tumor in the subject; and
   (ii) a lipid delivery vehicle, wherein the cancer is kidney cancer.
      63 The method of embodiment 62, wherein the cancer is renal cell carcinoma.
      64. The method of embodiment 62 or 63, wherein prior to the administering of (a), the subject has received a treatment for the cancer.
      65. The method of embodiment 64, wherein the treatment is nephrectomy and/or metastasectomy.
      66. The method of any one of embodiments 62-65, wherein the subject has not previously been treated with prior systemic therapy or radiotherapy.
      67. A method of treating a cancer in a human subject, the method comprising:
(a) administering to a subject an effective amount of an immune checkpoint inhibitor; and
(b) administering to the subject an effective amount of a personalized cancer vaccine, wherein the personalized cancer vaccine comprises:
   (i) an mRNA having an open reading frame encoding at least two cancer antigen epitopes expressed in the tumor in the subject; and
   (ii) a lipid delivery vehicle, wherein the cancer is muscle invasive urothelial carcinoma (MIUC).
      68. The method of embodiment 67, wherein the cancer is muscle-invasive bladder cancer (MIBC), or muscle-invasive urinary tract urothelial cancer (UTUC).
      69. The method of embodiment 67 or 68, wherein prior to the administering of (a), the subject has received a treatment for the cancer.
      70. The method of embodiment 69, wherein the treatment is radical resection.
      71. A method of treating a cancer in a human subject, the method comprising:
(a) administering to a subject an effective amount of an immune checkpoint inhibitor; and
(b) administering to the subject an effective amount of a personalized cancer vaccine, wherein the personalized cancer vaccine comprises:
   (i) an mRNA having an open reading frame encoding at least two cancer antigen epitopes expressed in the tumor in the subject; and
   (ii) a lipid delivery vehicle, wherein the cancer is cutaneous squamous cell carcinoma (cSCC).
      72. The method of embodiment 71, wherein the cancer is resectable.
      73. The method of embodiment 71 or 72, wherein the cancer is locally advanced.
      74. The method of any one of embodiments 71-73, Wherein the cancer is Stage II, Stage III, or Stage IV.
      75. The method of any one of embodiments 71-74, further comprising surgical resection.
      76. The method of any preceding embodiment, wherein the immune checkpoint inhibitor is an anti-PD-1 antibody or antigen-binding fragment thereof.
      77. The method of embodiment 76, wherein the anti-PD-1 antibody or antigen-binding fragment thereof comprises light chain complementarity determining regions (CDRs) comprising a sequence of amino acids as set forth in SEQ ID NOs: 43, 44 and 45 and heavy chain CDRs comprising a sequence of amino acids as set forth in SEQ ID NOs: 48, 49 and 50.
      78. The method of any one of embodiments 76 or 77, wherein the anti-PD-1 antibody or antigen-binding fragment thereof comprises a light chain variable region comprising SEQ ID NO:46 or a variant thereof, and a heavy chain variable region comprising SEQ ID NO:51.
      79. The method of any one of embodiments 76-78, wherein the anti-PD-1 antibody or antigen-binding fragment thereof comprises a light chain comprising SEQ ID NO: 47 and a heavy chain comprising SEQ ID NO:52.
      80. The method of any one of embodiments 76-79, the anti-PD-1 antibody or antigen-binding fragment thereof is pembrolizumab or a variant thereof.
      81. The method of any one of embodiments 76-80, the anti-PD-1 antibody or antigen-binding fragment thereof is pembrolizumab.
      82. The method of any preceding embodiment, the subject received at least 1 dose of chemotherapy (e.g., adjuvant chemotherapy (e.g., platinum doublet)) prior to the method.
      83. A method of inducing an immune response to a tumor in a subject, the method comprising:
(a) administering to a subject an effective amount of an immune checkpoint inhibitor; and
(b) administering to the subject an effective amount of a personalized cancer vaccine, wherein the subject has one or more biomarkers or biomarker levels associated with responsiveness to the personalized cancer vaccine, and wherein the personalized cancer vaccine comprises:
   (i) an mRNA having an open reading frame encoding at least two cancer antigen epitopes expressed in the tumor in the subject; and
   (ii) a lipid delivery vehicle, thereby inducing an immune response to the tumor in the subject.
      84. The method of embodiment 83, further comprising measuring the one or more biomarkers or biomarker levels in a biological sample collected from the subject and identifying the subject as likely to be responsive to the personalized cancer vaccine.
      85. The method of embodiment 84, wherein the measuring is conducted:
(i) at the time of the administering of (a);
(ii) at the time of the administering of (b);
(iii) within 90 days from the time of the administering of (a), optionally at day 90 following the administration of (a);
(iv) within 90 days prior to the time of the administering of (b);
(v) within 180 days from the time of the administering of (a), optionally at day 180 following the administration of (a); or
(vi) within 180 days prior to the time of the administering of (b).
   86 The method of embodiment 84 or embodiment 85, wherein the identifying comprises comparing the measurement of the one or more biomarkers or biomarker levels to a set value or range.
   87 The method of any one of embodiments 83-86, wherein the one or more biomarkers or biomarker levels is not associated with responsiveness to the immune checkpoint inhibitor.
   88. The method of any one of embodiments 83-86, wherein the one or more biomarkers or biomarker levels is associated with responsiveness to the immune checkpoint inhibitor.
   89 The method of any one of embodiments 83-88, wherein the one or more biomarkers comprise tumor mutational burden (TMB), T cell-inflamed gene expression profile (GEP) score, interferon-gamma (IFN-γ) signature score, immune gene signature score, T cell cytotoxicity activity (CYT) score, PD-L1 expression, minimal residual disease (MRD) level, γδ T cells or a sub-type of γδ T cells (e.g., regulatory γδ T cells), TCR clonotyping value (e.g., DE50 or Gini coefficient), and/or Th1 cell population level.
   90. The method of any one of embodiments 83-89, wherein the one or more biomarkers comprise TMB, optionally wherein the set value of TMB is 7, 10, 20, 30, 50, 100, 175, or 300 non-synonymous mutations with an allele frequency of at least 5% per exome.
   91 The method of embodiment 90, wherein the TMB value is higher than the set value.
   92 The method of embodiment 90, wherein the TMB value is lower than the set value.
   93. The method of any one of embodiments 83-92, wherein the subject has fewer than 175 non-synonymous mutations with an allele frequency of at least 5% per exome.
   94. The method of any one of embodiments 83-92, wherein the subject has 175 or more non-synonymous mutations with an allele frequency of at least 5% per exome.
   95 The method of any one of embodiments 83-94, wherein the one or more biomarkers comprise T cell-inflamed GEP score, optionally wherein the set value of T cell-inflamed GEP score is 2, 3, 4, 5, or 6.
   96 The method of embodiment 95, wherein the T cell-inflamed GEP score is higher than the set value.
   97 The method of embodiment 95, wherein the T cell-inflamed GEP score is lower than the set value.
   98. The method of any one of embodiments 83-97, wherein the one or more biomarkers comprise IFN-γ signature score, optionally wherein the set value of IFN-γ signature score is 2, 3, 4, 5, or 6.
   99. The method of embodiment 98, wherein the IFN-γ signature score is higher than the set value.
   100. The method of embodiment 98, wherein the IFN-γ signature score is lower than the set value.
   101. The method of any one of embodiments 83-100, wherein the one or more biomarkers comprise immune gene signature score, optionally wherein the set value of immune gene signature score is 2, 3, 4, 5, or 6.
   102. The method of embodiment 101, wherein the immune gene signature is higher than the set value.
   103. The method of embodiment 101, wherein the immune gene signature is lower than the set value.
   104. The method of any one of embodiments 86-103, wherein the one or more biomarkers comprise CYT score, optionally wherein the set value of CYT score is 2, 3, 4, 5, or 6.
   105. The method of embodiment 104, wherein the CYT score is higher than the set value.
   106. The method of embodiment 104, wherein the CYT score is lower than the set value.
   107. The method of any one of embodiments 83-106, wherein the one or more biomarkers comprise PD-L1 expression, optionally wherein the set value of PD-L1 expression is 1, 2, 3, 4, 5, or 6 when normalized relative to one or more housekeeping genes.
   108. The method of embodiment 107, wherein the PD-L1 expression is higher than the set value.
   109. The method of embodiment 107, wherein the PD-L1 expression is lower than the set value.
   110. The method of any one of embodiments 83-109, wherein the one or more biomarkers comprise MRD level, optionally wherein the set value of MRD level is 10,000 copies per mL, 5,000 copies per mL, 1,000 copies per mL, 500 copies per mL, 250 copies per mL, 125 copies per mL, 100 copies per mL, 75 copies per mL, 50 copies per mL, or 25 copies per mL of a mutated gene present in the tumor but not in healthy cells of the subject, in a biological sample comprising circulating tumor DNA (ctDNA).
   111. The method of embodiment 110, wherein the set value of MRD level is detectable ctDNA in a biological sample collected from the subject following primary treatment, optionally wherein the biological sample is a blood sample.
   112. The method of embodiment 110 or embodiment 111, wherein the MRD level is higher than the set value.
   113. The method of embodiment 110 or embodiment 111, wherein the MRD level is lower than the set value.
   114. The method of any one of embodiments 83-113, wherein the one or more biomarkers comprise γδ T cells or a sub-type of γδ T cells (e.g., regulatory γδ T cells), optionally wherein the set value of γδ T cells or the sub-type of γδ T cells is 20%, 15%, 10%, 5%, 4%, 3%, 2%, or 1% of T lymphocytes in peripheral blood mononuclear cells in a biological sample collected from the subject.
   115. The method of embodiment 114, wherein the yd T cells or sub-type of γδ T cells value is higher than the set value.
   116. The method of embodiment 114, wherein the γδ T cells or sub-type of γδ T cells value is lower than the set value.
   117. The method of any one of embodiments 83-116, wherein the one or more biomarkers comprise TCR clonotyping value (e.g., DE50 or Gini coefficient).
   118. The method of embodiment 117, wherein the TCR clonotyping value is higher than a set value of TCR clonotyping value.
   119. The method of embodiment 117, wherein the TCR clonotyping value is lower than a set value of TCR clonotyping value.
   120. The method of any one of embodiments 83-119, wherein the one or more biomarkers comprise Th1 cell population level, optionally wherein the set value of Th1 cell population is 5%, 10%, 15%, or 20% of T lymphocytes (e.g., CD4+ T lymphocytes) in a biological sample (e.g., in a blood sample, such as in peripheral blood mononuclear cells in a blood sample).
   121. The method of embodiment 120, wherein the Th1 cell population level is higher than the set value.
   122. The method of embodiment 120, wherein the Th1 cell population level is lower than the set value.
   123. A method of inducing an immune response to a tumor in a subject, the method comprising:
(a) administering to a subject an effective amount of an immune checkpoint inhibitor, wherein the subject has or is likely to have a low responsiveness to the immune checkpoint inhibitor therapy; and
(b) administering to the subject an effective amount of a personalized cancer vaccine, wherein the personalized cancer vaccine comprises:
   (i) an mRNA having an open reading frame encoding at least two cancer antigen epitopes expressed in the tumor in the subject; and
   (ii) a lipid delivery vehicle, thereby inducing an immune response to the tumor in the subject.
      124. The method of embodiment 123, wherein the subject has one or more biomarkers or biomarker levels associated with low responsiveness to the immune checkpoint inhibitor therapy, and/or wherein the subject previously demonstrated low responsiveness to the immune checkpoint inhibitory therapy.
      125. The method of embodiment 124, wherein the subject is determined to have the one or more biomarkers or biomarker levels by a method comprising measurement of the one or more biomarkers or biomarker levels in a biological sample collected from the subject.
      126. The method of embodiment 124 or embodiment 125, wherein the one or more biomarkers are selected from the group consisting of tumor mutational burden (TMB), PD-L1 expression, PD-L2 expression, T cell-inflamed gene expression (GEP) score, interferon-gamma (IFN-γ) signature score, immune gene signature score, T cell cytotoxicity activity (CYT) score, peripheral blood mononuclear cell (PBMC) content, TCR clonotyping value (e.g., DE50 or Gini coefficient), and Th1 cell population level.
      127. The method of any one of embodiments 83-126, wherein metastasis of the tumor has not been detected in the subject prior to administration of the immune checkpoint inhibitor and/or the personalized cancer vaccine to the subject.
      128. A method of inducing an immune response to a tumor in a subject, the method comprising:
(a) administering to a subject an effective amount of an immune checkpoint inhibitor, wherein the subject has an undetectable level of metastatic tumor cells; and
(b) administering to the subject an effective amount of a personalized cancer vaccine, wherein the personalized cancer vaccine comprises: (i) an mRNA having an open reading frame encoding at least two cancer antigen epitopes expressed in the tumor in the subject; and (ii) a lipid delivery vehicle, thereby inducing an immune response to the tumor in the subject.
   129. The method of any one of embodiments 83-128, wherein the lipid delivery vehicle comprises a lipid nanoparticle, a liposome, or a lipoplex.
   130. The method of any one of embodiments 83-129, wherein the lipid delivery vehicle comprises a lipid nanoparticle comprising an ionizable cationic lipid, a neutral lipid, cholesterol, and a PEG-modified lipid.
   131. The method of any one of embodiments 83-130, wherein the immune checkpoint inhibitor is an antibody or fragment thereof, optionally wherein the antibody or fragment thereof specifically binds to a molecule selected from the group consisting of PD-1, TIM-3, VISTA, A2AR, B7-H3, B7-H4, BTLA, CTLA-4, IDO, KIR and LAG3.
   132. The method of any one of embodiments 83-131, wherein the immune checkpoint inhibitor is an anti-PD-1 antibody or antigen-binding fragment thereof.
   133. The method of embodiment 132, wherein the anti-PD-1 antibody or antigen-binding fragment thereof comprises light chain complementarity determining regions (CDRs) comprising a sequence of amino acids as set forth in SEQ ID NOs: 43, 44 and 45 and heavy chain CDRs comprising a sequence of amino acids as set forth in SEQ ID NOs: 48, 49 and 50.
   134. The method of embodiment 132 or 133, wherein the anti-PD-1 antibody or antigen-binding fragment thereof comprises a light chain variable region comprising SEQ ID NO:46 or a variant thereof, and a heavy chain variable region comprising SEQ ID NO:51.
   135. The method of any one of embodiments 132-134, wherein the anti-PD-1 antibody or antigen-binding fragment thereof comprises a light chain comprising SEQ ID NO: 47 and a heavy chain comprising SEQ ID NO:52.
   136. The method of any one of embodiments 132-134, wherein the anti-PD-1 antibody or antigen-binding fragment thereof is pembrolizumab or a variant thereof.
   137. The method of any one of embodiments 132-134, wherein the anti-PD-1 antibody or antigen-binding fragment thereof is pembrolizumab.
   138. The method of any one of embodiments 83-137, wherein the immune checkpoint inhibitor and/or the personalized cancer vaccine is administered to the subject following surgical resection of a primary tumor from the subject.
   139. The method of any one of embodiments 83-138, wherein the personalized cancer vaccine is administered to the subject 6 weeks or fewer following the administration of the immune checkpoint inhibitor to the subject.
   140. The method of any one of embodiments 83-139, wherein the immune response to the tumor comprises an increase in a population of T cells specific to at least one of the cancer antigen epitopes in a biological sample collected from the subject, relative to the population of T cells in a comparable biological sample collected from the subject prior to induction of the immune response to the tumor.
   141. The method of embodiment 140, wherein the population of T cells is detectable in a pre-treatment biological sample collected from the subject prior to administration of the personalized cancer vaccine and/or the immune checkpoint inhibitor to the subject.
   142. The method of embodiment 140 or embodiment 141, wherein the biological sample comprises peripheral blood mononuclear cells.
   143. The method of any one of embodiments 83-142, wherein a first T cell response to one of the cancer antigen epitopes is detectable in the subject following administration of the personalized cancer vaccine to the subject,
optionally wherein additional T cell responses to an additional one or more of the cancer antigen epitopes are detectable in the subject following administration of the personalized cancer vaccine to the subject.
   144. The method of embodiment 143, wherein the first T cell response is not detectable in the subject prior to administration of the personalized cancer vaccine to the subject,
optionally wherein the additional T cell responses are not detectable in the subject prior to administration of the personalized cancer vaccine to the subject.
   145. The method of embodiment 143 or embodiment 144, wherein the first T cell response is a CD4 T cell response or a CD8 T cell response.
   146. The method of any one of embodiments 143-145, wherein the first T cell response can be detected and/or quantified by collecting a biological sample comprising peripheral blood mononuclear cells (PBMCs) from the subject, stimulating the PBMCs with the cancer antigen epitopes, and subsequently measuring cytokine production by the PBMCs.
   147. The method of any one of embodiments 83-146, wherein a preexisting T cell response to a first cancer antigen epitope of the cancer antigen epitopes is detectable in the subject prior to administration of the personalized cancer vaccine, and wherein the magnitude of the preexisting T cell response is increased following administration of the personalized cancer vaccine to the subject relative to the magnitude prior to administration of the personalized cancer vaccine.
   148. The method of embodiment 147, wherein the magnitude of the preexisting T cell response corresponds to a ratio of T cells responsive to the first cancer antigen epitope to a total number of T cells in a biological sample, or wherein the magnitude of the preexisting T cell response corresponds to an increased strength of response per cell to the first cancer antigen epitope.
   149. The method of embodiment 147 or embodiment 148, wherein the preexisting T cell response can be detected and/or quantified by collecting a biological sample comprising peripheral blood mononuclear cells (PBMCs) from the subject, stimulating the PBMCs with the cancer antigen epitopes, and subsequently measuring cytokine production by the PBMCs.
   150. The method of any one of embodiments 83-149, wherein administration of the personalized cancer vaccine to the subject reduces the likelihood of progression or recurrence of the tumor in the subject and/or delays the progression or recurrence of the tumor in the subject.
   151. The method of embodiment 150, wherein the reduction in the likelihood of progression or recurrence of the tumor is greater in magnitude than a corresponding reduction in a subject not treated with the personalized cancer vaccine, and/or the delay in the progression or recurrence of the tumor is longer than a corresponding delay in a subject not treated with the personalized cancer vaccine.
   152. The method of any one of embodiments 83-151, wherein the mRNA of the personalized cancer vaccine encodes 0, 1, 2, 3, 4, 5, 6, 7, 8, 9, 10, or more peptides corresponding to driver mutations.
   153. A method of preparing a secondary personalized cancer vaccine, the method comprising:
(a) administering to a subject an effective amount of a primary personalized cancer vaccine comprising: (i) an mRNA having an open reading frame encoding a first plurality of peptides, each peptide corresponding to a neoantigen of a first subset of neoantigens; and (ii) a lipid delivery vehicle;
(b) evaluating immune cell responses in the subject to the peptides encoded by the mRNA of the primary personalized cancer vaccine;
(c) preparing a secondary personalized cancer vaccine comprising: (i) an mRNA having an open reading frame encoding a second plurality of peptides, wherein the second plurality of peptides does not contain one or more peptides corresponding to non-immunogenic neoantigens from the primary cancer vaccine; and (ii) a lipid delivery vehicle.
   154. The method of embodiment 153, further comprising administering an effective amount of the secondary personalized cancer vaccine to the subject.
   155. The method of embodiment 153 or embodiment 154, further comprising, prior to (a), identifying a plurality of neoantigens present in a tumor in the subject and selecting the first subset of neoantigens from the plurality of neoantigens.
   156. The method of any one of embodiments 153-155, further comprising selecting a second subset of neoantigens based on the evaluating of (b), wherein the second plurality of peptides comprises peptides corresponding to the second subset of neoantigens.
   157. The method of any one of embodiments 153-156, wherein the second plurality of peptides comprises two or more peptides each corresponding to an immunogenic neoantigen, wherein the immunogenic neoantigen stimulates an immune cell response in the subject, optionally wherein the two or more peptides have identical amino acid sequences.
   158. The method of any one of embodiments 153-157, wherein the mRNA of the secondary personalized cancer vaccine encodes 0, 1, 2, 3, 4, 5, 6, 7, 8, 9, 10, or more peptides corresponding to driver mutations.
   159. The method of any one of embodiments 153-158, wherein the lipid delivery vehicle comprises a lipid nanoparticle, a liposome, or a lipoplex.
   160. The method of any one of embodiments 153-159, wherein the subject has one or more biomarkers or biomarker levels associated with responsiveness to the personalized cancer vaccine.
   161. The method of embodiment 160, further comprising measuring the one or more biomarkers or biomarker levels in a biological sample collected from the subject and identifying the subject as likely to be responsive to the personalized cancer vaccine.
   162. The method of embodiment 161, wherein the identifying comprises comparing the measurement of the one or more biomarkers or biomarker levels to a set value or range.
   163. The method of any one of embodiments 160-162, wherein the one or more biomarkers or biomarker levels is not associated with responsiveness to the immune checkpoint inhibitor.
   164. The method of any one of embodiments 160-162, wherein the one or more biomarkers or biomarker levels is associated with responsiveness to the immune checkpoint inhibitor.
   165. The method of any one of embodiments 160-164, wherein the one or more biomarkers comprise tumor mutational burden (TMB), T cell-inflamed gene expression profile (GEP) score, interferon-gamma (IFN-γ) signature score, immune gene signature score, T cell cytotoxicity activity (CYT) score, PD-L1 expression, minimal residual disease (MRD) level, γδ T cells or a sub-type of γδ T cells (e.g., regulatory γδ T cells), TCR clonotyping value (e.g., DE50 or Gini coefficient), and/or Th1 cell population level.
   166. The method of any one of embodiments 160-165, wherein the one or more biomarkers comprise TMB, optionally wherein the set value of TMB is 7, 10, 20, 30, 50, 100, 175, or 300 non-synonymous mutations with an allele frequency of at least 5% per exome.
   167. The method of any one of embodiments 160-166, wherein the subject has fewer than 175 non-synonymous mutations with an allele frequency of at least 5% per exome.
   168. The method of any one of embodiments 160-167, wherein the subject has 175 or more non-synonymous mutations with an allele frequency of at least 5% per exome.
   169. The method of any one of embodiments 160-168, wherein the one or more biomarkers comprise T cell-inflamed GEP score, optionally wherein the set value of T cell-inflamed GEP score is 2, 3, 4, 5, or 6.
   170. The method of any one of embodiments 160-169, wherein the one or more biomarkers comprise IFN-γ signature score, optionally wherein the set value of IFN-γ signature score is 2, 3, 4, 5, or 6.
   171. The method of any one of embodiments 160-170, wherein the one or more biomarkers comprise immune gene signature score, optionally wherein the sets value of immune gene signature score is 2, 3, 4, 5, or 6.
   172. The method of any one of embodiments 160-171, wherein the one or more biomarkers comprise CYT score, optionally wherein the set value of CYT score is 2, 3, 4, 5, or 6.
   173. The method of any one of embodiments 160-172, wherein the one or more biomarkers comprise PD-L1 expression, optionally wherein the set value of PD-L1 expression is 1, 2, 3, 4, 5, or 6 when normalized relative to one or more housekeeping genes.
   174. The method of any one of embodiments 160-173, wherein the one or more biomarkers comprise MRD level, optionally wherein the set value of MRD level is 10,000 copies per mL, 5,000 copies per mL, 1,000 copies per mL, 500 copies per mL, 250 copies per mL, 125 copies per mL, 100 copies per mL, 75 copies per mL, 50 copies per mL, or 25 copies per mL of a mutated gene present in the tumor but not in healthy cells of the subject, in a biological sample comprising circulating tumor DNA (ctDNA).
   175. The method of embodiment 174, wherein the set value of MRD level is detectable ctDNA in a biological sample collected from the subject following primary treatment, optionally wherein the biological sample is a blood sample.
   176. The method of any one of embodiments 160-175, wherein the one or more biomarkers comprise γδ T cells or a sub-type of γδ T cells (e.g., regulatory γδ T cells), optionally wherein the set value of γδ T cells or the sub-type of γδ T cells is 20%, 15%, 10%, 5%, 4%, 3%, 2%, or 1% of T lymphocytes in peripheral blood mononuclear cells in a biological sample collected from the subject.
   177. The method of any one of embodiments 160-176, wherein the one or more biomarkers comprise TCR clonotyping value (e.g., DE50 or Gini coefficient).
   178. The method of any one of embodiments 160-177, wherein the one or more biomarkers comprise Th1 cell population level, optionally wherein the set value of Th1 cell population is 5%, 10%, 15%, or 20% of T lymphocytes (e.g., CD4+ T lymphocytes) in a biological sample (e.g., in a blood sample, such as in peripheral blood mononuclear cells in a blood sample).
   179. The method of any one of embodiments 153-178, wherein the lipid delivery vehicle comprises a lipid nanoparticle comprising an ionizable cationic lipid, a neutral lipid, cholesterol, and a PEG-modified lipid.
   180. The method of any one of embodiments 153-179, wherein prior to (b), an effective amount of an immune checkpoint inhibitor is administered to the subject.
   181. The method of embodiment 180, wherein the immune checkpoint inhibitor is an antibody or fragment thereof, optionally wherein the antibody or fragment thereof specifically binds to a molecule selected from the group consisting of PD-1, TIM-3, VISTA, A2AR, B7-H3, B7-H4, BTLA, CTLA-4, IDO, KIR and LAG3.
   182. The method of embodiment 180 or embodiment 181, wherein the immune checkpoint inhibitor is an anti-PD-1 antibody.
   183. A method of inducing an immune response to a tumor in a subject, the method comprising administering to the subject an effective amount of a secondary personalized cancer vaccine prepared according to the method of any one of embodiments 153-182.
   184. The method of any one of embodiments 83-183, wherein the tumor comprises resected stage III or stage IV melanoma.
   185. The method of any one of embodiments 83-183, wherein the tumor comprises resected stage II melanoma.
   186. The method of any one of embodiments 83-183, wherein the tumor comprises resected cutaneous melanoma.
   187. The method of any one of embodiments 184-186, wherein the tumor has a BRAF mutation.
   188. The method of any one of embodiments 83-183, wherein the tumor comprises non-small cell lung cancer.
   189. The method of embodiment 188, wherein the tumor comprises resected stage II non-small cell lung cancer.
   190. The method of embodiment 188, wherein the tumor comprises resected stage III non-small cell lung cancer.
   191. The method of embodiment 190, wherein the tumor comprises resected stage IIIA non-small cell lung cancer.
   192. The method of embodiment 190, wherein the tumor comprises resected stage IIIB non-small cell lung cancer.
   193. A composition comprising a secondary personalized cancer vaccine, wherein the secondary personalized cancer vaccine is prepared according to the method of any one of embodiments 153-182.
   194. The composition of embodiment 193, wherein the mRNA of the secondary personalized cancer vaccine encodes 0, 1, 2, 3, 4, 5, 6, 7, 8, 9, 10, or more peptides corresponding to driver mutations.
   195. A method of inducing an immune response to a tumor in a subject, the method comprising administering to the subject an effective amount of the composition of embodiment 173 or embodiment 174.
   196. The method of embodiment 195, further comprising administering to the subject an effective amount of an immune checkpoint inhibitor.
   197. A combination of an immune checkpoint inhibitor and a personalized cancer vaccine for use in the manufacture of a medicament as set forth in any one of the methods of embodiments 1-192 and 195-196 or the composition of embodiment 193 or 194.
   198. Use of a combination of an immune checkpoint inhibitor and a personalized cancer vaccine as set forth in any one of the methods of embodiments 1-192 and 195-196 or the composition of embodiment 193 or 194.
   199. A pharmaceutical composition comprising an immune checkpoint inhibitor for use in combination with a personalized cancer vaccine as set forth in any one of the methods of embodiments 1-192 and 195-196 or the composition of embodiment 193 or 194.
   200. A pharmaceutical composition comprising a personalized cancer vaccine for use in combination with an immune checkpoint inhibitor as set forth in any one of the methods of embodiments 1-192 and 195-196 or the composition of embodiment 193 or 194.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in some embodiments, to A only (optionally including elements other than B); in some embodiments, to B only (optionally including elements other than A); in some embodiments, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in some embodiments, to at least one, optionally including more than one, B, with no A present (and optionally including elements other than A); in some embodiments, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. A method of inducing an immune response against a tumor in a subject, the method comprising:
(a) administering to a subject an effective amount of an immune checkpoint inhibitor;
(b) measuring one or more biomarkers or biomarker levels in a biological sample collected from the subject, wherein the measuring is conducted before or on the day of the administering of (c); and
(c) administering to the subject an effective amount of a personalized cancer vaccine, wherein the measurement of the one or more biomarkers or biomarker levels identifies the subject as likely to be responsive to the personalized cancer vaccine, and wherein the personalized cancer vaccine comprises:
(i) an mRNA comprising an open reading frame that encodes at least two cancer antigen epitopes expressed in the tumor in the subject; and
(ii) a lipid delivery vehicle,
wherein the administration of the immune checkpoint inhibitor and the personalized cancer vaccine induces an immune response against the tumor in the subject.

2. The method of claim 1, wherein the measuring is conducted:
(i) within 7 days prior to the administering of (c), optionally on the same day as the administering of (c);
(ii) within 90 days prior to the time of the administering of (c);
(iii) within 180 days prior to the time of the administering of (c);
(iv) within 90 days from the time of the administering of (a), optionally at or approximately at day 90 following the administering of (a); or
(v) within 180 days from the time of the administering of (a), optionally at or approximately at day 180 following the administering of (a).

3. (canceled)

4. The method of claim 1, wherein the method further comprises comparing the measurement of the one or more biomarkers or biomarker levels to predetermined reference values or ranges.

5. The method of claim 1, wherein the one or more biomarkers or biomarker levels comprise tumor mutational burden (TMB), T cell-inflamed gene expression profile (GEP) score, T cell cytotoxicity activity (CYT) score, PD-L1 expression, minimal residual disease (MRD) level, and/or γδ T cells or a sub-type of γδ T cells.

6. The method of claim 1, wherein one or more of:
(i) the one or more biomarkers comprise TMB, and the measurement of TMB in the biological sample collected from the subject is less than a predetermined reference value of TMB, optionally wherein the predetermined reference value of TMB is 175 non-synonymous mutations with an allele frequency of at least 5% per exome;
(ii) the one or more biomarkers comprise T cell-inflamed GEP score, and the measurement of T-cell inflamed GEP score in the biological sample collected from the subject is less than a predetermined reference value of T-cell inflamed GEP score, optionally wherein the predetermined reference value of T cell-inflamed GEP score is 4;
(iii) the one or more biomarkers comprise CYT score, and the measurement of CYT score in the biological sample collected from the subject is less than a predetermined reference value of CYT score, optionally wherein the predetermined reference value of CYT score is 4;
(iv) the one or more biomarkers comprise PD-L1 expression, and the measurement of PD-L1 expression in the biological sample collected from the subject is less than a predetermined reference value of PD-L1 expression, optionally wherein the predetermined reference value of PD-L1 expression is 4, when normalized relative to one or more housekeeping genes;
(v) the one or more biomarkers comprise MRD level, and the measurement of MRD level in the biological sample collected from the subject is greater than a predetermined reference value of MRD level, optionally wherein (a) the predetermined reference value of MRD level is 500 copies per mL of a mutated gene present in the tumor but not in healthy cells of the subject, in a biological sample comprising circulating tumor DNA (ctDNA), or (b) the predetermined reference value of MRD level is detectable ctDNA in a biological sample collected from the subject following primary treatment, optionally wherein the biological sample is a blood sample; and/or
(vi) the one or more biomarkers comprise γδ T cells or a sub-type of γδ T cells, and the measurement of γδ T cells or a sub-type of γδ T cells in the biological sample collected from the subject is less than a predetermined reference value of γδ T cells or a sub-type of γδ T cells, wherein the sub-type of γδ T cells is regulatory γδ T cells, optionally wherein the predetermined reference value of γδ T cells or the sub-type of γδ T cells is 10% of T lymphocytes in peripheral blood mononuclear cells in a biological sample collected from the subject.

7-11. (canceled)

12. The method of claim 4, wherein the measurement of at least one of the one or more biomarkers or biomarker levels is higher than a predetermined reference value or range for the biomarker or biomarker level.

13. The method of claim 4, wherein the measurement of at least one of the one or more biomarkers or biomarker levels is lower than a predetermined reference value or range for the biomarker or biomarker level.

14. The method of claim 1, wherein metastasis of the tumor has not been detected in the subject prior to administration of the immune checkpoint inhibitor and/or the personalized cancer vaccine to the subject.

15. The method of claim 1, wherein the lipid delivery vehicle comprises a lipid nanoparticle, a liposome, or a lipoplex.

16. The method of claim 1, wherein the lipid delivery vehicle comprises a lipid nanoparticle comprising an ionizable cationic lipid, a neutral lipid, cholesterol, and a PEG-modified lipid,
optionally wherein the ionizable cationic lipid, the neutral lipid, the cholesterol, and the PEG-modified lipid are in a molar ratio of 20-60 mol % ionizable cationic lipid: 5-25 mol % neutral lipid: 25-55 mol % cholesterol: 0.5-15 mol % PEG-modified lipid,
optionally wherein the ionizable cationic lipid comprises 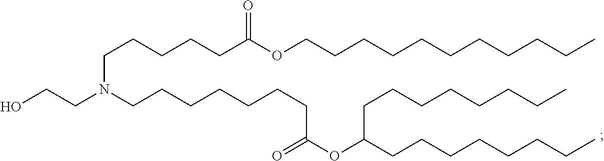 [SMILES: CCCCCCCCCCCOC(=O)CCCCCN(CCO)CCCCCCCC(=O)OC(CCCCCCCC)CCCCCCCC]
and
optionally wherein the neutral lipid comprises 1,2-distearoyl-sn-glycero-3-phosphocholine (DSPC); and
optionally wherein the PEG-modified lipid comprises 1,2-dimyristoyl-sn-glycerol methoxypolyethylene glycol (PEG-DMG).

17. The method of claim 1, wherein the immune checkpoint inhibitor is an antibody or fragment thereof, optionally wherein the antibody or fragment thereof specifically binds to a molecule selected from the group consisting of PD-1, TIM-3, VISTA, A2AR, B7-H3, B7-H4, BTLA, CTLA-4, IDO, KIR and LAG3.

18. The method of claim 1, wherein the immune checkpoint inhibitor is an anti-PD-1 antibody or antigen-binding fragment thereof, optionally wherein:
(a) the anti-PD-1 antibody or antigen-binding fragment thereof comprises:
(i) light chain complementarity determining regions (CDRs) comprising a sequence of amino acids as set forth in SEQ ID NOs: 43, 44 and 45 and heavy chain CDRs comprising a sequence of amino acids as set forth in SEQ ID NOs: 48, 49 and 50;
(ii) a light chain variable region comprising SEQ ID NO:46 and a heavy chain variable region comprising SEQ ID NO:51; and/or
(iii) a light chain comprising SEQ ID NO: 47 and a heavy chain comprising SEQ ID NO: 52; or
(b) the anti-PD-1 antibody or antigen-binding fragment thereof is pembrolizumab or a variant thereof.

19. (canceled)

20. The method of claim 1, wherein the immune checkpoint inhibitor and/or the personalized cancer vaccine is administered to the subject following surgical resection of a primary tumor from the subject.

21. The method of claim 1, wherein the immune response to the tumor comprises an increase in a population of T cells specific to at least one of the cancer antigen epitopes in a biological sample collected from the subject, relative to the population of T cells in a comparable biological sample collected from the subject prior to induction of the immune response to the tumor, optionally wherein the population of T cells is detectable in a pre-treatment biological sample collected from the subject prior to administration of the personalized cancer vaccine and/or the immune checkpoint inhibitor to the subject.

22. (canceled)

23. The method of claim 21, wherein the biological sample comprises peripheral blood mononuclear cells.

24. The method of claim 1, wherein a first T cell response to one of the cancer antigen epitopes is detectable in the subject following administration of the personalized cancer vaccine to the subject,
optionally wherein additional T cell responses to an additional one or more of the cancer antigen epitopes are detectable in the subject following administration of the personalized cancer vaccine to the subject.

25. The method of claim 24, wherein the first T cell response is not detectable in the subject prior to administration of the personalized cancer vaccine to the subject,
optionally wherein the additional T cell responses are not detectable in the subject prior to administration of the personalized cancer vaccine to the subject.

26. The method of claim 1, wherein a preexisting T cell response to a first cancer antigen epitope of the cancer antigen epitopes is detectable in the subject prior to administration of the personalized cancer vaccine, and wherein the magnitude of the preexisting T cell response is increased following administration of the personalized cancer vaccine to the subject relative to the magnitude prior to administration of the personalized cancer vaccine,
optionally wherein the magnitude of the preexisting T cell response corresponds to a ratio of the number of T cells responsive to the first cancer antigen epitope to a total number of T cells in a biological sample, or wherein the magnitude of the preexisting T cell response corresponds to an increased response per cell to the first cancer antigen epitope.

27. (canceled)

28. The method of claim 24, wherein the first T cell response and/or the preexisting T cell response can be detected and/or quantified by collecting a biological sample comprising peripheral blood mononuclear cells (PBMCs) from the subject, stimulating the PBMCs with the cancer antigen epitopes, and subsequently measuring cytokine production by the PBMCs.

29. The method of claim 1, wherein the mRNA of the personalized cancer vaccine encodes 0, 1, 2, 3, 4, 5, 6, 7, 8, 9, 10, or more peptides corresponding to driver mutations, and/or wherein the mRNA of the personalized cancer vaccine encodes 34 or about 34 cancer antigen epitopes expressed in the tumor in the subject.

30. The method of claim 1, wherein the tumor comprises:
(i) resected stage III or stage IV melanoma, resected stage II melanoma, or resected cutaneous melanoma, optionally wherein the tumor has a BRAF mutation;
(ii) non-small cell lung cancer, optionally wherein the non-small cell lung cancer comprises resected stage II non-small cell lung cancer, resected stage III non-small cell lung cancer, resected stage IIIA non-small cell lung cancer, or resected stage IIIB non-small cell lung cancer;
(iii) renal cell carcinoma;
(iv) muscle invasive urothelial carcinoma (MIUC), optionally wherein the tumor comprises muscle-invasive bladder cancer (MIBC), or muscle-invasive urinary tract urothelial cancer (UTUC); or
(v) cutaneous squamous cell carcinoma (cSCC), optionally wherein the cSCC comprises resectable locally advanced Stage II, III or IV.

31. A method of inducing an immune response against a tumor in a subject, the method comprising:
(a) administering to a subject an effective amount of an immune checkpoint inhibitor;
(b) administering to the subject an effective amount of a first personalized cancer vaccine, comprising: an mRNA comprising an open reading frame that encodes at least two cancer antigen epitopes expressed in the tumor in the subject, and a lipid delivery vehicle;
(c) measuring one or more biomarkers or biomarker levels in a biological sample collected from the subject, wherein the measuring is conducted after the administering of (b); and
(d) administering to the subject an effective amount of a second personalized cancer vaccine, comprising: an mRNA comprising an open reading frame that encodes at least two cancer antigen epitopes expressed in the tumor in the subject; and a lipid delivery vehicle; wherein the measurement of the one or more biomarkers or biomarker levels identifies the subject as likely to be responsive to the second personalized cancer vaccine,
wherein the administration of the immune checkpoint inhibitor and the first and second personalized cancer vaccine induces an immune response against the tumor in the subject.

32. (canceled)
